(19) United States
(12) Patent Application Publication
(10) Pub. No.: US 2018/0206495 A1
(43) Pub. Date: Jul. 26, 2018

(54) SUBSTITUTED ARYL AND HETEROARYL CARBOXYLIC ACID HYDRAZIDES OR SALTS THEREOF AND USE THEREOF TO INCREASE STRESS TOLERANCE IN PLANTS

(71) Applicant: BAYER CROPSCIENCE AKTIENGESELLSCHAFT, Monheim am Rhein (DE)

(72) Inventors: Jens FRACKENPOHL, Frankfurt (DE); Guido BOJACK, Wiesbaden-Naurod (DE); Marco BRÜNJES, Hattersheim am Main (DE); Hendrik HELMKE, Liederbach (DE); Isabelle ADELT, Haan (DE); Stefan LEHR, Liederbach (DE); Peter BRÜCHNER, Krefeld (DE); Jan DITTGEN, Frankfurt (DE); Dirk SCHMUTZLER, Hattersheim (DE); Ines HEINEMANN, Hofheim (DE); Udo BICKERS, Köln (DE); Martin Jeffrey HILLS, Idstein (DE); Juan Pedro RUIZ-SANTAELLA, Sant Joan Despi (ES); Harry STREK, Wiesbaden (DE); Philippe DESBORDES, Lyon (FR)

(21) Appl. No.: 15/744,284

(22) PCT Filed: Jul. 14, 2016
(86) PCT No.: PCT/EP2016/066715

§ 371 Date: Jan. 12, 2018

(30) Foreign Application Priority Data
Jul. 15, 2015 (EP) 15177239.9

(51) Int. Cl. A01N 43/42; A01N 37/38; A01N 37/28; A01N 37/48; C07D 401/12

(52) U.S. Cl. CPC A01N 43/42; A01N 37/38; C07D 401/12; A01N 37/48; A01N 37/28

(57) ABSTRACT

Substituted aryl- and heteroarylcarbonyl hydrazides

The invention relates to substituted aryl- and heteroarylcarbonyl hydrazides of the general formula (I) or salts thereof

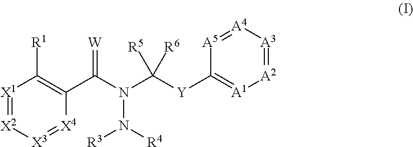

where the radicals of the formula (I) are each as defined in the description for enhancing stress tolerance in plants to abiotic stress, and for enhancing plant growth and/or for increasing plant yield.

Substituted aryl and heteroaryl carboxylic acid hydrazides or salts thereof and use thereof to increase stress tolerance in plants

### DESCRIPTION

The invention relates to substituted aryl- and heteroarylcarbonyl hydrazides or salts thereof and to the use thereof for enhancing stress tolerance in plants to abiotic stress, and for enhancing plant growth and/or for increasing plant yield.

It is known that particular substituted azinylcarboxamides, for example substituted 4-(trifluoromethyl)nicotinamides, have insecticidal properties (cf., for example, EP185256, WO2001/014373, WO2002/022583, JP07010841, JP07025853, WO2005/113553). N-substituted azinylalkylazinecarboxamides and the insecticidal action thereof are described in DE102008041214, wherein the N substituents described for the amides in question are, for example, alkyl, arylcarbonyl, alkylcarbonyl, alkoxycarbonyl and arylsulfonyl groups, but not amino substituents which lead to hydrazide structures. Arylalkyl-substituted pyridine-2-carboxamides and their nematicidal action are described in WO2015007626. It is also known that particular N-alkoxy-substituted heteroarylcarboxamides can be used as active ingredients for enhancing plant yield and for increasing against abiotic plant stress (cf. WO2013/167651).

The preparation of acylated hydrazides by reduction of hydrazones is described in Tetrahedron, 2003, 59, 773 and in IT2000/MI0292, while J. Org. Chem. 1975, 40, 19 describes photochemical reactions of benzoyl and acetyl hydrazides. The reaction of 1,1-dibenzoyl-2,2-dimethylhydrazine with hydride reagents is known from J. Org. Chem. 1956, 21, 1177, while the Raney nickel-mediated hydrogenolysis of the nitrogen-nitrogen bond in acyl hydrazines is described in J. Org. Chem. 1957, 22, 148. 1-Acyl- and 1,1-diacyl-2,2-dimethylhydrazines are additionally known from Org. Preparations and Procedures 1970, 2, 275. Particular benzoyl hydrazides without substituents in the benzoyl unit, but with specifically substituted side chains, are described, for example, in Helv. Chim. Acta 1964, 47, 1101-1113, in US2006/0020146, in U.S. Pat. No. 5,424,333, and as intermediates in WO94/22440 and WO94/22444, and are not encompassed by the substituted aryl- and heteroarylcarbonyl hydrazides of the general formula (I) that are described here. Likewise known, but not encompassed by the general formula (I), are particular bis(benzoyl hydrazides), as described, for example, in US2008/006757 and U.S. Pat. No. 6,013,836. Selected substituted cyclic benzoyl hydrazides are described in the context of studies relating to the use of RAMP and SAMP hydrazones in asymmetric organic syntheses, for example in Turkish J Org. Chem. 2013, 37, 492-518, in Org. Lett. 2001, 3, 1575-1577 and in Tetrahedron Lett. 1995, 36, 6709-4712. It is likewise known that specific highly substituted morpholine-based hydrazides can be used as chemokine receptor modulators (cf. WO2008/112156).

It is known that plants can react with specific or unspecific defense mechanisms to natural stress conditions, for example cold, heat, drought stress (stress caused by aridity and/or lack of water), injury, pathogenic attack (viruses, bacteria, fungi, insects) etc., but also to herbicides [Pflanzenbiochemie [Plant Biochemistry], p. 393-462, Spektrum Akademischer Verlag, Heidelberg, Berlin, Oxford, Hans W. Heldt, 1996.; Biochemistry and Molecular Biology of Plants, p. 1102-1203, American Society of Plant Physiologists, Rockville, Md., eds. Buchanan, Gruissem, Jones, 2000].

Numerous proteins in plants, and the genes that code for them, which are involved in defense reactions to abiotic stress (for example cold, heat, drought, salt, flooding) are known. Some of these form part of signal transduction chains (e.g. transcription factors, kinases, phosphatases) or cause a physiological response of the plant cell (e.g. ion transport, deactivation of reactive oxygen species). The signaling chain genes of the abiotic stress reaction include inter alia transcription factors of the DREB and CBF classes (Jaglo-Ottosen et al., 1998, Science 280: 104-106). Phosphatases of the ATPK and MP2C type are involved in the reaction to salt stress. In addition, in the event of salt stress, the biosynthesis of osmolytes such as proline or sucrose is frequently activated. This involves, for example, sucrose synthase and proline transporters (Hasegawa et al., 2000, Annu Rev Plant Physiol Plant Mol Biol 51: 463-499). The stress defense of the plants to cold and drought uses some of the same molecular mechanisms. There is a known accumulation of what are called late embryogenesis abundant proteins (LEA proteins), which include the dehydrins as an important class (Ingram and Bartels, 1996, Annu Rev Plant Physiol Plant Mol Biol 47: 277-403, Close, 1997, Physiol Plant 100: 291-296). These are chaperones which stabilize vesicles, proteins and membrane structures in stressed plants (Bray, 1993, Plant Physiol 103: 1035-1040). In addition, there is frequently induction of aldehyde dehydrogenases, which deactivate the reactive oxygen species (ROS) which form in the event of oxidative stress (Kirch et al., 2005, Plant Mol Biol 57: 315-332). Heat shock factors (HSF) and heat shock proteins (HSP) are activated in the event of heat stress and play a similar role here as chaperones to that of dehydrins in the event of cold and drought stress (Yu et al., 2005, Mol Cells 19: 328-333).

A number of signaling substances which are endogenous to plants and are involved in stress tolerance or pathogenic defense are already known. Mention should be made here, for example, of salicylic acid, benzoic acid, jasmonic acid or ethylene [Biochemistry and Molecular Biology of Plants, p. 850-929, American Society of Plant Physiologists, Rockville, Md., eds. Buchanan, Gruissem, Jones, 2000]. Some of these substances or the stable synthetic derivatives and derived structures thereof are also effective on external application to plants or in seed dressing, and activate defense reactions which cause elevated stress tolerance or pathogen tolerance of the plant [Sembdner, and Parthier, 1993, Ann. Rev. Plant Physiol. Plant Mol. Biol. 44: 569-589].

It is also known that chemical substances can increase the tolerance of plants to abiotic stress. Such substances are applied either by seed dressing, by leaf spraying or by soil treatment. For instance, an increase in the abiotic stress tolerance of crop plants by treatment with elicitors of systemic acquired resistance (SAR) or abscisic acid derivatives is described (Schading and Wei, WO2000/28055; Abrams and Gusta, U.S. Pat. No. 5,201,931; Abrams et al., WO97/23441, Churchill et al., 1998, Plant Growth Regul 25: 35-45). In addition, effects of growth regulators on the stress tolerance of crop plants have been described (Morrison and Andrews, 1992, J Plant Growth Regul 11: 113-117, RD-259027). In this context, it is likewise known that a growth-regulating naphthylsulfonamide (4-bromo-N-(pyridin-2-ylmethyl)naphthalene-1-sulfonamide) influences the germination of plant seeds in the same way as abscisic acid (Park et al. Science 2009, 324, 1068-1071). Furthermore, a naphthylsulfamidocarboxylic acid (N-[(4-bromo-1-naphthyl)sulfonyl]-5-methoxynorvaline) shows a mode of action in biochemical receptor tests which is comparable to 4-bromo-N-(pyridin-2-ylmethyl)naphthalene-1-sulfonamide (Melcher et al. Nature Structural & Molecular Biology 2010, 17, 1102-1108). It is also known that a further naphthylsulfonamide, N-(6-aminohexyl)-5-chloronaphthalene-1-sulfonamide, influences the calcium level in plants which have been exposed to cold shock (Cholewa et al. Can. J. Botany 1997, 75, 375-382).

Similar effects are also observed on application of fungicides, especially from the group of the strobilurins or of the succinate dehydrogenase inhibitors, and are frequently also accompanied by an increase in yield (Draber et al., DE3534948, Bartlett et al., 2002, Pest Manag Sci 60: 309). It is likewise known that the herbicide glyphosate in low dosage stimulates the growth of some plant species (Cedergreen, Env. Pollution 2008, 156, 1099).

In the event of osmotic stress, a protective effect has been observed as a result of application of osmolytes, for example glycine betaine or the biochemical precursors thereof, e.g. choline derivatives (Chen et al., 2000, Plant Cell Environ 23: 609-618, Bergmann et al., DE4103253). The effect of antioxidants, for example naphthols and xanthines, for increasing abiotic stress tolerance in plants has also already been described (Bergmann et al., DD277832, Bergmann et al., DD277835). However, the molecular causes of the antistress action of these substances are largely unknown.

It is also known that the tolerance of plants to abiotic stress can be increased by a modification of the activity of endogenous poly-ADP-ribose polymerases (PARP) or poly-(ADP-ribose) glycohydrolases (PARG) (de Block et al., The Plant Journal, 2004, 41, 95; Levine et al., FEBS Lett. 1998, 440, 1; WO00/04173; WO2004/090140).

It is thus known that plants possess several endogenous reaction mechanisms which can bring about an effective defense against a wide variety of different harmful organisms and/or natural abiotic stress. Since the environmental and economic demands on modern plant treatment compositions are increasing constantly, for example with respect to their toxicity, selectivity, application rate, formation of residues and favorable manufacture, there is a constant need to develop novel plant treatment compositions which have advantages over those known, at least in some areas. It was therefore an object of the present invention to provide compounds which further increase tolerance to abiotic stress in plants, bring about invigoration of plant growth and/or contribute to an increase in plant yield. In this context, tolerance to abiotic stress is understood to mean, for example, tolerance to cold, heat and drought stress (stress caused by drought and/or lack of water), salts and flooding.

It has now been found that, surprisingly, substituted aryl- and heteroarylcarbonyl hydrazides can be used to enhance stress tolerance in plants to abiotic stress, and to enhance plant growth and/or to increase plant yield.

The present invention accordingly provides substituted aryl- and heteroarylcarbonyl hydrazides of the general formula (I) or salts thereof

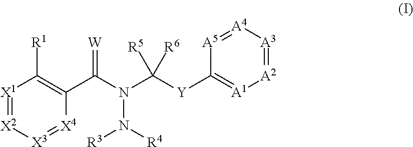

in which
R¹, R² and R⁷ are independently hydrogen, halogen, cyano, nitro, NR²¹R²², OR²³, S(O)ₙR²⁴, thiocyanato, isothiocyanato, (C₁-C₈)-alkyl, (C₂-C₈)-alkenyl, (C₂-C₈)-alkynyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, pentafluorothio, (C₁-C₈)-alkoxy-(C₁-C₈)-haloalkyl, (C₁-C₈)-haloalkoxy-(C₁-C₈-haloalkyl, (C₁-C₈)-haloalkoxy-(C₁-C₈)-alkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, heterocyclyl, heterocyclyl-(C₁-C₈)-alkyl, (C₁-C₈)-alkoxy-(C₁-C₈-alkyl, (C₁-C₈)-alkylthio-(C₁-C₈)-alkyl, (C₁-C₈)-haloalkylthio-(C₁-C₈-alkyl, (C₁-C₈)-alkylcarbonyl-(C₁-C₈)-alkyl, COOR²³, CONR²¹R²², COR²³, —C═NOR²³, R²³OOC-(C₁-C₈)-alkyl, aryl-(C₁-C₈)-alkynyl, heteroaryl-(C₁-C₈)-alkynyl, heterocyclyl-(C₁-C₈)-alkynyl, tris[(C₁-C₈)-alkyl]silyl-(C₂-C₈)-alkynyl, bis[(C₁-C₈)-alkyl](aryl)silyl-(C₂-C₈)-alkynyl, bisaryl[(C₁-C₈)-alkyl]silyl-(C₂-C₈)-alkynyl, (C₃-C₈)-cycloalkyl-(C₂-C₈)-alkynyl, aryl-(C₂-C₈)-alkenyl, heteroaryl-(C₂-C₈)-alkenyl, heterocyclyl-(C₂-C₈)-alkenyl, (C₃-C₈)-cycloalkyl-(C₂-C₈)-alkenyl, (C₁-C₈-alkoxy-(C₁-C₈)-alkoxy-(C₁-C₈)-alkyl, (C₁-C₈)-alkylaminosulfonylamino, (C₃-C₈-cycloalkylaminosulfonylamino, diazo, aryldiazo, tris[(C₁-C₈)-alkyl]silyl,bis[(C₁-C₈)-alkyl](aryl)silyl, bisaryl[(C₁-C₈)-alkyl]silyl,
X¹, X², X³ and X⁴ are the same or different and are independently N (nitrogen) or the C—R² moiety, but there are never more than two adjacent nitrogen atoms, and where R² in each C—R² moiety is the same or different as defined above, and where R¹ and R² are not both hydrogen when X¹, X², X³ and X⁴ are C—R²,
W is O (oxygen) or S (sulfur),
A¹, A², A³, A⁴ and A⁵ are the same or different and are each independently N (nitrogen) or the C—R⁷ moiety, but there are never more than two adjacent nitrogen atoms, and where R⁷ in each C—R⁷ moiety is the same or different as defined above,
R³ is (C₁-C₈)-alkyl, cyano-(C₁-C₈)-alkyl, (C₂-C₈)-alkenyl, (C₂-C₈)-alkynyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₃-C₈)-cycloalkyl-(C₁-C₈-alkyl, heterocyclyl, heterocyclyl-(C₁-C₈)-alkyl, (C₁-C₈)-alkoxy-(C₁-C₈-alkyl, (C₁-C₈)-alkylthio-(C₁-C₈)-alkyl, (C₁-C₈)-haloalkoxy-(C₁-C₈)-alkyl, (C₁-C₈)-haloalkylthio-(C₁-C₈)-alkyl, R²¹R²²N-(C₁-C₈)-alkyl, (C₁-C₈)-alkoxy-(C₁-C₈-alkoxy-(C₁-C₈)-alkyl,
R⁴ is hydrogen, (C₁-C₈)-alkyl, (C₂-C₈)-alkenyl, (C₂-C₈)-alkynyl, (C₁-C₁₀)-haloalkyl, (C₂-C₈)-haloalkenyl, (C₂-C₈)-haloalkynyl, (C₃-C₁₀)-cycloalkyl, (C₃-C₁₀-halocycloalkyl, (C₄-C₁₀)-cycloalkenyl, (C₄-C₁₀)-halocycloalkenyl, aryl, aryl-(C₁-C₈-alkyl, heteroaryl, heteroaryl-(C₁-C₈)-alkyl, (C₃-C₈)-cycloalkyl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio-(C₁-C₈-alkyl, (C₁-C₈)-haloalkoxy-(C₁-C₈)-alkyl, (C₁-C₈)-haloalkylthio-(C₁-C₈-alkyl, (C₁-C₈)-alkylamino-(C₁-C₈)-alkyl, bis[(C₁-C₈)-alkyl]amino-(C₁-C₈)-alkyl, (C₃-C₈)-cycloalkylamino-(C₁-C₈)-alkyl, (C₁-C₈)-alkoxy-(C₁-C₈)-alkoxy-(C₁-C₈-alkyl, (C₁-C₈)-alkoxycarbonyl, (C₂-C₈)-alkenyloxycarbonyl, (C₂-C₈)-alkynyloxycarbonyl, aryl-(C₁-C₈)-alkoxycarbonyl, heteroaryl-(C₁-C₈)-alkoxycarbonyl, (C₃-C₈)-cycloalkyl-(C₁-C₈)-alkoxycarbonyl, CONR²¹R²², SO₂R²⁴, hydroxycarbonyl-(C₁-C₈)-alkyl, (C₁-C₈)-alkoxycarbonyl-(C₁-C₈)-alkyl, (C₂-C₈)-alkenyloxycarbonyl-(C₁-C₈)-alkyl, (C₂-C₈)-alkynyloxycarbonyl-(C₁-C₈)-alkyl, aryl-(C₁-C₈)-alkoxycarbonyl-(C₁-C₈)-alkyl, heteroaryl-(C₁-C₈)-alkoxycarbonyl-(C₁-C₈)-alkyl, heterocyclyl-(C₁-C₈)-alkoxycarbonyl-(C₁-C₈)-alkyl, (C₁-C₈)-alkylcarbonyl-(C₁-C₈)-alkyl, (C₁-C₈)-alkylsulfonyl-(C₁-C₈)-alkyl, (C₁-C₈)-alkylsulfynyl-(C₁-C₈)-alkyl,
R⁵ and R⁶ are independently hydrogen, (C₁-C₈)-alkyl, (C₂-C₈)-alkenyl, (C₂-C₈)-alkynyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, aryl, aryl-(C₁-C₈)-alkyl, heteroaryl, heteroaryl-(C₁-C₈)-alkyl, (C₃-C₈)-cycloalkyl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio-(C₁-C₈)-alkyl, (C₁-C₈)-haloalkoxy-(C₁-C₈)-alkyl, (C₁-C₈)-haloalkylthio-(C₁-C₈)-alkyl, COOR²³, CONR²¹R²², hydroxycarbonyl-(C₁-C₈)-alkyl, (C₁-C₈)-alkoxycarbonyl-(C₁-C₈)-alkyl, (C₂-C₈)-alkenyloxycarbonyl-(C₁-C₈)-alkyl, (C₂-C₈)-alkynyloxycarbonyl-(C₁-C₈)-alkyl, aryl-(C₁-C₈)-alkoxycarbonyl-(C₁-C₈)-alkyl, heteroaryl-(C₁-C₈)-alkoxycarbonyl-(C₁-C₈)-alkyl, heterocyclyl-(C₁-C₈)-alkoxycarbonyl-(C₁-C₈)-alkyl,
R⁵ and R⁶ together with the atom to which they are bonded form a fully saturated or partly saturated 5-7-membered ring optionally interrupted by heteroatoms and optionally having further substitution,
R¹ and X¹, when X¹ is a C—R² group, together with the atoms to which they are bonded form a fully saturated, partly saturated or fully unsaturated 5-7-membered ring optionally interrupted by heteroatoms and optionally having further substitution,
X¹ and X², when each is a C—R² group, together with the atoms to which they are bonded form a fully saturated, partly saturated or fully unsaturated 5-7-membered ring optionally interrupted by heteroatoms and optionally having further substitution,

A¹ and A², when each is a C—R⁷ group, together with the atoms to which they are bonded form a fully saturated, partly saturated or fully unsaturated 5-7-membered ring optionally interrupted by heteroatoms and optionally having further substitution,
A² and A³, when each is a C—R⁷ group, together with the atoms to which they are bonded form a fully saturated, partly saturated or fully unsaturated 5-7-membered ring optionally interrupted by heteroatoms and optionally having further substitution,
A³ and A⁴, when each is a C—R⁷ group, together with the atoms to which they are bonded form a fully saturated, partly saturated or fully unsaturated 5-7-membered ring optionally interrupted by heteroatoms and optionally having further substitution,
Y is a bond or the Y-1 to Y-7 moieties

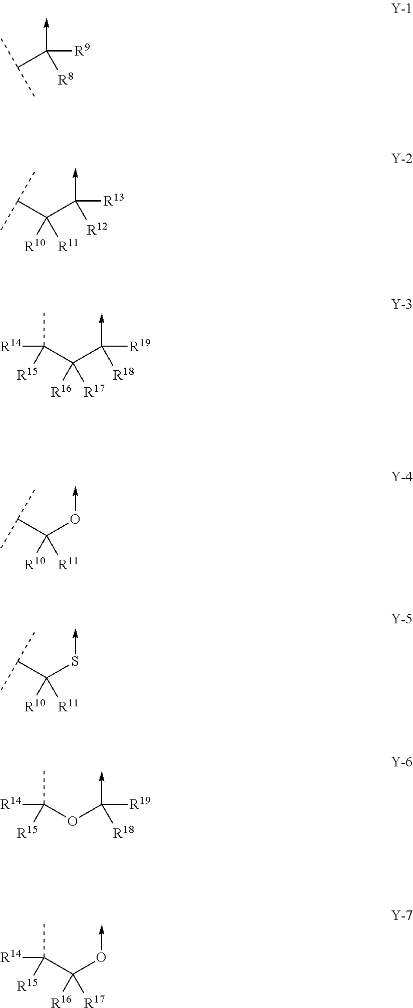

where R⁸, R⁹, R¹⁰, R¹¹, R¹², R¹³, R¹⁴, R¹⁵, R¹⁶, R¹⁷, R¹⁸ and R¹⁹ are each as per the definition below and where the arrow represents a bond to the 6-membered ring with the A¹, A², A³, A⁴ and A⁵ moieties,
R⁸, R⁹, R¹⁰, R¹¹, R¹², R¹³, R¹⁴, R¹⁵, R¹⁶, R¹⁷, R¹⁸ and R¹⁹ are independently hydrogen, (C₁-C₈)-alkyl, (C₂-C₈)-alkenyl, (C₂-C₈)-alkynyl, (C₁-C₁₀)-haloalkyl, (C₂-C₈)-haloalkenyl, (C₂-C₈)-haloalkynyl, (C₃-C₁₀)-cycloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio-(C₁-C₈)-alkyl, (C₁-C₈)-haloalkoxy-(C₁-C₈)-alkyl, (C₁-C₈)-haloalkylthio-(C₁-C₈)-alkyl, COOR²³,
n is 0, 1 or 2,
R²¹ and R²² are the same or different and are independently hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, COR²³, SO₂R²⁴, (C₁-C₈)-alkyl-HNO₂S—, (C₃-C₈)-cycloalkyl-HNO₂S—, heterocyclyl, (C₁-C₈)-alkoxycarbonyl-(C₁-C₈)-alkyl, (C₁-C₈)-alkoxycarbonyl, aryl-(C₁-C₈)-alkoxycarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, heterocyclyl-(C₁-C₈)-alkyl,
R²³ is hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, (C₁-C₈)-alkoxycarbonyl-(C₁-C₈)-alkyl, (C₂-C₈)-alkenyloxycarbonyl-(C₁-C₈)-alkyl, aryl-(C₁-C₈)-alkoxycarbonyl-(C₁-C₈)-alkyl, hydroxycarbonyl-(C₁-C₈)-alkyl, heterocyclyl, heterocyclyl-(C₁-C₈)-alkyl and
R²⁴ is hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₈)-cycloalkyl-(C₁-C₈)-alkyl, (C₄-C₁₀)-cycloalkenyl-(C₁-C₈)-alkyl, NR²¹R²².

The compounds of the general formula (I) can form salts by addition of a suitable inorganic or organic acid, for example mineral acids, for example HCl, HBr, H₂SO₄, H₃PO₄ or HNO₃, or organic acids, for example carboxylic acids such as formic acid, acetic acid, propionic acid, oxalic acid, lactic acid or salicylic acid or sulfonic acids, for example p-toluenesulfonic acid, onto a basic group, for example amino, alkylamino, dialkylamino, piperidino, morpholino or pyridino. In such a case, these salts will comprise the conjugate base of the acid as the anion. Suitable substituents in deprotonated form, for example sulfonic acids, particular sulfonamides or carboxylic acids, are capable of forming internal salts with groups, such as amino groups, which are themselves protonatable. Salts may also be formed by action of a base on compounds of the general formula (I). Examples of suitable bases are organic amines such as trialkylamines, morpholine, piperidine and pyridine, and the hydroxides, carbonates and hydrogencarbonates of ammonium, alkali metals or alkaline earth metals, especially sodium hydroxide, potassium hydroxide, sodium carbonate, potassium carbonate, sodium hydrogencarbonate and potassium hydrogencarbonate. These salts are compounds in which the acidic hydrogen is replaced by an agriculturally suitable cation, for example metal salts, especially alkali metal salts or alkaline earth metal salts, in particular sodium and potassium salts, or else ammonium salts, salts with organic amines or quaternary ammonium salts, for example with cations of the formula [NR^{a}R^{b}R^{c}R^{d}]⁺ in which R^{a} to R^{d} are each independently an organic radical, especially alkyl, aryl, aralkyl or alkylaryl. Also suitable are alkylsulfonium and alkylsulfoxonium salts, such as (C₁-C₄)-trialkylsulfonium and (C₁-C₄)-trialkylsulfoxonium salts.

The inventive aryl- and heteroarylcarbonyl hydrazides of the formula (I), depending on external conditions such as pH, solvent and temperature and X¹, X², X³ and X⁴, may take the form of various tautomeric structures, all of which are considered to be embraced by the formula (I).

The compounds of the formula (I) used in accordance with the invention and salts thereof are referred to hereinafter as “compounds of the general formula (I)”.

The invention preferably provides compounds of the general formula (I) in which
R¹, R² and R⁷ are independently hydrogen, halogen, cyano, nitro, NR²¹R²², OR²³, S(O)ₙR²⁴, thiocyanato, isothiocyanato, (C₁-C₇)-alkyl, (C₂-C₇)-alkenyl, (C₂-C₇)-alkynyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, pentafluorothio, (C₁-C₇)-alkoxy-(C₁-C₇)-haloalkyl, (C₁-C₇)-haloalkoxy-(C₁-C₇)-haloalkyl, (C₁-C₇)-haloalkoxy-(C₁-C₇)-alkyl, aryl, aryl-(C₁-C₇)-alkyl, heteroaryl, heteroaryl-(C₁-C₇)-alkyl, (C₃-C₇)-cycloalkyl-(C₁-C₇)-alkyl, (C₄-C₁₀)-cycloalkenyl-(C₁-C₇)-alkyl, heterocyclyl, heterocyclyl-(C₁-C₇)-alkyl, (C₁-C₇)-alkoxy-(C₁-C₇)-alkyl, (C₁-C₇)-alkylthio-(C₁-C₇)-alkyl, (C₁-C₇)-haloalkylthio-(C₁-C₇)-alkyl, (C₁-C₇)-alkylcarbonyl-(C₁-C₇)-alkyl, COOR²³, CONR²¹R²², COR²³, —C═NOR²³, R²³OOC-(C₁-C₇)-alkyl, aryl-(C₁-C₇)-alkynyl, heteroaryl-(C₁-C₇)-alkynyl, heterocyclyl-(C₁-C₇)-alkynyl, tris[(C₁-C₇)-alkyl]silyl-(C₂-C₇)-alkynyl, bis[(C₁-C₇)-alkyl](aryl)silyl-(C₂-C₇)-alkynyl, bisaryl[(C₁-C₇)-alkyl]silyl-(C₂-C₇)-alkynyl, (C₃-C₇)-cycloalkyl-(C₂-C₇)-alkynyl, aryl-(C₂-C₇)-alkenyl, heteroaryl-(C₂-C₇)-alkenyl, heterocyclyl-(C₂-C₇)-alkenyl, (C₃-C₇)-cycloalkyl-(C₂-C₇)-alkenyl, (C₁-C₇)-alkoxy-(C₁-C₇)-alkoxy-(C₁-C₇)-alkyl, (C₁-C₇)-alkylaminosulfonylamino, (C₃-C₇)-cycloalkylaminosulfonylamino, diazo, aryldiazo, tris[(C₁-C₇)-alkyl]silyl, bis[(C₁-C₇)-alkyl](aryl)silyl, bisaryl[(C₁-C₇)-alkyl]silyl,
X¹, X², X³ and X⁴ are the same or different and are independently N (nitrogen) or the C—R² moiety, but there are never more than two adjacent nitrogen atoms, and where R² in each C—R² moiety is the same or different as defined above, and where R¹ and R² are not both hydrogen when X¹, X², X³ and X⁴ are C—R²,
W is O (oxygen) or S (sulfur),
A¹, A², A³, A⁴ and A⁵ are the same or different and are each independently N (nitrogen) or the C—R⁷ moiety, but there are never more than two adjacent nitrogen atoms, and where R⁷ in each C—R⁷ moiety is the same or different as defined above,
R³ is (C₁-C₇)-alkyl, cyano-(C₁-C₇)-alkyl, (C₂-C₇)-alkenyl, (C₂-C₇)-alkynyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₃-C₇)-cycloalkyl-(C₁-C₇)-alkyl, heterocyclyl, heterocyclyl-(C₁-C₇)-alkyl, (C₁-C₇)-alkoxy-(C₁-C₇)-alkyl, (C₁-C₇)-alkylthio-(C₁-C₇)-alkyl, (C₁-C₇)-haloalkoxy-(C₁-C₇)-alkyl, (C₁-C₇)-haloalkylthio-(C₁-C₇)-alkyl, R²¹R²²N-(C₁-C₇)-alkyl, (C₁-C₇)-alkoxy-(C₁-C₇)-alkoxy-(C₁-C₇)-alkyl,
R⁴ is hydrogen, (C₁-C₇)-alkyl, (C₂-C₇)-alkenyl, (C₂-C₇)-alkynyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, aryl, aryl-(C₁-C₇)-alkyl, heteroaryl, heteroaryl-(C₁-C₇)-alkyl, (C₃-C₇)-cycloalkyl-(C₁-C₇)-alkyl, heterocyclyl, heterocyclyl-(C₁-C₇)-alkyl, (C₁-C₇)-alkoxy-(C₁-C₇)-alkyl, (C₁-C₇)-alkylthio-(C₁-C₇)-alkyl, (C₁-C₇)-haloalkoxy-(C₁-C₇)-alkyl, (C₁-C₇)-haloalkylthio-(C₁-C₇)-alkyl, (C₁-C₇)-alkylamino-(C₁-C₇)-alkyl, bis[(C₁-C₇)-alkyl]amino-(C₁-C₇)-alkyl, (C₃-C₇)-cycloalkylamino-(C₁-C₇)-alkyl, (C₁-C₇)-alkoxy-(C₁-C₇)-alkoxy-(C₁-C₇)-alkyl, (C₁-C₇)-alkoxycarbonyl, (C₂-C₇)-alkenyloxycarbonyl, (C₂-C₇)-alkynyloxycarbonyl, aryl-(C₁-C₇)-alkoxycarbonyl, heteroaryl-(C₁-C₇)-alkoxycarbonyl, (C₃-C₇)-cycloalkyl-(C₁-C₇)-alkoxycarbonyl, CONR²¹R²², SO₂R²⁴, hydroxycarbonyl-(C₁-C₇)-alkyl, (C₁-C₇)-alkoxycarbonyl-(C₁-C₇)-alkyl, (C₂-C₇)-alkenyloxycarbonyl-(C₁-C₇)-alkyl, (C₂-C₇)-alkynyloxycarbonyl-(C₁-C₇)-alkyl, aryl-(C₁-C₇)-alkoxycarbonyl-(C₁-C₇)-alkyl, heteroaryl-(C₁-C₇)-alkoxycarbonyl-(C₁-C₇)-alkyl, heterocyclyl-(C₁-C₇)-alkoxycarbonyl-(C₁-C₇)-alkyl, (C₁-C₇)-alkylcarbonyl-(C₁-C₇)-alkyl, (C₁-C₇)-alkylsulfonyl-(C₁-C₇)-alkyl, (C₁-C₇)-alkylsulfynyl-(C₁-C₇)-alkyl,
R⁵ and R⁶ are independently hydrogen, (C₁-C₇)-alkyl, (C₂-C₇)-alkenyl, (C₂-C₇)-alkynyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, aryl, aryl-(C₁-C₇)-alkyl, heteroaryl, heteroaryl-(C₁-C₇)-alkyl, (C₃-C₇)-cycloalkyl-(C₁-C₇)-alkyl, heterocyclyl, heterocyclyl-(C₁-C₇)-alkyl, (C₁-C₇)-alkoxy-(C₁-C₇)-alkyl, (C₁-C₇)-alkylthio-(C₁-C₇)-alkyl, (C₁-C₇)-haloalkoxy-(C₁-C₇)-alkyl, (C₁-C₇)-haloalkylthio-(C₁-C₇)-alkyl, COOR²³, CONR²¹R²², hydroxycarbonyl-(C₁-C₇)-alkyl, (C₁-C₇)-alkoxycarbonyl-(C₁-C₇)-alkyl, (C₂-C₇)-alkenyloxycarbonyl-(C₁-C₇)-alkyl, (C₂-C₇)-alkynyloxycarbonyl-(C₁-C₇)-alkyl, aryl-(C₁-C₇)-alkoxycarbonyl-(C₁-C₇)-alkyl, heteroaryl-(C₁-C₇)-alkoxycarbonyl-(C₁-C₇)-alkyl, heterocyclyl-(C₁-C₇)-alkoxycarbonyl-(C₁-C₇)-alkyl,
R⁵ and R⁶ together with the atom to which they are bonded form a fully saturated or partly saturated 5-7-membered ring optionally interrupted by heteroatoms and optionally having further substitution,
R¹ and X¹, when X¹ is a C—R² group, together with the atoms to which they are bonded form a fully saturated, partly saturated or fully unsaturated 5-7-membered ring optionally interrupted by heteroatoms and optionally having further substitution,
X¹ and X², when each is a C—R² group, together with the atoms to which they are bonded form a fully saturated, partly saturated or fully unsaturated 5-7-membered ring optionally interrupted by heteroatoms and optionally having further substitution,

A¹ and A², when each is a C—R⁷ group, together with the atoms to which they are bonded form a fully saturated, partly saturated or fully unsaturated 5-7-membered ring optionally interrupted by heteroatoms and optionally having further substitution,
A² and A³, when each is a C—R⁷ group, together with the atoms to which they are bonded form a fully saturated, partly saturated or fully unsaturated 5-7-membered ring optionally interrupted by heteroatoms and optionally having further substitution,
A³ and A⁴, when each is a C—R⁷ group, together with the atoms to which they are bonded form a fully saturated, partly saturated or fully unsaturated 5-7-membered ring optionally interrupted by heteroatoms and optionally having further substitution,
Y is a bond or the Y-1 to Y-7 moieties

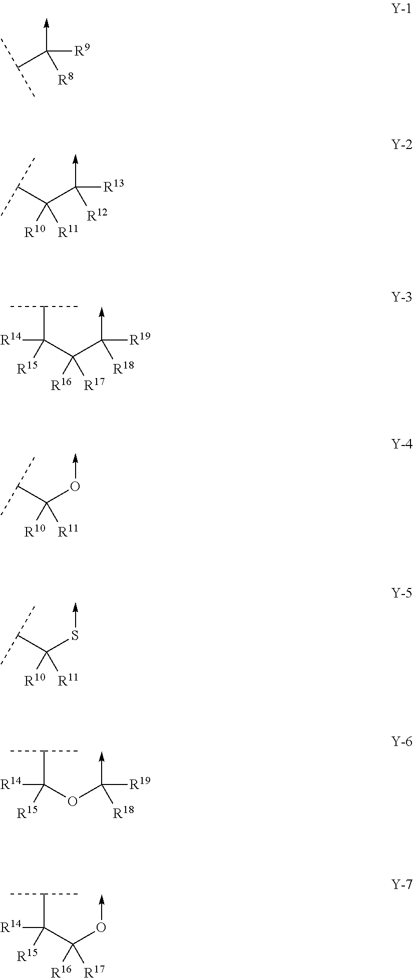

where R⁸, R⁹, R¹⁰, R¹¹, R¹², R¹³, R¹⁴, R¹⁵, R¹⁶, R¹⁷, R¹⁸ and R¹⁹ are each as per the definition below and where the arrow represents a bond to the 6-membered ring with the A¹, A², A³, A⁴ and A⁵ moieties,
R⁸, R⁹, R¹⁰, R¹¹, R¹², R¹³, R¹⁴, R¹⁵, R¹⁶, R¹⁷, R¹⁸ and R¹⁹ are independently hydrogen, (C₁-C₇)-alkyl, (C₂-C₇)-alkenyl, (C₂-C₇)-alkynyl, (C₁-C₁₀)-haloalkyl, (C₂-C₇)-haloalkenyl, (C₂-C₇)-haloalkynyl, (C₃-C₁₀)-cycloalkyl, aryl, aryl-(C₁-C₇)-alkyl, heteroaryl, heteroaryl-(C₁-C₇)-alkyl, (C₃-C₇)-cycloalkyl-(C₁-C₇)-alkyl, heterocyclyl, heterocyclyl-(C₁-C₇)-alkyl, (C₁-C₇)-alkoxy-(C₁-C₇)-alkyl, (C₁-C₇)-alkylthio-(C₁-C₇)-alkyl, (C₁-C₇)-haloalkoxy-(C₁-C₇)-alkyl, (C₁-C₇)-haloalkylthio-(C₁-C₇)-alkyl, COOR²³,
n is 0, 1 or 2,
R²¹ and R²² are the same or different and are independently hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-haloalkoxy-(C₁-C₇)-alkyl, (C₁-C₇)-alkylthio-(C₁-C₇)-alkyl, (C₁-C₇)-haloalkylthio-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, (C₃-C₇)-cycloalkyl-(C₁-C₇)-alkyl, (C₄-C₁₀)-cycloalkenyl-(C₁-C₇)-alkyl, COR²³, SO₂R²⁴, (C₁-C₇)-alkyl-HNO₂S-, (C₃-C₇)-cycloalkyl-HNO₂S—, heterocyclyl, (C₁-C₇)-alkoxycarbonyl-(C₁-C₇)-alkyl, (C₁-C₇)-alkoxycarbonyl, aryl-(C₁-C₇)-alkoxycarbonyl-(C₁-C₇)-alkyl, aryl-(C₁-C₇)-alkoxycarbonyl, heteroaryl-(C₁-C₇)-alkoxycarbonyl, (C₂-C₇)-alkenyloxycarbonyl, (C₂-C₇)-alkynyloxycarbonyl, heterocyclyl-(C₁-C₇)-alkyl,
R²³ is hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, (C₃-C₇)-cycloalkyl-(C₁-C₇)-alkyl, (C₄-C₁₀)-cycloalkenyl-(C₁-C₇)-alkyl, (C₁-C₇)-alkoxycarbonyl-(C₁-C₇)-alkyl, (C₂-C₇)-alkenyloxycarbonyl-(C₁-C₇)-alkyl, aryl-(C₁-C₇)-alkoxycarbonyl-(C₁-C₇)-alkyl, hydroxycarbonyl-(C₁-C₇)-alkyl, heterocyclyl, heterocyclyl-(C₁-C₇)-alkyl and
R²⁴ is hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, heterocyclyl-(C₁-C₇)-alkyl, (C₃-C₇)-cycloalkyl-(C₁-C₇)-alkyl, (C₄-C₁₀)-cycloalkenyl-(C₁-C₇)-alkyl, NR²¹R²².

Particular preference is given to compounds of the general formula (I) in which
R¹, R² and R⁷ are independently hydrogen, halogen, cyano, nitro, NR²¹R²², OR²³, S(O)ₙR²⁴, thiocyanato, isothiocyanato, (C₁-C₆)-alkyl, (C₂-C₆)-alkenyl, (C₂-C₆)-alkynyl, (C₁-C₁₀)-haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, pentafluorothio, (C₁-C₆)-alkoxy-(C₁-C₆)-haloalkyl, (C₁-C₆)-haloalkoxy-(C₁-C₆)-haloalkyl, (C₁-C₆)-haloalkoxy-(C₁-C₆)-alkyl, aryl, aryl-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, heterocyclyl, heterocyclyl-(C₁-C₆)-alkyl, (C₁-C₆)-alkoxy-(C₁-C₆)-alkyl, (C₁-C₆)-alkylthio-(C₁-C₆)-alkyl, (C₁-C₆)-haloalkylthio-(C₁-C₆)-alkyl, (C₁-C₆)-alkylcarbonyl-(C₁-C₆)-alkyl, COOR²³, CONR²¹R²², COR²³, —C═NOR²³, R²³OOC-(C₁-C₆)-alkyl, aryl-(C₁-C₆)-alkynyl, heteroaryl-(C₁-C₆)-alkynyl, heterocyclyl-(C₁-C₆)-alkynyl, tris[(C₁-C₆)-alkyl]silyl-(C₂-C₆)-alkynyl, bis[(C₁-C₆)-alkyl](aryl)silyl-(C₂-C₆)-alkynyl, bisaryl[(C₁-C₆)-alkyl]silyl-(C₂-C₆)-alkynyl, (C₃-C₆)-cycloalkyl-(C₂-C₆)-alkynyl, aryl-(C₂-C₆)-alkenyl, heteroaryl-(C₂-C₆)-alkenyl, heterocyclyl-(C₂-C₆)-alkenyl, (C₃-C₆)-cycloalkyl-(C₂-C₆)-alkenyl, (C₁-C₆)-alkoxy-(C₁-C₆)-alkoxy-(C₁-C₆)-alkyl, (C₁-C₆)-alkylaminosulfonylamino, (C₃-C₆)-cycloalkylaminosulfonylamino, diazo, aryldiazo, tris[(C₁-C₆)-alkyl]silyl, bis[(C₁-C₆)-alkyl](aryl)silyl, bisaryl[(C₁-C₆)-alkyl]silyl,
X¹, X², X³ and X⁴ are the same or different and are independently N (nitrogen) or the C—R² moiety, but there are never more than two adjacent nitrogen atoms, and where R² in each C—R² moiety is the same or different as defined above, and where R¹ and R² are not both hydrogen when X¹, X², X³ and X⁴ are C—R²,
W is O (oxygen) or S (sulfur),
A¹, A², A³, A⁴ and A⁵ are the same or different and are each independently N (nitrogen) or the C—R⁷ moiety, but there are never more than two adjacent nitrogen atoms, and where R⁷ in each C—R⁷ moiety is the same or different as defined above,
R³ is (C₁-C₆)-alkyl, cyano-(C₁-C₆)-alkyl, (C₂-C₆)-alkenyl, (C₂-C₆)-alkynyl, (C₁-C₆)-haloalkyl, (C₂-C₆)-haloalkenyl, (C₂-C₆)-haloalkynyl, (C₃-C₆)-cycloalkyl, (C₃-C₁₀)-halocycloalkyl, (C₄-C₁₀)-cycloalkenyl, (C₄-C₁₀)-halocycloalkenyl, (C₃-C₆)-cycloalkyl-(C₁-C₆)-alkyl, heterocyclyl, heterocyclyl-(C₁-C₆)-alkyl, (C₁-C₆)-alkoxy-(C₁-C₆)-alkyl, (C₁-C₆)-alkylthio-(C₁-C₆)-alkyl, (C₁-C₆)-haloalkoxy-(C₁-C₆)-alkyl, (C₁-C₆)-haloalkylthio-(C₁-C₆)-alkyl, R²¹R²²N-(C₁-C₆)-alkyl, (C₁-C₆)-alkoxy-(C₁-C₆)-alkoxy-(C₁-C₆)-alkyl,
R⁴ is hydrogen, (C₁-C₆)-alkyl, (C₂-C₆)-alkenyl, (C₂-C₆)-alkynyl, (C₁-C₁₀)-haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, aryl, aryl-(C₁-C₆)-alkyl, heteroaryl, heteroaryl-(C₁-C₆)-alkyl, (C₃-C₆)-cycloalkyl-(C₁-C₆)-alkyl, heterocyclyl, heterocyclyl-(C₁-C₆)-alkyl, (C₁-C₆)-alkoxy-(C₁-C₆)-alkyl, (C₁-C₆)-alkylthio-(C₁-C₆)-alkyl, (C₁-C₆)-haloalkoxy-(C₁-C₆)-alkyl, (C₁-C₆)-haloalkylthio-(C₁-C₆)-alkyl, (C₁-C₆)-alkylamino-(C₁-C₆)-alkyl, bis[(C₁-C₆)-alkyl]amino-(C₁-C₆)-alkyl, (C₃-C₆)-cycloalkylamino-(C₁-C₆)-alkyl, (C₁-C₆)-alkoxy-(C₁-C₆)-alkoxy-(C₁-C₆)-alkyl, (C₁-C₆)-alkoxycarbonyl, (C₂-C₆)-alkenyloxycarbonyl, (C₂-C₆)-alkynyloxycarbonyl, aryl-(C₁-C₆)-alkoxycarbonyl, heteroaryl-(C₁-C₆)-alkoxycarbonyl, (C₃-C₆)-cycloalkyl-(C₁-C₆)-alkoxycarbonyl, CONR²¹R²², SO₂R²⁴, hydroxycarbonyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, (C₂-C₆)-alkynyloxycarbonyl-(C₁-C₆)-alkyl, aryl-(C₁-C₆)-alkoxycarbonyl-(C₁-C₆)-alkyl, heteroaryl-(C₁-C₆)-alkoxycarbonyl-(C₁-C₆)-alkyl, heterocyclyl-(C₁-C₆)-alkoxycarbonyl-(C₁-C₆)-alkyl, (C₁-C₆)-alkylcarbonyl-(C₁-C₆)-alkyl, (C₁-C₆)-alkylsulfonyl-(C₁-C₆)-alkyl, (C₁-C₆)-alkylsulfynyl-(C₁-C₆)-alkyl,
R⁵ and R⁶ are independently hydrogen, (C₁-C₆)-alkyl, (C₂-C₆)-alkenyl, (C₂-C₆)-alkynyl, (C₁-C₁₀)-haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, aryl, aryl-(C₁-C₆)-alkyl, heteroaryl, heteroaryl-(C₁-C₆)-alkyl, (C₃-C₆)-cycloalkyl-(C₁-C₆)-alkyl, heterocyclyl, heterocyclyl-(C₁-C₆)-alkyl, (C₁-C₆)-alkoxy-(C₁-C₆)-alkyl, (C₁-C₆)-alkylthio-(C₁-C₆)-alkyl, (C₁-C₆)-haloalkoxy-(C₁-C₆)-alkyl, (C₁-C₆)-haloalkylthio-(C₁-C₆)-alkyl, COOR²³, CONR²¹R²², hydroxycarbonyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, (C₂-C₆)-alkynyloxycarbonyl-(C₁-C₆)-alkyl, aryl-(C₁-C₆)-alkoxycarbonyl-(C₁-C₆)-alkyl, heteroaryl-(C₁-C₆)-alkoxycarbonyl-(C₁-C₆)-alkyl, heterocyclyl-(C₁-C₆)-alkoxycarbonyl-(C₁-C₆)-alkyl,
R⁵ and R⁶ together with the atom to which they are bonded form a fully saturated or partly saturated 5-7-membered ring optionally interrupted by heteroatoms and optionally having further substitution,
R¹ and X¹, when X¹ is a C—R² group, together with the atoms to which they are bonded form a fully saturated, partly saturated or fully unsaturated 5-7-membered ring optionally interrupted by heteroatoms and optionally having further substitution,
X¹ and X², when each is a C—R² group, together with the atoms to which they are bonded form a fully saturated, partly saturated or fully unsaturated 5-7-membered ring optionally interrupted by heteroatoms and optionally having further substitution,
A¹ and A², when each is a C—R⁷ group, together with the atoms to which they are bonded form a fully saturated, partly saturated or fully unsaturated 5-7-membered ring optionally interrupted by heteroatoms and optionally having further substitution,
A² and A³, when each is a C—R⁷ group, together with the atoms to which they are bonded form a fully saturated, partly saturated or fully unsaturated 5-7-membered ring optionally interrupted by heteroatoms and optionally having further substitution,
A³ and A⁴, when each is a C—R⁷ group, together with the atoms to which they are bonded form a fully saturated, partly saturated or fully unsaturated 5-7-membered ring optionally interrupted by heteroatoms and optionally having further substitution,
Y is a bond or the Y-1 to Y-7 moieties

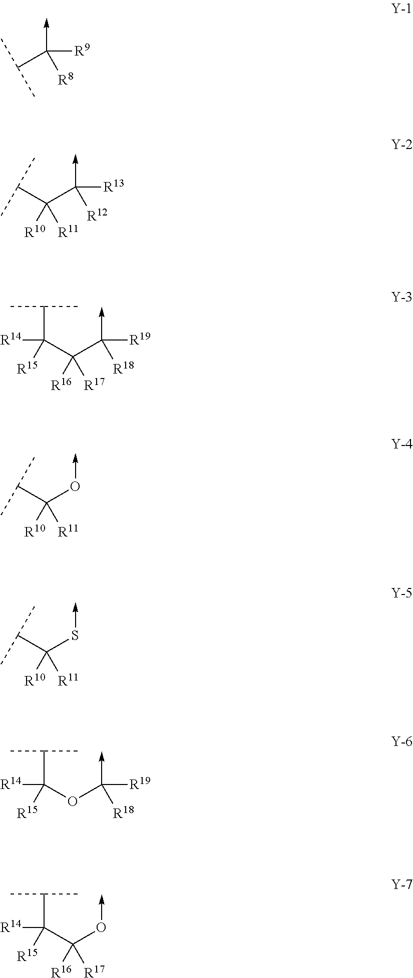

where R⁸, R⁹, R¹⁰, R¹¹, R¹², R¹³, R¹⁴, R¹⁵, R¹⁶, R¹⁷, R¹⁸ and R¹⁹ are each as per the definition below and where the arrow represents a bond to the 6-membered ring with the A¹, A², A³, A⁴ and A⁵ moieties,
R⁸, R⁹, R¹⁰, R¹¹, R¹², R¹³, R¹⁴, R¹⁵, R¹⁶, R¹⁷, R¹⁸ and R¹⁹ are independently hydrogen, (C₁-C₆)-alkyl, (C₂-C₆)-alkenyl, (C₂-C₆)-alkynyl, (C₁-C₁₀)-haloalkyl, (C₂-C₆)-haloalkenyl, (C₂-C₆)-haloalkynyl, (C₃-C₁₀)-cycloalkyl, aryl, aryl-(C₁-C₆)-alkyl, heteroaryl, heteroaryl-(C₁-C₆)-alkyl, (C₃-C₆)-cycloalkyl-(C₁-C₆)-alkyl, heterocyclyl, heterocyclyl-(C₁-C₆)-alkyl, (C₁-C₆)-alkoxy-(C₁-C₆)-alkyl, (C₁-C₆)-alkylthio-(C₁-C₆)-alkyl, (C₁-C₆)-haloalkoxy-(C₁-C₆)-alkyl, (C₁-C₆)-haloalkylthio-(C₁-C₆)-alkyl, COOR²³,
n is 0, 1 or 2,
R²¹ and R²² are the same or different and are independently hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-haloalkoxy-(C₁-C₆)-alkyl, (C₁-C₆)-alkylthio-(C₁-C₆)-alkyl, (C₁-C₆)-haloalkylthio-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, COR²³, SO₂R²⁴, (C₁-C₆)-alkyl-HNO₂S—, (C₃-C₆)-cycloalkyl-HNO₂S—, heterocyclyl, (C₁-C₆)-alkoxycarbonyl-(C₁-C₆)-alkyl, (C₁-C₆)-alkoxycarbonyl, aryl-(C₁-C₆)-alkoxycarbonyl-(C₁-C₆)-alkyl, aryl-(C₁-C₆)-alkoxycarbonyl, heteroaryl-(C₁-C₆)-alkoxycarbonyl, (C₂-C₆)-alkenyloxycarbonyl, (C₂-C₆)-alkynyloxycarbonyl, heterocyclyl-(C₁-C₆)-alkyl,
R²³ is hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, hydroxycarbonyl-(C₁-C₆)-alkyl, heterocyclyl, heterocyclyl-(C₁-C₆)-alkyl and
R²⁴ is hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, heterocyclyl-(C₁-C₆)-alkyl, (C₃-C₆)-cycloalkyl-(C₁-C₆)-alkyl, (C₄-C₁₀)-cycloalkenyl-(C₁-C₆)-alkyl, NR²¹R²².

Very particularly preference is given to compounds of the general formula (I) in which
R¹, R² and R⁷ are independently hydrogen, fluorine, chlorine, bromine, iodine, cyano, nitro, NR²¹R²², OR²³, S(O)ₙR²⁴, thiocyanato, isothiocyanato, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, cyclopropylmethyl, cyclobutylmethyl, cyclopentylmethyl, cyclohexylmethyl, optionally substituted phenyl, heteroaryl, heterocyclyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 1-cyanocyclobutyl, 2-cyanocyclobutyl, 3-cyanocyclobutyl, 1-allylcyclopropyl, 1-vynylcyclobutyl, 1-vynylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl , 1-ethyl-1-methyl-2-propenyl, 1-ethyl-2-methyl-1-propenyl and 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, difluoro-tert-butyl, trifluoromethoxymethyl, trifluoromethoxyethyl, trifluoromethoxy-n-propyl, difluoromethoxymethyl, difluoromethoxyethyl, difluoromethoxy-n-propyl, 2,2-difluoroethoxymethyl, 2,2-difluoroethoxyethyl, 2,2-difluoroethoxy-n-propyl, 2,2,2-trifluoroethoxymethyl, 2,2,2-trifluoroethoxyethyl, 2,2,2-trifluoroethoxy-n-propyl, pentafluoroethoxymethyl, pentafluoroethoxyethyl, pentafluoroethoxy-n-propyl, methylthiomethyl, methylthioethyl, ethylthioethyl, methylthio-n-propyl, ethylthio-n-propyl, trifluoromethylthiomethyl, trifluoromethylthioethyl, trifluoromethylthio-n-propyl, 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, methoxymethoxymethyl, methoxyethoxymethyl, methoxyethoxyethyl, methoxymethoxyethyl, ethoxy-n-propoxymethyl, ethoxy-n-propoxyethyl, ethoxyethoxymethyl, ethoxyethoxyethyl (C₂-C₆)-haloalkenyl, (C₂-C₆)-haloalkynyl, pentafluorothio, aryl-(C₁-C₆)-alkyl, heteroaryl-(C₁-C₆)-alkylheterocyclyl-(C₁-C₆)-alkyl, (C₁-C₆)-alkylcarbonyl-(C₁-C₆)-alkyl, COOR²³, CONR²¹R²², COR²³, —C═NOR²³, R²³OOC-(C₁-C₆)-alkyl, aryl-(C₁-C₆)-alkynyl, heteroaryl-(C₁-C₆)-alkynyl, heterocyclyl-(C₁-C₆)-alkynyl, trimethylsilylethynyl, triethylsilylethynyl, tris(isopropyl)silylethynyl, (C₃-C₆)-cycloalkyl-(C₂-C₆)-alkynyl, aryl-(C₂-C₆)-alkenyl, heteroaryl-(C₂-C₆)-alkenyl, heterocyclyl-(C₂-C₆)-alkenyl, (C₃-C₆)-cycloalkyl-(C₂-C₆)-alkenyl, (C₁-C₆)-alkylaminosulfonylamino, (C₃-C₆)-cycloalkylaminosulfonylamino, diazo, aryldiazo, trimethylsilyl, triethylsilyl, tris(isopropyl)silyl, diphenyl(methyl)silyl, dimethyl(phenyl)silyl, dimethyl(tert-butyl)silyl, diphenyl(tert-butyl)silyl,
X¹, X², X³ and X⁴ are the same or different and are independently N (nitrogen) or the C—R² moiety, but there are never more than two adjacent nitrogen atoms, and where R² in each C—R² moiety is the same or different as defined above, and where R¹ and R² are not both hydrogen when X¹, X², X³ and X⁴ are C—R²,
W is O (oxygen) or S (sulfur),
A¹, A², A³, A⁴ and A⁵ are the same or different and are each independently N (nitrogen) or the C—R⁷ moiety, but there are never more than two adjacent nitrogen atoms, and where R⁷ in each C—R⁷ moiety is the same or different as defined above,
R³ is methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, cyanomethyl, cyanoethyl, cyano-n-propyl, cyano-n-butyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl , 1-ethyl-1-methyl-2-propenyl, 1-ethyl-2-methyl-1-propenyl and 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 1,1-difluoroethyl, 2,2-difluoroethyl, 2,2,2-trifluoroethyl, 3,3-difluoro-n-propyl, 3,3,3-trifluoro-n-propyl, 4,4-difluoro-n-butyl, 4,4,4-trifluoro-n-butyl, difluoro-tert-butyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, trifluoromethoxymethyl, trifluoromethoxyethyl, trifluoromethoxy-n-propyl, difluoromethoxymethyl, difluoromethoxyethyl, difluoromethoxy-n-propyl, 2,2-difluoroethoxymethyl, 2,2-difluoroethoxyethyl, 2,2-difluoroethoxy-n-propyl, 2,2,2-trifluoroethoxymethyl, 2,2,2-trifluoroethoxyethyl, 2,2,2-trifluoroethoxy-n-propyl, pentafluoroethoxymethyl, pentafluoroethoxyethyl, pentafluoroethoxy-n-propyl, methylthiomethyl, methylthioethyl, ethylthioethyl, methylthio-n-propyl, ethylthio-n-propyl, trifluoromethylthiomethyl, trifluoromethylthioethyl, trifluoromethylthio-n-propyl, 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, methoxymethoxymethyl, methoxyethoxymethyl, methoxyethoxyethyl, methoxymethoxyethyl, ethoxy-n-propoxymethyl, ethoxy-n-propoxyethyl, ethoxyethoxymethyl, ethoxyethoxyethyl, methoxyethoxy-n-propyl, ethoxyethoxy-n-propyl, cyclopropylmethyl, cyclobutylmethyl, cyclopentylmethyl, cyclohexylmethyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, (C₂-C₆)-haloalkenyl, (C₂-C₆)-haloalkynyl, heterocyclyl, heterocyclyl-(C₁-C₆)-alkyl, R²¹R²²N—(C₁-C₆)-alkyl,
R⁴ is hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl , 1,2-dimethyl-3-butenyl , 1,3-dimethyl-1-butenyl , 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3,3-trifluoro-n-propyl, difluoro-tert-butyl, (C₂-C₆)-haloalkenyl, (C₂-C₆)-haloalkynyl, 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, methoxymethoxymethyl, methoxyethoxymethyl, methoxyethoxyethyl, methoxymethoxyethyl, ethoxy-n-propoxymethyl, ethoxy-n-propoxyethyl, ethoxyethoxymethyl, ethoxyethoxyethyl, methoxyethoxy-n-propyl, ethoxyethoxy-n-propyl, cyclopropylmethyl, cyclobutylmethyl, cyclopentylmethyl, cyclohexylmethyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, (C₃-C₆)-halocycloalkyl, (C₄-C₆)-halocycloalkenyl, optionally substituted phenyl, aryl-(C₁-C₆)-alkyl, heteroaryl, heteroaryl-(C₁-C₆)-alkyl, heterocyclyl-(C₁-C₆)-alkyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, trifluoromethoxymethyl, trifluoromethoxyethyl, trifluoromethoxy-n-propyl, difluoromethoxymethyl, difluoromethoxyethyl, difluoromethoxy-n-propyl, 2,2-difluoroethoxymethyl, 2,2-difluoroethoxyethyl, 2,2-difluoroethoxy-n-propyl, 2,2,2-trifluoroethoxymethyl, 2,2,2-trifluoroethoxyethyl, 2,2,2-trifluoroethoxy-n-propyl, pentafluoroethoxymethyl, pentafluoroethoxyethyl, pentafluoroethoxy-n-propyl, methylthiomethyl, methylthioethyl, ethylthioethyl, methylthio-n-propyl, ethylthio-n-propyl, trifluoromethylthiomethyl, trifluoromethylthioethyl, trifluoromethylthio-n-propyl, (C₁-C₅)-alkylamino-(C₁-C₅)-alkyl, bis[(C₁-C₅)-alkyl]amino-(C₁-C₅)-alkyl, (C₃-C₆)-cycloalkylamino-(C₁-C₅)-alkyl, (C₁-C₅)-alkoxy-(C₁-C₅)-alkoxy-(C₁-C₅)-alkyl, (C₁-C₅)-alkoxycarbonyl, (C₂-C₆)-alkenyloxycarbonyl, (C₂-C₆)-alkynyloxycarbonyl, aryl-(C₁-C₅)-alkoxycarbonyl, heteroaryl-(C₁-C₅)-alkoxycarbonyl, (C₃-C₆)-cycloalkyl-(C₁-C₅)-alkoxycarbonyl, CONR²¹R²², SO₂R²⁴, hydroxycarbonyl-(C₁-C₅)-alkyl, (C₁-C₅)-alkoxycarbonyl-(C₁-C₅)-alkyl, (C₂-C₆)-alkenyloxycarbonyl-(C₁-C₆)-alkyl, (C₂-C₆)-alkynyloxycarbonyl-(C₁-C₅)-alkyl, aryl-(C₁-C₅)-alkoxycarbonyl-(C₁-C₅)-alkyl, heteroaryl-(C₁-C₅)-alkoxycarbonyl-(C₁-C₅)-alkyl, heterocyclyl-(C₁-C₅)-alkoxycarbonyl-(C₁-C₅)-alkyl, (C₁-C₅)-alkylcarbonyl-(C₁-C₅)-alkyl, (C₁-C₅)-alkylsulfonyl-(C₁-C₅)-alkyl, (C₁-C₅)-alkylsulfinyl-(C₁-C₅)-alkyl,
R⁵ and R⁶ are independently hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 1,1-difluoroethyl, 2,2-difluoroethyl, 2,2,2-trifluoroethyl, 3,3,3-trifluoro-n-propyl, difluoro-tert-butyl, (C₂-C₆)-haloalkenyl, (C₂-C₆)-haloalkynyl, (C₃-C₆)-cycloalkyl, (C₃-C₆)-halocycloalkyl, (C₄-C₆)-cycloalkenyl, (C₄-C₆)-halocycloalkenyl, aryl, aryl-(C₁-C₅)-alkyl, heteroaryl, heteroaryl-(C₁-C₅)-alkyl, heterocyclyl, heterocyclyl-(C₁-C₅)-alkyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, trifluoromethoxymethyl, trifluoromethoxyethyl, trifluoromethoxy-n-propyl, difluoromethoxymethyl, difluoromethoxyethyl, difluoromethoxy-n-propyl, 2,2-difluoroethoxymethyl, 2,2-difluoroethoxyethyl, 2,2-difluoroethoxy-n-propyl, 2,2,2-trifluoroethoxymethyl, 2,2,2-trifluoroethoxyethyl, 2,2,2-trifluoroethoxy-n-propyl, pentafluoroethoxymethyl, pentafluoroethoxyethyl, pentafluoroethoxy-n-propyl, methylthiomethyl, methylthioethyl, ethylthioethyl, methylthio-n-propyl, ethylthio-n-propyl, trifluoromethylthiomethyl, trifluoromethylthioethyl, trifluoromethylthio-n-propyl, COOR²³, CONR²¹R²², hydroxycarbonyl-(C₁-C₅)-alkyl, (C₁-C₅)-alkoxycarbonyl-(C₁-C₅)-alkyl, (C₂-C₆)-alkenyloxycarbonyl-(C₁-C₅)-alkyl, (C₂-C₆)-alkynyloxycarbonyl-(C₁-C₅)-alkyl, aryl-(C₁-C₅)-alkoxycarbonyl-(C₁-C₅)-alkyl, heteroaryl-(C₁-C₅)-alkoxycarbonyl-(C₁-C₅)-alkyl, heterocyclyl-(C₁-C₅)-alkoxycarbonyl-(C₁-C₅)-alkyl,
R⁵ and R⁶ together with the atom to which they are bonded form a fully saturated or partly saturated 5-7-membered ring optionally interrupted by heteroatoms and optionally having further substitution,
R¹ and X¹, when X′ is a C—R² group, together with the atoms to which they are bonded form a fully saturated, partly saturated or fully unsaturated 5-7-membered ring optionally interrupted by heteroatoms and optionally having further substitution,
X¹ and X², when each is a C—R² group, together with the atoms to which they are bonded form a fully saturated, partly saturated or fully unsaturated 5-7-membered ring optionally interrupted by heteroatoms and optionally having further substitution,
A¹ and A², when each is a C—R⁷ group, together with the atoms to which they are bonded form a fully saturated, partly saturated or fully unsaturated 5-7-membered ring optionally interrupted by heteroatoms and optionally having further substitution,
A² and A³, when each is a C—R⁷ group, together with the atoms to which they are bonded form a fully saturated, partly saturated or fully unsaturated 5-7-membered ring optionally interrupted by heteroatoms and optionally having further substitution,
A³ and A⁴, when each is a C—R⁷ group, together with the atoms to which they are bonded form a fully saturated, partly saturated or fully unsaturated 5-7-membered ring optionally interrupted by heteroatoms and optionally having further substitution,
Y is a bond or the Y-1 to Y-7 moieties

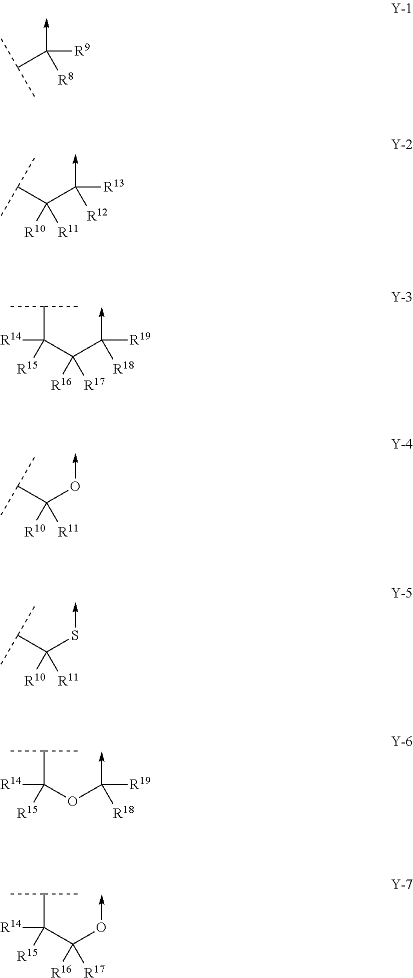

where R⁸, R⁹, R¹⁰, R¹¹, R¹², R¹³, R¹⁴, R¹⁵, R¹⁶, R¹⁷, R¹⁸ and R¹⁹ are each as per the definition below and where the arrow represents a bond to the 6-membered ring with the A¹, A², A³, A⁴ and A⁵ moieties,
R⁸, R⁹, R¹⁰, R¹¹, R¹², R¹³, R¹⁴, R15, R¹⁶, R¹⁷, R¹⁸ and R¹⁹ are independently hydrogen, fluorine, methyl, ethyl, n-propyl, isopropyl, n-butyl, iso-butyl, tert-butyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 1,1-difluoroethyl, 2,2-difluoroethyl, 2,2,2-trifluoroethyl, 3,3,3-trifluoro-n-propyl, difluoro-tert-butyl, cyclopropyl, cyclobutyl, cyclopentyl, cyclohexyl, cyclopropylmethyl, cyclobutylmethyl, cyclopentylmethyl, cyclohexylmethyl, optionally substituted phenyl, aryl-(C₁-C₆)-alkyl, heteroaryl, heteroaryl-(C₁-C₆)-alkyl, heterocyclyl, heterocyclyl-(C₁-C₆)-alkyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, trifluoromethoxymethyl, trifluoromethoxyethyl, trifluoromethoxy-n-propyl, difluoromethoxymethyl, difluoromethoxyethyl, difluoromethoxy-n-propyl, 2,2-difluoroethoxymethyl, 2,2-difluoroethoxyethyl, 2,2-difluoroethoxy-n-propyl, 2,2,2-trifluoroethoxymethyl, 2,2,2-trifluoroethoxyethyl, 2,2,2-trifluoroethoxy-n-propyl, pentafluoroethoxymethyl, pentafluoroethoxyethyl, pentafluoroethoxy-n-propyl, methylthiomethyl, methylthioethyl, ethylthioethyl, methylthio-n-propyl, ethylthio-n-propyl, trifluoromethylthiomethyl, trifluoromethylthioethyl, trifluoromethylthio-n-propyl, COOR²³,
n is 0, 1 or 2,
R²¹ and R²² are the same or different and are independently hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl , 1-ethyl-1-methyl-2-propenyl, 1-ethyl-2-methyl-1-propenyl and 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, (C₁-C₆)-haloalkyl, (C₂-C₆)-haloalkenyl, (C₂-C₆)-haloalkynyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi (cyclopropyl)-2-yl, 1-cyanocyclopropyl, 2-cyanocyclopropyl, 1-methylcyclobutyl, 2-methylcyclobutyl, 3-methylcyclobutyl, 1-cyanocyclobutyl, 2-cyanocyclobutyl, 3-cyanocyclobutyl, 3-methoxycyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, (C₃-C₆)-halocycloalkyl, (C₄-C₆)-cycloalkenyl, (C₄-C₆)-halocycloalkenyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, trifluoromethoxymethyl, trifluoromethoxyethyl, trifluoromethoxy-n-propyl, difluoromethoxymethyl, difluoromethoxyethyl, difluoromethoxy-n-propyl, 2,2-difluoroethoxymethyl, 2,2-difluoroethoxyethyl, 2,2-difluoroethoxy-n-propyl, 2,2,2-trifluoroethoxymethyl, 2,2,2-trifluoroethoxyethyl, 2,2,2-trifluoroethoxy-n-propyl, pentafluoroethoxymethyl, pentafluoroethoxyethyl, pentafluoroethoxy-n-propyl, methylthiomethyl, methylthioethyl, ethylthioethyl, methylthio-n-propyl, ethylthio-n-propyl, trifluoromethylthiomethyl, trifluoromethylthioethyl, trifluoromethylthio-n-propyl, optionally substituted phenyl, aryl-(C₁-C₅)-alkyl, heteroaryl, heteroaryl-(C₁-C₅)-alkyl, (C₄-C₆)-cycloalkenyl-(C₁-C₅)-alkyl, COR²³, SO₂R²⁴, —(C₁-C₆)-alkyl-HNO₂S—, (C₃-C₆)-cycloalkyl-HNO₂S—, heterocyclyl, (C₁-C₅)-alkoxycarbonyl-(C₁-C₅)-alkyl, (C₁-C₅)-alkoxycarbonyl, aryl-(C₁-C₅)-alkoxycarbonyl-(C₁-C₅)-alkyl, aryl-(C₁-C₅)-alkoxycarbonyl, heteroaryl-(C₁-C₅)-alkoxycarbonyl, (C₂-C₆)-alkenyloxycarbonyl, (C₂-C₆)-alkynyloxycarbonyl, heterocyclyl-(C₁-C₅)-alkyl,
R²³ is hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 2-cyanocyclopropyl, 1-methylcyclobutyl, 2-methylcyclobutyl, 3-methylcyclobutyl, 2-cyanocyclobutyl, 3-cyanocyclobutyl, 3-methoxycyclobutyl, 1-allylcyclopropyl, 1-ethylcyclopropyl, 1-methylcyclohexyl, 2-methylcyclohexyl, 3-methylcyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3-difluoro-n-propyl, 3,3,3-trifluoro-n-propyl, 4,4-difluoro-n-butyl, 4,4,4-trifluoro-n-butyl, (C₂-C₆)-haloalkenyl, (C₂-C₆)-haloalkynyl, (C₃-C₆)-halocycloalkyl, (C₄-C₆)-cycloalkenyl, (C₄-C₆)-halocycloalkenyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, optionally substituted phenyl, aryl-(C₁-C₆)-alkyl, heteroaryl, heteroaryl-(C₁-C₆)-alkyl, (C₄-C₆)-cycloalkenyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, hydroxycarbonyl-(C₁-C₆)-alkyl, heterocyclyl, heterocyclyl-(C₁-C₆)-alkyl and
R²⁴ is hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi (cyclopropyl)-2-yl, 2-cyanocyclopropyl, 1-methylcyclobutyl, 2-methylcyclobutyl, 3-methylcyclobutyl, 2-cyanocyclobutyl, 3-cyanocyclobutyl, 3-methoxycyclobutyl, 1-allylcyclopropyl, 1-ethylcyclopropyl, 1-methylcyclohexyl, 2-methylcyclohexyl, 3-methylcyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3,3-trifluoro-n-propyl, difluoro-tert-butyl, (C₂-C₆)-haloalkenyl, (C₂-C₆)-haloalkynyl, (C₃-C₆)-halocycloalkyl, (C₄-C₆)-cycloalkenyl, (C₄-C₆)-halocycloalkenyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, optionally substituted phenyl, aryl-(C₁-C₆)-alkyl, heteroaryl, heteroaryl-(C₁-C₆)-alkyl, heterocyclyl-(C₁-C₆)-alkyl, (C₄-C₆)-cycloalkenyl-(C₁-C₆)-alkyl, NR²¹R²².

Specifically preferred are compounds of the general formula (I) described by the formulae (Ia) to (Ip)

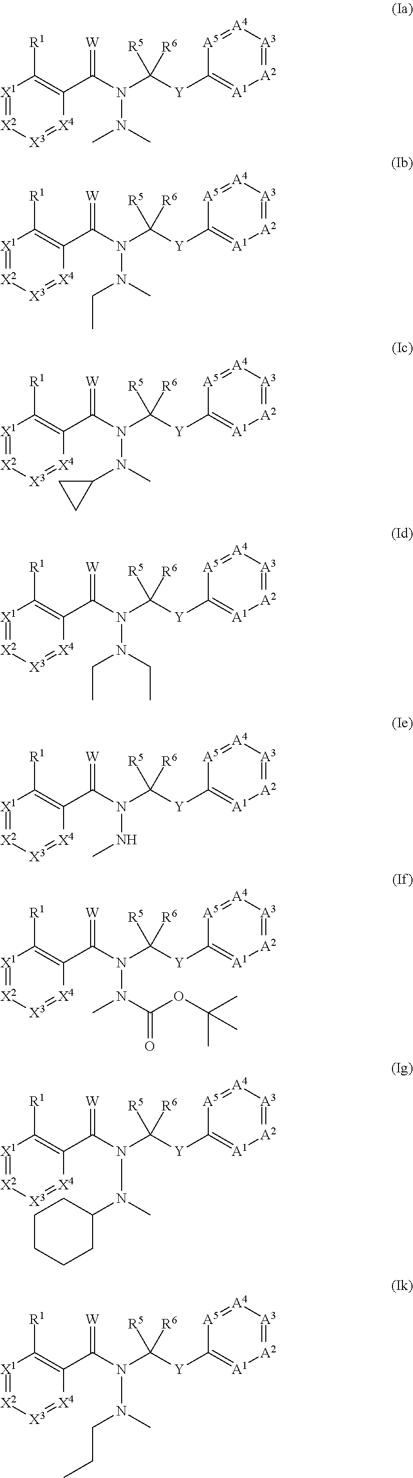

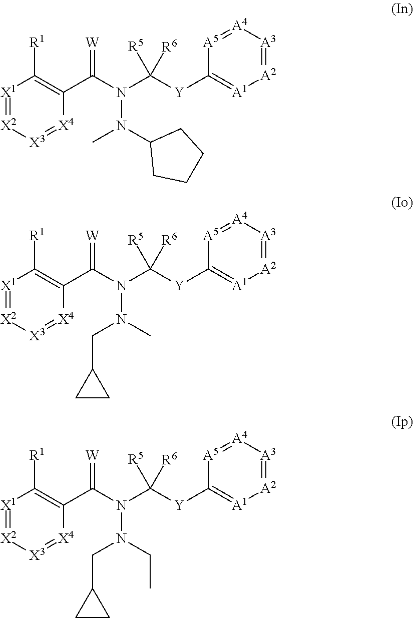

in which
R¹, R² and R⁷ are independently hydrogen, fluorine, chlorine, bromine, iodine, cyano, nitro, NR²¹R²², OR²³, S(O)ₙR²⁴, thiocyanato, isothiocyanato, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, cyclopropylmethyl, cyclobutylmethyl, cyclopentylmethyl, cyclohexylmethyl, optionally substituted phenyl, heteroaryl, heterocyclyl, cyclopropyl, cyclobutyl, cyclopentyl, cyclohexyl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, ethynyl, 1-propynyl, 2-propynyl, 1-butynyl, 2-butynyl, 3-butynyl, 1-methyl-2-propynyl, methoxymethyl, ethoxymethyl, ethoxyethyl, methoxyethyl, methoxy-n-propyl, ethoxy-n-propyl, methoxybutyl, trifluoromethyl, pentafluoroethyl, 1,1,2,2-tetrafluoroethyl, heptafluoropropyl, nonafluorobutyl, chlorodifluoromethyl, 1-fluoroethyl, 2-fluoroethyl, fluoromethyl, difluoromethyl, 2,2-difluoroethyl, 2,2,2-trifluoroethyl, difluoro-tert-butyl, trifluoromethoxymethyl, trifluoromethoxyethyl, trifluoromethoxy-n-propyl, difluoromethoxymethyl, difluoromethoxyethyl, difluoromethoxy-n-propyl, 2,2-difluoroethoxymethyl, 2,2-difluoroethoxyethyl, 2,2-difluoroethoxy-n-propyl, 2,2,2-trifluoroethoxymethyl, 2,2,2-trifluoroethoxyethyl, 2,2,2-trifluoroethoxy-n-propyl, pentafluoroethoxymethyl, pentafluoroethoxyethyl, pentafluoroethoxy-n-propyl, methylthiomethyl, methylthioethyl, ethylthioethyl, trifluoromethylthiomethyl, trifluoromethylthioethyl, 1-cyclopentenyl, 2-cyclopentenyl, 3-cyclopentenyl, 1-cyclohexenyl, 2-cyclohexenyl, 3-cyclohexenyl, methoxymethoxymethyl, methoxyethoxymethyl, methoxyethoxyethyl, methoxymethoxyethyl, ethoxy-n-propoxymethyl, ethoxy-n-propoxyethyl, ethoxyethoxymethyl, ethoxyethoxyethyl, fluoroethynyl, chloroethynyl, trifluoromethylethynyl, pentafluorothio, aryl-(C₁-C₆)-alkyl, heteroaryl-(C₁-C₆)-alkyl, heterocyclyl-(C₁-C₆)-alkyl, methylcarbonylmethyl, methylcarbonylethyl, methylcarbonyl-n-propyl, COOR²³, CONR²¹R²², COR²³, —C═NOR²³, R²³OOC—(C₁-C₆)-alkyl, aryl-(C₁-C₆)-alkynyl, heteroaryl-(C₁-C₆)-alkynyl, heterocyclyl-(C₁-C₆)-alkynyl, trimethylsilylethynyl, triethylsilylethynyl, tris(isopropyl)silylethynyl, cyclopropylethynyl, cyclobutylethynyl, cyclopentylethynyl, cyclohexylethynyl, aryl-(C₂-C₆)-alkenyl, heteroaryl-(C₂-C₆)-alkenyl, heterocyclyl-(C₂-C₆)-alkenyl, (C₁-C₆)-alkylaminosulfonylamino, (C₃-C₆)-cycloalkylaminosulfonylamino, diazo, aryldiazo, trimethylsilyl,
X¹, X², X³ and X⁴ are the same or different and are independently N (nitrogen) or the C—R² moiety, but there are never more than two adjacent nitrogen atoms, and where R² in each C—R² moiety is the same or different as defined above, and where R¹ and R² are not both hydrogen when X¹, X², X³ and X⁴ are C—R²,
W is O (oxygen) or S (sulfur), preferably O (oxygen),
A¹, A², A³, A⁴ and A⁵ are the same or different and are each independently N (nitrogen) or the C—R⁷ moiety, but there are never more than two adjacent nitrogen atoms, and where R⁷ in each C—R⁷ moiety is the same or different as defined above,
R⁵ and R⁶ are independently hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, trifluoromethyl, pentafluoroethyl, 1,1,2,2-tetrafluoroethyl, heptafluoropropyl, nonafluorobutyl, 1-fluoroethyl, 2-fluoroethyl, fluoromethyl, difluoromethyl, 1,1-difluoroethyl, 2,2-difluoroethyl, 2,2,2-trifluoroethyl, 3,3,3-trifluoro-n-propyl, difluoro-tert-butyl, cyclopropyl, cyclobutyl, cyclopentyl, cyclohexyl, optionally substituted phenyl, aryl, aryl-(C₁-C₅)-alkyl, heteroaryl, heteroaryl-(C₁-C₅)-alkyl, heterocyclyl, heterocyclyl-(C₁-C₅)-alkyl, methoxymethyl, ethoxymethyl, ethoxyethyl, methoxyethyl, methoxy-n-propyl, ethoxy-n-propyl, methoxybutyl, isopropoxymethyl, isopropoxyethyl, trifluoromethoxymethyl, trifluoromethoxyethyl, trifluoromethoxy-n-propyl, 2,2-difluoroethoxymethyl, 2,2-difluoroethoxyethyl, 2,2-difluoroethoxy-n-propyl, 2,2,2-trifluoroethoxymethyl, 2,2,2-trifluoroethoxyethyl, 2,2,2-trifluoroethoxy-n-propyl, pentafluoroethoxymethyl, pentafluoroethoxyethyl, pentafluoroethoxy-n-propyl, methylthiomethyl, methylthioethyl, ethylthioethyl, methylthio-n-propyl, ethylthio-n-propyl, trifluoromethylthiomethyl, trifluoromethylthioethyl, trifluoromethylthio-n-propyl, COOR²³, CONR²¹R²², hydroxycarbonylmethyl, hydroxycarbonylethyl, hydroxycarbonyl-n-propyl, methoxycarbonylmethyl, ethoxycarbonylmethyl, n-propyloxycarbonylm ethyl, isopropyloxycarbonylmethyl, tert-butyloxycarbonylm ethyl, methoxycarbonylethyl, ethoxycarbonylethyl, n-propyloxycarbonylethyl, isopropyloxycarbonylethyl, tert-butyloxycarbonylethyl, methoxycarbonyl-n-propyl, ethoxycarbonyl-n-propyl, n-propyloxycarbonyl-n-propyl, isopropyloxycarbonyl-n-propyl, tert-butyloxycarbonyl-n-propyl, allyloxycarbonylmethyl, allyloxycarbonylethyl, allyloxycarbonyl-n-propyl, propargyloxycarbonylmethyl, propargyloxycarbonylethyl, propargyloxycarbonyl-n-propyl, phenylmethyloxycarbonylmethyl, phenylmethyloxycarbonylethyl, phenylmethyloxycarbonyl-n-propyl, heteroaryl-(C₁-C₅)-alkoxycarbonyl-(C₁-C₅)-alkyl, heterocyclyl-(C₁-C₅)-alkoxycarbonyl-(C₁-C₅)-alkyl,
R⁵ and R⁶ together with the atom to which they are bonded form a fully saturated or partly saturated 5-7-membered ring optionally interrupted by heteroatoms and optionally having further substitution,
R¹ and X¹, when X¹ is a C—R² group, together with the atoms to which they are bonded form a fully saturated, partly saturated or fully unsaturated 5-7-membered ring optionally interrupted by heteroatoms and optionally having further substitution,
X¹ and X², when each is a C—R² group, together with the atoms to which they are bonded form a fully saturated, partly saturated or fully unsaturated 5-7-membered ring optionally interrupted by heteroatoms and optionally having further substitution,
A¹ and A², when each is a C—R⁷ group, together with the atoms to which they are bonded form a fully saturated, partly saturated or fully unsaturated 5-7-membered ring optionally interrupted by heteroatoms and optionally having further substitution,
A² and A³, when each is a C—R⁷ group, together with the atoms to which they are bonded form a fully saturated, partly saturated or fully unsaturated 5-7-membered ring optionally interrupted by heteroatoms and optionally having further substitution,
A³ and A⁴, when each is a C—R⁷ group, together with the atoms to which they are bonded form a fully saturated, partly saturated or fully unsaturated 5-7-membered ring optionally interrupted by heteroatoms and optionally having further substitution,
Y is a bond or the Y-1 to Y-7 moieties

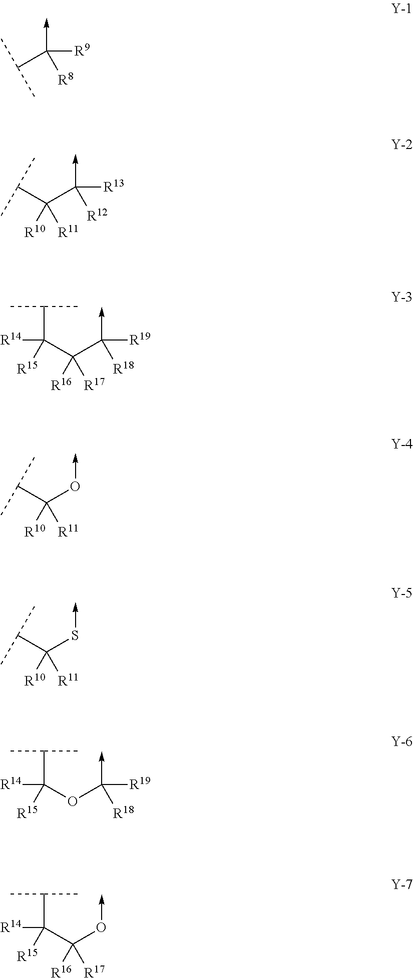

where R⁸, R⁹, R¹⁰, R¹¹, R¹², R¹³, R¹⁴, R¹⁵, R¹⁶, R¹⁷, R¹⁸ and R¹⁹ are each as per the definition below and where the arrow represents a bond to the 6-membered ring with the A¹, A², A³, A⁴ and A⁵ moieties,
R⁸, R⁹, R¹⁰, R¹¹, R¹², R¹³, R¹⁴, R¹⁵, R¹⁶, R¹⁷, R¹⁸ and R¹⁹ are independently hydrogen, fluorine, methyl, ethyl, n-propyl, isopropyl, n-butyl, isobutyl, tert-butyl, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, ethynyl, 1-propynyl, 2-propynyl, 1-butynyl, 2-butynyl, 3-butynyl, 1-methyl-2-propynyl, trifluoromethyl, pentafluoroethyl, 1,1,2,2-tetrafluoroethyl, heptafluoropropyl, nonafluorobutyl, chlorodifluoromethyl, 1-fluoroethyl, 2-fluoroethyl, fluoromethyl, difluoromethyl, 1,1-difluoroethyl, 2,2-difluoroethyl, 2,2,2-trifluoroethyl, 3,3,3-trifluoro-n-propyl, cyclopropyl, cyclobutyl, cyclopentyl, cyclohexyl, cyclopropylmethyl, cyclobutylmethyl, cyclopentylmethyl, cyclohexylmethyl, optionally substituted phenyl, aryl-(C₁-C₆)-alkyl, heteroaryl, heteroaryl-(C₁-C₆)-alkyl, heterocyclyl, heterocyclyl-(C₁-C₆)-alkyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, trifluoromethoxymethyl, trifluoromethoxyethyl, trifluoromethoxy-n-propyl, difluoromethoxymethyl, difluoromethoxyethyl, difluoromethoxy-n-propyl, 2,2,2-trifluoroethoxymethyl, 2,2,2-trifluoroethoxyethyl, 2,2,2-trifluoroethoxy-n-propyl, pentafluoroethoxymethyl, pentafluoroethoxyethyl, pentafluoroethoxy-n-propyl, methylthiomethyl, methylthioethyl, ethylthioethyl, methylthio-n-propyl, ethylthio-n-propyl, trifluoromethylthiomethyl, trifluoromethylthioethyl, trifluoromethylthio-n-propyl, COOR²³,
n is 0, 1 or 2,
R²¹ and R²² are the same or different and are independently hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 2,2-difluoromethyl, 2,2,2-trifluoroethyl, 3,3,3-trifluoro-n-propyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-methylcyclobutyl, 2-methylcyclobutyl, 3-methylcyclobutyl, 2-cyanocyclobutyl, 3-cyanocyclobutyl, 3-methoxycyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, trifluoromethoxymethyl, trifluoromethoxyethyl, trifluoromethoxy-n-propyl, difluoromethoxymethyl, difluoromethoxyethyl, difluoromethoxy-n-propyl, 2,2-difluoroethoxymethyl, 2,2-difluoroethoxyethyl, 2,2-difluoroethoxy-n-propyl, 2,2,2-trifluoroethoxymethyl, 2,2,2-trifluoroethoxyethyl, 2,2,2-trifluoroethoxy-n-propyl, pentafluoroethoxymethyl, pentafluoroethoxyethyl, pentafluoroethoxy-n-propyl, methylthiomethyl, methylthioethyl, ethylthioethyl, methylthio-n-propyl, ethylthio-n-propyl, trifluoromethylthiomethyl, trifluoromethylthioethyl, trifluoromethylthio-n-propyl, optionally substituted phenyl, aryl-(C₁-C₅)-alkyl, heteroaryl, heteroaryl-(C₁-C₅)-alkyl, COR²³, SO₂R²⁴, —(C₁-C₆)-alkyl-HNO₂S—, (C₃-C₆)-cycloalkyl-HNO₂S—, heterocyclyl, methoxycarbonylmethyl, ethoxycarbonylmethyl, n-propyloxycarbonylmethyl, isopropyloxycarbonylmethyl, tert-butyloxycarbonylmethyl, methoxycarbonylethyl, ethoxycarbonylethyl, n-propyloxycarbonylethyl, isopropyloxycarbonylethyl, tert-butyloxycarbonylethyl, methoxycarbonyl-n-propyl, ethoxycarbonyl-n-propyl, n-propyloxycarbonyl-n-propyl, isopropyloxycarbonyl-n-propyl, tert-butyloxycarbonyl-n-propyl, methoxycarbonyl, ethoxycarbonyl, n-propyloxycarbonyl, isopropyloxycarbonyl, tert-butyloxycarbonyl, aryl-(C₁-C₅)-alkoxycarbonyl-(C₁-C₅)-alkyl, aryl-(C₁-C₅)-alkoxycarbonyl, heteroaryl-(C₁-C₅)-alkoxycarbonyl, allyloxycarbonyl, propargyloxycarbonyl, heterocyclyl-(C₁-C₅)-alkyl,
R²³ is hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, cyclopropyl, cyclobutyl, cyclopentyl, cyclohexyl, adamantan-1-yl, adamantan-2-yl, 1-methylcyclopropyl, 2-methylcyclopropyl, 2,2-dimethylcyclopropyl, 2,3-dim ethylcyclopropyl, 1-methylcyclobutyl, 2-methylcyclobutyl, 3-methylcyclobutyl, 2-cyanocyclobutyl, 3-cyanocyclobutyl, 3-methoxycyclobutyl, 1-allylcyclopropyl, 1-ethylcyclopropyl, 1-methylcyclohexyl, 2-methylcyclohexyl, 3-methylcyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3,3-trifluoro-n-propyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, optionally substituted phenyl, aryl-(C₁-C₆)-alkyl, heteroaryl, heteroaryl-(C₁-C₆)-alkyl, methoxycarbonylmethyl, ethoxycarbonylmethyl, n-propyloxycarbonylmethyl, isopropyloxycarbonylmethyl, tert-butyloxycarbonylmethyl, methoxycarbonylethyl, ethoxycarbonylethyl, n-propyloxycarbonylethyl, isopropyloxycarbonylethyl, tert-butyloxycarbonylethyl, methoxycarbonyl-n-propyl, ethoxycarbonyl-n-propyl, n-propyloxycarbonyl-n-propyl, isopropyloxycarbonyl-n-propyl, tert-butyloxycarbonyl-n-propyl, allyloxycarbonylmethyl, allyloxycarbonylethyl, allyloxycarbonyl-n-propyl, aryl-(C₁-C₆)-alkoxycarbonyl-(C₁-C₆)-alkyl, hydroxycarbonyl-(C₁-C₆)-alkyl, heterocyclyl, heterocyclyl-(C₁-C₆)-alkyl and
R²⁴ is hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, ethynyl, 1-propynyl, 2-propynyl, 1-butynyl, 2-butynyl, 3-butynyl, 1-methyl-2-propynyl, cyclopropyl, cyclobutyl, cyclopentyl, cyclohexyl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, cyclopropylmethyl, cyclobutylmethyl, cyclopentylmethyl, cyclohexylmethyl, trifluoromethyl, pentafluoroethyl, 1,1,2,2-tetrafluoroethyl, heptafluoropropyl, nonafluorobutyl, 1-fluoroethyl, 2-fluoroethyl, fluoromethyl, difluoromethyl, 2,2-difluoroethyl, 2,2,2-trifluoroethyl, 3,3,3-trifluoro-n-propyl, difluoro-tert-butyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, optionally substituted phenyl, aryl-(C₁-C₆)-alkyl, heteroaryl, heteroaryl-(C₁-C₆)-alkyl, heterocyclyl-(C₁-C₆)-alkyl, NR²¹R²².

Very specifically preferred are compounds of the general formula (I) described by the formulae (Ia) to (Ip)

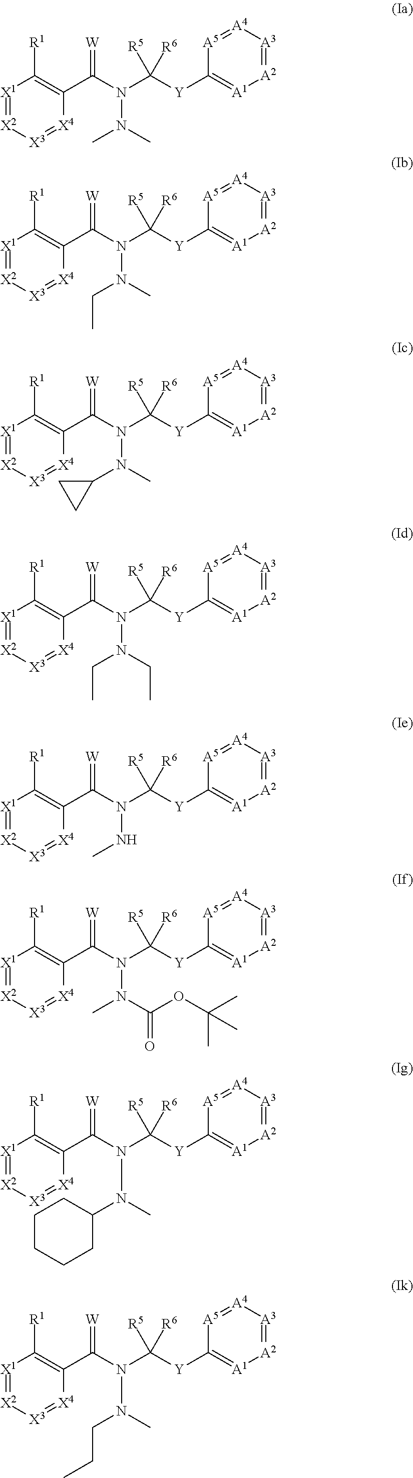

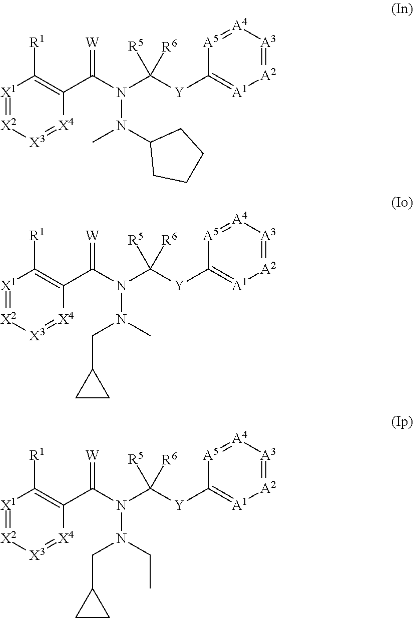

in which
R¹, R² and R⁷ are independently hydrogen, fluorine, chlorine, bromine, iodine, cyano, nitro, thiocyanato, isothiocyanato, methoxy, ethoxy, isopropyloxy, methylsulfonyl, hydrothio, hydroxy, amino, imino, diazo, methyl, ethyl, n-propyl, 1-methylethyl, n-butyl, 1-methylpropyl, tert-butyl, cyclopropylmethyl, cyclopropyl, cyclobutyl, cyclopentyl, ethenyl, 1-propenyl, 2-propenyl, 1-methylethenyl, methoxymethyl, trifluoromethyl, pentafluoroethyl, chlorodifluoromethyl, difluoromethyl, 2,2-difluoroethyl, 2,2,2-trifluoroethyl, trifluoromethoxy, 2,2,2-trifluoroethoxy, difluoromethoxy, trifluoromethylthio, methylthio, ethylthio, phenyl, tertbutyloxycarbonylamino, dimethylamino, hydroxycarbonyl, methoxycarbonyl, ethoxycarbonyl,
X¹, X², X³ and X⁴ are the same or different and are independently N (nitrogen) or the C—R² moiety, but there are never more than two adjacent nitrogen atoms, and where R² in each C—R² moiety is the same or different as defined above, and where R¹ and R² are not both hydrogen when X¹, X², X³ and X⁴ are C—R²,
W is O (oxygen) or S (sulfur), preferably O (oxygen),
A¹, A², A³, A⁴ and A⁵ are the same or different and are each independently N (nitrogen) or the C—R⁷ moiety, but there are never more than two adjacent nitrogen atoms, and where R⁷ in each C—R⁷ moiety is the same or different as defined above,
R⁵ and R⁶ are independently hydrogen, methyl, ethyl, n-propyl, 1-methylethyl, n-butyl, cyclopropylmethyl, cyclopropyl, cyclobutyl, cyclopentyl, methoxycarbonyl, ethoxycarbonyl, hydroxycarbonyl, methoxycarbonylmethyl, ethoxycarbonylmethyl,
R⁵ and R⁶ together with the atom to which they are bonded form a fully saturated or partly saturated 5-7-membered ring optionally interrupted by heteroatoms and optionally having further substitution,
R¹ and X¹, when X¹ is a C—R² group, together with the atoms to which they are bonded form a fully saturated, partly saturated or fully unsaturated 5-7-membered ring optionally interrupted by heteroatoms and optionally having further substitution,
X¹ and X², when each is a C—R² group, together with the atoms to which they are bonded form a fully saturated, partly saturated or fully unsaturated 5-7-membered ring optionally interrupted by heteroatoms and optionally having further substitution,
A¹ and A², when each is a C—R⁷ group, together with the atoms to which they are bonded form a fully saturated, partly saturated or fully unsaturated 5-7-membered ring optionally interrupted by heteroatoms and optionally having further substitution,
A² and A³, when each is a C—R⁷ group, together with the atoms to which they are bonded form a fully saturated, partly saturated or fully unsaturated 5-7-membered ring optionally interrupted by heteroatoms and optionally having further substitution,
A³ and A⁴, when each is a C—R⁷ group, together with the atoms to which they are bonded form a fully saturated, partly saturated or fully unsaturated 5-7-membered ring optionally interrupted by heteroatoms and optionally having further substitution,
Y is a bond or the Y-1 to Y-2 moieties

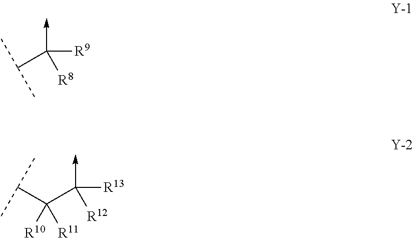

where R⁸, R⁹, R¹⁰, R¹¹, R¹², R¹³ are each as per the definition below and where the arrow represents a bond to the 6-membered ring with the A¹, A², A³, A⁴ and A⁵ moieties and
R⁸, R⁹, R¹⁰, R¹¹, R¹² and R¹³ are independently hydrogen, methyl.

The abovementioned general or preferred radical definitions apply both to the end products of the general formula (I) and, correspondingly, to the starting materials or the intermediates required in each case for the preparation. These radical definitions can be combined with one another as desired, i.e. including combinations between the given preferred ranges.

With regard to the compounds according to the invention, the terms used above and further below will be elucidated. These are familiar to the person skilled in the art and especially have the definitions elucidated hereinafter:

According to the invention, “arylsulfonyl” denotes optionally substituted phenylsulfonyl or optionally substituted polycyclic arylsulfonyl, here especially optionally substituted naphthylsulfonyl, for example substituted by fluorine, chlorine, bromine, iodine, cyano, nitro, alkyl, haloalkyl, haloalkoxy, amino, alkylamino, alkylcarbonylamino, dialkylamino or alkoxy groups.

According to the invention, “cycloalkylsulfonyl” alone or as part of a chemical group represents optionally substituted cycloalkylsulfonyl, preferably having 3 to 6 carbon atoms, for example cyclopropylsulfonyl, cyclobutylsulfonyl, cyclopentylsulfonyl or cyclohexylsulfonyl.

According to the invention, “alkylsulfonyl”—alone or as part of a chemical group—refers to straight-chain or branched alkylsulfonyl, preferably having 1 to 8 or 1 to 6 carbon atoms, for example (but not limited to) (C₁-C₆)-alkylsulfonyl such as methylsulfonyl, ethylsulfonyl, propylsulfonyl, 1-methylethylsulfonyl, butylsulfonyl, 1-methylpropylsulfonyl, 2-methylpropylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl.

According to the invention, “heteroarylsulfonyl” denotes optionally substituted pyridylsulfonyl, pyrimidinylsulfonyl, pyrazinylsulfonyl or optionally substituted polycyclic heteroarylsulfonyl, here in particular optionally substituted quinolinylsulfonyl, for example substituted by fluorine, chlorine, bromine, iodine, cyano, nitro, alkyl, haloalkyl, haloalkoxy, amino, alkylamino, alkylcarbonylamino, dialkylamino or alkoxy groups.

According to the invention, “alkylthio”—alone or as part of a chemical group—denotes straight-chain or branched S-alkyl, preferably having 1 to 8 or 1 to 6 carbon atoms, such as (C₁-C₁₀)-, (C₁-C₆)- or (C₁-C₄)-alkylthio, for example (but not limited to) (C₁-C₆)-alkylthio such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio.

According to the invention, alkenylthio denotes an alkenyl radical bonded via a sulfur atom, alkynylthio denotes an alkynyl radical bonded via a sulfur atom, cycloalkylthio denotes a cycloalkyl radical bonded via a sulfur atom, and cycloalkenylthio denotes a cycloalkenyl radical bonded via a sulfur atom.

According to the invention, alkylsulfinyl (alkyl-S(═O)—), unless defined differently elsewhere, denotes alkyl radicals which are bonded to the skeleton via —S(═O)—, such as (C₁-C₁₀)-, (C₁-C₆)- or (C₁-C₄)-alkylsulfinyl, for example (but not limited to) (C₁-C₆)-alkylsulfinyl such as methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl.

Analogously, alkenylsulfinyl and alkynylsulfinyl are defined in accordance with the invention respectively as alkenyl and alkynyl radicals bonded to the skeleton via —S(═O)—, such as (C₂-C₁₀)—, (C₂-C₆)- or (C₂-C₄)-alkenylsulfinyl or (C₃-C₁₀)-, (C₃-C₆)- or (C₃-C₄)-alkynylsulfinyl.

Analogously, alkenylsulfonyl and alkynylsulfonyl are defined in accordance with the invention respectively as alkenyl and alkynyl radicals bonded to the skeleton via —S(═O)₂—, such as (C₂-C₁₀)-, (C₂-C₆)- or (C₂-C₄)-alkenylsulfonyl or (C₃-C₁₀)-, (C₃-C₆)- or (C₃-C₄)-alkynylsulfonyl.

“Alkoxy” denotes an alkyl radical attached via an oxygen atom, for example (but not limited to) (C₁-C₆)-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy. Alkenyloxy denotes an alkenyl radical attached via an oxygen atom, and alkynyloxy denotes an alkynyl radical attached via an oxygen atom, such as (C₂-C₁₀)-, (C₂-C₆)- or (C₂-C₄)-alkenoxy and (C₃-C₁₀)-, (C₃-C₆)- or (C₃-C₄)-alkynoxy.

“Cycloalkyloxy” denotes a cycloalkyl radical attached via an oxygen atom and cycloalkenyloxy denotes a cycloalkenyl radical attached via an oxygen atom.

According to the invention, “alkylcarbonyl” (alkyl-C(═O)—), unless defined differently elsewhere, represents alkyl radicals bonded to the skeleton via —C(═O)—, such as (C₁-C₁₀)-, (C₁-C₆)- or (C₁-C₄)-alkylcarbonyl. Here, the number of the carbon atoms refers to the alkyl radical in the alkylcarbonyl group.

Analogously, “alkenylcarbonyl” and “alkynylcarbonyl”, unless defined differently elsewhere, in accordance with the invention, respectively represent alkenyl and alkynyl radicals bonded to the skeleton via —C(═O)—, such as (C₂-C₁₀)-, (C₂-C₆)- or (C₂-C₄)-alkenylcarbonyl and (C₂-C₁₀)-, (C₂-C₆)- and (C₂-C₄)-alkynylcarbonyl. Here, the number of the carbon atoms refers to the alkenyl or alkynyl radical in the alkenyl or alkynyl group.

Alkoxycarbonyl (alkyl-O—C(═O)—), unless defined differently elsewhere: alkyl radicals bonded to the skeleton via —O—C(═O)—, such as (C₁-C₁₀)-, (C₁-C₆)- or (C₁-C₄)-alkoxycarbonyl. Here, the number of the carbon atoms refers to the alkyl radical in the alkoxycarbonyl group.

Analogously, “alkenyloxycarbonyl” and “alkynyloxycarbonyl”, unless defined differently elsewhere, in accordance with the invention, respectively represent alkenyl and alkynyl radicals bonded to the skeleton via —O—C(═O)—, such as (C₂-C₁₀)-, (C₂-C₆)- or (C₂-C₄)-alkenyloxycarbonyl and (C₃-C₁₀)-, (C₃-C₆)- and (C₃-C₄)-alkynyloxycarbonyl. Here, the number of the carbon atoms refers to the alkenyl or alkynyl radical in the alkenyloxycarbonyl or alkynyloxycarbonyl group.

According to the invention, the term “alkylcarbonyloxy” (alkyl-C(═O)—O—), unless defined differently elsewhere, represents alkyl radicals bonded to the skeleton via the oxygen of a carbonyloxy group (—C(═O)—O—), such as (C₁-C₁₀)-, (C₁-C₆)- or (C₁-C₄)-alkylcarbonyloxy. Here, the number of the carbon atoms refers to the alkyl radical in the alkylcarbonyloxy group.

Analogously, “alkenylcarbonyloxy” and “alkynylcarbonyloxy” are defined in accordance with the invention respectively as alkenyl and alkynyl radicals bonded to the skeleton via the oxygen of (—C(═O)—O—), such as (C₂-C₁₀)-, (C₂-C₆)- or (C₂-C₄)-alkenylcarbonyloxy or (C₂-C₁₀)-, (C₂-C₆)- or (C₂-C₄)-alkynylcarbonyloxy. Here, the number of the carbon atoms refers to the alkenyl or alkynyl radical in the alkenyl- or alkynylcarbonyloxy group respectively.

The term “aryl” denotes an optionally substituted mono-, bi- or polycyclic aromatic system having preferably 6 to 14, especially 6 to 10, ring carbon atoms, for example phenyl, naphthyl, anthryl, phenanthrenyl and the like, preferably phenyl.

The term “optionally substituted aryl” also embraces polycyclic systems, such as tetrahydronaphthyl, indenyl, indanyl, fluorenyl, biphenylyl, where the bonding site is on the aromatic system. In systematic terms, “aryl” is generally also encompassed by the term “optionally substituted phenyl”. Preferred aryl substituents here are, for example, hydrogen, halogen, alkyl, cycloalkyl, cycloalkylalkyl, cycloalkenyl, halocycloalkyl, alkenyl, alkynyl, aryl, arylalkyl, arylalkenyl, heteroaryl, heteroarylalkyl, heterocyclyl, heterocyclylalkyl, alkoxyalkyl, alkylthio, haloalkylthio, haloalkyl, alkoxy, haloalkoxy, cycloalkoxy, cycloalkylalkoxy, aryloxy, heteroraryloxy, alkoxyalkoxy, alkynylalkoxy, alkenyloxy, bis-alkylaminoalkoxy, tris-[alkyl]silyl, bis-[alkyl]arylsilyl, bis-[alkyl]alkylsilyl, tris-[alkyl]silylalkynyl, arylalkynyl, heteroarylalkynyl, alkylalkynyl, cycloalkylalkynyl, haloalkylalkynyl, heterocyclyl-N-alkoxy, nitro, cyano, amino, alkylamino, bis-alkylamino, alkylcarbonylamino, cycloalkylcarbonylamino, arylcarbonylamino, alkoxycarbonylamino, alkoxycarbonylalkylamino, arylalkoxycarbonylalkylamino, hydroxycarbonyl, alkoxycarbonyl, am inocarbonyl, alkylaminocarbonyl, cycloalkylaminocarbonyl, bis-alkylaminocarbonyl, heteroarylalkoxy, arylalkoxy.

A heterocyclic radical (heterocyclyl) contains at least one heterocyclic ring (=carbocyclic ring in which at least one carbon atom has been replaced by a heteroatom, preferably by a heteroatom from the group of N, O, S, P) which is saturated, unsaturated, partly saturated or heteroaromatic and may be unsubstituted or substituted, in which case the bonding site is localized on a ring atom. If the heterocyclyl radical or the heterocyclic ring is optionally substituted, it may be fused to other carbocyclic or heterocyclic rings. In the case of optionally substituted heterocyclyl, polycyclic systems are also included, for example 8-azabicyclo[3.2.1]octanyl, 8-azabicyclo[2.2.2]octanyl or 1-azabicyclo[2.2.1]heptyl. Optionally substituted heterocyclyl also includes spirocyclic systems, such as, for example, 1-oxa-5-aza-spiro[2.3]hexyl. Unless defined otherwise, the heterocyclic ring preferably contains 3 to 9 ring atoms, in particular 3 to 6 ring atoms, and one or more, preferably 1 to 4, in particular 1, 2 or 3 heteroatoms in the heterocyclic ring, preferably from the group N, O and S, where, however, two oxygen atoms must not be directly adjacent to one another, for example having one heteroatom from the group consisting of N, O and S 1- or 2- or 3-pyrrolidinyl, 3,4-dihydro-2H-pyrrol-2- or -3-yl, 2,3-dihydro-1H-pyrrol-1- or -2- or -3- or -4- or -5-yl; 2,5-dihydro-1H-pyrrol-1- or -2- or -3-yl, 1- or 2- or 3- or 4-piperidinyl; 2,3,4,5-tetrahydropyridin-2- or -3- or -4- or -5-yl or -6-yl; 1,2,3,6-tetrahydropyridin-1- or -2- or -3- or -4- or -5- or -6-yl; 1,2,3,4-tetrahydropyridin-1- or -2- or -3- or -4- or -5- or -6-yl; 1,4-dihydropyridin-1- or -2- or -3- or -4-yl; 2,3-dihydropyridin-2- or -3- or -4- or -5- or -6-yl; 2,5-dihydropyridin-2- or -3- or -4- or -5- or -6-yl, 1- or 2- or 3- or 4-azepanyl; 2,3,4,5-tetrahydro-1H-azepin-1- or -2- or -3- or -4- or -5- or -6- or -7-yl; 2,3,4,7-tetrahydro-1H-azepin-1- or -2- or -3- or -4- or -5- or -6- or -7-yl; 2,3,6,7-tetrahydro-1H-azepin-1- or -2- or -3- or -4-yl; 3,4,5,6-tetrahydro-2H-azepin-2- or -3- or -4- or -5- or -6- or -7-yl; 4,5-dihydro-1H-azepin-1- or -2- or -3- or -4-yl; 2,5-dihydro-1H-azepin-1- or -2- or -3- or -4- or -5- or -6- or -7-yl; 2,7-dihydro-1H-azepin-1- or -2- or -3- or -4-yl; 2,3-dihydro-1H-azepin-1- or -2- or -3- or -4- or -5- or -6- or -7-yl; 3,4-dihydro-2H-azepin-2- or -3- or -4- or -5- or -6- or -7-yl; 3,6-dihydro-2H-azepin-2- or -3- or -4- or -5- or -6- or -7-yl; 5,6-dihydro-2H-azepin-2- or -3- or -4- or -5- or -6- or -7-yl; 4,5-dihydro-3H-azepin-2- or -3- or -4- or -5- or -6- or -7-yl; 1H-azepin-1- or -2- or -3- or -4- or -5- or -6- or -7-yl; 2H-azepin-2- or -3- or -4- or -5- or -6- or -7-yl; 3H-azepin-2- or -3- or -4- or -5- or -6- or -7-yl; 4H-azepin-2- or -3- or -4- or -5- or -6- or -7-yl, 2- or 3-oxolanyl (=2- or 3-tetrahydrofuranyl); 2,3-dihydrofuran-2- or -3- or -4- or -5-yl; 2,5-dihydrofuran-2- or -3-yl, 2- or 3- or 4-oxanyl (=2- or 3- or 4-tetrahydropyranyl); 3,4-dihydro-2H-pyran-2- or -3- or -4- or -5- or -6-yl; 3,6-dihydro-2H-pyran-2- or -3-or -4- or -5- or -6-yl; 2H-pyran-2- or -3- or -4- or -5- or -6-yl; 4H-pyran-2- or -3- or -4-yl, 2- or -3- or -4-oxepanyl; 2,3,4,5-tetrahydrooxepin-2- or -3- or -4- or -5- or -6- or -7-yl; 2,3,4,7-tetrahydrooxepin-2- or -3- or -4- or -5- or -6- or -7-yl; 2,3,6,7-tetrahydrooxepin-2- or -3- or -4-yl; 2,3-dihydrooxepin-2- or -3- or -4- or -5- or -6- or -7-yl; 4,5-dihydrooxepin-2- or -3- or -4-yl; 2,5-dihydrooxepin-2- or -3- or -4- or -5- or -6- or -7-yl; oxepin-2- or -3- or -4- or -5- or -6- or -7-yl; 2- or 3-tetrahydrothiophenyl; 2,3-dihydrothiophen-2- or -3- or -4- or -5-yl; 2,5-dihydrothiophen-2- or -3-yl; tetrahydro-2H-thiopyran-2- or -3- or -4-yl; 3,4-dihydro-2H-thiopyran-2- or -3- or -4- or -5- or -6-yl; 3,6-dihydro-2H-thiopyran-2- or -3- or -4- or -5- or -6-yl; 2H-thiopyran-2- or -3- or -4- or -5- or -6-yl; 4H-thiopyran-2- or -3- or -4-yl. Preferred 3-membered and 4-membered heterocycles are, for example, 1- or 2-aziridinyl, oxiranyl, thiiranyl, 1- or 2- or 3-azetidinyl, 2- or 3-oxetanyl, 2- or 3-thietanyl, 1,3-dioxetan-2-yl. Further examples of “heterocyclyl” are a partly or fully hydrogenated heterocyclic radical having two heteroatoms from the group of N, O and S, for example 1- or 2- or 3- or 4-pyrazolidinyl; 4,5-dihydro-3H-pyrazol-3- or 4- or 5-yl; 4,5-dihydro-1H-pyrazol-1- or 3- or 4- or 5-yl; 2,3-dihydro-1H-pyrazol-1- or 2- or 3- or 4- or 5-yl; 1- or 2- or 3- or 4-imidazolidinyl; 2,3-dihydro-1H-imidazol-1- or 2- or 3- or 4-yl; 2,5-dihydro-1H-imidazol-1- or 2- or 4- or 5-yl; 4,5-dihydro-1H-imidazol-1- or 2- or 4- or 5-yl; hexahydropyridazin-1- or 2- or 3- or 4-yl; 1,2,3,4-tetrahydropyridazin-1- or 2- or 3- or 4- or 5- or 6-yl; 1,2,3,6-tetrahydropyridazin-1- or 2- or 3- or 4- or 5- or 6-yl; 1,4,5,6-tetrahydropyridazin-1- or 3- or 4- or 5- or 6-yl; 3,4,5,6-tetrahydropyridazin-3- or 4- or 5-yl; 4,5-dihydropyridazin-3- or 4-yl; 3,4-dihydropyridazin-3- or 4- or 5- or 6-yl; 3,6-dihydropyridazin-3- or 4-yl; 1,6-dihydropyridazin-1- or 3- or 4- or 5- or 6-yl; hexahydropyrimidin-1- or 2- or 3- or 4-yl; 1,4,5,6-tetrahydropyrimidin-1- or 2- or 4- or 5- or 6-yl; 1,2,5,6-tetrahydropyrimidin-1- or 2- or 4- or 5- or 6-yl; 1,2,3,4-tetrahydropyrimidin-1- or 2- or 3- or 4- or 5- or 6-yl; 1,6-dihydropyrimidin-1- or 2- or 4- or 5- or 6-yl; 1,2-dihydropyrimidin-1- or 2- or 4- or 5- or 6-yl; 2,5-dihydropyrimidin-2- or 4- or 5-yl; 4,5-dihydropyrimidin- 4- or 5- or 6-yl; 1,4-dihydropyrimidin-1- or 2- or 4- or 5- or 6-yl; 1- or 2- or 3-piperazinyl; 1,2,3,6-tetrahydropyrazin-1- or 2- or 3- or 5- or 6-yl; 1,2,3,4-tetrahydropyrazin-1- or 2- or 3- or 4- or 5- or 6-yl; 1,2-dihydropyrazin-1- or 2- or 3- or 5- or 6-yl; 1,4-dihydropyrazin-1- or 2- or 3-yl; 2,3-dihydropyrazin-2- or 3- or 5- or 6-yl; 2,5-dihydropyrazin-2- or 3-yl; 1,3-dioxolan-2- or 4- or 5-yl; 1,3-dioxol-2- or 4-yl; 1,3-dioxan-2- or 4- or 5-yl; 4H-1,3-dioxin-2- or 4- or 5- or 6-yl; 1,4-dioxan-2- or 3- or 5- or 6-yl; 2,3-dihydro-1,4-dioxin-2- or 3- or 5- or 6-yl; 1,4-dioxin-2- or 3-yl; 1,2-dithiolan-3- or 4-yl; 3H-1,2-dithiol-3- or 4- or 5-yl; 1,3-dithiolan-2- or 4-yl; 1,3-dithiol-2- or 4-yl; 1,2-dithian-3- or 4-yl; 3,4-dihydro-1,2-dithiin-3- or 4- or 5- or 6-yl; 3,6-dihydro-1,2-dithiin-3- or 4-yl; 1,2-dithiin-3- or 4-yl; 1,3-dithian-2- or 4- or 5-yl; 4H-1,3-dithiin-2- or 4- or 5- or 6-yl; isoxazolidin-2- or 3- or 4- or 5-yl; 2,3-dihydroisoxazol-2- or 3- or 4- or 5-yl; 2,5-dihydroisoxazol-2- or 3- or 4- or 5-yl; 4,5-dihydroisoxazol-3- or 4- or 5-yl; 1,3-oxazolidin-2- or 3- or 4- or 5-yl; 2,3-dihydro-1,3-oxazol-2- or 3- or 4- or 5-yl; 2,5-dihydro-1,3-oxazol-2- or 4- or 5-yl; 4,5-dihydro-1,3-oxazol-2- or 4- or 5-yl; 1,2-oxazinan-2- or 3- or 4- or 5- or 6-yl; 3,4-dihydro-2H-1,2-oxazin-2- or 3- or 4- or 5- or 6-yl; 3,6-dihydro-2H-1,2-oxazin-2- or 3- or 4- or 5- or 6-yl; 5,6-dihydro-2H-1,2-oxazin-2- or 3- or 4- or 5- or 6-yl; 5,6-dihydro-4H-1,2-oxazin-3- or 4- or 5- or 6-yl; 2H-1,2-oxazin-2- or 3- or 4- or 5- or 6-yl; 6H-1,2-oxazin-3- or 4- or 5- or 6-yl; 4H-1,2-oxazin-3- or 4- or 5- or 6-yl; 1,3-oxazinan-2- or 3- or 4- or 5- or 6-yl; 3,4-dihydro-2H-1,3-oxazin-2- or 3- or 4- or 5- or 6-yl; 3,6-dihydro-2H-1,3-oxazin-2- or 3- or 4- or 5- or 6-yl; 5,6-dihydro-2H-1,3-oxazin-2- or 4- or 5- or 6-yl; 5,6-dihydro-4H-1,3-oxazin-2- or 4- or 5- or 6-yl; 2H-1,3-oxazin-2- or 4- or 5- or 6-yl; 6H-1,3-oxazin-2- or 4- or 5- or 6-yl; 4H-1,3-oxazin-2- or 4- or 5- or 6-yl; morpholin-2- or 3- or 4-yl; 3,4-dihydro-2H-1,4-oxazin-2- or 3- or 4- or 5- or 6-yl; 3,6-dihydro-2H-1,4-oxazin-2- or 3- or 5- or 6-yl; 2H-1,4-oxazin-2- or 3- or 5- or 6-yl; 4H-1,4-oxazin-2- or 3-yl; 1,2-oxazepan-2- or 3- or 4- or 5- or 6- or 7-yl; 2,3,4,5-tetrahydro-1,2-oxazepin-2- or 3- or 4- or 5- or 6- or 7-yl; 2,3,4,7-tetrahydro-1,2-oxazepin-2- or 3- or 4- or 5- or 6- or 7-yl; 2,3,6,7-tetrahydro-1,2-oxazepin-2- or 3- or 4- or 5- or 6- or 7-yl; 2,5,6,7-tetrahydro-1,2-oxazepin-2- or 3- or 4- or 5- or 6- or 7-yl; 4,5,6,7-tetrahydro-1,2-oxazepin-3- or 4- or 5- or 6- or 7-yl; 2,3-dihydro-1,2-oxazepin-2- or 3- or 4- or 5- or 6- or 7-yl; 2,5-dihydro-1,2-oxazepin-2- or 3- or 4- or 5- or 6- or 7-yl; 2,7-dihydro-1,2-oxazepin-2- or 3- or 4- or 5- or 6- or 7-yl; 4,5-dihydro-1,2-oxazepin-3- or 4- or 5- or 6- or 7-yl; 4,7-dihydro-1,2-oxazepin-3- or 4- or 5- or 6- or 7-yl; 6,7-dihydro-1,2-oxazepin-3- or 4- or 5- or 6- or 7-yl; 1,2-oxazepin-3- or 4- or 5- or 6- or 7-yl; 1,3-oxazepan-2- or 3- or 4- or 5- or 6- or 7-yl; 2,3,4,5-tetrahydro-1,3-oxazepin-2- or 3- or 4- or 5- or 6- or 7-yl; 2,3,4,7-tetrahydro-1,3-oxazepin-2- or 3- or 4- or 5- or 6- or 7-yl; 2,3,6,7-tetrahydro-1,3-oxazepin-2- or 3- or 4- or 5- or 6- or 7-yl; 2,5,6,7-tetrahydro-1,3-oxazepin-2- or 4- or 5- or 6- or 7-yl; 4,5,6,7-tetrahydro-1,3-oxazepin-2- or 4- or 5- or 6- or 7-yl; 2,3-dihydro-1,3-oxazepin-2- or 3- or 4- or 5- or 6- or 7-yl; 2,5-dihydro-1,3-oxazepin-2- or 4- or 5- or 6- or 7-yl; 2,7-dihydro-1,3-oxazepin-2- or 4- or 5- or 6- or 7-yl; 4,5-dihydro-1,3-oxazepin-2- or 4- or 5- or 6- or 7-yl; 4,7-dihydro-1,3-oxazepin-2- or 4- or 5- or 6- or 7-yl; 6,7-dihydro-1,3-oxazepin-2- or 4- or 5- or 6- or 7-yl; 1,3-oxazepin-2- or 4- or 5- or 6- or 7-yl; 1,4-oxazepan-2- or 3- or 5- or 6- or 7-yl; 2,3,4,5-tetrahydro-1,4-oxazepin-2- or 3- or 4- or 5- or 6- or 7-yl; 2,3,4,7-tetrahydro-1,4-oxazepin-2- or 3- or 4- or 5- or 6- or 7-yl; 2,3,6,7-tetrahydro-1,4-oxazepin-2- or 3- or 5- or 6- or 7-yl; 2,5,6,7-tetrahydro-1,4-oxazepin-2- or 3- or 5- or 6- or 7-yl; 4,5,6,7-tetrahydro-1,4-oxazepin-2- or 3- or 4- or 5- or 6- or 7-yl; 2,3-dihydro-1,4-oxazepin-2- or 3- or 5- or 6- or 7-yl; 2,5-dihydro-1,4-oxazepin-2- or 3- or 5- or 6- or 7-yl; 2,7-dihydro-1,4-oxazepin-2- or 3- or 5- or 6- or 7-yl; 4,5-dihydro-1,4-oxazepin-2- or 3- or 4- or 5- or 6- or 7-yl; 4,7-dihydro-1,4-oxazepin-2- or 3- or 4- or 5- or 6- or 7-yl; 6,7-dihydro-1,4-oxazepin-2- or 3- or 5- or 6- or 7-yl; 1,4-oxazepin-2- or 3- or 5- or 6- or 7-yl; isothiazolidin-2- or 3- or 4- or 5-yl; 2,3-dihydroisothiazol-2- or 3- or 4- or 5-yl; 2,5-dihydroisothiazol-2- or 3- or 4- or 5-yl; 4,5-dihydroisothiazol-3- or 4- or 5-yl; 1,3-thiazolidin-2- or 3- or 4- or 5-yl; 2,3-dihydro-1,3-thiazol-2- or 3- or 4- or 5-yl; 2,5-dihydro-1,3-thiazol-2- or 4- or 5-yl; 4,5-dihydro-1,3-thiazol-2- or 4- or 5-yl; 1,3-thiazinan-2- or 3- or 4- or 5- or 6-yl; 3,4-dihydro-2H-1,3-thiazin-2- or 3- or 4- or 5- or 6-yl; 3,6-dihydro-2H-1,3-thiazin-2- or 3- or 4- or 5- or 6-yl; 5,6-dihydro-2H-1,3-thiazin-2- or 4- or 5- or 6-yl; 5,6-dihydro-4H-1,3-thiazin-2- or 4- or 5- or 6-yl; 2H-1,3-thiazin-2- or 4- or 5- or 6-yl; 6H-1,3-thiazin-2- or 4- or 5- or 6-yl; 4H-1,3-thiazin-2- or 4- or 5- or 6-yl. Further examples of “heterocyclyl” are a partly or fully hydrogenated heterocyclic radical having 3 heteroatoms from the group of N, O and S, for example 1,4,2-dioxazolidin-2- or -3- or -5-yl; 1,4,2-dioxazol-3- or -5-yl; 1,4,2-dioxazinan-2- or -3- or -5- or -6-yl; 5,6-dihydro-1,4,2-dioxazin-3- or -5- or -6-yl; 1,4,2-dioxazin-3- or -5- or -6-yl; 1,4,2-dioxazepan-2- or -3- or -5- or -6- or -7-yl; 6,7-dihydro-5H-1,4,2-dioxazepin-3- or -5- or -6- or -7-yl; 2,3-dihydro-7H-1,4,2-dioxazepin-2- or -3- or -5- or -6- or -7-yl; 2,3-dihydro-5H-1,4,2-dioxazepin-2- or -3- or -5- or -6- or -7-yl; 5H-1,4,2-dioxazepin-3- or -5- or -6- or -7-yl; 7H-1,4,2-dioxazepin-3- or -5- or -6- or -7-yl. Structural examples of heterocycles which are optionally substituted further are also listed below:

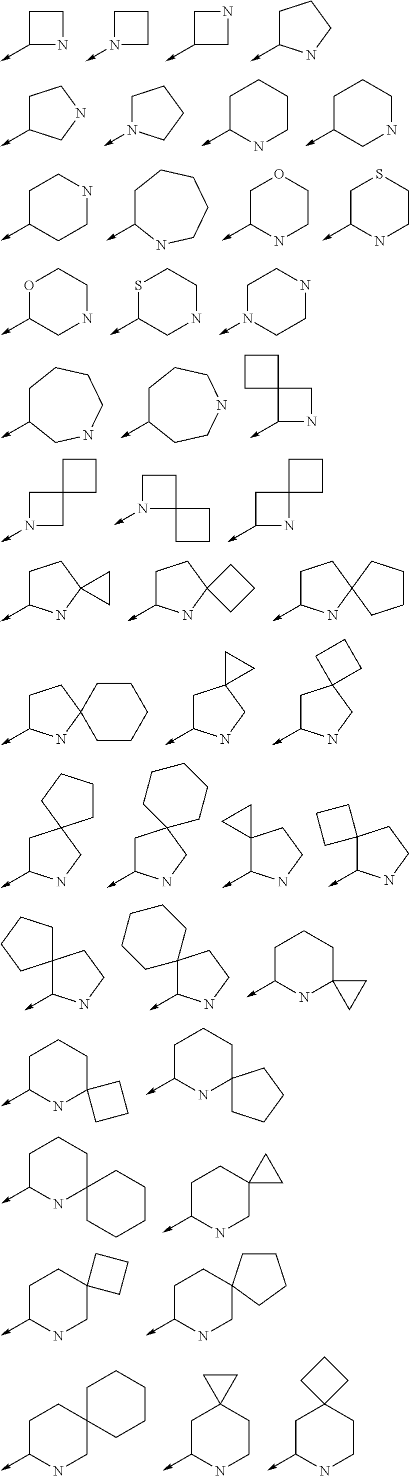

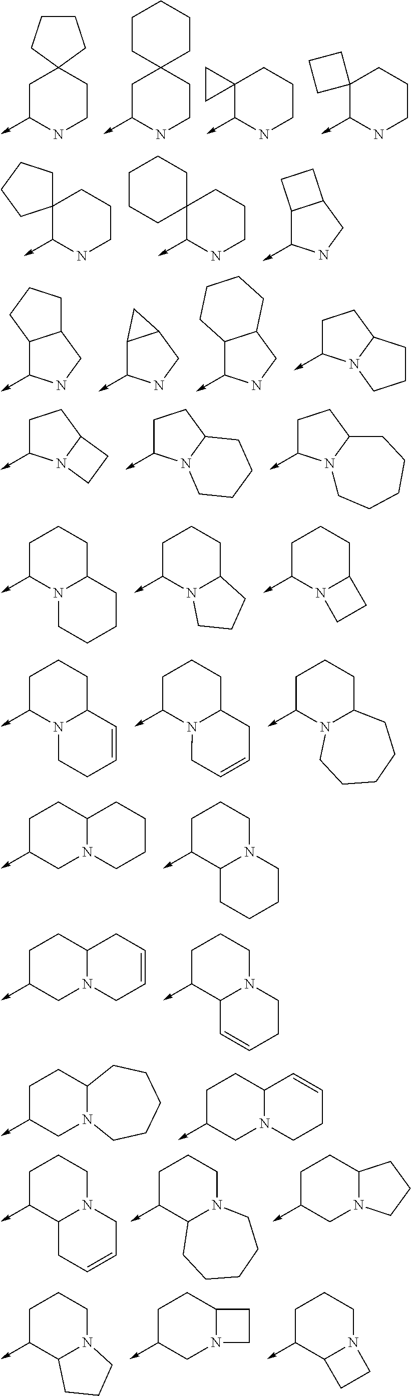

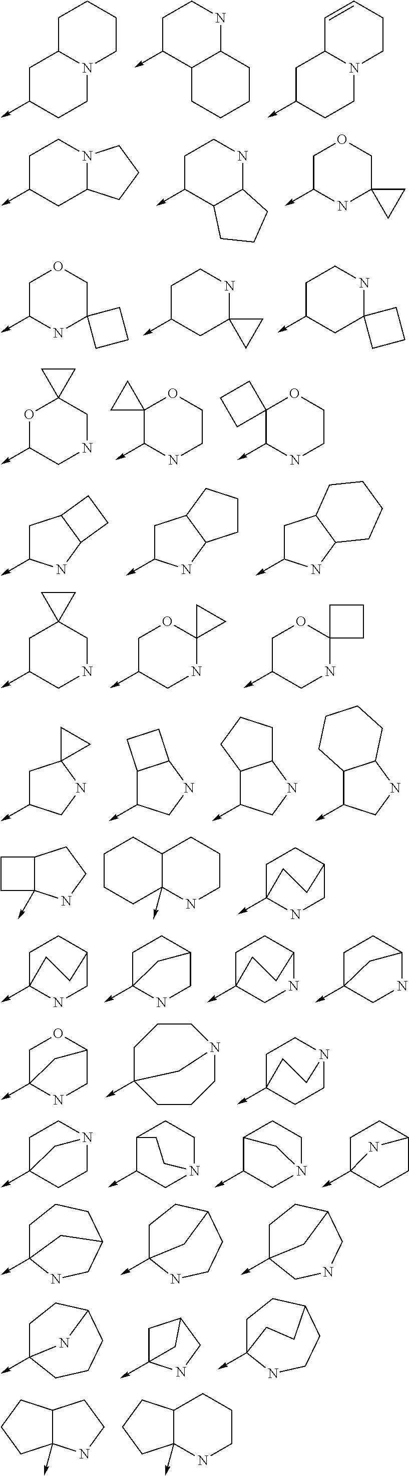

The heterocycles listed above are preferably substituted, for example, by hydrogen, halogen, alkyl, haloalkyl, hydroxyl, alkoxy, cycloalkoxy, aryloxy, alkoxyalkyl, alkoxyalkoxy, cycloalkyl, halocycloalkyl, aryl, arylalkyl, heteroaryl, heterocyclyl, alkenyl, alkylcarbonyl, cycloalkylcarbonyl, arylcarbonyl, heteroarylcarbonyl, alkoxycarbonyl, hydroxycarbonyl, cycloalkoxycarbonyl, cycloalkylalkoxycarbonyl, alkoxycarbonylalkyl, arylalkoxycarbonyl, arylalkoxycarbonylalkyl, alkynyl, alkynylalkyl, alkylalkynyl, trisalkylsilylalkynyl, nitro, amino, cyano, haloalkoxy, haloalkylthio, alkylthio, hydrothio, hydroxyalkyl, oxo, heteroarylalkoxy, arylalkoxy, heterocyclylalkoxy, heterocyclylalkylthio, heterocyclyloxy, heterocyclylthio, heteroaryloxy, bisalkylamino, alkylamino, cycloalkylamino, hydroxycarbonylalkylamino, alkoxycarbonylalkylamino, arylalkoxycarbonylalkylamino, alkoxycarbonylalkyl(alkyl)amino, am inocarbonyl, alkylaminocarbonyl, bisalkylaminocarbonyl, cycloalkylaminocarbonyl, hydroxycarbonylalkylaminocarbonyl, alkoxycarbonylalkylaminocarbonyl, arylalkoxycarbonylalkylaminocarbonyl.

When a base structure is substituted “by one or more radicals” from a list of radicals (=group) or a generically defined group of radicals, this in each case includes simultaneous substitution by a plurality of identical and/or structurally different radicals.

In the case of a partly or fully saturated nitrogen heterocycle, this may be joined to the remainder of the molecule either via carbon or via the nitrogen.

Suitable substituents for a substituted heterocyclic radical are the substituents specified further down, and additionally also oxo and thioxo. The oxo group as a substituent on a ring carbon atom is then, for example, a carbonyl group in the heterocyclic ring. As a result, lactones and lactams are preferably also included. The oxo group may also occur on the ring heteroatoms, which may exist in different oxidation states, for example in the case of N and S, and in that case form, for example, the divalent —N(O)—, —S(O)— (also SO for short) and —S(O)₂— (also SO₂ for short) groups in the heterocyclic ring. In the case of —N(O)— and —S(O)— groups, both enantiomers in each case are included.

According to the invention, the expression “heteroaryl” refers to heteroaromatic compounds, i.e. fully unsaturated aromatic heterocyclic compounds, preferably 5- to 7-membered rings having 1 to 4, preferably 1 or 2, identical or different heteroatoms, preferably O, S or N. Inventive heteroaryls are, for example, 1H-pyrrol-1-yl; 1H-pyrrol-2-yl; 1H-pyrrol-3-yl; furan-2-yl; furan-3-yl; thien-2-yl; thien-3-yl, 1H-imidazol-1-yl; 1H-imidazol-2-yl; 1H-imidazol-4-yl; 1H-imidazol-5-yl; 1H-pyrazol-1-yl; 1H-pyrazol-3-yl; 1H-pyrazol-4-yl; 1H-pyrazol-5-yl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 1H-1,2,4-triazol-1-yl, 1H-1,2,4-triazol-3-yl, 4H-1,2,4-triazol-4-yl, 1,2,4-oxadiazol-3-yl, 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl, azepynyl, pyridin-2-yl, pyridin-3-yl, pyridin-4-yl, pyrazin-2-yl, pyrazin-3-yl, pyrimidin-2-yl, pyrimidin-4-yl, pyrimidin-5-yl, pyridazin-3-yl, pyridazin-4-yl, 1,3,5-triazin-2-yl, 1,2,4-triazin-3-yl, 1,2,4-triazin-5-yl, 1,2,4-triazin-6-yl, 1,2,3-triazin-4-yl, 1,2,3-triazin-5-yl, 1,2,4-, 1,3,2-, 1,3,6- and 1,2,6-oxazynyl, isoxazol-3-yl, isoxazol-4-yl, isoxazol-5-yl, 1,3-oxazol-2-yl, 1,3-oxazol-4-yl, 1,3-oxazol-5-yl, isothiazol-3-yl, isothiazol-4-yl, isothiazol-5-yl, 1,3-thiazol-2-yl, 1,3-thiazol-4-yl, 1,3-thiazol-5-yl, oxepynyl, thiepynyl, 1,2,4-triazolonyl and 1,2,4-diazepynyl, 2H-1,2,3,4-tetrazol-5-yl, 1H-1,2,3,4-tetrazol-5-yl, 1,2,3,4-oxatriazol-5-yl, 1,2,3,4-thiatriazol-5-yl, 1,2,3,5-oxatriazol-4-yl, 1,2,3,5-thiatriazol-4-yl. The heteroaryl groups according to the invention may also be substituted by one or more identical or different radicals. If two adjacent carbon atoms are part of a further aromatic ring, the systems are fused heteroaromatic systems, such as benzofused or polyannulated heteroaromatics. Preferred examples are quinolines (e.g. quinolin-2-yl, quinolin-3-yl, quinolin-4-yl, quinolin-5-yl, quinolin-6-yl, quinolin-7-yl, quinolin-8-yl); isoquinolines (e.g. isoquinolin-1-yl, isoquinolin-3-yl, isoquinolin-4-yl, isoquinolin-5-yl, isoquinolin-6-yl, isoquinolin-7-yl, isoquinolin-8-yl); quinoxaline; quinazoline; cinnoline; 1,5-naphthyridine; 1,6-naphthyridine; 1,7-naphthyridine; 1,8-naphthyridine; 2,6-naphthyridine; 2,7-naphthyridine; phthalazine; pyridopyrazines; pyridopyrimidines; pyridopyridazines; pteridines; pyrimidopyrimidines. Examples of heteroaryl are also 5- or 6-membered benzofused rings from the group of 1H-indol-1-yl, 1H-indol-2-yl, 1H-indol-3-yl, 1H-indol-4-yl, 1H-indol-5-yl, 1H-indol-6-yl, 1H-indol-7-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, 2H-indazol-7-yl, 2H-isoindol-2-yl, 2H-isoindol-1-yl, 2H-isoindol-3-yl, 2H-isoindol-4-yl, 2H-isoindol-5-yl, 2H-isoindol-6-yl; 2H-isoindol-7-yl, 1H-benzimidazol-1-yl, 1H-benzimidazol-2-yl, 1H-benzimidazol-4-yl, 1H-benzimidazol-5-yl, 1H-benzimidazol-6-yl, 1H-benzimidazol-7-yl, 1,3-benzoxazol-2-yl, 1,3-benzoxazol-4-yl, 1,3-benzoxazol-5-yl, 1,3-benzoxazol-6-yl, 1,3-benzoxazol-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,2-benzisoxazol-3-yl, 1,2-benzisoxazol-4-yl, 1,2-benzisoxazol-5-yl, 1,2-benzisoxazol-6-yl, 1,2-benzisoxazol-7-yl, 1,2-benzisothiazol-3-yl, 1,2-benzisothiazol-4-yl, 1,2-benzisothiazol-5-yl, 1,2-benzisothiazol-6-yl, 1,2-benzisothiazol-7-yl.

The term “halogen” denotes, for example, fluorine, chlorine, bromine or iodine. If the term is used for a radical, “halogen” denotes, for example, a fluorine, chlorine, bromine or iodine atom.

According to the invention, “alkyl” means a straight-chain or branched open-chain, saturated hydrocarbon radical which is optionally mono- or polysubstituted, and in the latter case is referred to as “substituted alkyl”. Preferred substituents are halogen atoms, alkoxy, haloalkoxy, cyano, alkylthio, haloalkylthio, amino or nitro groups, particular preference being given to methoxy, methyl, fluoroalkyl, cyano, nitro, fluorine, chlorine, bromine or iodine. The prefix “bis” also includes the combination of different alkyl radicals, e.g. methyl(ethyl) or ethyl(methyl).

“Haloalkyl”, “-alkenyl” and “-alkynyl” respectively denote alkyl, alkenyl and alkynyl partly or fully substituted by identical or different halogen atoms, for example monohaloalkyl such as CH₂CH₂Cl, CH₂CH₂Br, CHClCH₃, CH₂Cl, CH₂F; perhaloalkyl such as CCl₃, CClF₂, CFCl₂, CF₂CClF₂, CF₂CClFCF₃; polyhaloalkyl such as CH₂CHFCl, CF₂CClFH, CF₂CBrFH, CH₂CF₃; the term perhaloalkyl also encompasses the term perfluoroalkyl.

Partly fluorinated alkyl denotes a straight-chain or branched, saturated hydrocarbon which is mono- or polysubstituted by fluorine, where the fluorine atoms in question may be present as substituents on one or more different carbon atoms of the straight-chain or branched hydrocarbon chain, for example CHFCH₃, CH₂CH₂F, CH₂CH₂CF₃, CHF₂, CH₂F, CHFCF₂CF₃.

Partly fluorinated haloalkyl denotes a straight-chain or branched, saturated hydrocarbon which is substituted by different halogen atoms with at least one fluorine atom, where any other halogen atoms optionally present are selected from the group consisting of fluorine, chlorine or bromine, iodine. The corresponding halogen atoms may be present as substituents on one or more different carbon atoms of the straight-chain or branched hydrocarbon chain. Partly fluorinated haloalkyl also includes full substitution of the straight or branched chain by halogen including at least one fluorine atom.

Haloalkoxy is, for example, OCF₃, OCHF₂, OCH₂F, OCF₂CF₃, OCH₂CF₃ and OCH₂CH₂Cl; the situation is equivalent for haloalkenyl and other halogen-substituted radicals.

The expression “(C₁-C₄)-alkyl” mentioned here by way of example is a brief notation for straight-chain or branched alkyl having one to 4 carbon atoms according to the range stated for carbon atoms, i.e. encompasses the methyl, ethyl, 1-propyl, 2-propyl, 1-butyl, 2-butyl, 2-methylpropyl or tert-butyl radicals. General alkyl radicals with a larger specified range of carbon atoms, e.g. “(C₁-C₆)-alkyl”, correspondingly also encompass straight-chain or branched alkyl radicals with a greater number of carbon atoms, i.e. according to the example also the alkyl radicals having 5 and 6 carbon atoms.

Unless stated specifically, preference is given to the lower carbon skeletons, for example having from 1 to 6 carbon atoms, or having from 2 to 6 carbon atoms in the case of unsaturated groups, in the case of the hydrocarbyl radicals such as alkyl, alkenyl and alkynyl radicals, including in composite radicals. Alkyl radicals, including in composite radicals such as alkoxy, haloalkyl, etc., are, for example, methyl, ethyl, n-propyl or i-propyl, n-, i-, t- or 2-butyl, pentyls, hexyls such as n-hexyl, i-hexyl and 1,3-dimethylbutyl, heptyls such as n-heptyl, 1-methylhexyl and 1,4-dimethylpentyl; alkenyl and alkynyl radicals are defined as the possible unsaturated radicals corresponding to the alkyl radicals, where at least one double bond or triple bond is present. Preference is given to radicals having one double bond or triple bond.

The term “alkenyl” also includes, in particular, straight-chain or branched open-chain hydrocarbon radicals having more than one double bond, such as 1,3-butadienyl and 1,4-pentadienyl, but also allenyl or cumulenyl radicals having one or more cumulated double bonds, for example allenyl (1,2-propadienyl), 1,2-butadienyl and 1,2,3-pentatrienyl. Alkenyl denotes, for example, vinyl which may optionally be substituted by further alkyl radicals, for example (but not limited thereto) (C₂-C₆)-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “alkynyl” also includes, in particular, straight-chain or branched open-chain hydrocarbon radicals having more than one triple bond, or else having one or more triple bonds and one or more double bonds, for example 1,3-butatrienyl or 3-penten-1-yn-1-yl. (C₂-C₆)-Alkynyl is, for example, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl.

The term “cycloalkyl” refers to a carbocyclic saturated ring system having preferably 3-8 ring carbon atoms, for example cyclopropyl, cyclobutyl, cyclopentyl or cyclohexyl, which optionally has further substitution, preferably by hydrogen, alkyl, alkoxy, cyano, nitro, alkylthio, haloalkylthio, halogen, alkenyl, alkynyl, haloalkyl, amino, alkylamino, bisalkylamino, alkoxycarbonyl, hydroxycarbonyl, arylalkoxycarbonyl, aminocarbonyl, alkylaminocarbonyl, cycloalkylaminocarbonyl. In the case of optionally substituted cycloalkyl, cyclic systems with substituents are included, also including substituents with a double bond on the cycloalkyl radical, for example an alkylidene group such as methylidene. In the case of optionally substituted cycloalkyl, polycyclic aliphatic systems are also included, for example bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl and adamantan-2-yl, but also systems such as 1,1′-bi(cyclopropyl)-1-yl, 1,1′-bi(cyclopropyl)-2-yl, for example. The term “(C₃-C₇)-cycloalkyl” is a brief notation for cycloalkyl having three to 7 carbon atoms, corresponding to the range specified for carbon atoms.

In the case of substituted cycloalkyl, spirocyclic aliphatic systems are also included, for example spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl.

“Cycloalkenyl” denotes a carbocyclic, nonaromatic, partly unsaturated ring system having preferably 4-8 carbon atoms, e.g. 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, also including substituents with a double bond on the cycloalkenyl radical, for example an alkylidene group such as methylidene. In the case of optionally substituted cycloalkenyl, the elucidations for substituted cycloalkyl apply correspondingly.

The term “alkylidene”, also, for example, in the form (C₁-C₁₀)-alkylidene, means the radical of a straight-chain or branched open-chain hydrocarbon radical which is attached via a double bond. Possible bonding sites for alkylidene are naturally only positions on the base structure where two hydrogen atoms can be replaced by the double bond; radicals are, for example, ═CH₂, ═CH—CH₃, ═C(CH₃)—CH₃, ═C(CH₃)—C₂H₅ or ═C(C₂H₅)—C₂H₅ Cycloalkylidene denotes a carbocyclic radical attached via a double bond.

If the compounds can form, through a hydrogen shift, tautomers whose structure is not formally covered by the formula (I), these tautomers are nevertheless covered by the definition of the inventive compounds of the formula (I), unless a particular tautomer is under consideration. For example, many carbonyl compounds may be present both in the keto form and in the enol form, both forms being encompassed by the definition of the compound of the formula (I).

Depending on the nature of the substituents and the manner in which they are attached, the compounds of the general formula (I) may be present as stereoisomers. The formula (I) embraces all possible stereoisomers defined by the specific three-dimensional form thereof, such as enantiomers, diastereomers, Z and E isomers. If, for example, one or more alkenyl groups are present, diastereomers (Z and E isomers) may occur. If, for example, one or more asymmetric carbon atoms are present, enantiomers and diastereomers may occur. Stereoisomers can be obtained from the mixtures obtained in the preparation by customary separation methods. The chromatographic separation can be effected either on the analytical scale to find the enantiomeric excess or the diastereomeric excess, or else on the preparative scale to produce test specimens for biological testing. It is likewise possible to selectively prepare stereoisomers by using stereoselective reactions with use of optically active starting materials and/or auxiliaries. The invention thus also relates to all stereoisomers which are embraced by the general formula (I) but are not shown in their specific stereomeric form, and to mixtures thereof.

Synthesis of substituted aryl- and heteroarylcarbonyl hydrazides:

Substituted aryl- and heteroarylcarbonyl hydrazides of the general formula (I) can be prepared by known methods (cf. Tetrahedron 2003, 59, 7733; J. Organomet. Chem. 2001, 617; J. Org. Chem. 1962, 27, 2640; IT2000MI0292; J. Heterocyclic Chem. 1981, 18, 319). Various literature preparation routes were used to form the core structure, and some were optimized (see Scheme 1). Selected detailed synthesis examples are cited in the next section. The synthesis routes used and examined for preparation of substituted aryl- and heteroarylcarbonyl hydrazides proceed from aryl- and heteroarylcarboxylic acids that are commercially available or readily preparable using synthesis routes described in the literature, or the corresponding aryl- and heteroarylcarbonyl chlorides. The optionally further-substituted aryl- or heteroarylcarboxylic acid in question is converted here with the aid of a suitable chlorinating agent (e.g. oxalyl chloride or thionyl chloride) in an aprotic solvent (e.g.

toluene) to the corresponding aryl- or heteroarylcarbonyl chloride if the latter is not commercially available, and then the latter is reacted with an appropriately 1,1′-disubstituted hydrazine using a suitable base (e.g. triethylamine (Et₃N), diisopropylethylamine) in a suitable polar aprotic solvent (e.g. tetrahydrofuran (THF) or dichloromethane (DCM)) to give an aryl- or heteroarylcarbonyl hydrazide (A). The optionally further-substituted aryl- or heteroarylcarbonyl hydrazide (A) can also be prepared by a direct coupling, mediated by suitable reagents (e.g. 1-hydroxybenzotriazole (HOBt), N-(3-dimethylaminopropyl)-N′-ethylcarbodiimide hydrochloride (EDC) or 2,4,6-tripropyl-1,3,5,2,4,6-trioxatriphosphorinane2,4,6-trioxide (T3P), together with a suitable base, for example triethylamine or diisopropylethylamine), of an optionally further-substituted aryl- or heteroarylcarboxylic acid with an appropriately 1,1′-disubstituted hydrazine in a suitable polar aprotic solvent (e.g. dichloromethane, acetonitrile or tetrahydrofuran). In the next step, the optionally further-substituted aryl- or heteroarylcarbonyl hydrazide (A) is converted using an appropriately substituted arylalkyl halide or heteroarylalkyl halide and a suitable base (e.g. sodium hydride or triethylamine (Et₃N)) in a suitable solvent (e.g. tetrahydrofuran, N,N-dimethylformamide (DMF) or dichloromethane) to a substituted aryl- or heteroarylcarbonyl hydrazide (I) (Scheme 1). R¹, R³, R⁴, R⁵ and R⁶ and also A¹, A², A³, A⁴, A⁵, X¹, X², X³ and Y are as defined above and W, by way of example but without restriction, in Scheme 1 below is oxygen.

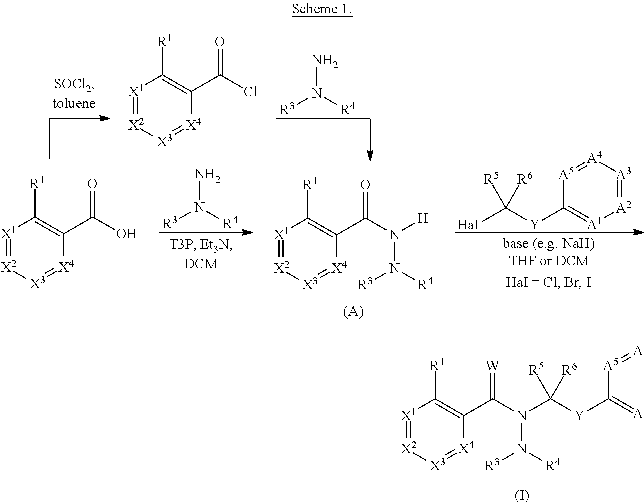

Substituted aryl- or heteroarylcarbonyl hydrazides of the general formula (I) can alternatively also be prepared via the reduction of hydrazones (B) when the R⁶ radical is hydrogen. In the course of this synthesis sequence, an appropriately substituted aldehyde or an appropriately substituted ketone is reacted with an appropriately 1,1′-disubstituted hydrazine to give the hydrazone intermediate (B), then converted with the aid of a suitable reagent (e.g. triethylsilane, BH₃, sodium cyanoborohydride, sodium borohydride or hydrogen on palladium/carbon) in a suitable solvent to the corresponding hydrazine intermediate (C), which, in the final reaction step, is converted with an optionally further-substituted aryl- or heteroarylcarbonyl chloride in a suitable polar aprotic solvent (e.g. tetrahydrofuran or dichloromethane) to a substituted aryl- or heteroarylcarbonyl hydrazide (I) (Scheme 2). R¹, R³, R⁴ and R⁵ and also A¹, A², A³, A⁴, A⁵, X¹, X², X³ and Y are as defined above and W, by way of example but without restriction, in Scheme 2 below is oxygen.

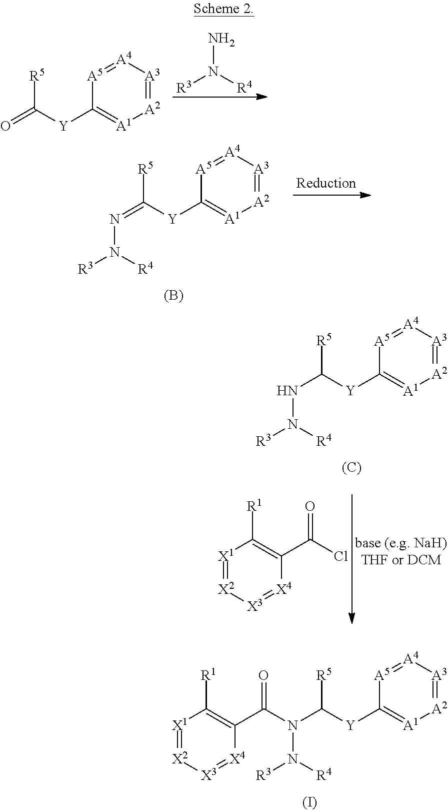

Aryl- or heteroarylcarbonyl hydrazides (I) having unsymmetric substitution on the second hydrazide nitrogen (NR³R⁴) can also be prepared using tert-butyl 1-methylhydrazinecarboxylate (Scheme 3). The optionally further-substituted aryl- or heteroarylcarboxylic acid in question is converted here with the aid of a suitable chlorinating agent (e.g. oxalyl chloride or thionyl chloride) in an aprotic solvent (e.g. toluene) to the corresponding aryl- or heteroarylcarbonyl chloride if the latter is not commercially available, and then the latter is reacted with tert-butyl 1-methylhydrazinecarboxylate using a suitable base (e.g. triethylamine (Et₃N), diisopropylethylamine) in a suitable polar aprotic solvent (e.g. tetrahydrofuran (THF) or dichloromethane (DCM)) to give the aryl- or heteroarylcarbonyl hydrazide (D). The optionally further-substituted aryl- or heteroarylcarbonyl hydrazide (D) can also be prepared by a direct coupling, mediated by suitable reagents (e.g. 1-hydroxybenzotriazole (HOBt), N-(3-dimethylaminopropyl)-N′-ethylcarbodiimide hydrochloride (EDC) or 2,4,6-tripropyl-1,3,5,2,4,6-trioxatriphosphorinane 2,4,6-trioxide (T3P), together with a suitable base, for example triethylamine or diisopropylethylamine), of an optionally further-substituted aryl- or heteroarylcarboxylic acid with tert-butyl 1-methylhydrazinecarboxylate in a suitable polar aprotic solvent (e.g. dichloromethane, acetonitrile or tetrahydrofuran). In the next step, the optionally further-substituted heteroarylcarbonyl hydrazide (D) is converted using an appropriately substituted arylalkyl halide or heteroarylalkyl halide and a suitable base (e.g. sodium hydride or triethylamine (Et3N)) in a suitable solvent (e.g. tetrahydrofuran, N,N-dimethylformamide (DMF) or dichloromethane) to a substituted aryl- or heteroarylcarbonyl hydrazide (If) (Scheme 3). The tert-butyloxycarbonyl protecting group can then be converted by treatment of the substituted aryl- or heteroarylcarbonyl hydrazide (If) with a suitable acid (e.g. trifluoroacetic acid (TFA)) in a polar aprotic solvent (e.g. dichloromethane) to give the N-monosubstituted aryl- or heteroarylcarbonyl hydrazide (Ie). With the aid of a suitable alkyl halide (e.g. ethyl iodide in Scheme 3 below), using a suitable base (e.g. sodium hydride or potassium carbonate) in a suitable solvent, the N-monosubstituted aryl- or heteroarylcarbonyl hydrazide (Ie) in question can be converted to an optionally further-substituted aryl- or heteroarylcarbonyl hydrazide of the invention, by way of example but without restriction (Ib) in Scheme 3 below, having unsymmetric groups on the second hydrazide nitrogen. R¹, R⁵ and R⁶ and also A¹, A², A³, A⁴, A⁵, X¹, X², X³ and Y are as defined above, R³, by way of example but without restriction, in Scheme 3 below is methyl and W, by way of example but without restriction, is oxygen.

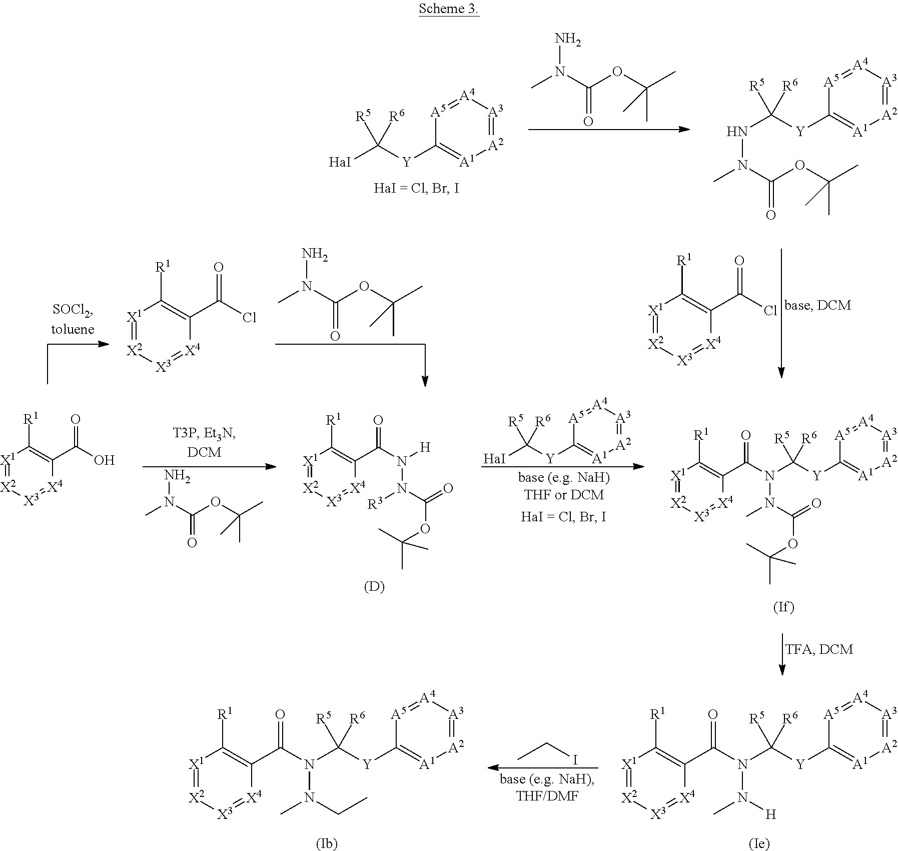

Substituted N-Boc-protected aryl- or heteroarylcarbonyl hydrazides (If) can also be prepared proceeding from a reaction of an optionally substituted arylalkyl halide or heteroarylalkyl halide with tert-butyl 1-methylhydrazinecarboxylate using a suitable base in a suitable polar aprotic solvent (e.g. dichloromethane, acetonitrile or tetrahydrofuran).

Selected detailed synthesis examples for the inventive compounds of the general formula (I) are given below. The example numbers mentioned correspond to the numbering scheme in Tables A¹ to H1 below. The ¹H NMR,¹³C NMR and ¹⁹F NMR spectroscopy data reported for the chemical examples described in the sections which follow (400 MHz for ¹H NMR and 150 MHz for ¹³NMR and 375 MHz for ¹⁹F NMR, solvent CDCl₃, CD₃OD or d₆-DMSO, internal standard: tetramethylsilane δ=0.00 ppm) were obtained on a Bruker instrument, and the signals listed have the meanings given below: br=broad; s=singlet, d=doublet, t=triplet, dd=doublet of doublets, ddd=doublet of a doublet of doublets, m=multiplet, q=quartet, quint=quintet, sext=sextet, sept=septet, dq=doublet of quartets, dt=doublet of triplets. In the case of diastereomer mixtures, either the significant signals for each of the two diastereomers are reported or the characteristic signal of the main diastereomer is reported. The abbreviations used for chemical groups have, for example, the following meanings: Me=CH₃, Et=CH₂CH₃, t-Hex=C(CH₃)₂CH(CH₃)₂, t-Bu=C(CH₃)₃, n-Bu=unbranched butyl, n-Pr=unbranched propyl, i-Pr=branched propyl, c-Pr=cyclopropyl, c-Hex=cyclohexyl.

### SYNTHESIS EXAMPLES

### No. A1-38: N′,N′-Dimethyl-N-[(3-methylpyridin-2-yl)methyl]-2-(trifluoromethyl)benzohydrazide

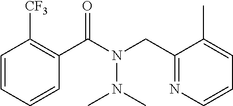 [SMILES: Cc1cccnc1CN(C(=O)c1ccccc1C(F)(F)F)N(C)C]

2-(Trifluoromethyl)benzoyl chloride (500 mg, 2.39 mmol) was dissolved in abs. dichloromethane (5 ml), and triethylamine (0.40 ml, 0.29 mmol) was added. After stirring at room temperature for 5 minutes and then cooling the reaction solution to 0° C., a solution of N,N-dimethylhydrazine (144 mg, 2.39 mmol) in abs. dichloromethane (5 ml) was added dropwise. The resulting reaction mixture was stirred at room temperature for a further 3 h, and then water, sat. sodium hydrogencarbonate solution and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N′,N′-dimethyl-2-(trifluoromethyl)benzohydrazide in the form of a colorless solid (405 mg, 69% of theory). ¹H-NMR (400 MHz, CDCl₃ δ, ppm) 7.71 (m, 1H), 7.61-7.52 (m, 3H), 6.38 (br. s, 1H, NH), 2.71 (m, 2H). N′,N′-Dimethyl-2-(trifluoromethyl)benzohydrazide (150 mg, 0.65 mmol) was dissolved in abs. tetrahydrofuran (5 ml) under argon, and sodium hydride (28 mg, 0.71 mmol, 60% dispersion) was added at room temperature. Stirring at room temperature for 30 minutes was followed by the addition of 2-(chloromethyl)-3-methylpyridine (91 mg, 0.65 mmol), and the resulting reaction mixture was stirred under reflux conditions for 3 hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N′,N′-dimethyl-N-[(3-methylpyridin-2-yl)methyl]-2-(trifluoromethyl)benzohydrazide in the form of a colorless solid (193 mg, 89% of theory). ¹H-NMR (400 MHz, d₆-DMSO δ, ppm) 8.51 (d, 1H), 7.62-7.55 (m, 3H), 7.50-7.47 (m, 1H), 7.38-7.34 (m, 1H), 7.27-7.24 (m, 1H), 5.21 (s, 2H), 3.53 (s, 6H), 2.58 (s, 3H).

### No. A1-40: N-[(3,4-Dichloropyridin-2-yl)methyl]-N′,N′-dimethyl-2-(trifluoromethyl)benzohydrazide

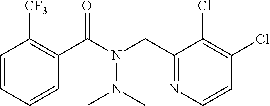 [SMILES: CN(C)N(Cc1nccc(Cl)c1Cl)C(=O)c1ccccc1C(F)(F)F]

2-(Trifluoromethyl)benzoyl chloride (500 mg, 2.39 mmol) was dissolved in abs. dichloromethane (5 ml), and triethylamine (0.40 ml, 0.29 mmol) was added. After stirring at room temperature for 5 minutes and then cooling the reaction solution to 0° C., a solution of N,N-dimethylhydrazine (144 mg, 2.39 mmol) in abs. dichloromethane (5 ml) was added dropwise. The resulting reaction mixture was stirred at room temperature for a further 3 h, and then water, sat. sodium hydrogencarbonate solution and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N′,N′-dimethyl-2-(trifluoromethyl)benzohydrazide in the form of a colorless solid (405 mg, 69% of theory). ¹H-NMR (400 MHz, CDCl₃ δ, ppm) 7.71 (m, 1H), 7.61-7.52 (m, 3H), 6.38 (br. s, 1H, NH), 2.71 (m, 2H). N′,N′-Dimethyl-2-(trifluoromethyl)benzohydrazide (350 mg, 1.51 mmol) was dissolved in abs. N,N-dimethylformamide (5 ml) under argon, and potassium carbonate (250 mg, 1.81 mmol) was added at room temperature. Stirring at room temperature for 30 minutes was followed by the addition of 3,4-dichloro-2-(chloromethyl)pyridine (296 mg, 1.51 mmol), and the resulting reaction mixture was stirred at a temperature of 80° C. for 14 hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N-[(3,4-dichloropyridin-2-yl)methyl]-N′,N′-dimethyl-2-(trifluoromethyl)benzohydrazide in the form of a colorless solid (301 mg, 50% of theory). ¹H-NMR (400 MHz, d₆-DMSO δ, ppm) 8.49 (d, 1H), 7.63-7.59 (m, 2H), 7.51-7.48 (m, 2H), 7.39-7.35 (m, 1H), 5.47 (s, 2H), 3.56 (s, 6H).

### No. A1-94: N-[(6-Chloropyridin-3-yl)methyl]-N′,N′-dimethyl-2-(trifluoromethyl)benzohydrazide

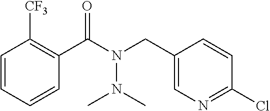 [SMILES: CN(C)N(Cc1ccc(Cl)nc1)C(=O)c1ccccc1C(F)(F)F]

N′,N′-Dimethyl-2-(trifluoromethyl)benzohydrazide (100 mg, 0.43 mmol) was dissolved in a mixture of abs. acetone and abs. N,N-dimethylformamide (20:1, 10.5 ml) under argon, and potassium carbonate (119 mg, 0.86 mmol) was added at room temperature. Stirring at room temperature for 30 minutes was followed by the addition of 2-chloro-5-(chloromethyl)pyridine (84 mg, 0.52 mmol), and the resulting reaction mixture was stirred under reflux conditions for six hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N-[(6-chloropyridin-3-yl)methyl]-N′,N′-dimethyl-2-(trifluoromethyl)benzohydrazide in the form of a colorless solid (76 mg, 49% of theory). ¹H-NMR (400 MHz, CDCl₃ δ, ppm) 8.51 (m, 1H), 8.00 (m, 1H), 7.62 (m, 1H), 7.49-7.39 (m, 3H), 5.11 (s, 2H), 3.47 (s, 6H).

### No. A1-121: N-(Isoquinolin-3-ylmethyl)-N′,N′-dimethyl-2-(trifluoromethyl)benzohydrazide

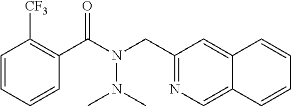 [SMILES: CN(C)N(Cc1cc2ccccc2cn1)C(=O)c1ccccc1C(F)(F)F]

N′,N′-Dimethyl-2-(trifluoromethyl)benzohydrazide (150 mg, 0.65 mmol) was dissolved in abs. tetrahydrofuran (7 ml) under argon, and sodium hydride (28 mg, 0.71 mmol, 60% dispersion) was added at room temperature. Stirring at room temperature for 30 minutes was followed by the addition of 3-(chloromethyl)isoquinoline (115 mg, 0.65 mmol), and the resulting reaction mixture was stirred under reflux conditions for two-and-a-half hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N-[(isoquinolin-3-Amethyl]-N′,N′-dimethyl-2-(trifluoromethyl)benzohydrazide in the form of a colorless solid (128 mg, 53% of theory). ¹H-NMR (400 MHz, d₆-DMSO δ, ppm) 8.27 (m, 1H), 8.10 (m, 1H), 7.88 (m, 1H), 7.82 (m, 1H), 7.78-7.75 (m, 1H), 7.64-7.61 (m, 2H), 7.45-7.42 (m, 2H), 7.38-7.34 (m, 1H), 5.35 (s, 2H), 3.56 (s, 6H).

### No. A1-159: N-[1-(4-Fluorophenypethyl]-N′,N′-dimethyl-2-(trifluoromethyl)benzohydrazide

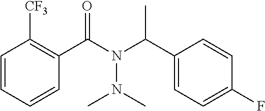 [SMILES: CC(c1ccc(F)cc1)N(C(=O)c1ccccc1C(F)(F)F)N(C)C]

N′,N′-Dimethyl-2-(trifluoromethyl)benzohydrazide (100 mg, 0.43 mmol) was dissolved in a mixture of abs. acetone and abs. N,N-dimethylformamide (20:1, 10.5 ml) under argon, and potassium carbonate (119 mg, 0.86 mmol) and catalytic amounts of potassium iodide (7 mg) were added at room temperature. Stirring at room temperature for 30 minutes was followed by the addition of 1-(1-chloroethyl)-4-fluorobenzene (82 mg, 0.52 mmol), and the resulting reaction mixture was stirred under reflux conditions for six hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N-[1-(4-fluorophenypethyl]-N′,N′-dimethyl-2-(trifluoromethyl)benzohydrazide in the form of a colorless solid (66 mg, 43% of theory). ¹H-NMR (400 MHz, CDCl₃ δ, ppm) 7.68 (m, 1H), 7.38-7.32 (m, 3H), 7.12 (m, 2H), 7.03 (m, 2H), 4.89 (m, 1H), 3.40 (s, 3H), 3.18 (s, 3H), 1.83/1.49 (d, 3H).

### No. A1-454: N-[3-(4-Chlorophenyl)propyl]-N′,N′-dimethyl-2-(trifluoromethyl)benzohydrazide

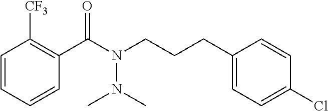 [SMILES: CN(C)N(CCCc1ccc(Cl)cc1)C(=O)c1ccccc1C(F)(F)F]

N′,N′-Dimethyl-2-(trifluoromethyl)benzohydrazide (200 mg, 0.86 mmol) was dissolved in abs. tetrahydrofuran (10 ml) under argon, and sodium hydride (38 mg, 0.95 mmol, 60% dispersion) was added at room temperature. Stirring at room temperature for 30 minutes was followed by the addition of 3-(4-chlorophenyl)propyl bromide (201 mg, 0.86 mmol), and the resulting reaction mixture was stirred under reflux conditions for two-and-a-half hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N-[3-(4-chlorophenyl)propyl]-N′,N′-dimethyl-2-(trifluoromethyl)benzohydrazide in the form of a viscous oil (137 mg, 41% of theory). ¹H-NMR (400 MHz, CDCl₃ δ, ppm) 7.62 (m, 1H), 7.53 (m, 1H), 7.47 (m, 1H), 7.37 (m, 1H), 7.28 (d, 2H), 7.14 (d, 2H), 3.76-3.71 (m, 2H), 3.40 (s, 6H), 2.70 (m, 2H, 2.19 (m, 2H).

### No. A1-462: N-[3-(2,6-Difluorophenyl)propyl]-N′,N′-dimethyl-2-(trifluoromethyl)benzohydrazide

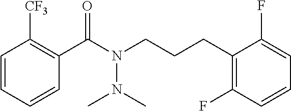 [SMILES: CN(C)N(CCCc1c(F)cccc1F)C(=O)c1ccccc1C(F)(F)F]

N′,N′-Dimethyl-2-(trifluoromethyl)benzohydrazide (300 mg, 1.29 mmol) was dissolved in abs. N,N-dimethylformamide (10 ml) under argon, and sodium hydride (62 mg, 1.55 mmol, 60% dispersion) was added at room temperature. Stirring at room temperature for 30 minutes was followed by the addition of 3-(2,6-difluorophenyl)propyl bromide (304 mg, 1.29 mmol), and the resulting reaction mixture was stirred under reflux conditions for two-and-a-half hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N-[3-(2,6-difluorophenyl)propyl]-N′,N′-dimethyl-2-(trifluoromethyl)benzohydrazide in the form of a viscous oil (189 mg, 38% of theory). ¹H-NMR (400 MHz, CDCl₃ δ, ppm) 7.68 (m, 1H), 7.57-7.53 (m, 3H), 7.23-7.18 (m, 1H), 6.92-6.87 (m, 2H), 4.07-4.02 (m, 2H), 3.72 (s, 6H), 2.83 (m, 2H), 2.27-2.21 (m, 2H).

### No. A2-1: N-(2-Chlorobenzyl)-N′,N′-diethyl-2-(trifluoromethyl)benzohydrazide

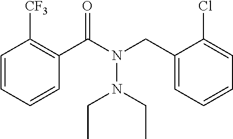 [SMILES: CCN(CC)N(Cc1ccccc1Cl)C(=O)c1ccccc1C(F)(F)F]

2-(Trifluoromethyl)benzoic acid (740 mg, 3.89 mmol) was dissolved in abs. dichloromethane (5 ml), and triethylamine (1.63 ml, 11.67 mmol) was added. After stirring at room temperature for 5 minutes, N,N-diethylhydrazine (412 mg, 4.67 mmol) and 2,4,6-tripropyl-1,3,5,2,4,6-trioxatriphosphorinane 2,4,6-trioxide (3.47 ml, 5.84 mmol, 50% solution in tetrahydrofuran) were added. The resulting reaction mixture was stirred at room temperature for a further 30 minutes, and then water, sat. sodium hydrogencarbonate solution and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N′,N′-diethyl-2-(trifluoromethyl)benzohydrazide in the form of a colorless solid (900 mg, 84% of theory). ¹H-NMR (400 MHz, CDCl₃ δ, ppm) 7.71 (m, 1H), 7.61-7.54 (m, 3H), 6.23 (br. s, 1H, NH), 3.14 (m, 2H), 2.89 (m, 2H), 1.33 (m, 3H), 1.21 (m, 3H). N′,N′-Diethyl-2-(trifluoromethyl)benzohydrazide (150 mg, 0.58 mmol) was dissolved in abs. tetrahydrofuran (10 ml) under argon, and sodium hydride (25 mg, 0.63 mmol, 60% purity) was added at room temperature. Stirring at room temperature for 30 minutes was followed by the addition of 2-chlorobenzyl chloride (93 mg, 0.58 mmol), and the resulting reaction mixture was stirred under reflux conditions for 4 hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N-(2-chlorobenzyl)-N′,N′-diethyl-2-(trifluoromethyl)benzohydrazide in the form of a colorless viscous oil (93 mg, 42% of theory). ¹H-NMR (400 MHz, d₆-DMSO δ, ppm) 7.85 (m, 1H), 7.64 (m, 1H), 7.59-7.56 (m, 2H), 7.52-7.41 (m, 4H), 5.12 (s, 2H), 3.85-3.78 (m, 2H), 3.57-3.48 (m, 2H), 1.26 (t, 6H).

### No. A2-4: N-(2,3-Dichlorobenzyl)-N′,N′-diethyl-2-(trifluoromethyl)benzohydrazide

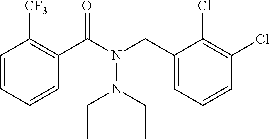 [SMILES: CCN(CC)N(Cc1cccc(Cl)c1Cl)C(=O)c1ccccc1C(F)(F)F]

N′,N′-Diethyl-2-(trifluoromethyl)benzohydrazide (300 mg, 1.15 mmol) was dissolved in abs. ethanol (10 ml) under argon, and potassium carbonate (239 mg, 1.73 mmol) was added at room temperature. Stirring at room temperature for 15 minutes was followed by the addition of 2,3-dichlorobenzyl chloride (332 mg, 1.36 mmol), and the resulting reaction mixture was stirred under reflux conditions for 8 hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product via preparative HPLC gave N-(2,3-dichlorobenzyl)-N′,N′-diethyl-2-(trifluoromethyl)benzohydrazide in the form of a colorless viscous oil (79 mg, 16% of theory). ¹H-NMR (400 MHz, CDCl₃ δ, ppm) 7.84 (m, 1H), 7.68 (m, 1H), 7.65-7.41 (m, 4H), 7.32 (m, 1H), 5.41 (s, 2H), 4.11-4.02 (m, 2H), 3.83-3.75 (m, 2H), 1.45 (t, 6H).

### No. A30-462: 2-(Difluoromethyl)-N-[3-(2,6-difluorophenyl)propyl]-N′,N′-dimethylbenzohydrazide

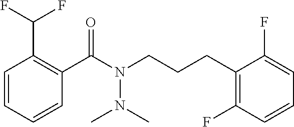 [SMILES: CN(C)N(CCCc1c(F)cccc1F)C(=O)c1ccccc1C(F)F]

2-(Difluoromethyl)benzoic acid (1 equiv.) was dissolved in abs. dichloromethane (10 ml/mmol), and triethylamine (3 equiv.) was added. After stirring at room temperature for 5 minutes, N,N-dimethylhydrazine (1.3 equiv.) and 2,4,6-tripropyl-1,3,5,2,4,6-trioxatriphosphorinane 2,4,6-trioxide (1.5 equiv., 50% solution in tetrahydrofuran) were added. The resulting reaction mixture was stirred at room temperature for a further 30 minutes, and then water, sat. sodium hydrogencarbonate solution and dichloromethane were added. The aqueous phase was repeatedly extracted vigorously with dichloromethane, and the combined organic phases were then dried over magnesium sulfate, filtered and concentrated. Final purification of the resulting crude product by column chromatography gave 2-(difluoromethyl)-N′,N′-dimethylbenzohydrazide in the form of a colorless solid. 2-(Difluoromethyl)-N′,N′-dimethylbenzohydrazide (250 mg, 1.17 mmol) was then dissolved in abs. N,N-dimethylformamide (10 ml) under argon, and sodium hydride (75 mg, 1.87 mmol, 60% purity) was added at room temperature. Stirring at room temperature for 15 minutes was followed by the addition of 3-(2,6-difluorophenyl)propyl methanesulfonate (380 mg, 1.52 mmol), and the resulting reaction mixture was stirred under reflux conditions for two hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave 2-(difluoromethyl)-N-[3-(2,6-difluorophenyl)-propyl]-N′,N′-dimethylbenzohydrazide in the form of a colorless viscous oil (118 mg, 26% of theory). ¹H-NMR (400 MHz, CDCl₃ δ, ppm) 7.69 (m, 2H), 7.54 (m, 1H), 7.48 (m, 1H), 7.27-7.00 (br. t, 1H, CHF2), 7.22-7.16 (m, 1H), 6.91-6.86 (m, 2H), 4.06-4.01 (m, 2H), 3.70 (s, 6H), 2.86-2.81 (m, 2H), 2.23-2.19 (m, 2H).

### No. A50-36: N-[3-Chloro-5-(trifluoromethyl)pyridin-2-yl-methyl]-N′,N′-dimethyl-2-(trifluoromethyl)-4-methylbenzohydrazide

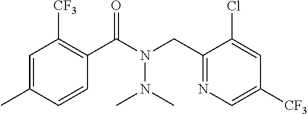 [SMILES: Cc1ccc(C(=O)N(Cc2ncc(C(F)(F)F)cc2Cl)N(C)C)c(C(F)(F)F)c1]

2-(Trifluoromethyl)-4-methylbenzoic acid (500 mg, 2.45 mmol) was dissolved in abs. tetrahydrofuran (15 ml), and triethylamine (1.02 ml, 7.35 mmol) was added. After stirring at room temperature for 5 minutes, N,N-dimethylhydrazine (177 mg, 2.94 mmol) and 2,4,6-tripropyl-1,3,5,2,4,6-trioxatriphosphorinane 2,4,6-trioxide (2.19 ml, 3.67 mmol, 50% solution in tetrahydrofuran) were added. The resulting reaction mixture was stirred at room temperature for a further 30 minutes, and then water, sat. sodium hydrogencarbonate solution and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N′,N′-dimethyl-2-(trifluoromethyl)-4-methylbenzohydrazide in the form of a colorless solid (250 mg, 42% of theory). ¹H-NMR (400 MHz, CDCl₃ δ, ppm) 7.49 (m, 1H), 7.44 (m, 1H), 7.39 (m, 1H), 6.38 (br. s, 1H, NH), 2.70 (s, 6H), 2.43 (s, 3H). N′,N′-Dimethyl-2-(trifluoromethyl)-4-methylbenzohydrazide (100 mg, 0.41 mmol) was dissolved in abs. tetrahydrofuran (4 ml) under argon, and sodium hydride (19 mg, 0.49 mmol, 60% purity) was added at room temperature. Stirring at room temperature for 30 minutes was followed by the addition of 2-chloromethyl-3-chloro-5-(trifluoromethyl)pyridine (93 mg, 0.41 mmol), and the resulting reaction mixture was stirred at a temperature of 50° C. for one-and-a-half hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N-[3-chloro-5-(trifluoromethyppyridin-2-ylmethyl]-N′,N′-dimethyl-2-(trifluoromethyl)-4-methylbenzohydrazide in the form of a colorless, viscous oil (46 mg, 26% of theory).

¹H-NMR (400 MHz, CDCl₃ δ, ppm) 8.86 (d, 1H), 8.06 (d, 1H), 7.47 (m, 1H), 7.42 (m, 1H), 7.31-7.26 (m, 1H), 5.49 (s, 2H), 3.57 (s, 6H), 2.37 (s, 3H).

### No. A53-22: N′,N′-Dimethyl-N-(pyridin-3-ylmethyl)-4-(1,2,3-benzothiadiazolyl)carbonyl hydrazide

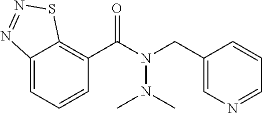 [SMILES: CN(C)N(Cc1cccnc1)C(=O)c1cccc2nnsc12]

4-(1,2,3-benzothiadiazolyl)carboxylic acid (800 mg, 4.44 mmol) was dissolved in abs. dichloromethane (30 ml) in a baked-out round-bottom flask under argon and at room temperature, and triethylamine (1.86 ml, 13.32 mmol) was added. After stirring at room temperature for 5 minutes, N,N-dimethylhydrazine (320 mg, 5.33 mmol) and 2,4,6-tripropyl-1,3,5,2,4,6-trioxatriphosphorinane 2,4,6-trioxide (1.5 equiv., 50% solution in tetrahydrofuran) were added. The resulting reaction mixture was stirred at room temperature for a further 60 minutes, and then water, sat. sodium hydrogencarbonate solution and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N′,N′-dimethyl-4-(1,2,3-benzothiadiazolyl)carbonyl hydrazide in the form of a colorless solid (310 mg, 31% of theory). ¹H-NMR (400 MHz, CDCl₃ δ, ppm) 8.81 (m, 1H), 7.91 (br. s, 1H, NH), 7.74 (m, 1H), 7.22 (m, 1H), 2.84 (s, 6H). N′,N′-Dimethyl-4-(1,2,3-benzothiadiazolyl)carbonyl hydrazide (120 mg, 0.54 mmol) was dissolved in abs. tetrahydrofuran (5 ml) under argon, and sodium hydride (43 mg, 1.08 mmol, 60% purity) was added at room temperature. Stirring at room temperature for 30 minutes was followed by the addition of 3-picolyl chloride hydrochloride (93 mg, 0.57 mmol), and the resulting reaction mixture was stirred under reflux conditions for 30 minutes. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N′,N′-dimethyl-N-(pyridin-3-ylmethyl)-4-(1,2,3-benzothiadiazolyl)carbonyl hydrazide in the form of a colorless solid (83 mg, 49% of theory). ¹H-NMR (400 MHz, CDCl₃ δ, ppm) 8.69 (m, 1H), 8.67-8.63 (m, 2H), 8.35 (d, 1H), 7.80 (m, 1H), 7.68 (m, 1H), 7.29 (m, 1H), 5.15 (s, 2H), 3.50 (s, 6H).

### No. A53-61: N′,N′-Dimethyl-N-(2-cyanobenzyl)-4-(1,2,3-benzothiadiazolyl)carbonyl hydrazide

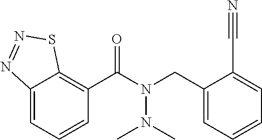 [SMILES: CN(C)N(Cc1ccccc1C#N)C(=O)c1cccc2nnsc12]

N′,N′-Dimethyl-4-(1,2,3-benzothiadiazolyl)carbonyl hydrazide (110 mg, 0.49 mmol) was dissolved in abs. tetrahydrofuran (4 ml) under argon, and sodium hydride (24 mg, 0.59 mmol, 60% purity) was added at room temperature. Stirring at room temperature for 30 minutes was followed by the addition of 2-chloromethylbenzonitrile (79 mg, 0.52 mmol), and the resulting reaction mixture was stirred under reflux conditions for two-and-a-half hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N′,N′-dimethyl-N-(2-cyanobenzyl)-4-(1,2,3-benzothiadiazolyl)carbonyl hydrazide in the form of a colorless solid (122 mg, 73% of theory). ¹H-NMR (400 MHz, CDCl₃ δ, ppm) 8.66 (m, 1H), 8.36 (d, 1H), 7.79 (m, 1H), 7.68 (m, 1H), 7.62 (m, 1H), 7.53-7.45 (m, 2H), 5.40 (s, 2H), 3.57 (s, 6H).

### No. B2-21: N′,N′-Diethyl-N-(pyridin-2-ylmethyl)-4-(trifluoromethyl)nicotinohydrazide

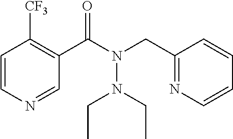 [SMILES: CCN(CC)N(Cc1ccccn1)C(=O)c1cnccc1C(F)(F)F]

4-(Trifluoromethyl)nicotinic acid (1000 mg, 5.23 mmol) was dissolved in abs. dichloromethane (10 ml), and triethylamine (2.19 ml, 15.69 mmol) was added. After stirring at room temperature for 5 minutes, N,N-diethylhydrazine (554 mg, 6.28 mmol) and 2,4,6-tripropyl-1,3,5,2,4,6-trioxatriphosphorinane 2,4,6-trioxide (4.67 ml, 7.85 mmol, 50% solution in tetrahydrofuran) were added. The resulting reaction mixture was stirred at room temperature for a further 30 minutes, and then water, sat. sodium hydrogencarbonate solution and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N′,N′-diethyl-4-(trifluoromethyl)nicotinohydrazide in the form of a colorless solid (880 mg, 64% of theory). ¹H-NMR (400 MHz, d₆-DMSO δ, ppm) 9.27 (br. s, 1H, NH), 8.93 (m, 1H), 8.78 (m, 1H), 7.86 (m, 1H), 2.80 (m, 4H), 1.07 (m, 6H). N′,N′-Diethyl-4-(trifluoromethyl)nicotinohydrazide (110 mg, 0.42 mmol) was dissolved in abs. tetrahydrofuran (10 ml) under argon, and sodium hydride (37 mg, 0.93 mmol, 60% purity) was added at room temperature. Stirring at room temperature for 30 minutes was followed by the addition of picolyl chloride (69 mg, 0.42 mmol), and the resulting reaction mixture was stirred under reflux conditions for two-and-a-half hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N′,N′-diethyl-N-(pyridin-2-ylmethyl)-4-(trifluoromethyl)nicotinohydrazide in the form of a colorless viscous oil (27 mg, 25% of theory). ¹H-NMR (400 MHz, CDCl₃ δ, ppm) 8.66 (m, 3H), 7.76 (m, 1H), 7.70 (m, 1H), 7.48 (m, 1H), 7.35 (m, 1H), 5.07 (s, 2H), 3.97-3.94 (m, 2H), 3.84-3.80 (m, 2H), 1.45 (t, 6H).

### No. B4-21: N′,N′-Dimethyl-N-(pyridin-2-ylmethyl)-4-[(trifluoromethyl)sulfanyl]nicotinohydrazide

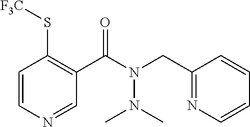 [SMILES: CN(C)N(Cc1ccccn1)C(=O)c1cnccc1SC(F)(F)F]

4-[(Trifluoromethyl)sulfanyl]nicotinic acid (1000 mg) was dissolved in abs. dichloromethane (30 ml) in a baked-out round-bottom flask under argon and at room temperature, and oxalyl chloride was added. The resulting reaction solution was then stirred at room temperature for 2 h and under reflux conditions for 30 minutes. After cooling to room temperature, the reaction mixture was concentrated and coevaporated with a little abs. toluene. By thorough removal of solvent residues, 4-[(trifluoromethyl)sulfanylnicotinoyl chloride (1080 mg, 4.47 mmol) was obtained, which was then, without further purification, dissolved again in abs. dichloromethane (50 ml) and added dropwise to a solution, cooled to 0° C., of N,N-dimethylhydrazine (0.35 ml, 4.47 mmol) and triethylamine (0.75 ml, 5.36 mmol) in dichloromethane (10 ml) under argon. The resulting reaction mixture was stirred at room temperature for a further 30 minutes, and then water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N′,N′-dimethyl-4-[(trifluoromethyl)sulfanyl]nicotinohydrazide in the form of a colorless solid (450 mg, 38% of theory). ¹H-NMR (400 MHz, CDCl₃ δ, ppm) 8.73 (m, 1H), 8.67 (m, 1H), 7.63 (m, 1H), 6.72 (br. s, 1H, NH), 2.76 (s, 6H). N′,N′-Dimethyl-4-[(trifluoromethyl)sulfanyl]nicotinohydrazide (100 mg, 0.38 mmol) was dissolved in abs. tetrahydrofuran (5 ml) under argon, and sodium hydride (17 mg, 0.42 mmol, 60% purity) was added at room temperature. Stirring at room temperature for 30 minutes was followed by the addition of 2-picolyl chloride (48 mg, 0.38 mmol), and the resulting reaction mixture was stirred under reflux conditions for two-and-a-half hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N′,N′-dimethyl-N-(pyridin-2-ylmethyl)-4-[(trifluoromethyl)sulfanyl]nicotinohydrazide in the form of a colorless solid (49 mg, 36% of theory). ¹H-NMR (400 MHz, CDCl₃ δ, ppm) 8.89 (m, 1H), 8.66 (m, 1H), 8.45 (m, 1H), 7.72 (m, 1H), 7.65 (m, 1H), 7.45 (m, 1H), 7.35 (m, 1H), 5.10 (s, 2H), 3.53 (s, 6H).

### No. B7-1: N-(2-Chlorobenzyl)-6-[chloro(difluoro)methyl]-N′,N′-dimethylnicotinohydrazide

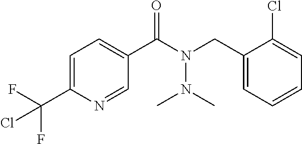 [SMILES: CN(C)N(Cc1ccccc1Cl)C(=O)c1ccc(C(F)(F)Cl)nc1]

6-Chlorodifluoronicotinic acid (500 mg, 2.41 mmol) was dissolved in abs. dichloromethane (15 ml) in a baked-out round-bottom flask under argon and at room temperature, and oxalyl chloride (260 mg, 2.05 mmol) and catalytic amounts of N,N-dimethylformamide were added. The resulting reaction solution was then stirred at room temperature for 2 h and under reflux conditions for 45 minutes. After cooling to room temperature, the reaction mixture was concentrated and coevaporated with a little abs. toluene. By thorough removal of solvent residues, 6-chlorodifluoronicotinoyl chloride (545 mg, 2.41 mmol) was obtained, which was then, without further purification, dissolved again in abs. dichloromethane (10 ml) and added dropwise to a solution, cooled to 0° C., of N,N-dimethylhydrazine (145 mg, 2.41 mmol) and triethylamine (0.37 ml, 2.65 mmol) in dichloromethane (10 ml) under argon. The resulting reaction mixture was stirred at room temperature for a further 30 minutes, and then water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave 6-[chloro(difluoro)methyl]-N′,N′-dimethylnicotinohydrazide in the form of a colorless solid (200 mg, 33% of theory). ¹H-NMR (400 MHz, CDCl₃ δ, ppm) 9.01 (s, 1H), 8.29 (d, 1H), 7.76 (d, 1H), 6.42-6.35 (br. s, 1H, NH), 2.81 (s, 6H). 6-[Chloro(difluoro)methyl]-N′,N′-dimethylnicotinohydrazide (100 mg, 0.40 mmol) was dissolved in abs. tetrahydrofuran (3 ml) under argon, and sodium hydride (18 mg, 0.44 mmol, 60% purity) was added at room temperature. Stirring at room temperature for 30 minutes was followed by the addition of 2-chlorobenzyl chloride (65 mg, 0.40 mmol), and the resulting reaction mixture was stirred under reflux conditions for 3 hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave N-(2-chlorobenzyl)-6-[chloro(difluoro)methyl]-N′,N′-dimethylnicotinohydrazide in the form of a colorless solid (99 mg, 66% of theory). ¹H-NMR (400 MHz, CDCl₃ δ, ppm) 9.25 (s, 1H), 8.40 (m, 1H), 7.69-7.66 (m, 1H), 7.64-7.62 (m, 1H), 7.48 (m, 1H), 7.41-7.38 (m, 1H), 7.34-7.27 (m, 2H), 5107 (s, 2H), 3.48 (s, 6H); ¹⁹F-NMR (377 MHz, CDCl₃ δ, ppm) 54.97.

### No. B16-462: 2-Chloro-N-[3-(2,6-difluorophenyl)propyl]-N′,N′-dimethylnicotinohydrazide

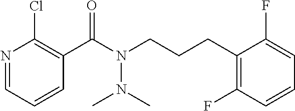 [SMILES: CN(C)N(CCCc1c(F)cccc1F)C(=O)c1cccnc1Cl]

2-Chloronicotinic acid (1 equiv.) was dissolved in abs. dichloromethane (10 ml/mmol), and triethylamine (3 equiv.) was added. After stirring at room temperature for 5 minutes, N,N-dimethylhydrazine (1.3 equiv.) and 2,4,6-tripropyl-1,3,5,2,4,6-trioxatriphosphorinane 2,4,6-trioxide (1.5 equiv., 50% solution in tetrahydrofuran) were added. The resulting reaction mixture was stirred at room temperature for a further 30 minutes, and then water, sat. sodium hydrogencarbonate solution and dichloromethane were added. The aqueous phase was repeatedly extracted vigorously with dichloromethane, and the combined organic phases were then dried over magnesium sulfate, filtered and concentrated. Final purification of the resulting crude product by column chromatography gave 2-chloro-N′,N′-dimethylnicotinohydrazide in the form of a colorless solid. 2-Chloro-N′,N′-dimethylnicotinohydrazide (160 mg, 0.80 mmol) was then dissolved in abs. N,N-dimethylformamide (10 ml) under argon, and sodium hydride (48 mg, 1.20 mmol, 60% purity) was added at room temperature. Stirring at room temperature for 15 minutes was followed by the addition of 3-(2,6-difluorophenyl)propyl bromide (226 mg, 0.96 mmol), and the resulting reaction mixture was stirred under reflux conditions for two hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave 2-chloro-N-[3-(2,6-difluorophenyl)propyl]-N′,N′-dimethylnicotinohydrazide in the form of a colorless viscous oil (153 mg, 54% of theory). ¹H-NMR (400 MHz, CDCl₃ δ, ppm) 8.44 (m, 1H), 7.80 (m, 1H), 7.27-7.18 (m, 2H), 6.92-6.88 (m, 2H), 4.02-3.98 (m, 2H), 3.67 (s, 6H), 2.86-2.82 (m, 2H), 2.28-2.24 (m, 2H).

### No. G2-61: 3,6-Dichloro-N-(2-cyanobenzyl)-N′,N′-dimethylpyridazin-4-ylcarbohydrazide

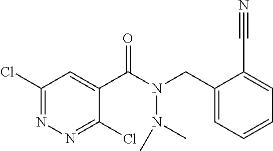 [SMILES: CN(C)N(Cc1ccccc1C#N)C(=O)c1cc(Cl)nnc1Cl]

3,6-Dichloropyridazine-4-carboxylic acid (700 mg, 3.63 mmol) was dissolved in abs. dichloromethane (10 ml), and triethylamine (0.33 ml, 1.52 mmol) was added. After stirring at room temperature for 5 minutes, N,N-dimethylhydrazine (0.33 ml, 4.35 mmol) and 2,4,6-tripropyl-1,3,5,2,4,6-trioxatriphosphorinane 2,4,6-trioxide (3.24 ml, 5.44 mmol, 50% solution in tetrahydrofuran) were added. The resulting reaction mixture was stirred at room temperature for a further 30 minutes, and then water, sat. sodium hydrogencarbonate solution and dichloromethane were added. The aqueous phase was repeatedly extracted vigorously with dichloromethane, and the combined organic phases were then dried over magnesium sulfate, filtered and concentrated. Final purification of the resulting crude product by column chromatography gave 3,6-dichloro-N′,N′-dimethylpyridazin-2-ylcarbohydrazide in the form of a colorless solid (270 mg, 32% of theory). ¹H-NMR (400 MHz, CDCl₃ δ, ppm) 10.01 (br. S, 1H, NH), 8.29 (s, 1H), 2.71 (s, 6H). 3,6-Dichloro-N′,N′-dimethylpyridazin-2-ylcarbohydrazide (135 mg, 0.57 mmol) was then dissolved in abs. tetrahydrofuran (4 ml) under argon, and sodium hydride (28 mg, 0.69 mmol, 60% purity) was added at room temperature. Stirring at room temperature for 15 minutes was followed by the addition of 2-chloromethylbenzonitrile (87 mg, 0.57 mmol), and the resulting reaction mixture was stirred at 60° C. for two-and-a-half hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave 3,6-dichloro-N-(2-cyanobenzyl)-N′,N′-dimethylpyridazin-4-ylcarbohydrazide in the form of a colorless viscous oil (10 mg, 5% of theory). ¹H-NMR (400 MHz, CDCl₃ δ, ppm) 8.29 (s, 1H), 7.68 (m, 1H), 7.64-7.57 (m, 2H), 7.47-7.43 (m, 1H), 4.77 (s, 2H), 3.51 (s, 6H).

### No. H1-462: 3-Chloro-N-[3-(2,6-difluorophenyl)propyl]-N′,N′-dimethypyrazine-2-carbohydrazide

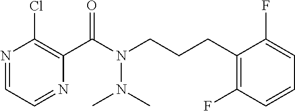 [SMILES: CN(C)N(CCCc1c(F)cccc1F)C(=O)c1nccnc1Cl]

3-Chloropyrazin-2-carboxylic acid (1 equiv.) was dissolved in abs. dichloromethane (10 ml/mmol), and triethylamine (3 equiv.) was added. After stirring at room temperature for 5 minutes, N,N-dimethylhydrazine (1.3 equiv.) and 2,4,6-tripropyl-1,3,5,2,4,6-trioxatriphosphorinane 2,4,6-trioxide (1.5 equiv., 50% solution in tetrahydrofuran) were added. The resulting reaction mixture was stirred at room temperature for a further 30 minutes, and then water, sat. sodium hydrogencarbonate solution and dichloromethane were added. The aqueous phase was repeatedly extracted vigorously with dichloromethane, and the combined organic phases were then dried over magnesium sulfate, filtered and concentrated. Final purification of the resulting crude product by column chromatography gave 3-chloro-N′,N′-dimethylpyrazine-2-carbohydrazide in the form of a colorless solid. 3-Chloro-N′,N′-dimethylpyrazine-2-carbohydrazide (250 mg, 1.25 mmol) was then dissolved in abs. N,N-dimethylformamide (10 ml) under argon, and sodium hydride (60 mg, 1.50 mmol, 60% purity) was added at room temperature. Stirring at room temperature for 15 minutes was followed by the addition of 3-(2,6-difluorophenyl)propyl bromide (352 mg, 1.50 mmol), and the resulting reaction mixture was stirred under reflux conditions for two hours. After cooling to room temperature, sat. sodium hydrogencarbonate solution, water and dichloromethane were added. The aqueous phase was repeatedly extracted vigorously with dichloromethane, and the combined organic phases were then dried over magnesium sulfate, filtered and concentrated. Final purification of the resulting crude product by column chromatography gave 3-chloro-N-[3-(2,6-difluorophenyl)propyl]-N′,N′-dimethylpyrazine-2-carbohydrazide in the form of a colorless viscous oil (118 mg, 26% of theory). ¹H-NMR (400 MHz, CDCl₃ δ, ppm) 8.46 (m, 1H), 8.39 (m, 1H), 7.22-7.16 (m, 1H), 6.90-6.86 (m, 2H), 3.95-3.90 (m, 2H), 3.61 (s, 6H), 2.84-2.80 (m, 2H), 2.28-2.23 (m, 2H).

In analogy to the preparation examples cited above and recited at the appropriate point, and taking account of the general details relating to the preparation of substituted heteroarylcarbonyl hydrazides of the general formula (I), the compounds cited below are obtained. In table 1 below, Y=“—” represents a direct bond.

A1. Compounds A1-1 to A1681-681 of the general formula (Ia1) in which R⁵, R⁶, Y, A¹, A², A³, A⁴, A⁵ correspond to the definitions (Nos. 1 to 670; corresponding to compounds A1-1 to A1-681) in table 1 below.

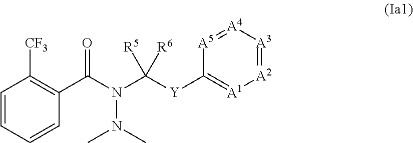

TABLE 1

| No. | R⁵ | R⁶ | Y | A¹ | A² | A³ | A⁴ | A⁵ |
|---|---|---|---|---|---|---|---|---|
| 1 | H | H | — | C—Cl | C—H | C—H | C—H | C—H |
| 2 | H | H | — | C—H | C—Cl | C—H | C—H | C—H |
| 3 | H | H | — | C—H | C—H | C—Cl | C—H | C—H |
| 4 | H | H | — | C—Cl | C—Cl | C—H | C—H | C—H |
| 5 | H | H | — | C—Cl | C—H | C—Cl | C—H | C—H |
| 6 | H | H | — | C—Cl | C—H | C—H | C—Cl | C—H |
| 7 | H | H | — | C—Cl | C—H | C—H | C—H | C—Cl |
| 8 | H | H | — | C—H | C—Cl | C—Cl | C—H | C—H |
| 9 | H | H | — | C—H | C—Cl | C—H | C—Cl | C—H |
| 10 | H | H | — | C—Cl | C—H | C—Cl | C—H | C—Cl |
| 11 | H | H | — | C—F | C—H | C—H | C—H | C—H |
| 12 | H | H | — | C—H | C—F | C—H | C—H | C—H |
| 13 | H | H | — | C—H | C—H | C—F | C—H | C—H |
| 14 | H | H | — | C—F | C—F | C—H | C—H | C—H |
| 15 | H | H | — | C—F | C—H | C—F | C—H | C—H |
| 16 | H | H | — | C—F | C—H | C—H | C—F | C—H |
| 17 | H | H | — | C—F | C—H | C—H | C—H | C—F |
| 18 | H | H | — | C—H | C—F | C—F | C—H | C—H |
| 19 | H | H | — | C—H | C—F | C—H | C—F | C—H |
| 20 | H | H | — | C—F | C—H | C—F | C—H | C—F |
| 21 | H | H | — | N | C—H | C—H | C—H | C—H |
| 22 | H | H | — | C—H | N | C—H | C—H | C—H |
| 23 | H | H | — | C—H | C—H | N | C—H | C—H |
| 24 | H | H | — | N | N | C—H | C—H | C—H |
| 25 | H | H | — | N | C—H | N | C—H | C—H |
| 26 | H | H | — | N | C—H | C—H | N | C—H |
| 27 | H | H | — | N | C—H | C—H | C—H | N |
| 28 | H | H | — | C—H | N | N | C—H | C—H |
| 29 | H | H | — | C—H | N | C—H | N | C—H |
| 30 | H | H | — | N | C—OCH₃ | C—H | C—OCH₃ | N |
| 31 | H | H | — | N | C—Cl | C—H | C—H | C—H |
| 32 | H | H | — | N | C—H | C—Cl | C—H | C—H |
| 33 | H | H | — | N | C—H | C—H | C—Cl | C—H |
| 34 | H | H | — | N | C—H | C—H | C—H | C—Cl |
| 35 | H | H | — | N | C—H | C—Cl | C—H | C—Cl |
| 36 | H | H | — | N | C—H | C—CF₃ | C—H | C—Cl |
| 37 | H | H | — | N | C—H | C—CH₃ | C—OCH₃ | C—CH₃ |
| 38 | H | H | — | N | C—H | C—H | C—H | C—CH₃ |
| 39 | H | H | — | N | C—H | C—H | C—CH₃ | C—H |
| 40 | H | H | — | N | C—H | C—H | C—Cl | C—Cl |
| 41 | H | H | — | C—Br | C—H | C—H | C—H | C—H |
| 42 | H | H | — | C—H | C—Br | C—H | C—H | C—H |
| 43 | H | H | — | C—H | C—H | C—Br | C—H | C—H |
| 44 | H | H | — | C—F | C—H | C—Br | C—H | C—H |
| 45 | H | H | — | C—Cl | C—H | C—Br | C—H | C—H |
| 46 | H | H | — | C—Br | C—H | C—F | C—H | C—H |
| 47 | H | H | — | C—Br | C—H | C—Cl | C—H | C—H |
| 48 | H | H | — | C—H | C—Cl | C—Br | C—H | C—H |
| 49 | H | H | — | C—H | C—Br | C—Cl | C—H | C—H |
| 50 | H | H | — | C—H | N | C—H | C—Br | C—H |
| 51 | H | H | — | C—I | C—H | C—H | C—H | C—H |
| 52 | H | H | — | C—H | C—I | C—H | C—H | C—H |
| 53 | H | H | — | C—H | C—H | C—I | C—H | C—H |
| 54 | H | H | — | C—F | C—H | C—I | C—H | C—H |
| 55 | H | H | — | C—F | C—H | C—Cl | C—H | C—H |
| 56 | H | H | — | C—F | C—H | C—H | C—Cl | C—H |
| 57 | H | H | — | C—H | C—F | C—Cl | C—H | C—H |
| 58 | H | H | — | C—H | C—Cl | C—F | C—H | C—H |
| 59 | H | H | — | C—F | C—H | C—H | C—H | C—Cl |
| 60 | H | H | — | C—H | C—F | C—H | C—Cl | C—H |
| 61 | H | H | — | C—CN | C—H | C—H | C—H | C—H |
| 62 | H | H | — | C—H | C—CN | C—H | C—H | C—H |
| 63 | H | H | — | C—H | C—H | C—CN | C—H | C—H |
| 64 | H | H | — | C—Cl | C—H | C—CN | C—H | C—H |
| 65 | H | H | — | C—H | C—Cl | C—H | C—CN | C—H |
| 66 | H | H | — | C—OEt | C—H | C—H | C—H | C—H |
| 67 | H | H | — | C—H | C—OEt | C—H | C—H | C—H |
| 68 | H | H | — | C—H | C—H | C—OEt | C—H | C—H |
| 69 | H | H | — | C—Cl | C—H | C—OEt | C—H | C—H |
| 70 | H | H | — | C—H | C—Cl | C—H | C—OEt | C—H |
| 71 | H | H | — | C—CH₃ | C—H | C—H | C—H | C—H |
| 72 | H | H | — | C—H | C—CH₃ | C—H | C—H | C—H |
| 73 | H | H | — | C—H | C—H | C—CH₃ | C—H | C—H |
| 74 | H | H | — | C—CH₃ | C—CH₃ | C—H | C—H | C—H |
| 75 | H | H | — | C—CH₃ | C—H | C—CH₃ | C—H | C—H |
| 76 | H | H | — | C—CH₃ | C—H | C—H | C—CH₃ | C—H |
| 77 | H | H | — | C—CH₃ | C—H | C—H | C—H | C—CH₃ |
| 78 | H | H | — | C—H | C—CH₃ | C—CH₃ | C—H | C—H |
| 79 | H | H | — | C—H | C—CH₃ | C—H | C—CH₃ | C—H |
| 80 | H | H | — | C—CH₃ | C—H | C—CH₃ | C—H | C—CH₃ |
| 81 | H | H | — | C—CF₃ | C—H | C—H | C—H | C—H |
| 82 | H | H | — | C—H | C—CF₃ | C—H | C—H | C—H |
| 83 | H | H | — | C—H | C—H | C—CF₃ | C—H | C—H |
| 84 | H | H | — | C—CF₃ | C—Cl | C—H | C—H | C—H |
| 85 | H | H | — | C—CF₃ | C—H | C—CF₃ | C—H | C—H |
| 86 | H | H | — | C—Cl | C—H | C—H | C—CF₃ | C—H |
| 87 | H | H | — | C—Cl | C—H | C—H | C—H | C—CF₃ |
| 88 | H | H | — | C—H | C—CF₃ | C—CF₃ | C—H | C—H |
| 89 | H | H | — | C—H | C—CF₃ | C—H | C—CF₃ | C—H |
| 90 | H | H | — | C—Cl | C—H | C—Cl | C—H | C—CF₃ |
| 91 | H | H | — | C—Cl | C—H | C—CF₃ | C—H | C—H |
| 92 | H | H | — | C—CF₃ | C—H | C—Cl | C—H | C—H |
| 93 | H | H | — | C—H | C—Cl | C—CF₃ | C—H | C—H |
| 94 | H | H | — | C—H | N | C—Cl | C—H | C—H |
| 95 | H | H | — | C—H | N | C—F | C—H | C—H |
| 96 | H | H | — | C—H | N | C—CF₃ | C—H | C—H |
| 96 | H | H | — | C—H | N | C—H | C—CF₃ | C—H |
| 97 | H | H | — | C—Cl | N | C—H | C—H | C—H |
| 98 | H | H | — | C—H | N | C—H | C—Cl | C—H |
| 99 | H | H | — | C—Cl | N | C—Cl | C—H | C—H |
| 100 | H | H | — | C—Cl | N | C—H | C—H | C—Cl |
| 101 | H | H | — | C—OCH₃ | C—H | C—H | C—H | C—H |
| 102 | H | H | — | C—H | C—OCH₃ | C—H | C—H | C—H |
| 103 | H | H | — | C—H | C—H | C—OCH₃ | C—H | C—H |
| 104 | H | H | — | C—OCH₃ | C—OCH₃ | C—H | C—H | C—H |
| 105 | H | H | — | C—OCH₃ | C—H | C—OCH₃ | C—H | C—H |
| 106 | H | H | — | C—OCH₃ | C—H | C—H | C—OCH₃ | C—H |
| 107 | H | H | — | C—OCH₃ | C—H | C—H | C—H | C—Cl |
| 108 | H | H | — | C—H | C—OCH₃ | C—OCH₃ | C—H | C—H |
| 109 | H | H | — | C—H | C—OCH₃ | C—H | C—OCH₃ | C—H |
| 110 | H | H | — | C—H | C—OCH₃ | C—OCH₃ | C—OCH₃ | C—H |
| 101 | H | H | — | C—SCH₃ | C—H | C—H | C—H | C—H |
| 102 | H | H | — | C—H | C—SCH₃ | C—H | C—H | C—H |
| 103 | H | H | — | C—H | C—H | C—SCH₃ | C—H | C—H |
| 104 | H | H | — | C—SCF₃ | C—H | C—H | C—H | C—H |
| 105 | H | H | — | C—H | C—SCF₃ | C—H | C—H | C—H |
| 106 | H | H | — | C—H | C—H | C—SCF₃ | C—H | C—H |
| 107 | H | H | — | C—OCF₃ | C—H | C—H | C—H | C—H |
| 108 | H | H | — | C—H | C—OCF₃ | C—H | C—H | C—H |
| 109 | H | H | — | C—H | C—H | C—OCF₃ | C—H | C—H |
| 110 | H | H | — | C—NO₂ | C—H | C—H | C—H | C—H |
| 111 | H | H | — | C—H | C—NO₂ | C—H | C—H | C—H |
| 112 | H | H | — | C—H | C—H | C—NO₂ | C—H | C—H |
| 113 | H | H | — | C—Cl | C—H | C—NO₂ | C—H | C—H |
| 114 | H | H | — | C—Cl | C—H | C—OCH₃ | C—H | C—H |
| 115 | H | H | — | C—H | C—Cl | C—OCH₃ | C—H | C—H |
| 116 | H | H | — | C—SCN | C—H | C—H | C—H | C—H |
| 117 | H | H | — | C—H | C—SCN | C—H | C—H | C—H |
| 118 | H | H | — | C—H | C—H | C—SCN | C—H | C—H |
| 119 | H | H | — | C—H | N | C—CH₃ | C—H | C—H |
| 120 | H | H | — | C—H | N | C—OCH₃ | C—H | C—H |
| 121 | H | H | — | N | C—H | 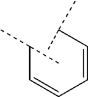 | | C—H |
| 122 | H | H | — | N | 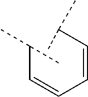 | | C—H | C—H |
| 123 | H | H | — | C—H | N | C—OCH₃ | C—H | C—H |
| 124 | H | H | — | C—H | N | C—SCH₃ | C—H | C—H |
| 125 | H | H | — | C—H | N | C—OCF₃ | C—H | C—H |
| 126 | H | H | — | C—Et | C—H | C—Et | C—H | C—CH₃ |
| 127 | H | H | — | C—Et | C—H | C—Et | C—H | C—H |
| 128 | H | H | — | C—H | C—H | C—Et | C—H | C—H |
| 129 | H | H | — | C—H | C—H | C—i-Pr | C—H | C—H |
| 130 | H | H | — | 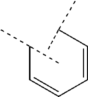 | | C—H | C—H | N |
| 131 | H | H | — | C—H | C—Cl | N | C—H | C—H |
| 132 | H | H | — | C—Cl | C—H | N | C—H | C—H |
| 133 | H | H | — | C—H | C—Cl | N | C—Cl | C—H |
| 134 | H | H | — | C—Cl | N | N | C—Cl | C—H |
| 135 | H | H | — | C—Cl | C—H | N | C—H | C—Cl |
| 136 | H | H | — | C—H | C—H | C—H | C—H | C—H |
| 137 | H | H | — | C—Cl | C—H | C—F | C—H | C—H |
| 138 | H | H | — | C—Cl | C—H | C—Cl | C—Cl | C—H |
| 139 | H | H | — | C—Cl | C—H | 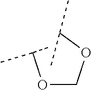 | | C—H |
| 140 | H | H | — | C—H | C—H | 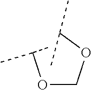 | | C—H |
| 141 | H | H | — | C—H | C—H | 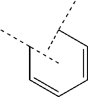 | | C—H |
| 142 | H | H | — | 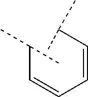 | | C—H | | C—H |
| 143 | H | H | — | C—H | C—H | C—t-Bu | C—H | C—H |
| 144 | H | H | — | N | C—H | C—H | C—OCH₂CF₃ | C—CH₃ |
| 145 | H | H | — | N | C—H | C—CH₃ | C—OCH₃ | C—CH₃ |
| 146 | H | H | — | N | C—H | C—H | C—OCH₃ | C—H |
| 147 | H | H | — | C—Cl | C—H | C—OCH₃ | C—OCH₃ | C—H |
| 148 | H | H | — | N | C—H | C—H | C—Cl | C—OCH₃ |
| 149 | H | H | — | C—Cl | C—H | C—H | C—Cl | C—Cl |
| 150 | H | H | — | C—H | C—Cl | C—Cl | C—Cl | C—H |
| 151 | CH₃ | H | — | C—H | C—H | C—H | C—H | C—H |
| 152 | CH₃ | H | — | C—Cl | C—H | C—H | C—H | C—H |
| 153 | CH₃ | H | — | C—H | C—Cl | C—H | C—H | C—H |
| 154 | CH₃ | H | — | C—H | C—H | C—Cl | C—H | C—H |
| 155 | CH₃ | H | — | C—Cl | C—H | C—Cl | C—H | C—H |
| 156 | CH₃ | H | — | C—Cl | C—H | C—H | C—Cl | C—H |
| 157 | CH₃ | H | — | C—F | C—H | C—H | C—H | C—H |
| 158 | CH₃ | H | — | C—H | C—F | C—H | C—H | C—H |
| 159 | CH₃ | H | — | C—H | C—H | C—F | C—H | C—H |
| 160 | CH₃ | H | — | C—F | C—H | C—F | C—H | C—H |
| 161 | CH₃ | H | — | C—H | C—F | C—H | C—F | C—H |
| 162 | CH₃ | H | — | C—F | C—H | C—H | C—H | C—F |
| 163 | CH₃ | H | — | C—Br | C—H | C—H | C—H | C—H |
| 164 | CH₃ | H | — | C—H | C—Br | C—H | C—H | C—H |
| 165 | CH₃ | H | — | C—H | C—H | C—Br | C—H | C—H |
| 166 | CH₃ | H | — | C—I | C—H | C—H | C—H | C—H |
| 167 | CH₃ | H | — | C—OCH₃ | C—H | C—H | C—H | C—H |
| 168 | CH₃ | H | — | C—CF₃ | C—H | C—H | C—H | C—H |
| 169 | CH₃ | H | — | C—CH₃ | C—H | C—H | C—H | C—H |
| 170 | CH₃ | H | — | C—CN | C—H | C—H | C—H | C—H |
| 171 | CH₃ | H | — | C—H | C—H | C—I | C—H | C—H |
| 172 | CH₃ | H | — | C—H | C—H | C—OCH₃ | C—H | C—H |
| 173 | CH₃ | H | — | C—H | C—H | C—CF₃ | C—H | C—H |
| 174 | CH₃ | H | — | C—H | C—H | C—CH₃ | C—H | C—H |
| 175 | CH₃ | H | — | C—H | C—H | C—CN | C—H | C—H |
| 176 | CH₃ | H | — | C—H | C—H | C—SCF₃ | C—H | C—H |
| 177 | CH₃ | H | — | C—H | C—H | C—OCF₃ | C—H | C—H |
| 178 | CH₃ | H | — | C—H | C—H | C—OEt | C—H | C—H |
| 179 | CH₃ | H | — | C—H | C—H | C—t-Bu | C—H | C—H |
| 180 | CH₃ | H | — | C—F | C—H | C—Br | C—H | C—H |
| 181 | CH₃ | H | — | N | C—H | C—H | C—H | C—H |
| 182 | CH₃ | H | — | N | C—Cl | C—H | C—H | C—H |
| 183 | CH₃ | H | — | N | C—H | C—Cl | C—H | C—H |
| 184 | CH₃ | H | — | N | C—H | C—H | C—H | C—Cl |
| 185 | CH₃ | H | — | C—H | N | C—H | C—H | C—H |
| 186 | CH₃ | H | — | C—H | N | C—Cl | C—H | C—H |
| 187 | CH₃ | H | — | C—H | C—H | N | C—H | C—H |
| 188 | CH₃ | H | — | N | C—H | C—H | C—H | N |
| 189 | CH₃ | H | — | N | C—H | C—CF₃ | C—H | C—H |
| 190 | CH₃ | H | — | C—Cl | C—H | N | C—H | C—H |
| 191 | CH₃ | H | — | C—H | C—I | C—H | C—H | C—H |
| 192 | CH₃ | H | — | C—H | C—OCH₃ | C—H | C—H | C—H |
| 193 | CH₃ | H | — | C—H | C—CF₃ | C—H | C—H | C—H |
| 194 | CH₃ | H | — | C—H | C—CH₃ | C—H | C—H | C—H |
| 195 | CH₃ | H | — | C—H | C—CN | C—H | C—H | C—H |
| 196 | CH₃ | H | — | C—H | C—CH₃ | C—H | C—H | C—H |
| 197 | CH₃ | H | — | C—H | C—Cl | C—H | C—Cl | C—H |
| 198 | CH₃ | H | — | C—H | C—SCF₃ | C—H | C—H | C—H |
| 199 | CH₃ | H | — | C—H | C—OCF₃ | C—H | C—H | C—H |
| 200 | CH₃ | H | — | C—H | C—OEt | C—H | C—H | C—H |
| 201 | Et | H | — | C—H | C—H | C—H | C—H | C—H |
| 202 | Et | H | — | C—Cl | C—H | C—H | C—H | C—H |
| 203 | Et | H | — | C—H | C—Cl | C—H | C—H | C—H |
| 204 | Et | H | — | C—H | C—H | C—Cl | C—H | C—H |
| 205 | Et | H | — | C—Cl | C—H | C—Cl | C—H | C—H |
| 206 | Et | H | — | C—Cl | C—H | C—H | C—Cl | C—H |
| 207 | Et | H | — | C—F | C—H | C—H | C—H | C—H |
| 208 | Et | H | — | C—H | C—F | C—H | C—H | C—H |
| 209 | Et | H | — | C—H | C—H | C—F | C—H | C—H |
| 210 | Et | H | — | C—F | C—H | C—F | C—H | C—H |
| 211 | Et | H | — | C—H | C—F | C—H | C—F | C—H |
| 212 | Et | H | — | C—F | C—H | C—H | C—H | C—F |
| 213 | Et | H | — | C—Br | C—H | C—H | C—H | C—H |
| 214 | Et | H | — | C—H | C—Br | C—H | C—H | C—H |
| 215 | Et | H | — | C—H | C—H | C—Br | C—H | C—H |
| 216 | Et | H | — | C—I | C—H | C—H | C—H | C—H |
| 217 | Et | H | — | C—OCH₃ | C—H | C—H | C—H | C—H |
| 218 | Et | H | — | C—CF₃ | C—H | C—H | C—H | C—H |
| 219 | Et | H | — | C—CH₃ | C—H | C—H | C—H | C—H |
| 220 | Et | H | — | C—CN | C—H | C—H | C—H | C—H |
| 221 | Et | H | — | C—H | C—H | C—I | C—H | C—H |
| 222 | Et | H | — | C—H | C—H | C—OCH₃ | C—H | C—H |
| 223 | Et | H | — | C—H | C—H | C—CF₃ | C—H | C—H |
| 224 | Et | H | — | C—H | C—H | C—CH₃ | C—H | C—H |
| 225 | Et | H | — | C—H | C—H | C—CN | C—H | C—H |
| 226 | Et | H | — | C—H | C—H | C—SCF₃ | C—H | C—H |
| 227 | Et | H | — | C—H | C—H | C—OCF₃ | C—H | C—H |
| 228 | Et | H | — | C—H | C—H | C—OEt | C—H | C—H |
| 229 | Et | H | — | C—H | C—H | C—t-Bu | C—H | C—H |
| 230 | Et | H | — | C—F | C—H | C—Br | C—H | C—H |
| 231 | Et | H | — | N | C—H | C—H | C—H | C—H |
| 232 | Et | H | — | N | C—Cl | C—H | C—H | C—H |
| 233 | Et | H | — | N | C—H | C—Cl | C—H | C—H |
| 234 | Et | H | — | N | C—H | C—H | C—H | C—Cl |
| 235 | Et | H | — | C—H | N | C—H | C—H | C—H |
| 236 | Et | H | — | C—H | N | C—Cl | C—H | C—H |
| 237 | Et | H | — | C—H | C—H | N | C—H | C—H |
| 238 | Et | H | — | N | C—H | C—H | C—H | N |
| 239 | Et | H | — | N | C—H | C—H | N | C—H |
| 240 | Et | H | — | C—Cl | C—H | N | C—H | C—H |
| 241 | Et | H | — | C—H | C—I | C—H | C—H | C—H |
| 242 | Et | H | — | C—H | C—OCH₃ | C—H | C—H | C—H |
| 243 | Et | H | — | C—H | C—CF₃ | C—H | C—H | C—H |
| 244 | Et | H | — | C—H | C—CH₃ | C—H | C—H | C—H |
| 245 | Et | H | — | C—H | C—CN | C—H | C—H | C—H |
| 246 | Et | H | — | C—H | C—CH₃ | C—H | C—H | C—H |
| 247 | Et | H | — | C—H | C—Cl | C—H | C—Cl | C—H |
| 248 | Et | H | — | C—H | C—SCF₃ | C—H | C—H | C—H |
| 249 | Et | H | — | C—H | C—OCF₃ | C—H | C—H | C—H |
| 250 | Et | H | — | C—H | C—OEt | C—H | C—H | C—H |
| 251 | i-Pr | H | — | C—H | C—H | C—H | C—H | C—H |
| 252 | i-Pr | H | — | C—Cl | C—H | C—H | C—H | C—H |
| 253 | i-Pr | H | — | C—H | C—Cl | C—H | C—H | C—H |
| 254 | i-Pr | H | — | C—H | C—H | C—Cl | C—H | C—H |
| 255 | i-Pr | H | — | C—Cl | C—H | C—Cl | C—H | C—H |
| 256 | i-Pr | H | — | C—Cl | C—H | C—H | C—Cl | C—H |
| 257 | i-Pr | H | — | C—F | C—H | C—H | C—H | C—H |
| 258 | i-Pr | H | — | C—H | C—F | C—H | C—H | C—H |
| 259 | i-Pr | H | — | C—H | C—H | C—F | C—H | C—H |
| 260 | i-Pr | H | — | C—F | C—H | C—F | C—H | C—H |
| 261 | i-Pr | H | — | C—H | C—F | C—H | C—F | C—H |
| 262 | i-Pr | H | — | C—F | C—H | C—H | C—H | C—F |
| 263 | i-Pr | H | — | C—Br | C—H | C—H | C—H | C—H |
| 264 | i-Pr | H | — | C—H | C—Br | C—H | C—H | C—H |
| 265 | i-Pr | H | — | C—H | C—H | C—Br | C—H | C—H |
| 266 | i-Pr | H | — | C—I | C—H | C—H | C—H | C—H |
| 267 | i-Pr | H | — | C—OCH₃ | C—H | C—H | C—H | C—H |
| 268 | i-Pr | H | — | C—CF₃ | C—H | C—H | C—H | C—H |
| 269 | i-Pr | H | — | C—CH₃ | C—H | C—H | C—H | C—H |
| 270 | i-Pr | H | — | C—CN | C—H | C—H | C—H | C—H |
| 271 | i-Pr | H | — | C—H | C—H | C—I | C—H | C—H |
| 272 | i-Pr | H | — | C—H | C—H | C—OCH₃ | C—H | C—H |
| 273 | i-Pr | H | — | C—H | C—H | C—CF₃ | C—H | C—H |
| 274 | i-Pr | H | — | C—H | C—H | C—CH₃ | C—H | C—H |
| 275 | i-Pr | H | — | C—H | C—H | C—CN | C—H | C—H |
| 276 | i-Pr | H | — | C—H | C—H | C—SCF₃ | C—H | C—H |
| 277 | i-Pr | H | — | C—H | C—H | C—OCF₃ | C—H | C—H |
| 278 | i-Pr | H | — | C—H | C—H | C—OEt | C—H | C—H |
| 279 | i-Pr | H | — | C—H | C—H | C—t-Bu | C—H | C—H |
| 280 | i-Pr | H | — | C—F | C—H | C—Br | C—H | C—H |
| 281 | i-Pr | H | — | N | C—H | C—H | C—H | C—H |
| 282 | i-Pr | H | — | N | C—Cl | C—H | C—H | C—H |
| 283 | i-Pr | H | — | N | C—H | C—Cl | C—H | C—H |
| 284 | i-Pr | H | — | N | C—H | C—H | C—H | C—Cl |
| 285 | i-Pr | H | — | C—H | N | C—H | C—H | C—H |
| 286 | i-Pr | H | — | C—H | N | C—Cl | C—H | C—H |
| 287 | i-Pr | H | — | C—H | C—H | N | C—H | C—H |
| 288 | i-Pr | H | — | N | C—H | C—H | C—H | N |
| 289 | i-Pr | H | — | N | C—H | C—H | N | C—H |
| 290 | i-Pr | H | — | C—Cl | C—H | N | C—H | C—H |
| 291 | i-Pr | H | — | C—H | C—I | C—H | C—H | C—H |
| 292 | i-Pr | H | — | C—H | C—OCH₃ | C—H | C—H | C—H |
| 293 | i-Pr | H | — | C—H | C—CF₃ | C—H | C—H | C—H |
| 294 | i-Pr | H | — | C—H | C—CH₃ | C—H | C—H | C—H |
| 295 | i-Pr | H | — | C—H | C—CN | C—H | C—H | C—H |
| 296 | i-Pr | H | — | C—H | C—CH₃ | C—H | C—H | C—H |
| 297 | i-Pr | H | — | C—H | C—Cl | C—H | C—Cl | C—H |
| 298 | i-Pr | H | — | C—H | C—SCF₃ | C—H | C—H | C—H |
| 299 | i-Pr | H | — | C—H | C—OCF₃ | C—H | C—H | C—H |
| 300 | i-Pr | H | — | C—H | C—OEt | C—H | C—H | C—H |
| 301 | c-Pr | H | — | C—H | C—H | C—H | C—H | C—H |
| 302 | c-Pr | H | — | C—Cl | C—H | C—H | C—H | C—H |
| 303 | c-Pr | H | — | C—H | C—Cl | C—H | C—H | C—H |
| 304 | c-Pr | H | — | C—H | C—H | C—Cl | C—H | C—H |
| 305 | c-Pr | H | — | C—Cl | C—H | C—Cl | C—H | C—H |
| 306 | c-Pr | H | — | C—Cl | C—H | C—H | C—Cl | C—H |
| 307 | c-Pr | H | — | C—F | C—H | C—H | C—H | C—H |
| 308 | c-Pr | H | — | C—H | C—F | C—H | C—H | C—H |
| 309 | c-Pr | H | — | C—H | C—H | C—F | C—H | C—H |
| 310 | c-Pr | H | — | C—F | C—H | C—F | C—H | C—H |
| 311 | c-Pr | H | — | C—H | C—F | C—H | C—F | C—H |
| 312 | c-Pr | H | — | C—F | C—H | C—H | C—H | C—F |
| 313 | c-Pr | H | — | C—Br | C—H | C—H | C—H | C—H |
| 314 | c-Pr | H | — | C—H | C—Br | C—H | C—H | C—H |
| 315 | c-Pr | H | — | C—H | C—H | C—Br | C—H | C—H |
| 316 | c-Pr | H | — | C—I | C—H | C—H | C—H | C—H |
| 317 | c-Pr | H | — | C—OCH₃ | C—H | C—H | C—H | C—H |
| 318 | c-Pr | H | — | C—CF₃ | C—H | C—H | C—H | C—H |
| 319 | c-Pr | H | — | C—CH₃ | C—H | C—H | C—H | C—H |
| 320 | c-Pr | H | — | C—CN | C—H | C—H | C—H | C—H |
| 321 | c-Pr | H | — | C—H | C—H | C—I | C—H | C—H |
| 322 | c-Pr | H | — | C—H | C—H | C—OCH₃ | C—H | C—H |
| 323 | c-Pr | H | — | C—H | C—H | C—CF₃ | C—H | C—H |
| 324 | c-Pr | H | — | C—H | C—H | C—CH₃ | C—H | C—H |
| 325 | c-Pr | H | — | C—H | C—H | C—CN | C—H | C—H |
| 326 | c-Pr | H | — | C—H | C—H | C—SCF₃ | C—H | C—H |
| 327 | c-Pr | H | — | C—H | C—H | C—OCF₃ | C—H | C—H |
| 328 | c-Pr | H | — | C—H | C—H | C—OEt | C—H | C—H |
| 329 | c-Pr | H | — | C—H | C—H | C—t-Bu | C—H | C—H |
| 330 | c-Pr | H | — | C—F | C—H | C—Br | C—H | C—H |
| 331 | c-Pr | H | — | N | C—H | C—H | C—H | C—H |
| 332 | c-Pr | H | — | N | C—Cl | C—H | C—H | C—H |
| 333 | c-Pr | H | — | N | C—H | C—Cl | C—H | C—H |
| 334 | c-Pr | H | — | N | C—H | C—H | C—H | C—Cl |
| 335 | c-Pr | H | — | C—H | N | C—H | C—H | C—H |
| 336 | c-Pr | H | — | C—H | N | C—Cl | C—H | C—H |
| 337 | c-Pr | H | — | C—H | C—H | N | C—H | C—H |
| 338 | c-Pr | H | — | N | C—H | C—H | C—H | N |
| 339 | c-Pr | H | — | N | C—H | C—H | N | C—H |
| 340 | c-Pr | H | — | C—Cl | C—H | N | C—H | C—H |
| 341 | c-Pr | H | — | C—H | C—I | C—H | C—H | C—H |
| 342 | c-Pr | H | — | C—H | C—OCH₃ | C—H | C—H | C—H |
| 343 | c-Pr | H | — | C—H | C—CF₃ | C—H | C—H | C—H |
| 344 | c-Pr | H | — | C—H | C—CH₃ | C—H | C—H | C—H |
| 345 | c-Pr | H | — | C—H | C—CN | C—H | C—H | C—H |
| 346 | c-Pr | H | — | C—H | C—CH₃ | C—H | C—H | C—H |
| 347 | c-Pr | H | — | C—H | C—Cl | C—H | C—Cl | C—H |
| 348 | c-Pr | H | — | C—H | C—SCF₃ | C—H | C—H | C—H |
| 349 | c-Pr | H | — | C—H | C—OCF₃ | C—H | C—H | C—H |
| 350 | c-Pr | H | — | C—H | C—OEt | C—H | C—H | C—H |
| 351 | CH₃ | H | CH₂ | C—H | C—H | C—H | C—H | C—H |
| 352 | CH₃ | H | CH₂ | C—Cl | C—H | C—H | C—H | C—H |
| 353 | CH₃ | H | CH₂ | C—H | C—Cl | C—H | C—H | C—H |
| 354 | CH₃ | H | CH₂ | C—H | C—H | C—Cl | C—H | C—H |
| 355 | CH₃ | H | CH₂ | C—Cl | C—H | C—Cl | C—H | C—H |
| 356 | CH₃ | H | CH₂ | C—Cl | C—H | C—H | C—Cl | C—H |
| 357 | CH₃ | H | CH₂ | C—F | C—H | C—H | C—H | C—H |
| 358 | CH₃ | H | CH₂ | C—H | C—F | C—H | C—H | C—H |
| 359 | CH₃ | H | CH₂ | C—H | C—H | C—F | C—H | C—H |
| 360 | CH₃ | H | CH₂ | C—F | C—H | C—F | C—H | C—H |
| 361 | CH₃ | H | CH₂ | C—H | C—F | C—H | C—F | C—H |
| 362 | CH₃ | H | CH₂ | C—F | C—H | C—H | C—H | C—F |
| 363 | CH₃ | H | CH₂ | C—Br | C—H | C—H | C—H | C—H |
| 364 | CH₃ | H | CH₂ | C—H | C—Br | C—H | C—H | C—H |
| 365 | CH₃ | H | CH₂ | C—H | C—H | C—Br | C—H | C—H |
| 366 | CH₃ | H | CH₂ | C—I | C—H | C—H | C—H | C—H |
| 367 | CH₃ | H | CH₂ | C—OCH₃ | C—H | C—H | C—H | C—H |
| 368 | CH₃ | H | CH₂ | C—CF₃ | C—H | C—H | C—H | C—H |
| 369 | CH₃ | H | CH₂ | C—CH₃ | C—H | C—H | C—H | C—H |
| 370 | CH₃ | H | CH₂ | C—CN | C—H | C—H | C—H | C—H |
| 371 | CH₃ | H | CH₂ | C—H | C—H | C—I | C—H | C—H |
| 372 | CH₃ | H | CH₂ | C—H | C—H | C—OCH₃ | C—H | C—H |
| 373 | CH₃ | H | CH₂ | C—H | C—H | C—CF₃ | C—H | C—H |
| 374 | CH₃ | H | CH₂ | C—H | C—H | C—CH₃ | C—H | C—H |
| 375 | CH₃ | H | CH₂ | C—H | C—H | C—CN | C—H | C—H |
| 376 | CH₃ | H | CH₂ | C—H | C—H | C—SCF₃ | C—H | C—H |
| 377 | CH₃ | H | CH₂ | C—H | C—H | C—OCF₃ | C—H | C—H |
| 378 | CH₃ | H | CH₂ | C—H | C—H | C—OEt | C—H | C—H |
| 379 | CH₃ | H | CH₂ | C—H | C—H | C—t-Bu | C—H | C—H |
| 380 | CH₃ | H | CH₂ | C—F | C—H | C—Br | C—H | C—H |
| 381 | CH₃ | H | CH₂ | N | C—H | C—H | C—H | C—H |
| 382 | CH₃ | H | CH₂ | N | C—Cl | C—H | C—H | C—H |
| 383 | CH₃ | H | CH₂ | N | C—H | C—Cl | C—H | C—H |
| 384 | CH₃ | H | CH₂ | N | C—H | C—H | C—H | C—Cl |
| 385 | CH₃ | H | CH₂ | C—H | N | C—H | C—H | C—H |
| 386 | CH₃ | H | CH₂ | C—H | N | C—Cl | C—H | C—H |
| 387 | CH₃ | H | CH₂ | C—H | C—H | N | C—H | C—H |
| 388 | CH₃ | H | CH₂ | N | C—H | C—H | C—H | N |
| 389 | CH₃ | H | CH₂ | N | C—H | C—H | N | C—H |
| 390 | CH₃ | H | CH₂ | C—Cl | C—H | N | C—H | C—H |
| 391 | CH₃ | H | CH₂ | C—H | C—I | C—H | C—H | C—H |
| 392 | CH₃ | H | CH₂ | C—H | C—OCH₃ | C—H | C—H | C—H |
| 393 | CH₃ | H | CH₂ | C—H | C—CF₃ | C—H | C—H | C—H |
| 394 | CH₃ | H | CH₂ | C—H | C—CN | C—H | C—H | C—H |
| 395 | CH₃ | H | CH₂ | C—CH₃ | C—H | C—H | C—H | C—CH₃ |
| 396 | CH₃ | H | CH₂ | C—Cl | C—H | C—H | C—H | C—Cl |
| 397 | CH₃ | H | CH₂ | C—H | C—Cl | C—H | C—Cl | C—H |
| 398 | CH₃ | H | CH₂ | C—H | C—SCF₃ | C—H | C—H | C—H |
| 399 | CH₃ | H | CH₂ | C—H | C—OCF₃ | C—H | C—H | C—H |
| 400 | CH₃ | H | CH₂ | C—H | C—OEt | C—H | C—H | C—H |
| 401 | CH₃ | H | CH₂CH₂ | C—H | C—H | C—H | C—H | C—H |
| 402 | CH₃ | H | CH₂CH₂ | C—Cl | C—H | C—H | C—H | C—H |
| 403 | CH₃ | H | CH₂CH₂ | C—H | C—Cl | C—H | C—H | C—H |
| 404 | CH₃ | H | CH₂CH₂ | C—H | C—H | C—Cl | C—H | C—H |
| 405 | CH₃ | H | CH₂CH₂ | C—Cl | C—H | C—Cl | C—H | C—H |
| 406 | CH₃ | H | CH₂CH₂ | C—Cl | C—H | C—H | C—Cl | C—H |
| 407 | CH₃ | H | CH₂CH₂ | C—F | C—H | C—H | C—H | C—H |
| 408 | CH₃ | H | CH₂CH₂ | C—H | C—F | C—H | C—H | C—H |
| 409 | CH₃ | H | CH₂CH₂ | C—H | C—H | C—F | C—H | C—H |
| 410 | CH₃ | H | CH₂CH₂ | C—F | C—H | C—F | C—H | C—H |
| 411 | CH₃ | H | CH₂CH₂ | C—H | C—F | C—H | C—F | C—H |
| 412 | CH₃ | H | CH₂CH₂ | C—F | C—H | C—H | C—H | C—F |
| 413 | CH₃ | H | CH₂CH₂ | C—Br | C—H | C—H | C—H | C—H |
| 414 | CH₃ | H | CH₂CH₂ | C—H | C—Br | C—H | C—H | C—H |
| 415 | CH₃ | H | CH₂CH₂ | C—H | C—H | C—Br | C—H | C—H |
| 416 | CH₃ | H | CH₂CH₂ | C—I | C—H | C—H | C—H | C—H |
| 417 | CH₃ | H | CH₂CH₂ | C—OCH₃ | C—H | C—H | C—H | C—H |
| 418 | CH₃ | H | CH₂CH₂ | C—CF₃ | C—H | C—H | C—H | C—H |
| 419 | CH₃ | H | CH₂CH₂ | C—CH₃ | C—H | C—H | C—H | C—H |
| 420 | CH₃ | H | CH₂CH₂ | C—CN | C—H | C—H | C—H | C—H |
| 421 | CH₃ | H | CH₂CH₂ | C—H | C—H | C—I | C—H | C—H |
| 422 | CH₃ | H | CH₂CH₂ | C—H | C—H | C—OCH₃ | C—H | C—H |
| 423 | CH₃ | H | CH₂CH₂ | C—H | C—H | C—CF₃ | C—H | C—H |
| 424 | CH₃ | H | CH₂CH₂ | C—H | C—H | C—CH₃ | C—H | C—H |
| 425 | CH₃ | H | CH₂CH₂ | C—H | C—H | C—CN | C—H | C—H |
| 426 | CH₃ | H | CH₂CH₂ | C—H | C—H | C—SCF₃ | C—H | C—H |
| 427 | CH₃ | H | CH₂CH₂ | C—H | C—H | C—OCF₃ | C—H | C—H |
| 428 | CH₃ | H | CH₂CH₂ | C—H | C—H | C—OEt | C—H | C—H |
| 429 | CH₃ | H | CH₂CH₂ | C—H | C—H | C—t-Bu | C—H | C—H |
| 430 | CH₃ | H | CH₂CH₂ | C—F | C—H | C—Br | C—H | C—H |
| 431 | CH₃ | H | CH₂CH₂ | N | C—H | C—H | C—H | C—H |
| 432 | CH₃ | H | CH₂CH₂ | N | C—Cl | C—H | C—H | C—H |
| 433 | CH₃ | H | CH₂CH₂ | N | C—H | C—Cl | C—H | C—H |
| 434 | CH₃ | H | CH₂CH₂ | N | C—H | C—H | C—H | C—Cl |
| 435 | CH₃ | H | CH₂CH₂ | C—H | N | C—H | C—H | C—H |
| 436 | CH₃ | H | CH₂CH₂ | C—H | N | C—Cl | C—H | C—H |
| 437 | CH₃ | H | CH₂CH₂ | C—H | C—H | N | C—H | C—H |
| 438 | CH₃ | H | CH₂CH₂ | N | C—H | C—H | C—H | N |
| 439 | CH₃ | H | CH₂CH₂ | N | C—H | C—H | N | C—H |
| 440 | CH₃ | H | CH₂CH₂ | C—Cl | C—H | N | C—H | C—H |
| 441 | CH₃ | H | CH₂CH₂ | C—H | C—I | C—H | C—H | C—H |
| 442 | CH₃ | H | CH₂CH₂ | C—H | C—OCH₃ | C—H | C—H | C—H |
| 443 | CH₃ | H | CH₂CH₂ | C—H | C—CF₃ | C—H | C—H | C—H |
| 444 | CH₃ | H | CH₂CH₂ | C—H | C—CN | C—H | C—H | C—H |
| 445 | CH₃ | H | CH₂CH₂ | C—CH₃ | C—H | C—H | C—H | C—CH₃ |
| 446 | CH₃ | H | CH₂CH₂ | C—Cl | C—H | C—H | C—H | C—Cl |
| 447 | CH₃ | H | CH₂CH₂ | C—H | C—Cl | C—H | C—Cl | C—H |
| 448 | CH₃ | H | CH₂CH₂ | C—H | C—SCF₃ | C—H | C—H | C—H |
| 449 | CH₃ | H | CH₂CH₂ | C—H | C—OCF₃ | C—H | C—H | C—H |
| 450 | CH₃ | H | CH₂CH₂ | C—H | C—OEt | C—H | C—H | C—H |
| 451 | H | H | CH₂CH₂ | C—H | C—H | C—H | C—H | C—H |
| 452 | H | H | CH₂CH₂ | C—Cl | C—H | C—H | C—H | C—H |
| 453 | H | H | CH₂CH₂ | C—H | C—Cl | C—H | C—H | C—H |
| 454 | H | H | CH₂CH₂ | C—H | C—H | C—Cl | C—H | C—H |
| 455 | H | H | CH₂CH₂ | C—Cl | C—H | C—Cl | C—H | C—H |
| 456 | H | H | CH₂CH₂ | C—Cl | C—H | C—H | C—Cl | C—H |
| 457 | H | H | CH₂CH₂ | C—F | C—H | C—H | C—H | C—H |
| 458 | H | H | CH₂CH₂ | C—H | C—F | C—H | C—H | C—H |
| 459 | H | H | CH₂CH₂ | C—H | C—H | C—F | C—H | C—H |
| 460 | H | H | CH₂CH₂ | C—F | C—H | C—F | C—H | C—H |
| 461 | H | H | CH₂CH₂ | C—H | C—F | C—H | C—F | C—H |
| 462 | H | H | CH₂CH₂ | C—F | C—H | C—H | C—H | C—F |
| 463 | H | H | CH₂CH₂ | C—Br | C—H | C—H | C—H | C—H |
| 464 | H | H | CH₂CH₂ | C—H | C—Br | C—H | C—H | C—H |
| 465 | H | H | CH₂CH₂ | C—H | C—H | C—Br | C—H | C—H |
| 466 | H | H | CH₂CH₂ | C—I | C—H | C—H | C—H | C—H |
| 467 | H | H | CH₂CH₂ | C—OCH₃ | C—H | C—H | C—H | C—H |
| 468 | H | H | CH₂CH₂ | C—CF₃ | C—H | C—H | C—H | C—H |
| 469 | H | H | CH₂CH₂ | C—CH₃ | C—H | C—H | C—H | C—H |
| 470 | H | H | CH₂CH₂ | C—CN | C—H | C—H | C—H | C—H |
| 471 | H | H | CH₂CH₂ | C—H | C—H | C—I | C—H | C—H |
| 472 | H | H | CH₂CH₂ | C—H | C—H | C—OCH₃ | C—H | C—H |
| 473 | H | H | CH₂CH₂ | C—H | C—H | C—CF₃ | C—H | C—H |
| 474 | H | H | CH₂CH₂ | C—H | C—H | C—CH₃ | C—H | C—H |
| 475 | H | H | CH₂CH₂ | C—H | C—H | C—CN | C—H | C—H |
| 476 | H | H | CH₂CH₂ | C—H | C—H | C—SCF₃ | C—H | C—H |
| 477 | H | H | CH₂CH₂ | C—H | C—H | C—OCF₃ | C—H | C—H |
| 478 | H | H | CH₂CH₂ | C—H | C—H | C—OEt | C—H | C—H |
| 479 | H | H | CH₂CH₂ | C—H | C—H | C—t-Bu | C—H | C—H |
| 480 | H | H | CH₂CH₂ | C—F | C—H | C—Br | C—H | C—H |
| 481 | H | H | CH₂CH₂ | N | C—H | C—H | C—H | C—H |
| 482 | H | H | CH₂CH₂ | N | C—Cl | C—H | C—H | C—H |
| 483 | H | H | CH₂CH₂ | N | C—H | C—Cl | C—H | C—H |
| 484 | H | H | CH₂CH₂ | N | C—H | C—H | C—H | C—Cl |
| 485 | H | H | CH₂CH₂ | C—H | N | C—H | C—H | C—H |
| 486 | H | H | CH₂CH₂ | C—H | N | C—Cl | C—H | C—H |
| 487 | H | H | CH₂CH₂ | C—H | C—H | N | C—H | C—H |
| 488 | H | H | CH₂CH₂ | N | C—H | C—H | C—H | N |
| 489 | H | H | CH₂CH₂ | N | C—H | C—H | N | C—H |
| 490 | H | H | CH₂CH₂ | C—Cl | C—H | N | C—H | C—H |
| 491 | H | H | CH₂CH₂ | C—H | C—I | C—H | C—H | C—H |
| 492 | H | H | CH₂CH₂ | C—H | C—OCH₃ | C—H | C—H | C—H |
| 493 | H | H | CH₂CH₂ | C—H | C—CF₃ | C—H | C—H | C—H |
| 494 | H | H | CH₂CH₂ | C—H | C—CN | C—H | C—H | C—H |
| 495 | H | H | CH₂CH₂ | C—CH₃ | C—H | C—H | C—H | C—CH₃ |
| 496 | H | H | CH₂CH₂ | C—Cl | C—H | C—H | C—H | C—Cl |
| 497 | H | H | CH₂CH₂ | C—H | C—Cl | C—H | C—Cl | C—H |
| 498 | H | H | CH₂CH₂ | C—H | C—SCF₃ | C—H | C—H | C—H |
| 499 | H | H | CH₂CH₂ | C—H | C—OCF₃ | C—H | C—H | C—H |
| 500 | H | H | CH₂CH₂ | C—H | C—OEt | C—H | C—H | C—H |
| 501 | H | H | — | C—H | C—H | C—OH | C—H | C—H |
| 502 | H | H | — | C—H | C—OH | C—OCH₃ | C—Cl | C—H |
| 503 | CH₃ | CH₃ | — | C—H | C—H | C—H | C—H | C—H |
| 504 | H | H | — | C—H | C—H | C—NMe₂ | C—H | C—H |
| 505 | H | H | — | C—H | C—CH₃ | C—OCH₃ | C—H | C—H |
| 506 | H | H | — | C—H | C—Cl | C—OCH₃ | C—H | C—H |
| 507 | H | H | — | N | C—H | C—CH₃ | N | C—H |
| 508 | H | H | — | N | C—H | C—Cl | N | C—H |
| 509 | H | H | — | C—Cl | C—H | 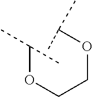 | | C—H |
| 510 | c-Bu | H | CH₂CH₃ | C—H | C—H | C—H | C—H | C—H |
| 511 | c-Pr | H | CH₂CH₃ | C—H | C—H | C—H | C—H | C—H |
| 512 | CO₂CH₃ | H | — | C—H | C—H | C—H | C—H | C—H |
| 513 | CO₂CH₃ | H | — | C—Cl | C—H | C—H | C—H | C—H |
| 514 | CO₂CH₃ | H | — | C—H | C—Cl | C—H | C—H | C—H |
| 515 | CO₂CH₃ | H | — | C—H | C—H | C—Cl | C—H | C—H |
| 516 | CO₂CH₃ | H | — | C—F | C—H | C—H | C—H | C—H |
| 517 | CO₂CH₃ | H | — | C—H | C—F | C—H | C—H | C—H |
| 518 | CO₂CH₃ | H | — | C—H | C—H | C—F | C—H | C—H |
| 519 | CO₂CH₃ | H | — | C—Cl | C—H | C—Cl | C—H | C—Cl |
| 520 | CO₂CH₃ | H | — | C—H | C—H | C—Br | C—H | C—H |
| 521 | CO₂CH₃ | H | — | C—H | C—H | C—I | C—H | C—H |
| 522 | CO₂CH₃ | H | — | C—H | C—H | C—OCH₃ | C—H | C—H |
| 523 | CO₂CH₃ | H | — | C—H | C—H | C—CN | C—H | C—H |
| 524 | CO₂CH₃ | H | — | C—H | C—Cl | C—H | C—Cl | C—H |
| 525 | CO₂Et | H | — | C—H | C—H | C—H | C—H | C—H |
| 526 | CO₂Et | H | — | C—Cl | C—H | C—H | C—H | C—H |
| 527 | CO₂Et | H | — | C—H | C—Cl | C—H | C—H | C—H |
| 528 | CO₂Et | H | — | C—H | C—H | C—Cl | C—H | C—H |
| 529 | CO₂Et | H | — | C—F | C—H | C—H | C—H | C—H |
| 530 | CO₂Et | H | — | C—H | C—F | C—H | C—H | C—H |
| 531 | CO₂Et | H | — | C—H | C—H | C—F | C—H | C—H |
| 532 | CO₂Et | H | — | C—Cl | C—H | C—Cl | C—H | C—Cl |
| 533 | CO₂Et | H | — | C—H | C—H | C—Br | C—H | C—H |
| 534 | CO₂Et | H | — | C—H | C—H | C—I | C—H | C—H |
| 535 | CO₂Et | H | — | C—H | C—H | C—OCH₃ | C—H | C—H |
| 536 | CO₂Et | H | — | C—H | C—H | C—CN | C—H | C—H |
| 537 | CO₂Et | H | — | C—H | C—Cl | C—H | C—Cl | C—H |
| 538 | CO₂CH₃ | H | CH₂ | C—H | C—H | C—H | C—H | C—H |
| 539 | CO₂CH₃ | H | CH₂ | C—Cl | C—H | C—H | C—H | C—H |
| 540 | CO₂CH₃ | H | CH₂ | C—H | C—Cl | C—H | C—H | C—H |
| 541 | CO₂CH₃ | H | CH₂ | C—H | C—H | C—Cl | C—H | C—H |
| 542 | CO₂CH₃ | H | CH₂ | C—F | C—H | C—H | C—H | C—H |
| 543 | CO₂CH₃ | H | CH₂ | C—H | C—F | C—H | C—H | C—H |
| 544 | CO₂CH₃ | H | CH₂ | C—H | C—H | C—F | C—H | C—H |
| 545 | CO₂CH₃ | H | CH₂ | C—Cl | C—H | C—Cl | C—H | C—Cl |
| 546 | CO₂CH₃ | H | CH₂ | C—H | C—H | C—Br | C—H | C—H |
| 547 | CO₂CH₃ | H | CH₂ | C—H | C—H | C—I | C—H | C—H |
| 548 | CO₂CH₃ | H | CH₂ | C—H | C—H | C—OCH₃ | C—H | C—H |
| 549 | CO₂CH₃ | H | CH₂ | C—H | C—H | C—CN | C—H | C—H |
| 550 | CO₂CH₃ | H | CH₂ | C—H | C—Cl | C—H | C—Cl | C—H |
| 551 | CO₂Et | H | CH₂ | C—H | C—H | C—H | C—H | C—H |
| 552 | CO₂Et | H | CH₂ | C—Cl | C—H | C—H | C—H | C—H |
| 553 | CO₂Et | H | CH₂ | C—H | C—Cl | C—H | C—H | C—H |
| 554 | CO₂Et | H | CH₂ | C—H | C—H | C—Cl | C—H | C—H |
| 555 | CO₂Et | H | CH₂ | C—F | C—H | C—H | C—H | C—H |
| 556 | CO₂Et | H | CH₂ | C—H | C—F | C—H | C—H | C—H |
| 557 | CO₂Et | H | CH₂ | C—H | C—H | C—F | C—H | C—H |
| 558 | CO₂Et | H | CH₂ | C—Cl | C—H | C—Cl | C—H | C—Cl |
| 559 | CO₂Et | H | CH₂ | C—H | C—H | C—Br | C—H | C—H |
| 560 | CO₂Et | H | CH₂ | C—H | C—H | C—I | C—H | C—H |
| 561 | CO₂Et | H | CH₂ | C—H | C—H | C—OCH₃ | C—H | C—H |
| 562 | CO₂Et | H | CH₂ | C—H | C—H | C—CN | C—H | C—H |
| 563 | CO₂Et | H | CH₂ | C—H | C—Cl | C—H | C—Cl | C—H |
| 564 | CO₂Et | H | CH₂ | N | C—H | C—H | C—H | C—H |
| 565 | CO₂CH₃ | H | CH₂ | N | C—H | C—H | C—H | C—H |
| 566 | CO₂Et | H | — | N | C—H | C—H | C—H | C—H |
| 567 | CO₂CH₃ | H | — | N | C—H | C—H | C—H | C—H |
| 568 | CO₂Et | H | CH₂ | C—H | N | C—H | C—H | C—H |
| 569 | CO₂CH₃ | H | CH₂ | C—H | N | C—H | C—H | C—H |
| 570 | CO₂Et | H | — | C—H | N | C—H | C—H | C—H |
| 571 | CO₂CH₃ | H | — | C—H | N | C—H | C—H | C—H |
| 572 | CO₂CH₃ | H | CH₂ | C—Cl | C—H | C—Cl | C—H | C—H |
| 573 | CO₂CH₃ | H | — | C—Cl | C—H | C—Cl | C—H | C—H |
| 574 | CO₂Et | H | CH₂ | C—Cl | C—H | C—Cl | C—H | C—H |
| 575 | CO₂Et | H | — | C—Cl | C—H | C—Cl | C—H | C—H |
| 576 | 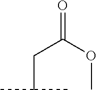 | H | — | C—H | C—H | C—H | C—H | C—H |
| 577 | 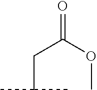 | H | — | C—H | C—H | C—Cl | C—H | C—H |
| 578 | 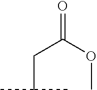 | H | — | C—H | C—H | C—Br | C—H | C—H |
| 579 | 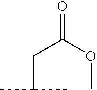 | H | — | C—H | C—H | C—I | C—H | C—H |
| 580 | 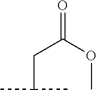 | H | — | C—H | C—Cl | C—H | C—H | C—H |
| 581 | 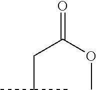 | H | — | C—Cl | C—H | C—Cl | C—H | C—H |
| 582 | 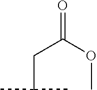 | H | CH₂ | C—H | C—H | C—H | C—H | C—H |
| 583 | 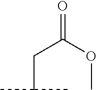 | H | CH₂ | C—H | C—H | C—Cl | C—H | C—H |
| 584 | 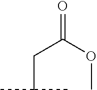 | H | CH₂ | C—H | C—H | C—Br | C—H | C—H |
| 585 | 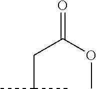 | H | CH₂ | C—H | C—H | C—I | C—H | C—H |
| 586 | 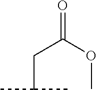 | H | CH₂ | C—H | C—Cl | C—H | C—H | C—H |
| 587 | 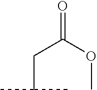 | H | CH₂ | C—Cl | C—H | C—Cl | C—H | C—H |
| 588 | 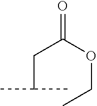 | H | — | C—H | C—H | C—H | C—H | C—H |
| 589 | 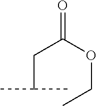 | H | — | C—H | C—H | C—Cl | C—H | C—H |
| 590 | 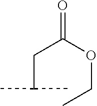 | H | — | C—H | C—H | C—Br | C—H | C—H |
| 591 | 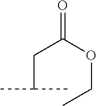 | H | — | C—H | C—H | C—I | C—H | C—H |
| 592 | 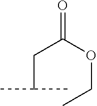 | H | — | C—H | C—Cl | C—H | C—H | C—H |
| 593 | 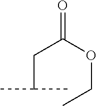 | H | — | C—Cl | C—H | C—Cl | C—H | C—H |
| 594 | 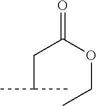 | H | CH₂ | C—H | C—H | C—H | C—H | C—H |
| 595 | 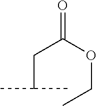 | H | CH₂ | C—H | C—H | C—Cl | C—H | C—H |
| 596 | 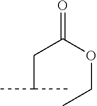 | H | CH₂ | C—H | C—H | C—Br | C—H | C—H |
| 597 | 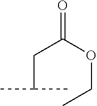 | H | CH₂ | C—H | C—H | C—I | C—H | C—H |
| 598 | 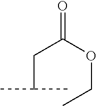 | H | CH₂ | C—H | C—Cl | C—H | C—H | C—H |
| 599 | 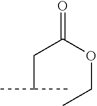 | H | CH₂ | C—Cl | C—H | C—Cl | C—H | C—H |
| 600 | 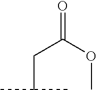 | H | — | N | C—H | C—H | C—H | C—H |
| 601 | CO₂CH₃ | H | CH₂ | C—Cl | C—H | C—CF₃ | C—H | C—H |
| 602 | CO₂Et | H | CH₂ | C—Cl | C—H | C—CF₃ | C—H | C—H |
| 603 | H | H | — | C—CO₂CH₃ | C—H | C—H | C—H | C—H |
| 604 | H | H | — | C—H | C—CO₂CH₃ | C—H | C—H | C—H |
| 605 | H | H | — | C—H | C—H | C—CO₂CH₃ | C—H | C—H |
| 606 | H | H | — | C—CO₂Et | C—H | C—H | C—H | C—H |
| 607 | H | H | — | C—H | C—CO₂Et | C—H | C—H | C—H |
| 608 | H | H | — | C—Cl | C—H | C—CF₃ | C—F | C—F |
| 609 | H | H | — | C—CO₂H | C—H | C—H | C—H | C—H |
| 610 | H | H | — | C—H | C—CO₂H | C—H | C—H | C—H |
| 611 | H | H | CH₂ | C—H | C—H | C—H | C—H | C—H |
| 612 | H | H | CH₂ | C—Cl | C—H | C—H | C—H | C—H |
| 613 | H | H | CH₂ | C—H | C—Cl | C—H | C—H | C—H |
| 614 | H | H | CH₂ | C—H | C—H | C—Cl | C—H | C—H |
| 615 | H | H | CH₂ | C—Cl | C—H | C—Cl | C—H | C—H |
| 616 | H | H | CH₂ | C—Cl | C—H | C—H | C—Cl | C—H |
| 617 | H | H | CH₂ | C—F | C—H | C—H | C—H | C—H |
| 618 | H | H | CH₂ | C—H | C—F | C—H | C—H | C—H |
| 619 | H | H | CH₂ | C—H | C—H | C—F | C—H | C—H |
| 620 | H | H | CH₂ | C—F | C—H | C—F | C—H | C—H |
| 621 | H | H | CH₂ | C—H | C—F | C—H | C—F | C—H |
| 622 | H | H | CH₂ | C—F | C—H | C—H | C—H | C—F |
| 623 | H | H | CH₂ | C—Br | C—H | C—H | C—H | C—H |
| 624 | H | H | CH₂ | C—H | C—Br | C—H | C—H | C—H |
| 625 | H | H | CH₂ | C—H | C—H | C—Br | C—H | C—H |
| 626 | H | H | CH₂ | C—I | C—H | C—H | C—H | C—H |
| 627 | H | H | CH₂ | C—OCH₃ | C—H | C—H | C—H | C—H |
| 628 | H | H | CH₂ | C—CF₃ | C—H | C—H | C—H | C—H |
| 629 | H | H | CH₂ | C—CH₃ | C—H | C—H | C—H | C—H |
| 630 | H | H | CH₂ | C—CN | C—H | C—H | C—H | C—H |
| 631 | H | H | CH₂ | C—H | C—H | C—I | C—H | C—H |
| 632 | H | H | CH₂ | C—H | C—H | C—OCH₃ | C—H | C—H |
| 633 | H | H | CH₂ | C—H | C—H | C—CF₃ | C—H | C—H |
| 634 | H | H | CH₂ | C—H | C—H | C—CH₃ | C—H | C—H |
| 635 | H | H | CH₂ | C—H | C—H | C—CN | C—H | C—H |
| 636 | H | H | CH₂ | C—H | C—H | C—SCF₃ | C—H | C—H |
| 637 | H | H | CH₂ | C—H | C—H | C—OCF₃ | C—H | C—H |
| 638 | H | H | CH₂ | C—H | C—H | C—OEt | C—H | C—H |
| 639 | H | H | CH₂ | C—H | C—H | C—t-Bu | C—H | C—H |
| 640 | H | H | CH₂ | C—F | C—H | C—Br | C—H | C—H |
| 641 | H | H | CH₂ | N | C—H | C—H | C—H | C—H |
| 642 | H | H | CH₂ | N | C—Cl | C—H | C—H | C—H |
| 643 | H | H | CH₂ | N | C—H | C—Cl | C—H | C—H |
| 644 | H | H | CH₂ | N | C—H | C—H | C—H | C—Cl |
| 645 | H | H | CH₂ | C—H | N | C—H | C—H | C—H |
| 646 | H | H | CH₂ | C—H | N | C—Cl | C—H | C—H |
| 647 | H | H | CH₂ | C—H | C—H | N | C—H | C—H |
| 648 | H | H | CH₂ | N | C—H | C—H | C—H | N |
| 649 | H | H | CH₂ | N | C—H | C—H | N | C—H |
| 650 | H | H | CH₂ | C—Cl | C—H | N | C—H | C—H |
| 651 | H | H | CH₂ | C—H | C—I | C—H | C—H | C—H |
| 652 | H | H | CH₂ | C—H | C—OCH₃ | C—H | C—H | C—H |
| 653 | H | H | CH₂ | C—H | C—CF₃ | C—H | C—H | C—H |
| 654 | H | H | CH₂ | C—H | C—CN | C—H | C—H | C—H |
| 655 | H | H | CH₂ | C—CH₃ | C—H | C—H | C—H | C—CH₃ |
| 656 | H | H | CH₂ | C—Cl | C—H | C—H | C—H | C—Cl |
| 657 | H | H | CH₂ | C—H | C—Cl | C—H | C—Cl | C—H |
| 658 | H | H | CH₂ | C—H | C—SCF₃ | C—H | C—H | C—H |
| 659 | H | H | CH₂ | C—H | C—OCF₃ | C—H | C—H | C—H |
| 660 | H | H | CH₂ | C—H | C—OEt | C—H | C—H | C—H |
| 661 | H | H | CH₂ | C—H | C—OCH₃ | C—OCH₃ | C—OCH₃ | C—H |
| 662 | H | H | CH₂ | C—H | C—OCH₃ | C—OCH₃ | C—H | C—H |
| 663 | H | H | CH₂ | C—H | C—CH₃ | C—CH₃ | C—H | C—H |
| 664 | H | H | CH₂ | C—CH₃ | C—H | C—CH₃ | C—H | C—H |
| 665 | H | H | CH₂ | C—CH₃ | C—H | C—CH₃ | C—H | C—CH₃ |
| 666 | H | H | CH₂ | C—Et | C—H | C—CH₃ | C—H | C—H |
| 667 | H | H | CH₂ | C—NO₂ | C—H | C—H | C—H | C—H |
| 668 | H | H | CH₂ | C—H | C—NO₂ | C—H | C—H | C—H |
| 669 | H | H | CH₂ | C—H | C—H | C—NO₂ | C—H | C—H |
| 670 | H | H | CH₂ | 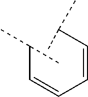 | | C—H | C—H | C—H |
| 671 | H | H | — | C—CHF₂ | C—H | C—H | C—H | C—H |
| 672 | H | H | — | C—H | C—CHF₂ | C—H | C—H | C—H |
| 673 | H | H | — | C—H | C—H | C—CHF₂ | C—H | C—H |
| 674 | H | H | — | C—CH₃ | C—CH₃ | C—CH₃ | C—CH₃ | C—CH₃ |
| 675 | H | H | — | C—F | C—F | C—F | C—F | C—F |
| 676 | H | H | — | C—OCHF₂ | C—H | C—H | C—H | C—H |
| 677 | H | H | — | C—H | C—OCHF₂ | C—H | C—H | C—H |
| 678 | H | H | — | C—H | C—H | C—OCHF₂ | C—H | C—H |
| 679 | H | H | — | C—CN | N | C—H | C—H | C—H |
| 680 | H | H | — | C—Cl | C—Cl | C—CF₃ | C—Cl | C—Cl |
| 681 | H | H | — | C—H | N | C—Cl | C—H | C—Cl |

A2. Compounds A2-1 to A2-681 of the general formula (Id1) in which R⁵, R⁶, Y, A¹, A², A³, A⁴, A⁵ correspond to the definitions (Nos. 1 to 681; corresponding to compounds A2-1 to A2-681) in table 1 above.

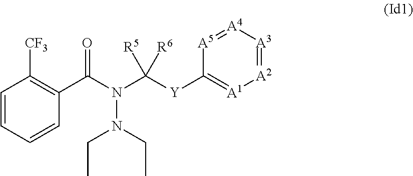

A3. Compounds A3-1 to A3-681 of the general formula (If1) in which R⁵, R⁶, Y, A¹, A², A³, A⁴, A⁵ correspond to the definitions (Nos. 1 to 681; corresponding to compounds A3-1 to A3-681) in table 1 above.

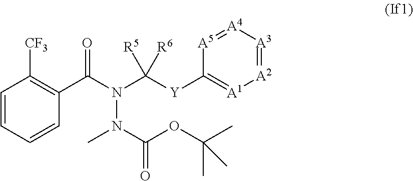

A4. Compounds A4-1 to A4-681 of the general formula (Ie1) in which R⁵, R⁶, Y, A¹, A², A³, A⁴, A⁵ correspond to the definitions (Nos. 1 to 681; corresponding to compounds A4-1 to A4-681) in table 1 above.

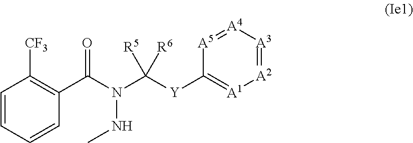

A5. Compounds A5-1 to A5-681 of the general formula (Ib1) in which R⁵, R⁶, Y, A¹, A², A³, A⁴, A⁵ correspond to the definitions (Nos. 1 to 681; corresponding to compounds A5-1 to A5-681) in table 1 above.

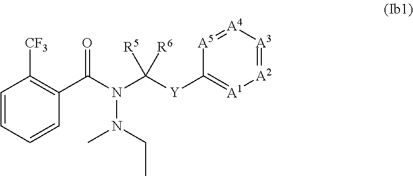

A6. Compounds A6-1 to A6-681 of the general formula (Ia2) in which R⁵, R⁶, Y, A¹, A², A³, A⁴, A⁵ correspond to the definitions (Nos. 1 to 681; corresponding to compounds A6-1 to A6-681) in table 1 above.

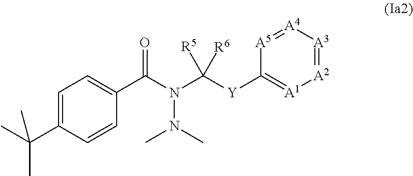

A7. Compounds A7-1 to A7-681 of the general formula (Ia3) in which R⁵, R⁶, Y, A¹, A², A³, A⁴, A⁵ correspond to the definitions (Nos. 1 to 681; corresponding to compounds A7-1 to A7-681) in table 1 above.

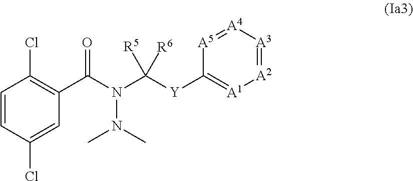

A8. Compounds A8-1 to A8-681 of the general formula (Ia4) in which R⁵, R⁶, Y, A¹, A², A³, A⁴, A⁵ correspond to the definitions (Nos. 1 to 681; corresponding to compounds A8-1 to A8-681) in table 1 above.

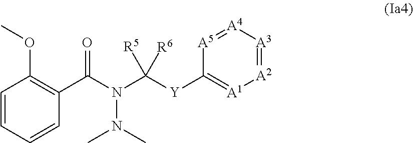

A9. Compounds A9-1 to A9-681 of the general formula (Ia5) in which R⁵, R⁶, Y, A¹, A², A³, A⁴, A⁵ correspond to the definitions (Nos. 1 to 681; corresponding to compounds A9-1 to A9-681) in table 1 above.

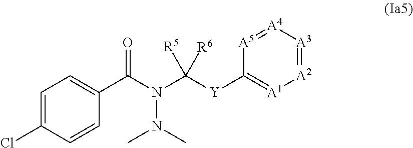

A10. Compounds A10-1 to A10-681 of the general formula (Ia6) in which R⁵, R⁶, Y, A¹, A², A³, A⁴, A⁵ correspond to the definitions (Nos. 1 to 681; corresponding to compounds A10-1 to A10-681) in table 1 above.

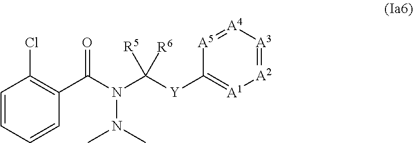

A11. Compounds A11-1 to A11-681 of the general formula (Ia7) in which R⁵, R⁶, Y, A¹, A², A³, A⁴, A⁵ correspond to the definitions (Nos. 1 to 681; corresponding to compounds A11-1 to A11-681) in table 1 above.

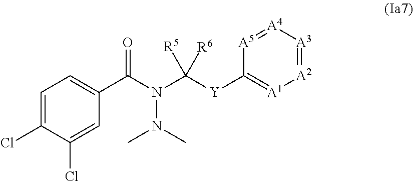

A12. Compounds A12-1 to A12-681 of the general formula (Ia8) in which R⁵, R⁶, Y, A¹, A², A³, A⁴, A⁵ correspond to the definitions (Nos. 1 to 681; corresponding to compounds A12-1 to A12-681) in table 1 above.

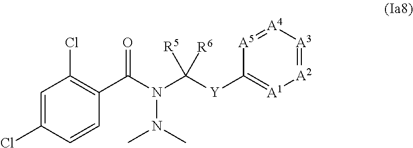

A13. Compounds A13-1 to A13-681 of the general formula (Ia9) in which R⁵, R⁶, Y, A¹, A², A³, A⁴, A⁵ correspond to the definitions (Nos. 1 to 681; corresponding to compounds A13-1 to A13-681) in table 1 above.

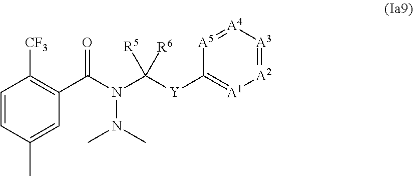

A14. Compounds A14-1 to A14-681 of the general formula (Ia10) in which R⁵, R⁶, Y, A¹, A², A³, A⁴, A⁵ correspond to the definitions (Nos. 1 to 681; corresponding to compounds A14-1 to A14-681) in table 1 above.

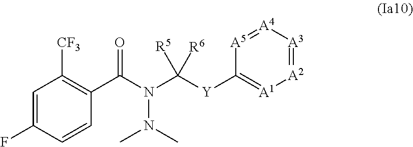

A15. Compounds A15-1 to A15-681 of the general formula (Ia11) in which R⁵, R⁶, Y, A¹, A², A³, A⁴, A⁵ correspond to the definitions (Nos. 1 to 681; corresponding to compounds A15-1 to A15-681) in table 1 above.

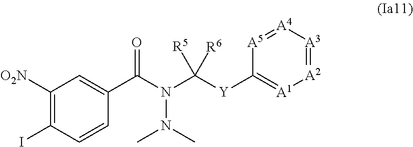

A16. Compounds A16-1 to A16-681 of the general formula (Ia12) in which R⁵, R⁶, Y, A¹, A², A³, A⁴, A⁵ correspond to the definitions (Nos. 1 to 681; corresponding to compounds A16-1 to A16-681) in table 1 above.

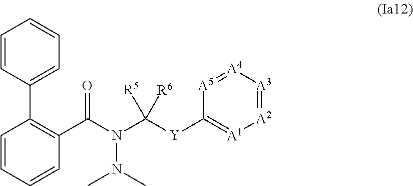

A17. Compounds A17-1 to A17-681 of the general formula (Ia13) in which R⁵, R⁶, Y, A¹, A², A³, A⁴, A⁵ correspond to the definitions (Nos. 1 to 681; corresponding to compounds A17-1 to A17-681) in table 1 above.

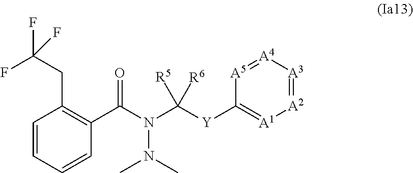

A18. Compounds A18-1 to A18-681 of the general formula (Ia14) in which R⁵, R⁶, Y, A¹, A², A³, A⁴, A⁵ correspond to the definitions (Nos. 1 to 681; corresponding to compounds A18-1 to A18-681) in table 1 above.

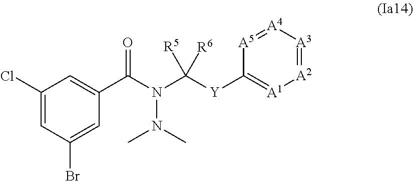

A19. Compounds A19-1 to A19-681 of the general formula (Ia15) in which R⁵, R⁶, Y, A¹, A², A³, A⁴, A⁵ correspond to the definitions (Nos. 1 to 681; corresponding to compounds A19-1 to A19-681) in table 1 above.

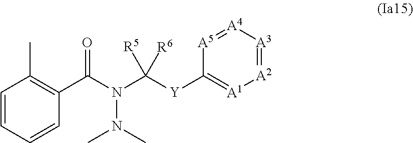

A20. Compounds A20-1 to A20-681 of the general formula (Ia16) in which R⁵, R⁶, Y, A¹, A², A³, A⁴, A⁵ correspond to the definitions (Nos. 1 to 681; corresponding to compounds A20-1 to A20-681) in table 1 above.

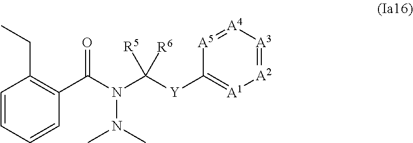

A21. Compounds A21-1 to A21-681 of the general formula (Ia17) in which R⁵, R⁶, Y, A¹, A², A³, A⁴, A⁵ correspond to the definitions (Nos. 1 to 681; corresponding to compounds A21-1 to A21-681) in table 1 above.

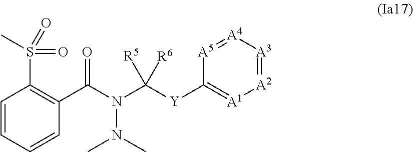

A22. Compounds A22-1 to A22-681 of the general formula (Ia18) in which R⁵, R⁶, Y, A¹, A², A³, A⁴, A⁵ correspond to the definitions (Nos. 1 to 681; corresponding to compounds A22-1 to A22-681) in table 1 above.

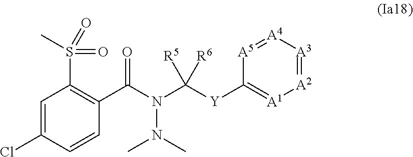

A23. Compounds A23-1 to A23-681 of the general formula (Ia19) in which R⁵, R⁶, Y, A¹, A², A³, A⁴, A⁵ correspond to the definitions (Nos. 1 to 681; corresponding to compounds A23-1 to A23-681) in table 1 above.

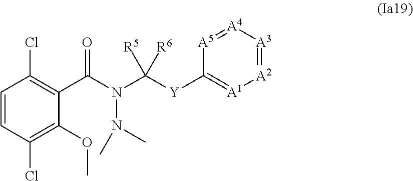

A24. Compounds A24-1 to A24-681 of the general formula (Ia19) in which R⁵, R⁶, Y, A¹, A², A³, A⁴, A⁵ correspond to the definitions (Nos. 1 to 681; corresponding to compounds A24-1 to A24-6811 in table 1 above.

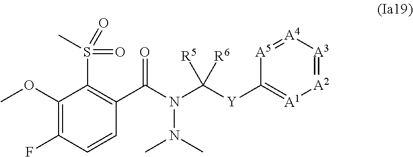

A25. Compounds A25-1 to A25-681 of the general formula (Ia20) in which R⁵, R⁶, Y, A¹, A², A³, A⁴, A⁵ correspond to the definitions (Nos. 1 to 681; corresponding to compounds A25-1 to A25-681) in table 1 above.

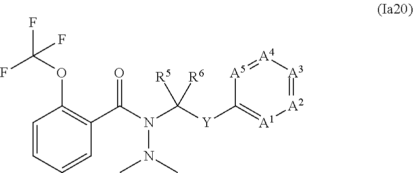

A26. Compounds A26-1 to A26-681 of the general formula (Ia21) in which R⁵, R⁶, Y, A¹, A², A³, A⁴, A⁵ correspond to the definitions (Nos. 1 to 681; corresponding to compounds A26-1 to A26-681) in table 1 above.

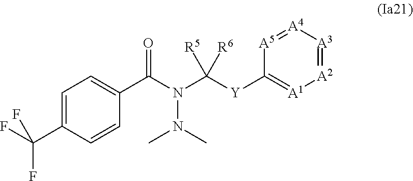

A27. Compounds A27-1 to A27-681 of the general formula (Ia22) in which R⁵, R⁶, Y, A¹, A², A³, A⁴, A⁵ correspond to the definitions (Nos. 1 to 681; corresponding to compounds A27-1 to A27-681) in table 1 above.

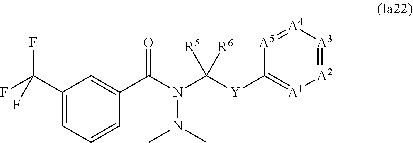

A28. Compounds A28-1 to A28-681 of the general formula (Ia23) in which R⁵, R⁶, Y, A¹, A², A³, A⁴, A⁵ correspond to the definitions (Nos. 1 to 681; corresponding to compounds A28-1 to A28-681) in table 1 above.

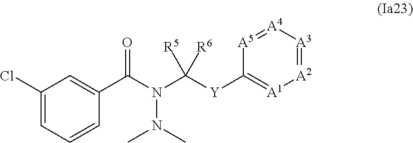

A29. Compounds A29-1 to A29-681 of the general formula (Ia24) in which R⁵, R⁶, Y, A¹, A², A³, A⁴, A⁵ correspond to the definitions (Nos. 1 to 681; corresponding to compounds A29-1 to A29-681) in table 1 above.

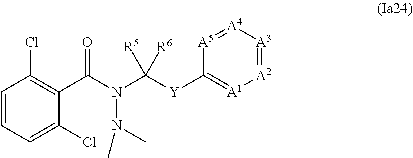

A30. Compounds A30-1 to A30-681 of the general formula (Ia25) in which R⁵, R⁶, Y, A¹, A², A³, A⁴, A⁵ correspond to the definitions (Nos. 1 to 681; corresponding to compounds A30-1 to A30-681) in table 1 above.

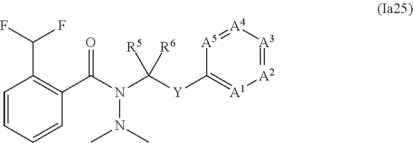

A31. Compounds A31-1 to A31-681 of the general formula (Ia26) in which R⁵, R⁶, Y, A¹, A², A³, A⁴, A⁵ correspond to the definitions (Nos. 1 to 681; corresponding to compounds A31-1 to A31-681) in table 1 above.

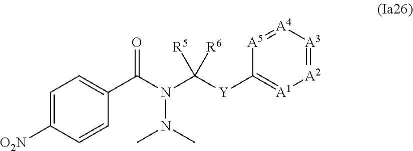

A32. Compounds A32-1 to A32-681 of the general formula (Ia27) in which R⁵, R⁶, Y, A¹, A², A³, A⁴, A⁵ correspond to the definitions (Nos. 1 to 681; corresponding to compounds A32-1 to A32-681) in table 1 above.

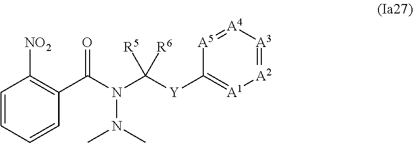

A33. Compounds A33-1 to A33-681 of the general formula (Ia28) in which R⁵, R⁶, Y, A¹, A², A³, A⁴, A⁵ correspond to the definitions (Nos. 1 to 681; corresponding to compounds A33-1 to A33-681) in table 1 above.

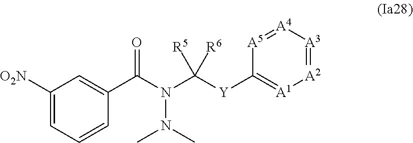

A34. Compounds A34-1 to A34-681 of the general formula (Ia29) in which R⁵, R⁶, Y, A¹, A², A³, A⁴, A⁵ correspond to the definitions (Nos. 1 to 681; corresponding to compounds A34-1 to A34-681) in table 1 above.

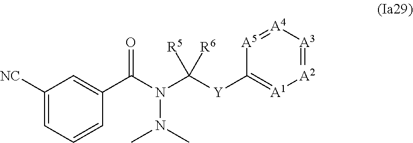

A35. Compounds A35-1 to A35-681 of the general formula (Ia30) in which R⁵, R⁶, Y, A¹, A², A³, A⁴, A⁵ correspond to the definitions (Nos. 1 to 681; corresponding to compounds A35-1 to A35-681) in table 1 above.

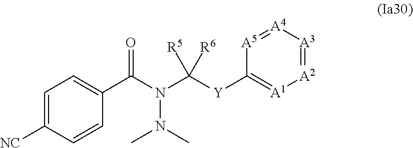

A36. Compounds A36-1 to A36-681 of the general formula (Ia31) in which R⁵, R⁶, Y, A¹, A², A³, A⁴, A⁵ correspond to the definitions (Nos. 1 to 681; corresponding to compounds A36-1 to A36-681) in table 1 above.

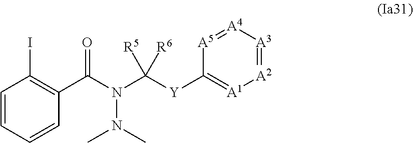

A37. Compounds A37-1 to A37-681 of the general formula (Ia32) in which R⁵, R⁶, Y, A¹, A², A³, A⁴, A⁵ correspond to the definitions (Nos. 1 to 681; corresponding to compounds A37-1 to A37-681) in table 1 above.

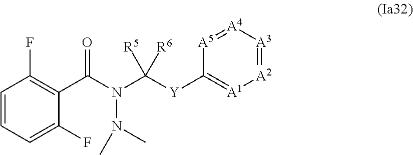

A38. Compounds A38-1 to A38-681 of the general formula (If2) in which R⁵, R⁶, Y, A¹, A², A³, A⁴, A⁵ correspond to the definitions (Nos. 1 to 681; corresponding to compounds A38-1 to A38-681) in table 1 above.

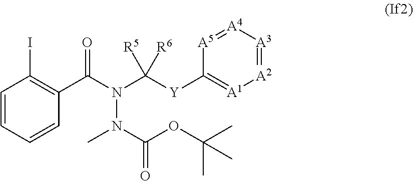

A39. Compounds A39-1 to A39-681 of the general formula (If3) in which R⁵, R⁶, Y, A¹, A², A³, A⁴, A⁵ correspond to the definitions (Nos. 1 to 681; corresponding to compounds A39-1 to A39-681) in table 1 above.

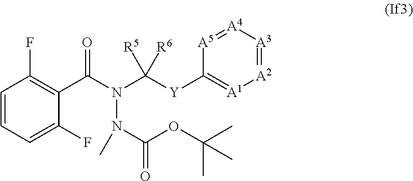

A40. Compounds A40-1 to A40-681 of the general formula (Ie2) in which R⁵, R⁶, Y, A¹, A², A³, A⁴, A⁵ correspond to the definitions (Nos. 1 to 681; corresponding to compounds A40-1 to A40-681) in table 1 above.

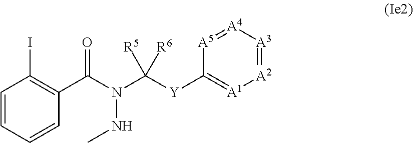

A41. Compounds A41-1 to A41-681 of the general formula (Ie3) in which R⁵, R⁶, Y, A¹, A², A³, A⁴, A⁵ correspond to the definitions (Nos. 1 to 681; corresponding to compounds A41-1 to A41-681) in table 1 above.

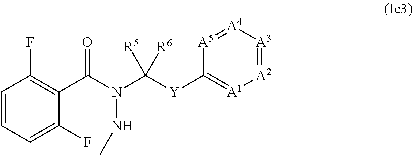

A42. Compounds A42-1 to A42-681 of the general formula (Ia33) in which R⁵, R⁶, Y, A¹, A², A³, A⁴, A⁵ correspond to the definitions (Nos. 1 to 681; corresponding to compounds A42-1 to A42-681) in table 1 above.

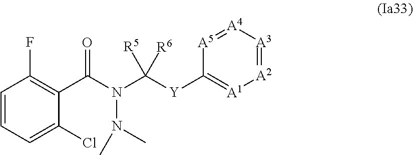

A43. Compounds A43-1 to A43-681 of the general formula (Ia34) in which R⁵, R⁶, Y, A¹, A², A³, A⁴, A⁵ correspond to the definitions (Nos. 1 to 681; corresponding to compounds A43-1 to A43-681) in table 1 above.

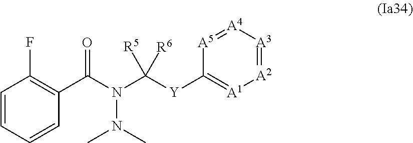

A44. Compounds A44-1 to A44-681 of the general formula (Ie4) in which R⁵, R⁶, Y, A¹, A², A³, A⁴, A⁵ correspond to the definitions (Nos. 1 to 681; corresponding to compounds A44-1 to A44-681) in table 1 above.

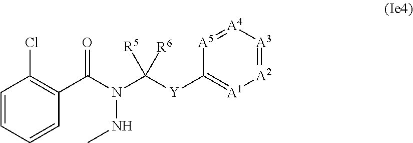

A45. Compounds A45-1 to A45-681 of the general formula (Ie5) in which R⁵, R⁶, Y, A¹, A², A³, A⁴, A⁵ correspond to the definitions (Nos. 1 to 681; corresponding to compounds A45-1 to A45-681) in table 1 above.

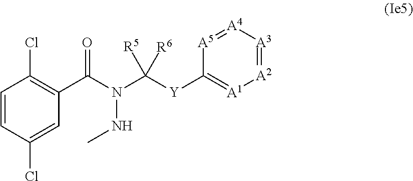

A46. Compounds A46-1 to A46-681 of the general formula (Ie6) in which R⁵, R⁶, Y, A¹, A², A³, A⁴, A⁵ correspond to the definitions (Nos. 1 to 681; corresponding to compounds A46-1 to A46-681) in table 1 above.

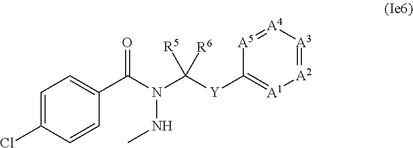

A47. Compounds A47-1 to A47-681 of the general formula (Ig1) in which R⁵, R⁶, Y, A¹, A², A³, A⁴, A⁵ correspond to the definitions (Nos. 1 to 681; corresponding to compounds A47-1 to A47-681) in table 1 above.

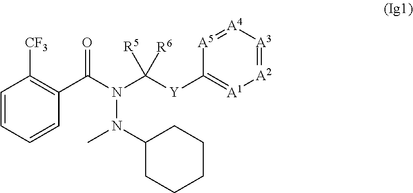

A48. Compounds A48-1 to A48-681 of the general formula (Ia35) in which R⁵, R⁶, Y, A¹, A², A³, A⁴, A⁵ correspond to the definitions (Nos. 1 to 681; corresponding to compounds A48-1 to A48-681) in table 1 above.

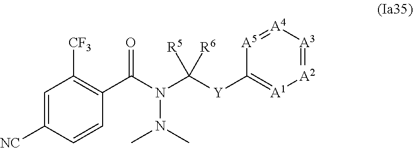

A49. Compounds A49-1 to A49-681 of the general formula (Ia36) in which R⁵, R⁶, Y, A¹, A², A³, A⁴, A⁵ correspond to the definitions (Nos. 1 to 681; corresponding to compounds A49-1 to A49-681) in table 1 above.

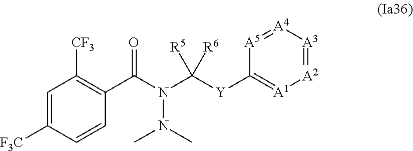

A50. Compounds A50-1 to A50-681 of the general formula (Ia37) in which R⁵, R⁶, Y, A¹, A², A³, A⁴, A⁵ correspond to the definitions (Nos. 1 to 681; corresponding to compounds A50-1 to A50-681) in table 1 above.

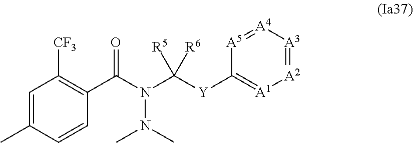

A51. Compounds A51-1 to A51-681 of the general formula (Ia38) in which R⁵, R⁶, Y, A¹, A², A³, A⁴, A⁵ correspond to the definitions (Nos. 1 to 681; corresponding to compounds A51-1 to A51-681) in table 1 above.

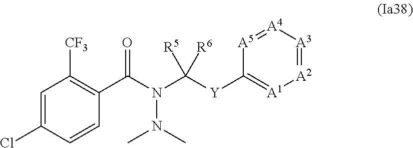

A52. Compounds A52-1 to A52-681 of the general formula (Ia39) in which R⁵, R⁶, Y, A¹, A², A³, A⁴, A⁵ correspond to the definitions (Nos. 1 to 681; corresponding to compounds A52-1 to A52-681) in table 1 above.

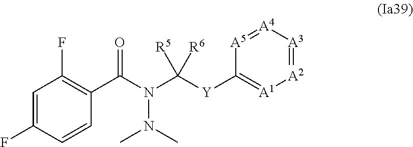

A53. Compounds A53-1 to A53-681 of the general formula (Ia40) in which R⁵, R⁶, Y, A¹, A², A³, A⁴, A⁵ correspond to the definitions (Nos. 1 to 681; corresponding to compounds A53-1 to A53-681) in table 1 above.

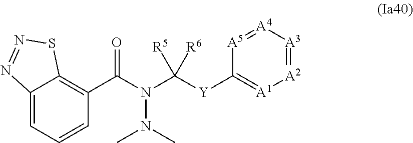

B1. Compounds B1-1 to B1-681 of the general formula (Ia41) in which R⁵, R⁶, Y, A¹, A², A³, A⁴, A⁵ correspond to the definitions (Nos. 1 to 681; corresponding to compounds B1-1 to B1-681) in table 1 above.

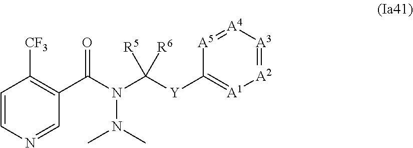

B2. Compounds B2-1 to B2-681 of the general formula (Id2) in which R⁵, R⁶, Y, A¹, A², A³, A⁴, A⁵ correspond to the definitions (Nos. 1 to 681; corresponding to compounds B2-1 to B2-681) in table 1 above.

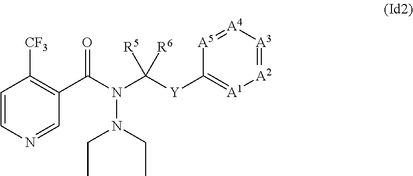

B3. Compounds B3-1 to B3-681 of the general formula (Ia42) in which R⁵, R⁶, Y, A¹, A², A³, A⁴, A⁵ correspond to the definitions (Nos. 1 to 681; corresponding to compounds B3-1 to B3-681) in table 1 above.

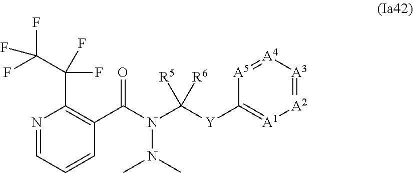

B4. Compounds B4-1 to B4-681 of the general formula (Ia43) in which R⁵, R⁶, Y, A¹, A², A³, A⁴, A⁵ correspond to the definitions (Nos. 1 to 681; corresponding to compounds B4-1 to B4-681) in table 1 above.

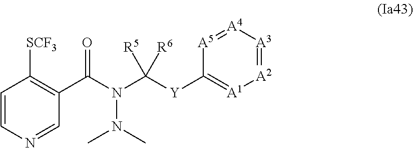

B5. Compounds B5-1 to B5-681 of the general formula (Ia44) in which R⁵, R⁶, Y, A¹, A², A³, A⁴, A⁵ correspond to the definitions (Nos. 1 to 681; corresponding to compounds B5-1 to B5-681) in table 1 above.

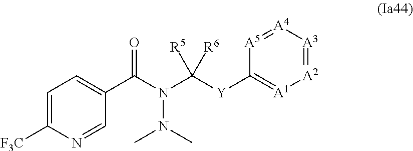

B6. Compounds B6-1 to B6-681 of the general formula (Ia45) in which R⁵, R⁶, Y, A¹, A², A³, A⁴, A⁵ correspond to the definitions (Nos. 1 to 681; corresponding to compounds B6-1 to B6-681) in table 1 above.

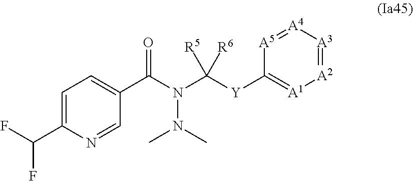

B7. Compounds B7-1 to B7-681 of the general formula (Ia46) in which R⁵, R⁶, Y, A¹, A², A³, A⁴, A⁵ correspond to the definitions (Nos. 1 to 681; corresponding to compounds B7-1 to B7-681) in table 1 above.

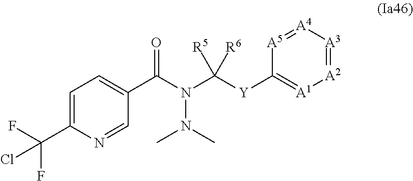

B8. Compounds B8-1 to B8-681 of the general formula (Ia47) in which R⁵, R⁶, Y, A¹, A², A³, A⁴, A⁵ correspond to the definitions (Nos. 1 to 681; corresponding to compounds B8-1 to B8-681) in table 1 above.

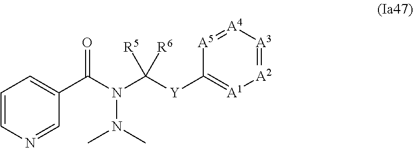

B9. Compounds B9-1 to B9-681 of the general formula (Ia48) in which R⁵, R⁶, Y, A¹, A², A³, A⁴, A⁵ correspond to the definitions (Nos. 1 to 681; corresponding to compounds B9-1 to B9-681) in table 1 above.

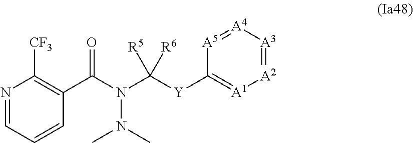

B10. Compounds B10-1 to B10-681 of the general formula (Ia49) in which R⁵, R⁶, Y, A¹, A², A³, A⁴, A⁵ correspond to the definitions (Nos. 1 to 681; corresponding to compounds B10-1 to B10-681) in table 1 above.

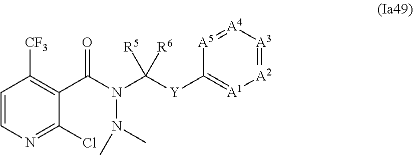

B11. Compounds B11-1 to B11-681 of the general formula (If4) in which R⁵, R⁶, Y, A¹, A², A³, A⁴, A⁵ correspond to the definitions (Nos. 1 to 681; corresponding to compounds B11-1 to B11-681) in table 1 above.

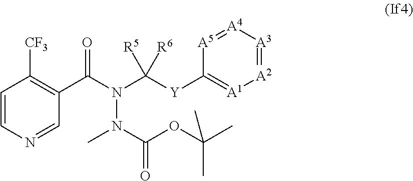

B12. Compounds B12-1 to B12-681 of the general formula (Ie7) in which R⁵, R⁶, Y, A¹, A², A³, A⁴, A⁵ correspond to the definitions (Nos. 1 to 681; corresponding to compounds B12-1 to B12-681) in table 1 above.

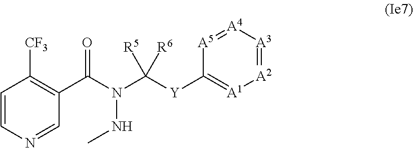

B13. Compounds B13-1 to B13-681 of the general formula (Ie8) in which R⁵, R⁶, Y, A¹, A², A³, A⁴, A⁵ correspond to the definitions (Nos. 1 to 681; corresponding to compounds B13-1 to B13-681) in table 1 above.

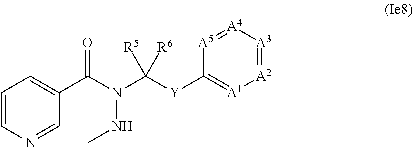

B14. Compounds B14-1 to B14-681 of the general formula (Ie9) in which R⁵, R⁶, Y, A¹, A², A³, A⁴, A⁵ correspond to the definitions (Nos. 1 to 681; corresponding to compounds B14-1 to B14-681) in table 1 above.

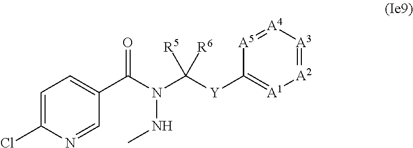

B15. Compounds B15-1 to B15-681 of the general formula (Ia50) in which R⁵, R⁶, Y, A¹, A², A³, A⁴, A⁵ correspond to the definitions (Nos. 1 to 681; corresponding to compounds B15-1 to B15-681) in table 1 above.

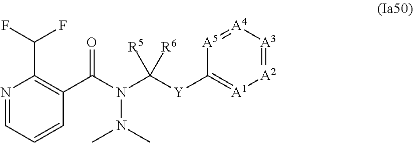

B16. Compounds B16-1 to B16-681 of the general formula (Ia51) in which R⁵, R⁶, Y, A¹, A², A³, A⁴, A⁵ correspond to the definitions (Nos. 1 to 681; corresponding to compounds B16-1 to B16-681) in table 1 above.

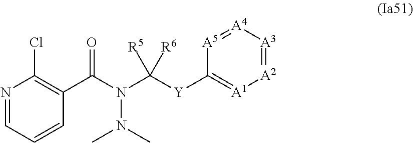

B17. Compounds B17-1 to B17-681 of the general formula (Ib2) in which R⁵, R⁶, Y, A¹, A², A³, A⁴, A⁵ correspond to the definitions (Nos. 1 to 681; corresponding to compounds B17-1 to B17-681) in table 1 above.

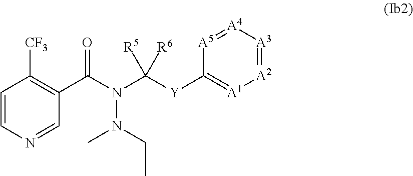

B18. Compounds B18-1 to B18-681 of the general formula (Ig2) in which R⁵, R⁶, Y, A¹, A², A³, A⁴, A⁵ correspond to the definitions (Nos. 1 to 681; corresponding to compounds B18-1 to B18-681) in table 1 above.

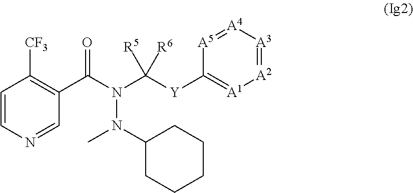

C1. Compounds C1-1 to C1-681 of the general formula (Ia52) in which R⁵, R⁶, Y, A¹, A², A³, A⁴, A⁵ correspond to the definitions (Nos. 1 to 681; corresponding to compounds C1-1 to C1-681) in table 1 above.

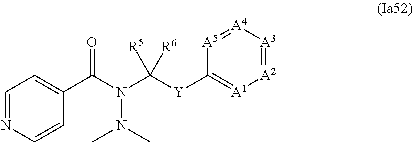

C2. Compounds C2-1 to C2-681 of the general formula (Ia53) in which R⁵, R⁶, Y, A¹, A², A³, A⁴, A⁵ correspond to the definitions (Nos. 1 to 681; corresponding to compounds C2-1 to C2-681) in table 1 above.

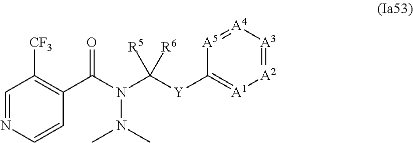

C3. Compounds C3-1 to C3-681 of the general formula (Ie10) in which R⁵, R⁶, Y, A¹, A², A³, A⁴, A⁵ correspond to the definitions (Nos. 1 to 681; corresponding to compounds C3-1 to C3-681) in table 1 above.

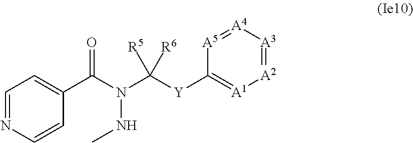

D1. Compounds D1-1 to D1-681 of the general formula (Ia54) in which R⁵, R⁶, Y, A¹, A², A³, A⁴, A⁵ correspond to the definitions (Nos. 1 to 681; corresponding to compounds D1-1 to D1-681) in table 1 above.

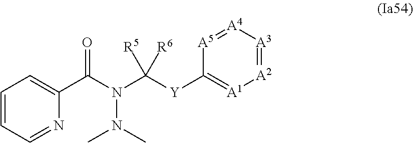

D2. Compounds D2-1 to D2-681 of the general formula (Ia55) in which R⁵, R⁶, Y, A¹, A², A³, A⁴, A⁵ correspond to the definitions (Nos. 1 to 681; corresponding to compounds D2-1 to D2-681) in table 1 above.

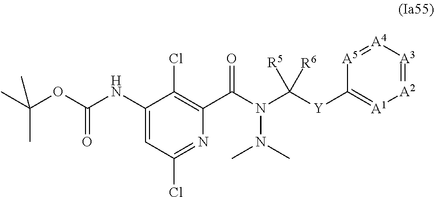

D3. Compounds D3-1 to D3-681 of the general formula (Ia56) in which R⁵, R⁶, Y, A¹, A², A³, A⁴, A⁵ correspond to the definitions (Nos. 1 to 681; corresponding to compounds D3-1 to D3-681) in table 1 above.

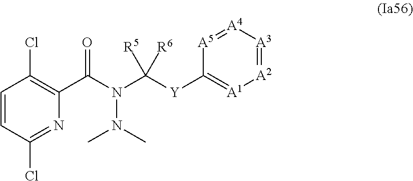

D4. Compounds D4-1 to D4-681 of the general formula (Ia57) in which R⁵, R⁶, Y, A¹, A², A³, A⁴, A⁵ correspond to the definitions (Nos. 1 to 681; corresponding to compounds D4-1 to D4-681) in table 1 above.

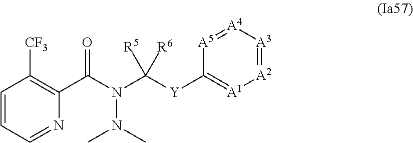

D5. Compounds D5-1 to D5-681 of the general formula (Ia58) in which R⁵, R⁶, Y, A¹, A², A³, A⁴, A⁵ correspond to the definitions (Nos. 1 to 681; corresponding to compounds D5-1 to D5-681) in table 1 above.

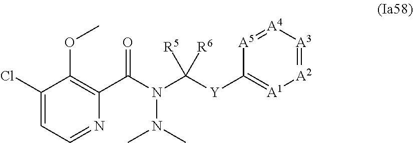

D6. Compounds D6-1 to D6-681 of the general formula (Ia59) in which R⁵, R⁶, Y, A¹, A², A³, A⁴, A⁵ correspond to the definitions (Nos. 1 to 681; corresponding to compounds D6-1 to D6-681) in table 1 above.

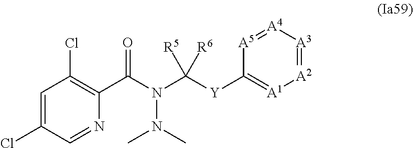

D7. Compounds D7-1 to D7-681 of the general formula (Ie11) in which R⁵, R⁶, Y, A¹, A², A³, A⁴, A⁵ correspond to the definitions (Nos. 1 to 681; corresponding to compounds D7-1 to D7-681) in table 1 above.

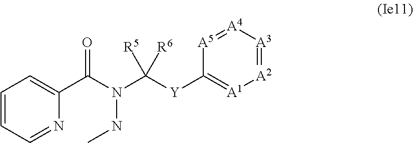

E1. Compounds E1-1 to E1-681 of the general formula (Ia60) in which R⁵, R⁶, Y, A¹, A², A³, A⁴, A⁵ correspond to the definitions (Nos. 1 to 681; corresponding to compounds E1-1 to El -681) in table 1 above.

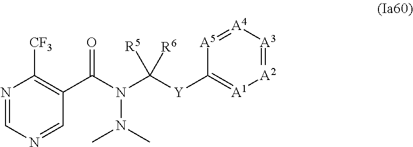

E2. Compounds E2-1 to E2-681 of the general formula (Ia61) in which R⁵, R⁶, Y, A¹, A², A³, A⁴, A⁵ correspond to the definitions (Nos. 1 to 681; corresponding to compounds E2-1 to E2-681) in table 1 above.

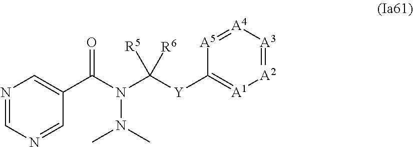

E3. Compounds E3-1 to E3-681 of the general formula (Ia62) in which R⁵, R⁶, Y, A¹, A², A³, A⁴, A⁵ correspond to the definitions (Nos. 1 to 681; corresponding to compounds E3-1 to E3-681) in table 1 above.

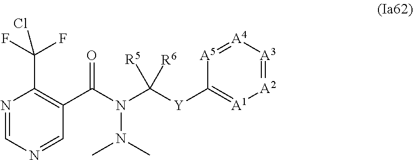

E4. Compounds E4-1 to E4-681 of the general formula (Ia63) in which R⁵, R⁶, Y, A¹, A², A³, A⁴, A⁵ correspond to the definitions (Nos. 1 to 681; corresponding to compounds E4-1 to E4-681) in table 1 above.

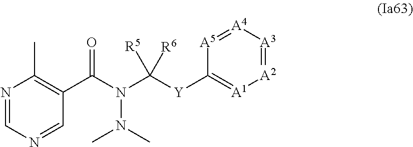

E5. Compounds E5-1 to E5-681 of the general formula (Ia64) in which R⁵, R⁶, Y, A¹, A², A³, A⁴, A⁵ correspond to the definitions (Nos. 1 to 681; corresponding to compounds E5-1 to E5-681) in table 1 above.

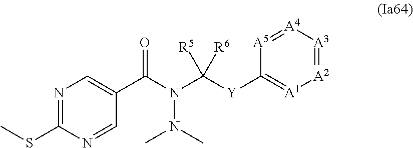

F1. Compounds F1-1 to F1-681 of the general formula (Ia65) in which R⁵, R⁶, Y, A¹, A², A³, A⁴, A⁵ correspond to the definitions (Nos. 1 to 681; corresponding to compounds F1-1 to F1-681) in table 1 above.

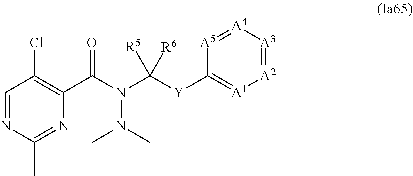

F2. Compounds F2-1 to F2-681 of the general formula (Ia66) in which R⁵, R⁶, Y, A¹, A², A³, A⁴, A⁵ correspond to the definitions (Nos. 1 to 681; corresponding to compounds F2-1 to F2-681) in table 1 above.

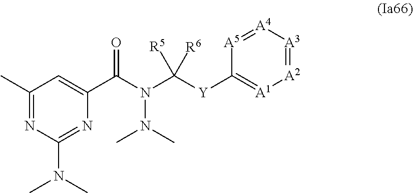

G1. Compounds G1-1 to G1-681 of the general formula (Ia67) in which R⁵, R⁶, Y, A¹, A², A³, A⁴, A⁵ correspond to the definitions (Nos. 1 to 681; corresponding to compounds G1-1 to G1-681) in table 1 above.

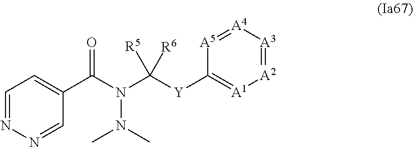

G2. Compounds G2-1 to G2-681 of the general formula (Ia68) in which R⁵, R⁶, Y, A¹, A², A³, A⁴, A⁵ correspond to the definitions (Nos. 1 to 681; corresponding to compounds G2-1 to G2-681) in table 1 above.

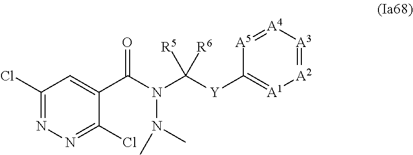

G3. Compounds G3-1 to G3-681 of the general formula (Ia69) in which R⁵, R⁶, Y, A¹, A², A³, A⁴, A⁵ correspond to the definitions (Nos. 1 to 681; corresponding to compounds G3-1 to G3-681) in table 1 above.

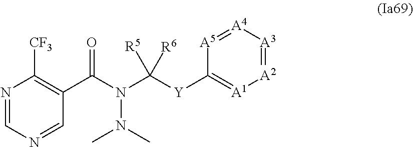

H1. Compounds H1-1 to H1-681 of the general formula (Ia70) in which R⁵, R⁶, Y, A¹, A², A³, A⁴, A⁵ correspond to the definitions (Nos. 1 to 681; corresponding to compounds H1-1 to H1-681) in table 1 above.

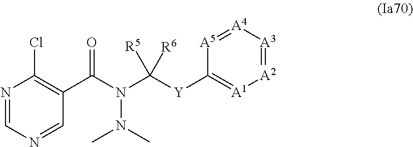

Spectroscopic Data of Selected Table Examples:

The spectroscopic data listed hereinafter for selected table examples were evaluated via conventional ¹H NMR interpretation or via NMR peak list methods.

a) Conventional ¹H NMR Interpretation

### Example No. A1-612

¹H-NMR (400 MHz, CDCl₃ δ, ppm) 7.74 (m, 1H), 7.65 (m, 1H), 7.62-7.57 (m, 2H), 7.41-7.34 (m, 2H), 7.29-7.23 (m, 2H), 4.19-4.13 (m, 2H), 3.82 (s, 6H), 3.40-3.36 (m, 2H).

### Example No. A2-61

¹H-NMR (400 MHz, CDCl₃ δ, ppm) 8.32 (m, 1H), 7.82-7.71 (m, 3H), 7.68-7.53 (m, 3H), 7.18 (m, 1H), 5.38 (s, 2H), 4.17-4.09 (m, 2H), 3.99-3.92 (m, 2H), 1.51 (t, 6H).

### Example No. A3-1

¹H-NMR (400 MHz, CDCl₃ δ, ppm) 7.68 (m, 1H), 7.58 (m, 1H), 7.51-7.46 (m, 2H), 7.37 (m, 1H), 7.31-7.18 (m, 3H), 4.79 (s, 3H), 3.12-2.99 (m, 1H), 2.55-2.42 (m, 1H), 1.43 (s, 9H).

### Example No. A5-40

¹H-NMR (400 MHz, CDCl₃ δ, ppm) 8.48 (d, 1H), 7.63-7.59 (m, 2H), 7.52-7.47 (m, 2H), 7.39-7.36 (m, 1H), 5.62 (d, 1H), 5.32 (d, 1H), 4.22-4.12 (m, 1H), 3.82-3.75 (m, 1H), 3.54 (s, 3H), 1.45 (m, 3H).

### Example No. A5-61

¹H-NMR (400 MHz, CDCl₃ δ, ppm) 8.01 (m, 1H), 7.78 (m, 1H), 7.73-7.64 (m, 2H), 7.60-7.55 (m, 2H), 7.52-7.47 (m, 1H), 7.42-7.37 (m, 1H), 5.46 (d, 1H), 5.14 (d, 1H), 4.19-4.12 (m, 1H), 3.78-3.71 (m, 1H), 3.29 (s, 3H), 1.48 (m, 3H).

### Example No. A7-1

¹H-NMR (400 MHz, CDCl₃ δ, ppm) 7.80 (m, 1H), 7.54 (m, 1H), 7.48-7.39 (m, 3H), 7.31-7.25 (m, 2H), 5.38 (s, 2H), 3.59 (s, 6H).

### Example No. A7-6

¹H-NMR (400 MHz, CDCl₃ δ, ppm) 7.83 (m, 1H), 7.47 (m, 1H), 7.44 (m, 2H), 7.32-7.24 (m, 2H), 5.30 (s, 2H), 3.55 (s, 6H).

### Example No. A7-11

¹H-NMR (400 MHz, CDCl₃ δ, ppm) 7.71-7.66 (m, 1H), 7.56-7.50 (m, 1H), 7.42 (m, 1H), 7.31-7.19 (m, 4H), 5.24 (s, 2H), 3.55 (s, 6H).

### Example No. A7-15

¹H-NMR (400 MHz, CDCl₃ δ, ppm) 7.77-7.70 (m, 1H), 7.39 (m, 1H), 7.31-7.27 (m, 2H), 7.07-7.00 (m, 1H), 6.98-6.94 (m, 1H), 5.21 (s, 2H), 3.57 (s, 6H).

### Example No. A7-37

¹H-NMR (400 MHz, CDCl₃ δ, ppm) 8.33 (m, 1H), 7.43 (m, 1H), 7.31-7.27 (m, 2H), 5.32 (s, 2H), 3.83 (s, 3H), 3.77 (s, 6H), 2.45 (s, 3H), 2.34 (s, 3H).

### Example No. A7-44

¹H-NMR (400 MHz, CDCl₃ δ, ppm) 8.08 (m, 1H), 7.72-7.65 (m, 2H), 7.42 (m, 1H), 7.31-7.23 (m, 2H), 5.34 (s, 2H), 3.56 (s, 6H).

### Example No. A7-61

¹H-NMR (400 MHz, CDCl₃ δ, ppm) 8.03 (m, 1H), 7.81-7.74 (m, 2H), 7.66-7.62 (m, 1H), 7.41 (m, 1H), 7.32-7.24 (m, 2H), 5.34 (s, 2H), 3.59 (s, 6H).

### Example No. A7-122

¹H-NMR (400 MHz, CDCl₃ δ, ppm) 8.34 (d, 1H), 8.12 (m, 1H), 7.93 (m, 1H), 7.84-7.78 (m, 2H), 7.69-7.65 (m, 1H), 7.37 (m, 1H), 7.36-7.27 (m, 2H), 5.49 (s, 2H), 3.80 (s, 6H).

### Example No. A8-1

¹H-NMR (400 MHz, CDCl₃ δ, ppm) 7.82 (m, 1H), 7.58-7.54 (m, 2H), 7.51-7.39 (m, 3H), 7.02-6.95 (m, 2H), 5.57 (s, 2H), 3.90 (s, 3H), 3.82 (s, 6H).

### Example No. A8-37

¹H-NMR (400 MHz, CDCl₃ δ, ppm) 8.25 (s, 1H), 7.90 (m, 1H), 7.55-7.50 (m, 1H), 7.09-7.05 (m, 1H), 7.02-6.99 (m, 1H), 5.48 (s, 2H), 4.02 (s, 6H), 4.01 (s, 3H), 3.81 (s, 3H), 2.49 (s, 3H), 2.31 (s, 3H).

### Example No. A8-44

¹H-NMR (400 MHz, CDCl₃ δ, ppm) 7.68-7.64 (m, 1H), 7.58 (m, 1H), 7.48-7.40 (m, 3H), 7.01-6.94 (m, 2H), 5.40 (s, 2H), 3.88 (s, 3H), 3.79 (s, 6H).

### Example No. A8-61

¹H-NMR (400 MHz, CDCl₃ δ, ppm) 8.03 (m, 1H), 7.83 (m, 1H), 7.78-7.74 (m, 1H), 7.69-7.66 (m, 1H), 7.54 (m, 1H), 7.47-7.43 (m, 1H), 7.01-6.95 (m, 2H), 5.55 (s, 2H), 3.89 (s, 3H), 3.84 (s, 6H).

### Example No. A8-86

¹H-NMR (400 MHz, CDCl₃ δ, ppm) 8.02 (s, 1H), 7.75-7.67 (m, 2H), 7.58 (m, 1H), 7.47-7.43 (m, 1H), 7.00-6.95 (m, 2H), 5.59 (s, 2H), 3.87 (s, 3H), 3.82 (s, 6H).

### Example No. A8-122

¹H-NMR (400 MHz, CDCl₃ δ, ppm) 8.35 (m, 1H), 8.24 (m, 1H), 8.01-7.97 (m, 1H), 7.93 (m, 1H), 7.88 (m, 1H), 7.78-7.74 (m, 1H), 7.67-7.57 (m, 2H), 7.15-7.05 (m, 2H), 5.98 (s, 2H), 4.18 (s, 6H), 3.96 (s, 3H).

### Example No. A8-144

¹H-NMR (400 MHz, CDCl₃ δ, ppm) 8.42 (d, 1H), 7.85 (m, 1H), 7.54-7.49 (m, 1H), 7.07 (m, 1H), 7.01 (m, 1H), 6.85 (m, 1H), 5.54 (s, 2H), 4.49-4.44 (m, 2H), 4.02 (s, 6H), 3.98 (s, 3H), 2.46 (s, 3H).

### Example No. A9-1

¹H-NMR (400 MHz, CDCl₃ δ, ppm) 7.92 (d, 2H), 7.62 (m, 1H), 7.57 (m, 1H), 7.49-7.46 (m, 1H), 7.43-7.36 (m, 3H), 5.48 (s, 2H), 3.75 (s, 6H).

### Example No. A9-6

¹H-NMR (400 MHz, CDCl₃ δ, ppm) 7.91 (d, 2H), 7.59-7.55 (m, 2H), 7.41 (d, 2H), 7.36 (m, 1H), 5.42 (s, 2H), 3.70 (s, 6H).

### Example No. A9-11

¹H-NMR (400 MHz, CDCl₃ δ, ppm) 7.88 (d, 2H), 7.62-7.55 (m, 1H), 7.42 (d, 2H), 7.33 (m, 1H), 7.03-6.97 (m, 2H), 5.33 (s, 2H), 3.73 (s, 6H).

### Example No. A9-15

¹H-NMR (400 MHz, CDCl₃ δ, ppm) 7.88 (d, 2H), 7.61-7.56 (m, 1H), 7.42 (d, 2H), 7.03-6.97 (m, 2H), 5.33 (s, 2H), 3.73 (s, 6H).

### Example No. A9-21

¹H-NMR (400 MHz, CDCl₃ δ, ppm) 8.70 (m, 1H), 7.86 (d, 2H), 7.84-7.74 (m, 1H), 7.66 (m, 1H), 7.45-7.38 (m, 3H), 5.38 (s, 2H), 3.86 (s, 6H).

### Example No. A9-44

¹H-NMR (400 MHz, CDCl₃ δ, ppm) 7.88 (d, 2H), 7.47-7.38 (m, 5H), 5.30 (s, 2H), 3.70 (s, 6H).

### Example No. A9-37

¹H-NMR (400 MHz, CDCl₃ δ, ppm) 8.30 (s, 1H), 7.92 (d, 2H), 7.43 (d, 2H), 5.42 (s, 2H), 3.87 (s, 6H), 3.81 (s, 3H), 2.41 (s, 3H), 2.32 (s, 3H).

### Example No. A9-61

¹H-NMR (400 MHz, CDCl₃ δ, ppm) 7.88-7.82 (m, 3H), 7.76-7.63 (m, 3H), 7.40 (d, 2H), 5.45 (s, 2H), 3.76 (s, 6H).

### Example No. A9-122

¹H-NMR (400 MHz, CDCl₃ δ, ppm) 8.29 (m, 1H), 8.09-8.06 (m, 1H), 7.91-7.80 (m, 4H), 7.76-7.65 (m, 2H), 7.42 (d, 2H), 5.57 (s, 2H), 3.93 (s, 6H).

### Example No. A10-1

¹H-NMR (400 MHz, CDCl₃ δ, ppm) 7.81 (m, 1H), 7.56 (m, 1H), 7.52-7.47 (m, 2H), 7.45-7.34 (m, 3H), 7.30-7.27 (m, 1H), 5.49 (s, 2H), 3.72 (s, 6H).

### Example No. A10-6

¹H-NMR (400 MHz, CDCl₃ δ, ppm) 7.83 (s, 1H), 7.50-7.45 (m, 3H), 7.41-7.39 (m, 1H), 7.35-7.29 (m, 2H), 5.39 (s, 2H), 3.66 (s, 6H).

### Example No. A10-11

¹H-NMR (400 MHz, CDCl₃ δ, ppm) 7.72-7.68 (m, 1H), 7.59-7.53 (m, 1H), 7.46-7.44 (m, 1H), 7.42-7.21 (m, 5H), 5.36 (s, 2H), 3.70 (s, 6H).

### Example No. A10-15

¹H-NMR (400 MHz, CDCl₃ δ, ppm) 7.78-7.72 (m, 1H), 7.44-7.34 (m, 3H), 7.28 (m, 1H), 7.08-6.97 (m, 2H), 5.32 (s, 2H), 3.69 (s, 6H).

### Example No. A10-37

¹H-NMR (400 MHz, CDCl₃ δ, ppm) 8.31 (s, 1H), 7.51 (m, 1H), 7.42-7.35 (m, 2H), 7.31-7.27 (m, 1H), 5.42 (s, 2H), 3.86 (s, 6H), 3.82 (s, 3H), 2.45 (s, 3H), 2.32 (s, 3H).

### Example No. A10-44

¹H-NMR (400 MHz, CDCl₃ δ, ppm) 8.04 (m, 1H), 7.84-7.82 (m, 1H), 7.80-7.76 (m, 1H), 7.69-7.65 (m, 1H), 7.45-7.39 (m, 2H), 7.37-7.33 (m, 1H), 5.44 (s, 2H), 3.73 (s, 6H).

### Example No. A10-61

¹H-NMR (400 MHz, CDCl₃ δ, ppm) 8.04 (m, 1H), 7.84-7.82 (m, 1H), 7.80-7.76 (m, 1H), 7.69-7.65 (m, 1H), 7.45-7.37 (m, 2H), 7.35-7.32 (m, 1H), 7.29-7.25 (m, 1H), 5.44 (s, 2H), 3.73 (s, 6H).

### Example No. A10-122

¹H-NMR (400 MHz, CDCl₃ δ, ppm) 8.34 (m, 1H), 8.10-8.07 (m, 1H), 7.93 (m, 1H), 7.84-7.80 (m, 2H), 7.69-7.66 (m, 1H), 7.43-7.35 (m, 3H), 7.29-7.24 (m, 1H), 5.57 (s, 2H), 3.92 (s, 6H).

### Example No. A13-21

¹H-NMR (400 MHz, CDCl₃ δ, ppm) 8.67 (m, 1H), 7.80-7.76 (m, 1H), 7.74-7.71 (m, 1H), 7.48 (m, 1H), 7.38-7.35 (m, 1H), 7.18 (s, 1H), 7.14 (m, 1H), 5.17 (s, 2H), 3.48 (s, 6H), 2.33 (s, 3H).

### Example No. A13-121

¹H-NMR (400 MHz, CDCl₃ δ, ppm) 8.24 (m, 1H), 8.12 (m, 1H), 7.87 (m, 1H), 7.82 (m, 1H), 7.80-7.76 (m, 1H), 7.63-7.59 (m, 1H), 7.48 (m, 1H), 7.23 (s, 1H), 7.15 (m, 1H), 5.34 (s, 2H), 4.57 (s, 6H), 2.33 (s, 3H).

### Example No. A13-144

¹H-NMR (400 MHz, CDCl₃ δ, ppm) 8.44 (m, 1H), 7.50 (m, 1H), 7.37 (m, 1H), 7.17 (m, 1H), 6.77 (m, 1H), 5.25 (s, 1H), 4.47-4.41 (m, 1H), 3.48 (s, 6H), 2.48 (s, 3H).

### Example No. A14-5

¹H-NMR (400 MHz, CDCl₃ δ, ppm) 7.73 (m, 1H), 7.55 (m, 1H), 7.51 (m, 1H), 7.37-7.33 (m, 2H), 7.21-7.18 (m, 1H), 5.20 (s, 2H), 3.40 (s, 6H).

### Example No. A14-35

¹H-NMR (400 MHz, CDCl₃ δ, ppm) 8.58 (m, 1H), 7.86 (m, 1H), 7.63-7.58 (m, 1H), 7.33-7.30 (m, 1H), 7.22-7.17 (m, 1H), 5.35 (s, 2H), 3.52 (s, 6H).

### Example No. A14-36

¹H-NMR (400 MHz, CDCl₃ δ, ppm) 8.87 (m, 1H), 8.07 (m, 1H), 7.66-7.54 (m, 2H), 7.21-7.16 (m, 1H), 5.47 (s, 2H), 3.59 (s, 6H).

Example No. A14-91

¹H-NMR (400 MHz, CDCl₃ δ, ppm) 8.10 (m, 1H), 7.77 (m, 1H), 7.66-7.55 (m, 2H), 7.36-7.33 (m, 1H), 7.22-7.18 (m, 1H), 5.29 (s, 2H), 3.44 (s, 6H).

### Example No. A14-99

¹H-NMR (400 MHz, CDCl₃ δ, ppm) 8.15 (m, 1H), 7.50 (m, 1H), 7.38-7.33 (m, 2H), 7.21-7.18 (m, 1H), 5.16 (s, 2H), 3.47 (s, 6H).

### Example No. A14-138

¹H-NMR (400 MHz, CDCl₃ δ, ppm) 7.03 (s, 1H), 7.63-7.57 (m, 2H), 7.36-7.33 (m, 1H), 7.22-7.18 (m, 1H), 5.19 (s, 2H), 3.42 (s, 6H).

### Example No. A14-144

¹H-NMR (400 MHz, CDCl₃ δ, ppm) 8.45 (m, 1H), 7.59-7.56 (m, 1H), 7.33 (m, 1H), 7.19 (m, 1H), 6.78 (m, 1H), 5.21 (s, 1H), 4.47-4.41 (m, 1H), 3.48 (s, 6H), 2.47 (s, 3H).

### Example No. A14-608

¹H-NMR (400 MHz, CDCl₃ δ, ppm) 7.65-7.59 (m, 2H), 7.35-7.31 (m, 1H), 7.23-7.18 (m, 1H), 5.42 (s, 2H), 3.46 (s, 6H).

### Example No. A14-680

¹H-NMR (400 MHz, CDCl₃ δ, ppm) 7.64-7.59 (m, 1H), 7.35-7.31 (m, 1H), 7.23-7.18 (m, 1H), 5.79 (s, 2H), 3.48 (s, 6H).

### Example No. A14-681

¹H-NMR (400 MHz, CDCl₃ δ, ppm) 8.75 (s, 1H), 7.57-7.54 (m, 1H), 7.51 (s, 1H), 7.35-7.31 (m, 1H), 7.22-7.18 (m, 1H), 5.20 (s, 2H), 3.44 (s, 6H).

### Example No. A47-1

¹H-NMR (400 MHz, CDCl₃ δ, ppm) 8.02 (m, 1H), 7.67 (m, 1H), 7.55 (m, 2H), 7.46 (m, 1H), 7.38-7.28 (m, 3H), 5.30 (m, 1H), 5.12-5.04 (m, 1H), 4.85 (m, 1H), 3.08 (s, 3H), 2.63-2.45 (m, 2H), 2.02-1.93 (m, 2H), 1.87-1.72 (m, 1H), 1.65-1.47 (m, 3H), 1.30-1.12 (m, 2H).

### Example No. A47-21

¹H-NMR (400 MHz, CDCl₃ δ, ppm) 8.64 (m, 1H), 7.86 (m, 1H), 7.72 (m, 1H), 7.61 (m, 1H), 7.37-7.29 (m, 3H), 7.13 (m, 1H), 5.33 (m, 1H), 4.79 (m, 1H), 4.64-4.56 (m, 1H), 3.32 (s, 3H), 2.63-2.45 (m, 2H), 2.02-1.85 (m, 2H), 1.83-1.38 (m, 4H), 1.30-1.08 (m, 2H).

### Example No. A48-35

¹H-NMR (400 MHz, CDCl₃ δ, ppm) 8.58 (m, 1H), 7.92 (m, 1H), 7.87 (m, 1H), 7.80 (m, 1H), 7.72 (m, 1H), 5.31 (s, 2H), 3.52 (s, 6H).

### Example No. A49-35

¹H-NMR (400 MHz, CDCl₃ δ, ppm) 8.59 (m, 1H), 7.90-7.86 (m, 2H), 7.77-7.72 (m, 2H), 5.34 (s, 2H), 3.53 (s, 6H).

### Example No. A51-35

¹H-NMR (400 MHz, CDCl₃ δ, ppm) 8.58 (m, 1H), 7.86 (m, 1H), 7.61 (m, 1H), 7.56 (m, 1H), 7.48 (m, 1H), 5.34 (s, 2H), 3.51 (s, 6H).

### Example No. B1-1

¹H-NMR (400 MHz, CDCl₃ δ, ppm) 8.88 (s, 1H), 8.70 (m, 1H), 7.78 (m, 1H), 7.52-7.48 (m, 2H), 7.44-7.36 (m, 2H), 5.23 (s, 2H), 3.44 (s, 6H).

### Example No. B1-61

¹H-NMR (400 MHz, CDCl₃ δ, ppm) 8.81 (s, 1H), 8.70 (m, 1H), 7.97 (m, 1H), 7.78 (m, 1H), 7.75-7.70 (m, 1H), 7.61 (m, 1H), 7.51 (m, 1H), 5.24 (s, 2H), 3.50 (s, 6H).

### Example No. B1-94

¹H-NMR (400 MHz, CDCl₃ δ, ppm) 8.78 (s, 1H), 8.71 (m, 1H), 8.48 (m, 1H), 7.96 (m, 1H), 7.51 (m, 1H), 7.45 (m, 1H), 4.96 (s, 2H), 3.43 (s, 6H).

### Example No. B1-351

¹H-NMR (400 MHz, CDCl₃ δ, ppm) 8.87 (m, 1H), 8.82 (m, 1H), 7.58 (m, 1H), 7.34-7.28 (m, 3H), 7.24-7.18 (m, 2H), 4.33-4.28 (m, 1H), 3.26-3.20 (m, 1H), 3.10-3.04 (m, 1H), 2.84 (s, 3H), 2.48 (s, 3H), 1.71 (d, 3H).

### Example No. B1-451

¹H-NMR (400 MHz, CDCl₃ δ, ppm) 8.84 (m, 1H), 8.75 (m, 1H), 7.54 (m, 1H), 7.32 (m, 2H), 7.24-7.18 (m, 2H), 3.81-3.76 (m, 2H), 3.48 (s, 6H), 2.75-2.70 (m, 2H), 2.25-2.20 (m, 2H).

### Example No. B1-454

¹H-NMR (400 MHz, CDCl₃ δ, ppm) 8.84 (m, 1H), 8.69 (m, 1H), 7.49 (m, 1H), 7.29 (d, 2H), 7.13 (d, 2H), 3.76-3.71 (m, 2H), 3.41 (s, 6H), 2.69 (m, 2H), 2.19 (m, 2H).

### Example No. B1-612

¹H-NMR (400 MHz, CDCl₃ δ, ppm) 8.92 (m, 1H), 8.79 (m, 1H), 7.60 (m, 1H), 7.39 (m, 1H), 7.35-7.30 (m, 2H), 7.24 (m, 1H), 4.01-3.96 (m, 2H), 3.62 (s, 6H), 3.35-3.31 (m, 2H).

### Example No. B5-1

¹H-NMR (400 MHz, CDCl₃ δ, ppm) 9.26 (s, 1H), 8.42 (m, 1H), 7.67 (m, 2H), 7.50 (m, 1H), 7.43-7.37 (m, 1H), 7.33-7.29 (m, 1H), 5.18 (s, 2H), 3.48 (s, 6H).

### Example No. B5-21

¹H-NMR (400 MHz, CDCl₃ δ, ppm) 9.18 (s, 1H), 8.65 (m, 1H), 8.36 (m, 1H), 7.74-7.70 (m, 1H), 7.65-7.58 (m, 2H), 7.37-7.33 (m, 1H), 5.07 (s, 2H), 3.53 (s, 6H).

### Example No. B5-35

¹H-NMR (400 MHz, CDCl₃ δ, ppm) 9.20 (s, 1H), 8.55 (m, 1H), 8.41 (m, 1H), 7.88 (m, 1H), 7.65 (d, 1H), 5.17 (s, 2H), 3.53 (s, 6H).

### Example No. B5-61

¹H-NMR (400 MHz, CDCl₃ δ, ppm) 9.22 (s, 1H), 8.41 (m, 1H), 7.79 (m, 2H), 7.68-7.57 (m, 3H), 5.21 (s, 2H), 3.54 (s, 6H).

### Example No. B5-94

¹H-NMR (400 MHz, CDCl₃ δ, ppm) 9.20 (s, 1H), 8.45 (m, 1H), 8.38 (m, 1H), 7.85 (m, 1H), 7.67 (m, 1H), 7.39 (m, 1H), 4.95 (s, 2H), 3.46 (s, 6H).

### Example No. B6-1

¹H-NMR (400 MHz, CDCl₃ δ, ppm) 9.19 (m, 1H), 8.39 (m, 1H), 7.67 (m, 1H), 7.62 (m, 1H), 7.50 (m, 1H), 7.41-7.37 (m, 1H), 7.32-7.27 (m, 1H), 6.80-6.53 (br. t, 1H, CHF₂), 5.20 (s, 2H), 3.47 (s, 6H).

### Example No. B6-21

¹H-NMR (400 MHz, CDCl₃ δ, ppm) 9.11 (m, 1H), 8.66 (m, 1H), 8.33 (m, 1H), 7.72-7.68 (m, 1H), 7.61-7.57 (m, 1H), 7.36 (m, 1H), 6.79-6.51 (br. t, 1H, CHF₂), 5.09 (s, 2H), 3.52 (s, 6H).

### Example No. B6-35

¹H-NMR (400 MHz, CDCl₃ δ, ppm) 9.15 (m, 1H), 8.55 (m, 1H), 8.37 (m, 1H), 7.87 (m, 1H), 7.60 (m, 1H), 7.36 (m, 1H), 6.79-6.51 (br. t, 1H, CHF₂), 5.19 (s, 2H), 3.61 (s, 6H).

### Example No. B6-61

¹H-NMR (400 MHz, CDCl₃ δ, ppm) 9.16 (m, 1H), 8.37 (m, 1H), 7.79 (m, 2H), 7.62-7.55 (m, 3H), 6.80-6.52 (br. t, 1H, CHF₂), 5.23 (s, 2H), 3.53 (s, 6H).

### Example No. B6-94

¹H-NMR (400 MHz, CDCl₃ δ, ppm) 9.13 (m, 1H), 8.45 (m, 1H), 8.34 (m, 1H), 7.84 (m, 1H), 7.62 (m, 1H), 7.38 (m, 1H), 6.80-6.52 (br. t, 1H, CHF₂), 4.97 (s, 2H), 3.48 (s, 6H).

### Example No. B16-94

¹H-NMR (400 MHz, CDCl₃ δ, ppm) 8.50 (m, 1H), 8.34 (m, 1H), 8.02 (m, 1H), 7.73 (m, 1H), 7.44 (m, 1H), 7.20 (m, 1H), 5.03 (s, 2H), 3.44 (s, 6H).

### Example No. B17-40

¹H-NMR (400 MHz, CDCl₃ δ, ppm) 8.94 (s, 1H), 8.77 (m, 1H), 8.40 (m, 1H), 7.52 (d, 1H), 7.36 (d, 1H), 5.02 (d, 1H), 4.92 (d, 1H), 2.95-2.87 (m, 1H), 2.61-2.50 (m, 1H), 2.45 (s, 3H), 1.48 (m, 3H).

### Example No. B17-61

¹H-NMR (400 MHz, CDCl₃ δ, ppm) 8.85 (s, 1H), 8.71 (m, 1H), 7.97 (m, 1H), 7.78 (m, 1H), 7.71 (m, 1H), 7.63-7.57 (m, 1H), 7.52 (m, 1H), 5.42 (d, 1H), 5.07 (d, 1H), 4.19-4.12 (m, 1H), 3.84-3.77 (m, 1H), 3.30 (s, 3H), 1.48 (m, 3H).

### Example No. B18-1

¹H-NMR (400 MHz, CD₃OD δ, ppm) 8.78 (m, 1H), 8.68 (m, 1H), 7.87 (m, 1H), 7.72 (m, 1H), 7.58 (m, 1H), 7.52 (m, 1H), 7.40 (m, 1H), 5.07 (m, 1H), 4.94 (m, 1H), 4.88-4.82 (m, 1H), 3.25 (s, 3H), 2.60-2.53 (m, 1H), 2.49-2.42 (m, 1H), 2.08-2.02 (m, 2H), 1.82-1.74 (m, 2H), 1.58-1.42 (m, 2H), 1.38-1.24 (m, 2H).

### Example No. B18-21

¹H-NMR (400 MHz, CDCl₃ δ, ppm) 8.64 (m, 2H), 8.39 (m, 1H), 7.80 (m, 1H), 7.74 (m, 1H), 7.47 (m, 1H), 7.37 (m, 1H), 5.49 (m, 1H), 4.75 (m, 1H), 4.69-4.61 (m, 1H), 3.35 (s, 3H), 2.59-2.48 (m, 2H), 2.02-1.90 (m, 2H), 1.78-1.35 (m, 5H), 1.28-1.12 (m, 1H).

### Example No. E1-1

¹H-NMR (400 MHz, CDCl₃ δ, ppm) 9.28 (s, 1H), 9.02 (s, 1H), 7.74 (m, 1H), 7.78 (m, 1H), 7.52-7.48 (m, 1H), 7.44-7.36 (m, 1H), 5.23 (s, 2H), 3.44 (s, 6H).

### Example No. E1-21

¹H-NMR (400 MHz, CDCl₃ δ, ppm) 9.25 (s, 1H), 8.81 (s, 1H), 8.69 (m, 1H), 7.81-7.76 (m, 1H), 7.68 (m, 1H), 7.42-7.37 (m, 1H), 5.08 (s, 2H), 3.51 (s, 6H).

### Example No. E1-61

¹H-NMR (400 MHz, CDCl₃ δ, ppm) 9.28 (s, 1H), 8.98 (s, 1H), 7.92 (m, 1H), 7.81 (m, 1H), 7.72-7.68 (m, 1H), 7.65-7.62 (m, 1H), 5.16 (s, 2H), 3.52 (s, 6H).

### Example No. E1-94

¹H-NMR (400 MHz, CDCl₃ δ, ppm) 9.28 (s, 1H), 8.94 (s, 1H), 8.49 (m, 1H), 7.92 (m, 1H), 7.45 (m, 1H), 4.89 (s, 2H), 3.45 (s, 6H).

### Example No. E3-1

¹H-NMR (400 MHz, CDCl₃ δ, ppm) 9.27 (s, 1H), 8.96 (s, 1H), 7.75 (m, 1H), 7.53 (m, 1H), 7.47-7.37 (m, 2H), 5.19 (s, 2H), 3.45 (s, 6H).

### Example No. E3-21

¹H-NMR (400 MHz, CDCl₃ δ, ppm) 9.24 (s, 1H), 8.73 (s, 1H), 8.69 (m, 1H), 7.84-7.79 (m, 1H), 7.70 (m, 1H), 7.43-7.39 (m, 1H), 5.11 (s, 2H), 3.53 (s, 6H).

### Example No. E3-61

¹H-NMR (400 MHz, CDCl₃ δ, ppm) 9.27 (s, 1H), 8.90 (s, 1H), 7.94 (m, 1H), 7.82 (m, 1H), 7.76-7.71 (m, 1H), 7.65-7.62 (m, 1H), 5.18 (s, 2H), 3.51 (s, 6H).

### Example No. G1-61

¹H-NMR (400 MHz, CDCl₃ δ, ppm) 9.62 (m, 1H), 9.24 (m, 1H), 7.98 (m, 1H), 7.80-7.74 (m, 2H), 7.66-7.57 (m, 2H), 5.19 (s, 2H), 3.54 (s, 6H).

b) NMR Peak List Method:

The ¹H NMR data of selected examples are stated in the form of ¹H NMR peak lists. For each signal peak, first the 6 value in ppm and then the signal intensity in round brackets are listed. The pairs of 6 valuesignal intensity numbers for different signal peaks are listed with separation from one another by semicolons. The peak list for a corresponding example therefore takes the form of: δ₁ (intensity₁); δ₂ (intensity₂); . . . ; δᵢ (intensityᵢ); . . . ; δₙ (intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum. To calibrate the chemical shift of ¹H-NMR spectra, we used tetramethylsilane and/or the chemical shift of the solvent, particularly in the case of spectra measured in DMSO. Therefore, the tetramethylsilane peak may but need not occur in NMR peak lists. The lists of the ¹H NMR peaks are similar to the conventional ¹H NMR printouts and thus usually contain all peaks listed in a conventional NMR interpretation. In addition, like conventional ¹H NMR printouts, they may show solvent signals, signals of stereoisomers of the target compounds which are likewise provided by the invention, and/or peaks of impurities. In the reporting of compound signals within the delta range of solvents and/or water, our lists of ¹H NMR peaks show the standard solvent peaks, for example peaks of DMSO in DMSO-d₆ and the peak of water, which usually have a high intensity on average. The peaks of stereoisomers of the target compounds and/or peaks of impurities usually have a lower intensity on average than the peaks of the target compounds (for example with a purity of >90%).

Such stereoisomers and/or impurities may be typical of the particular preparation process. Their peaks can thus help in identifying reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the peak picking in question in conventional ¹H NMR interpretation.

### Example No. A1-1

### ¹H-NMR (400 MHz, CDCl₃ δ, ppm)

7.792(0.7);7.787(0.6);7.774(1.0);7.768(0.8);7.645(0.6);7.643(0.7);7.642(0.6);7.625(0.7); 7.624(0.8);7.622(0.7);7.591(0.6);7.590(0.5);7.574(0.7);7.572(0.8);7.571(0.8);7.501(0.7); 7.497(1.0);7.492(0.8);7.490(0.7);7.483(0.7);7.481(0.8);7.478(1.1);7.411(1.1);7.406(1.1); 7.404(0.5);7.3924(1.2);7.3917(1.1);7.387(1.9);7.373(1.0);7.369(0.9);7.264(8.1);5.276(4.6);3.409(16.0);2.002(2.2);1.732(0.5);0.000(3.5)

### Example No. A1-3

### ¹H-NMR (400 MHz, CDCl₃ δ, ppm)

7.636(0.7);7.634(0.7);7.616(0.8);7.615(0.8);7.507(1.0);7.506(0.9);7.504(0.9);7.503(1.0); 7.501(0.8);7.500(0.8);7.492(1.4);7.487(0.6);7.481(0.8);7.476(1.1);7.471(3.2);7.465(0.6); 7.444(0.6);7.439(3.6);7.433(0.8);7.422(0.6);7.417(1.5);7.380(0.6);7.378(0.6);7.376(0.5); 7.263(11.7);5.017(4.4);3.342(16.0);2.004(2.3);0.000(5.1)

### Example No. A1-4

### ¹H-NMR (400 MHz, CDCl₃ δ, ppm)

7.867(0.8);7.863(0.8);7.847(0.9);7.843(0.9);7.791(0.6);7.745(1.1);7.741(1.2);7.725(1.4); 7.721(1.4);7.622(0.6);7.566(0.9);7.546(1.5);7.526(0.7);5.309(1.9);3.534(4.0);2.511(5.0); 2.506(11.0);2.502(15.5);2.497(11.1);2.493(5.1);2.074(16.0);0.000(8.4)

### Example No. A1-5

### ¹H-NMR (400 MHz, CDCl₃ δ, ppm)

7.861(1.4);7.856(1.5);7.802(0.6);7.767(1.6);7.746(2.3);7.660(1.1);7.655(1.0);7.639(1.0); 7.634(1.2);5.242(1.8);3.534(4.1);2.511(4.2);2.506(9.4);2.502(13.2);2.497(9.5);2.493(4.4); 2.074(16.0);0.000(9.6)

### Example No. A1-7

### ¹H-NMR (400 MHz, d₆-DMSO δ, ppm)

7.812(0.6);7.793(0.8);7.754(0.6);7.736(0.7);7.710(2.8);7.708(2.8);7.689(4.8);7.675(0.5); 7.623(1.8);7.605(1.3);7.600(1.1);7.582(0.8);5.451(3.4);3.586(4.9);2.511(5.3);2.506(12.1); 2.502(17.3);2.497(12.6);2.493(5.9);2.074(16.0);0.000(11.7)

### Example No. A1-10

### ¹H-NMR (400 MHz, CDCl₃ δ, ppm)

7.637(0.8);7.620(1.4);7.605(0.8);7.603(0.9);7.522(0.7);7.518(0.6);7.514(8.3);7.503(0.9); 7.385(0.7);7.384(0.7);7.263(20.2);5.554(5.1);3.449(16.0);2.955(2.1);2.883(1.8);2.882(1.8);2.710(0.8);1.629(1.6);0.000(7.9)

### Example No. A1-17

### ¹H-NMR (400 MHz, CDCl₃ δ, ppm)

7.630(2.2);7.628(2.2);7.608(2.9);7.517(0.8);7.516(0.8);7.511(0.9);7.506(0.8);7.499(1.5); 7.497(1.5);7.490(1.6);7.484(0.6);7.481(0.8);7.479(0.8);7.474(0.8);7.468(0.9);7.398(0.6); 7.395(0.9);7.377(1.1);7.375(1.2);7.357(0.5);7.261(34.7);7.080(2.2);7.060(3.2);7.040(1.9); 5.203(5.1);3.405(16.0);1.607(5.4);0.000(15.4)

### Example No. A1-21

### ¹H-NMR (400 MHz, CDCl₃ δ, ppm)

8.667(0.8);8.657(0.8);8.655(0.9);7.805(0.3);7.785(0.9);7.782(0.8);7.767(0.7);7.763(0.6); 7.740(1.4);7.738(1.5);7.721(0.7);7.719(0.7);7.618(1.0);7.598(1.2);7.558(0.5);7.436(0.9); 7.418(0.9);7.392(1.3);7.384(0.8);7.381(0.6);7.372(1.8);7.369(1.4);7.353(1.5);7.333(0.3); 7.265(8.5);7.263(8.8);7.262(9.6);5.300(0.6);5.298(0.6);5.297(0.7);5.164(5.3);3.476(16.0); 2.710(3.3);2.460(1.2);2.044(0.7);2.042(0.8);1. 663(0.8);1.435(0.6);1.432(0.7);1.276(0.4);1.259(1.2);1.240(0.3);0.073(0.9);0.071(0.9);0.003(3.1);0.001 (3.2);0.000(3.4)

### Example No. A1-27

### ¹H-NMR (400 MHz, CDCl₃ δ, ppm)

8.849(3.7);8.837(3.8);7.608(0.6);7.589(0.8);7.566(0.5);7.547(0.8);7.470(0.7);7.378(1.2); 7.365(1.8);7.353(1.4);7.261(44.4);5.327(4.5);3.591(16.0);2.888(0.5);2.044(0.5);1.591(1.0); 0.000(19.2);-0.009(0.6)

### Example No. A1-30

### ¹H-NMR (400 MHz, CDCl₃ δ, ppm)

7.739(0.9);7.720(1.1);7.621(1.8);7.615(1.2);7.609(1.3);7.580(0.5);7.518(1.1);7.259(176.7); 6.995(1.0);5.980(2.5);5.928(0.7);5.907(0.6);3.963(2.5);3.940(16.0);3.127(1.1);2.941(1.1);2.425(5.9);1.537(16.8);0.008(2.1);0.000(69.4);-0.009(2.0)

### Example No. A1-33

### ¹H-NMR (400 MHz, d₆-DMSO δ, ppm)

8.751(1.8);8.738(1.8);7.779(6.6);7.775(7.8);7.746(2.4);7.735(2.4);7.410(0.6);5.198(2.2); 3.580(7.7);3.168(2.5);2.674(1.7);2.670(2.4);2.665(1.7);2.553(1.7);2.540(0.8);2.523(7.1); 2.519(10.4);2.510(135.3);2.505(292.9);2.501(406.8);2.496(284.9);2.492(127.4);2.467(1.2);2.451(0.9);2.446(0.8);2.363(0.9);2.337(0.9);2.332(1.8);2.328(2.5);2.323(1.8);2.318(0.8);2.073(16.0);0.146(0.7);0.008(6.8);0.000(239.3);-0.009(7.2);-0.034(0.6);−0.050(0.6);−0.150(0.8)

### Example No. A1-37

### ¹H-NMR (400 MHz, CDCl₃ δ, ppm)

8.296(1.7);7.621(0.7);7.620(0.7);7.619(0.7);7.600(0.9);7.599(0.8);7.569(0.6);7.568(0.5); 7.552(0.9);7.550(0.9);7.549(0.8);7.479(0.8);7.477(0.7);7.363(0.7);7.262(21.9);5.298(3.4); 5.168(4.1);3.790(16.0);3.481(13.8);2.710(3.8);2.485(8.1);2.460(1.3);2.305(6.5);1.257(0.7);1.219(0.9);0.000(7.7)

### Example No. A1-38

### ¹H-NMR (400 MHz, CDCl₃ δ, ppm)

8.509(0.7);8.506(0.7);8.504(0.7);8.497(0.7);8.494(0.7);7.624(0.8);7.623(0.8);7.605(1.5); 7.603(1.6);7.585(0.7);7.584(0.8);7.581(0.8);7.580(0.7);7.566(0.5);7.565(0.6);7.563(0.6); 7.547(0.9);7.546(1.0);7.544(0.9);7.502(0.5);7.501(0.6);7.483(0.9);7.482(0.9);7.369(0.8); 7.274(0.9);7.263(17.0);7.255(0.9);7.243(0.8);5.297(3.7);5.215(4.9);3.530(16.0);2.70.

### Example No. A1-59

### ¹H-NMR (400 MHz, d₆-DMSO δ, ppm)

7.827(0.6);7.807(0.9);7.764(0.7);7.746(0.6);7.707(0.8);7.691(1.1);7.687(1.4);7.671(2.0); 7.666(1.5);7.650(1.3);7.577(1.6);7.557(1.1);7.496(0.6);7.493(0.6);7.472(1.0);7.450(0.5); 7.448(0.5);5.316(2.6);3.576(5.4);2.511(4.5);2.506(10.1);2.502(14.3);2.497(10.2);2.492(4.7);2.074(16.0);0.000(10.9)

### Example No. A1-61

### ¹H-NMR (400 MHz, CDCl₃ δ, ppm)

8.000(0.8);7.982(0.9);7.783(0.7);7.780(0.7);7.764(0.8);7.761(0.9);7.718(1.0);7.714(0.8); 7.698(0.6);7.649(0.7);7.629(0.8);7.611(0.7);7.608(0.7);7.591(1.0);7.588(1.0);7.520(1.0); 7.484(0.8);7.392(0.6);7.261(11.8);5.302(4.2);5.297(1.6);3.469(16.0);1.617(1.3);0.000(5.1)

### Example No. A1-62

### ¹H-NMR (400 MHz, CDCl₃ δ, ppm)

7.874(0.7);7.870(1.3);7.867(0.9);7.839(0.7);7.823(0.6);7.819(0.8);7.815(0.5);7.784(0.5); 7.780(0.7);7.777(0.5);7.764(0.7);7.760(0.9);7.757(0.6);7.640(0.8);7.621(0.9);7.601(0.8); 7.600(0.8);7.581(1.3);7.580(1.3);7.562(0.6);7.561(0.5);7.508(0.7);7.505(1.2);7.503(1.1); 7.495(2.6);7.492(2.3);7.263(31.9);5.070(4.2);3.394(16.0);2.956(0.7);2.955(0.7);2.884(0.6);2.883(0.6);1.625(0.8);0.000(13.1)

### Example No. A1-63

### ¹H-NMR (400 MHz, CDCl₃ δ, ppm)

7.763(1.4);7.758(0.6);7.747(0.8);7.742(3.3);7.738(0.6);7.706(3.0);7.701(0.7);7.690(0.5); 7.685(1.3);7.643(0.7);7.624(0.9);7.490(0.6);7.473(1.4);7.472(1.5);7.467(1.2);7.393(0.5); 7.261(41.7);5.100(4.4);3.391(16.0);2.044(0.6);1.570(0.7);0.000(18.1);-0.009(0.5)

### Example No. A1-86

### ¹H-NMR (400 MHz, d₆-DMSO δ, ppm)

8.109(1.3);8.104(1.3);7.924(0.9);7.920(0.9);7.895(1.5);7.874(0.6);7.750(0.6);7.659(0.5); 7.639(0.5);7.512(0.5);5.250(1.9);3.514(4.9);2.511(5.2);2.507(11.7);2.502(16.8);2.498(12.1);2.493(5.7);2.074(16.0);0.000(10.1)

### Example No. A1-94

### ¹H-NMR (400 MHz, CDCl₃ δ, ppm)

8.511(3.4);8.506(3.3);8.008(1.5);8.002(1.5);7.987(2.4);7.981(1.7);7.642(2.0);7.624(2.4); 7.513(0.8);7.495(2.0);7.477(2.0);7.453(1.6);7.435(5.4);7.415(4.9);7.402(1.6);7.265(30.3); 5.105(2.8);3.468(16.0);2.949(6.1);2.868(5.2);2.702(4.2);2.464(1.5);1.253(1.0);0.000(11.2);−0.009(0.6)

### Example No. A1-121

### ¹H-NMR (400 MHz, CDCl₃ δ, ppm)

8.265(1.0);8.245(1.1);8.115(0.6);8.113(0.6);8.094(0.7);8.092(0.7);7.901(0.6);7.897(0.6); 7.880(0.7);7.879(0.7);7.877(0.7);7.845(1.9);7.824(1.8);7.800(0.5);7.797(0.5);7.783(0.7); 7.779(1.0);7.762(0.6);7.758(0.5);7.646(0.7);7.643(0.7);7.629(1.2);7.626(1.2);7.623(0.6); 7.609(1.0);7.606(0.5);7.448(0.5);7.445(0.6);7.439(0.9);7.433(1.8);7.431(2.3);7.263(11.0); 5.353(5.2);5.300(0.6);3.560(16.0);0.000(3.7)

8(1.1);2.581(7.9);1.432(1.3);1.257(0.6);0.070(1.0);0.000(6.2).

### Example No. A1-137

### ¹H-NMR (400 MHz, d₆-DMSO δ, ppm)

7.823(1.4);7.807(1.4);7.801(1.6);7.786(1.0);7.747(0.6);7.702(1.0);7.695(1.1);7.680(1.2); 7.673(1.0);7.629(0.6);7.452(0.7);7.446(0.7);5.234(1.9);3.528(4.5);2.511(5.1);2.506(11.3); 2.502(15.9);2.497(11.4);2.493(5.2);2.074(16.0);0.000(9.4)

### Example No. A1-144

### ¹H-NMR (400 MHz, d₆-DMSO δ, ppm)

8.614(0.9);8.600(1.0);7.839(0.9);7.819(0.5);7.677(0.6);7.315(1.0);7.301(0.9);5.305(2.0); 5.002(1.2);4.980(1.2);3.710(6.1);2.511(2.0);2.507(4.4);2.502(6.1);2.497(4.3);2.493(2.0); 2.322(5.4);2.074(16.0);0.000(4.1)

### Example No. A1-147

### ¹H-NMR (400 MHz, d₆-DMSO δ, ppm)

7.311(2.6);7.221(2.2);5.176(1.1);4.478(0.8);3.853(7.8);3.760(1.9);3.754(2.2);3.747(6.1); 3.561(3.1);2.570(1.6);2.511(5.0);2.506(11.0);2.502(15.5);2.497(11.1);2.492(5.1);2.364(0.7);2.074(16.0);0.000(9.9)

### Example No. A1-462

### ¹H-NMR (400 MHz, CDCl₃ δ, ppm)

7.698(0.5);7.691(0.7);7.684(0.6);7.569(4.4);7.562(3.1);7.261(24.8);7.217(0.9);7.196(0.6); 6.916(1.3);6.896(1.7);6.876(1.1);4.068(1.0);4.056(0.7);4.047(0.8);4.037(0.7);4.026(1.0); 3.724(16.0);2.852(0.8);2.833(1.6);2.815(0.8);2.262(0.7);2.250(0.6);2.240(0.7);2.232(0.6);2.220(0.7);0.000(8.0)

### Example No. A1-603

### ¹H-NMR (400 MHz, CDCl₃ δ, ppm)

8.035(0.8);8.032(0.8);8.015(0.9);8.012(0.8);7.862(0.7);7.859(0.8);7.842(0.8);7.839(0.9); 7.632(0.7);7.631(0.7);7.627(0.5);7.623(0.5);7.613(0.9);7.611(0.8);7.608(1.1);7.604(1.0); 7.589(0.7);7.585(0.6);7.564(0.5);7.562(0.5);7.546(0.8);7.545(0.9);7.543(0.8);7.536(0.8); 7.533(0.8);7.517(0.9);7.514(0.9);7.498(0.9);7.495(0.6);7.481(0.8);7.479(0.7);7.372(0.6); 7.261(23.8);5.586(4.6);3.886(16.0);3.383(15.2);0.000(9.8)

### Example No. A1-612

### ¹H-NMR (400 MHz, CDCl₃ δ, ppm)

7.642(0.7);7.641(0.6);7.624(1.5);7.607(0.7);7.606(0.8);7.604(0.7);7.494(0.7);7.493(0.7); 7.383(1.3);7.376(0.8);7.373(0.5);7.369(1.0);7.365(1.2);7.362(0.7);7.359(1.2);7.352(0.8); 7.263(7.3);7.238(1.3);7.234(1.4);7.233(1.0);7.225(2.0);7.215(1.1);7.211(0.9);3.976(1.0); 3.963(0.5);3.956(0.7);3.954(0.7);3.946(0.6);3.934(1.2);3.499(16.0);3.336(1.1);3.323(0.5);3.314(0.7);3.293(1.0);0.000(3.0)

### Example No. A1-613

### ¹H-NMR (400 MHz, CDCl₃ δ, ppm)

7.641(0.7);7.640(0.6);7.623(0.7);7.622(0.8);7.620(0.7);7.599(0.6);7.597(0.5);7.581(0.7); 7.580(0.8);7.578(0.7);7.494(0.7);7.492(0.7);7.384(0.6);7.263(9.4);7.259(2.6);7.254(0.8); 7.249(1.0);7.245(2.1);7.242(2.5);7.239(1.0);7.181(0.5);7.175(0.7);4.000(1.0);3.986(0.5); 3.978(0.8);3.971(0.5);3.957(1.2);3.462(16.0);3.188(1.0);3.166(0.8);3.145(0.9);0.000(3.9)

### Example No. A1-614

### ¹H-NMR (400 MHz, CDCl₃ δ, ppm)

7.640(0.6);7.638(0.7);7.637(0.6);7.620(0.7);7.619(0.8);7.617(0.7);7.590(0.6);7.588(0.5); 7.572(0.7);7.571(0.8);7.569(0.8);7.490(0.7);7.488(0.7);7.382(0.6);7.302(1.9);7.297(0.7); 7.286(0.8);7.281(3.2);7.265(6.4);7.215(2.5);7.210(0.7);7.199(0.5);7.194(1.5);3.994(1.1); 3.980(0.5);3.973(0.8);3.965(0.5);3.952(1.2);3.450(16.0);3.175(1.0);3.153(0.8);3.133(0.9);1.795(0.5);0.000(2.7)

### Example No. A1-615

### ¹H-NMR (400 MHz, CDCl₃ δ, ppm)

7.645(0.7);7.626(0.8);7.610(0.6);7.593(0.7);7.591(0.8);7.590(0.7);7.496(0.7);7.495(0.7); 7.400(1.7);7.395(1.8);7.389(0.7);7.320(1.1);7.299(1.8);7.262(14.6);7.235(1.3);7.230(1.2); 7.215(0.8);7.209(0.8);3.956(1.0);3.942(0.5);3.936(0.7);3.934(0.7);3.926(0.5);3.913(1.1); 3.490(16.0);3.305(1.1);3.284(0.7);3.263(0.9);1.647(1.0);0.000(6.3)

### Example No. A1-634

### ¹H-NMR (400 MHz, CDCl₃ δ, ppm)

7.634(0.6);7.632(0.7);7.631(0.6);7.614(0.7);7.612(0.8);7.611(0.7);7.602(0.5);7.600(0.6); 7.598(0.5);7.583(0.7);7.581(0.8);7.579(0.7);7.484(0.7);7.482(0.7);7.373(0.6);7.261(7.0); 7.166(0.6);7.151(0.5);7.145(3.3);7.134(2.8);7.114(0.5);3.981(1.1);3.967(0.6);3.961(0.8); 3.953(0.6);3.939(1.2);3.451(16.0);3.159(0.9);3.137(0.8);3.117(0.8);2.324(6.3);1.998(0.8);1.765(0.7);0.000(3.3)

### Example No. A1-639

### ¹H-NMR (400 MHz, CDCl₃ δ, ppm)

7.353(0.9);7.332(1.1);7.260(3.6);7.209(0.9);7.188(0.7);3.459(6.5);1.305(16.0);0.000(1.7)

### Example No. A1-663

### ¹H-NMR (400 MHz, CDCl₃ δ, ppm)

7.633(0.6);7.631(0.7);7.630(0.6);7.613(0.8);7.612(0.8);7.610(0.8);7.603(0.5);7.602(0.6); 7.600(0.5);7.584(0.7);7.583(0.8);7.581(0.7);7.484(0.8);7.482(0.7);7.371(0.6);7.261(5.7); 7.087(0.7);7.068(1.1);7.032(1.0);6.997(0.7);6.993(0.6);3.968(1.1);3.954(0.6);3.947(0.8); 3.939(0.6);3.925(1.2);3.450(16.0);3.127(0.9);3.106(0.7);3.085(0.8);2.237(6.0);2.232(5.7);0.000(2.6)

### Example No. A1-664

### ¹H-NMR (400 MHz, CDCl₃ δ, ppm)

7.627(0.7);7.607(1.4);7.606(1.4);7.588(0.8);7.587(0.9);7.586(0.8);7.486(0.8);7.484(0.7); 7.370(0.7);7.260(5.2);7.083(0.9);7.064(1.3);6.991(1.1);6.978(0.7);3.889(1.0);3.876(0.6); 3.869(0.7);3.867(0.7);3.859(0.6);3.846(1.2);3.472(16.0);3.158(0.9);3.137(0.7);3.115(0.8); 2.339(6.6);2.288(6.0);0.000(2.3)

### Example No. A1-665

### ¹H-NMR (400 MHz, CDCl₃ δ, ppm)

7.626(0.7);7.608(1.4);7.592(0.9);7.489(0.8);7.371(0.6);7.269(0.5);7.260(75.0);6.859(2.9); 3.804(0.9);3.791(0.6);3.783(0.6);3.771(0.6);3.760(1.9);3.497(16.0);3.199(1.0);3.188(0.5);3.177(0.6);3.155(0.9);2.354(15.2);2.250(6.1);1.566(5.9);0.008(1.0);0.000(31.4);−0.009(0.9)

### Example No. A1-666

### ¹H-NMR (400 MHz, CDCl₃ δ, ppm)

7.632(0.6);7.631(0.7);7.629(0.6);7.610(1.3);7.591(0.7);7.589(0.8);7.587(0.7);7.487(0.7); 7.486(0.7);7.373(0.6);7.260(12.6);7.100(1.0);7.081(1.3);7.016(1.1);6.984(0.7);6.981(0.6); 3.904(1.0);3.890(0.5);3.883(0.7);3.882(0.7);3.874(0.5);3.861(1.1);3.478(16.0);3.176(0.9);3.155(0.7);3.133(0.8);2.698(0.5);2.679(1.7);2.660(1.7);2.641(0.6);2.308(5.9);2.001(0.9);1.686(0.7);1.251(2.9);1.232(6.5);1.213(2.8);0.000(5.4)

### Example No. A1-679

### ¹H-NMR (400 MHz, CDCl₃ δ, ppm)

8.799(1.0);8.795(1.1);8.788(1.1);8.784(1.1);8.381(0.9);8.376(0.9);8.360(0.9);8.356(0.9); 7.659(0.7);7.639(0.8);7.634(1.1);7.622(1.0);7.613(1.0);7.602(0.9);7.494(0.5);7.491(0.5); 7.482(0.9);7.479(1.5);7.476(2.4);7.261(47.7);5.281(3.9);3.524(16.0);1.563(1.6);0.008(0.5);0.000(21.2);-0.009(0.6)

### Example No. A2-1

### ¹H-NMR (400 MHz, d₆-DMSO δ, ppm)

7.850(1.7);7.845(1.6);7.831(2.0);7.826(1.9);7.641(1.7);7.621(2.1);7.596(0.6);7.580(3.4); 7.576(3.3);7.560(3.7);7.557(3.4);7.523(2.2);7.505(1.2);7.496(1.0);7.491(1.2);7.478(3.1); 7.473(2.1);7.458(2.9);7.453(1.7);7.449(1.9);7.445(2.3);7.430(2.1);7.426(2.1);7.412(0.8); 7.408(0.7);5.752(0.7);5.115(8.7);3.842(1.5);3.825(1.8);3.810(2.1);3.792(1.9);3.546(1.7); 3.528(2.0);3.513(1.7);3.496(1.4);3.305(69.4);2.674(0.6);2.669(0.9);2.665(0.6);2.523(2.7);2.518(3.9);2.509(48.7);2.505(104.7);2.500(145.2);2.495(100.5);2.491(44.8);2.332(0.6);2.327(0.8);2.322(0.6);1.908(0.7);1.282(7.0);1.265(16.0);1.247(6.7);0.000(4.8)

### Example No. A2-22

### ¹H-NMR (400 MHz, CDCl₃ δ, ppm)

8.845(4.7);8.832(4.7);7.519(0.7);7.389(1.8);7.379(1.2);7.368(2.7);7.354(1.1);7.334(2.1); 7.329(2.3);7.2744(0.6);7.2737(0.6);7.273(0.6);7.272(0.7);7.2713(0.8);7.2705(0.8);7.270(0.9);7.269(1.0);7.268(1.1);7.2672(1.3);7.2665(1.5);7.266(1.7);7.265(2.1);7.260(123.4); 7.257(1.7);7.256(1.1);7.255(0.8);7.254(0.5);7.210(0.7);7.185(1.7);7.180(1.5);7.165(1.4); 7.160(1.4);6.996(0.6);5.334(5.1);3.605(16.0);2.956(1.5);2.885(1.3);2.883(1.3);2.044(0.6);1.554(6.0);0.008(1.6);0.005(0.6);0.000(51.5);−0.006(0.6);−0.009(1.5)

### Example No. A3-1

### ¹H-NMR (600 MHz, d₆-DMSO δ, ppm)

7.926(0.5);7.848(2.1);7.834(2.2);7.787(0.5);7.752(0.5);7.701(1.8);7.688(2.4);7.676(1.0); 7.526(3.7);7.462(0.8);7.450(1.0);7.423(3.2);7.418(2.9);7.412(3.1);7.399(0.9);7.388(0.8); 7.375(0.9);7.369(0.9);7.358(1.0);5.022(0.4);4.970(0.4);4.536(0.4);3.377(23.7);3.371(26.9); 3.366(33.2);3.362(39.1);3.359(43.8);3.356(60.2);2.888(0.3);2.844(1.4);2.756(0.4); 2.624(0.5);2.571(0.9);2.533(0.7);2.530(0.8);2.527(0.8);2.515(49.0);2.512(67.6);2.509(49.0); 2.506(22.9);2.396(0.8);1.511(0.3);1.419(4.5);1.289(16.0);0.007(0.4)

### Example No. A3-5

### ¹H-NMR (600 MHz, d₆-DMSO δ, ppm)

15.483(0.8);8.036(0.8);7.925(1.0);7.921(0.8);7.903(1.0);7.889(0.9);7.852(2.0);7.840(2.3); 7.790(0.9);7.766(1.1);7.762(1.1);7.757(1.1);7.706(2.5);7.695(2.6);7.682(1.5);7.658(1.3); 7.565(0.8);7.533(6.1);7.516(2.1);7.481(1.0);7.384(0.8);7.132(0.9);5.300(0.9);5.107(0.8);4.524(0.8);4.189(0.8);3.931(1.2);3.452(1.9);3.374(166.6);3.372(170.6);3.369(175.7); 3.363(207.3);3.360(227.7);3.356(274.7);3.352(311.5);3.333(2.4);2.901(1.0);2.880(1.5); 2.625(4.5);2.534(4.8);2.531(6.9);2.528(8.0);2.519(234.1);2.516(519.5);2.513(729.6); 2.510(532.5);2.507(246.9);2.452(0.9);2.403(1.8);2.400(2.9);2.397(4.0);2.394(2.9);2.383(0.8);1.510(4.7);1.490(0.9);1.407(4.7);1.401(1.1);1.274(16.0);1.226(1.1);1.035(0.8);0.010(6.9);-3.559(0.8)

### Example No. A3-612

### ¹H-NMR (600 MHz, d₆-DMSO δ, ppm)

19.969(0.4);7.897(0.9);7.882(1.3);7.865(1.5);7.847(1.1);7.833(1.1);7.760(1.0);7.747(1.5); 7.734(1.1);7.723(0.9);7.701(0.9);7.691(1.1);7.678(0.6);7.566(0.4);7.528(1.0);7.514(1.0); 7.488(2.1);7.475(2.6);7.422(1.0);7.410(1.1);7.362(0.7);7.348(1.2);7.333(1.6);7.324(2.2);7.318(1.7);7.312(1.5);7.300(0.7);7.246(4.0);7.238(3.2);3.926(0.4);3.717(0.4);3.383(27.6);3.377(30.0);3.370(34.3);3.362(47.0);3.356(41.4);3.353(52.4);3.352(53.3);3.298(0.7);3.160(1.6);3.131(2.1);3.065(5.5);2.986(1.1);2.972(1.6);2.958(0.9);2.899(0.4);2.785(6.3);2.624(0.6);2.530(1.2);2.527(1.3);2.515(78.8);2.512(109.2);2.509(80.3);2.396(0.7); 1.506(5.1);1.489(16.0);1.431(5.5);1.361(2.1);0.007(0.7)

### Example No. A3-615

### ¹H-NMR (600 MHz, d₆-DMSO δ, ppm)

7.961(0.4);7.896(1.0);7.882(1.6);7.867(1.2);7.843(1.0);7.829(1.1);7.748(1.2);7.728(1.1); 7.715(0.7);7.701(1.1);7.688(1.2);7.647(1.7);7.577(0.4);7.539(2.4);7.525(3.6);7.509(1.1); 7.497(3.0);7.494(3.1);7.471(0.6);7.442(1.1);7.425(1.2);7.408(0.9);7.367(0.3);7.336(1.7); 7.328(1.5);7.315(3.5);7.302(1.2);5.430(0.4);3.931(0.4);3.925(0.4);3.919(0.5);3.909(0.5); 3.902(0.5);3.892(0.4);3.745(0.4);3.734(0.5);3.718(0.5);3.711(0.4);3.695(0.4);3.450(0.5);3.438(0.5);3.425(0.5);3.364(31.6);3.363(32.1);3.357(61.6);3.355(77.4);3.354(71.6); 3.288(0.5);3.275(0.5);3.263(0.5);3.148(1.6);3.134(0.9);3.121(2.4);3.109(1.1);3.056(5.6); 3.002(0.4);2.971(1.0);2.958(1.3);2.944(0.8);2.899(2.2);2.800(6.5);2.739(1.8);2.623(0.5); 2.533(0.7);2.530(0.9);2.526(0.9);2.518(28.8);2.515(62.4);2.512(85.6);2.509(60.8);2.506(27.2);2.396(0.5);1.499(5.5);1.481(16.0);1.427(4.9);1.375(1.5);1.273(0.8);1.244(0.4); 0.006(0.6)

### Example No. A4-1

### ¹H-NMR (400 MHz, d₆-DMSO δ, ppm)

7.849(0.3);7.840(0.4);7.835(0.4);7.826(0.4);7.747(3.1);7.728(4.0);7.714(1.4);7.695(3.4); 7.677(2.3);7.659(1.0);7.651(0.7);7.645(0.8);7.636(1.0);7.593(2.1);7.574(3.1);7.555(1.3); 7.522(3.8);7.516(3.6);7.512(3.3);7.503(3.7);7.497(4.6);7.493(4.6);7.485(2.9);7.470(3.3); 7.466(3.6);7.422(1.4);7.419(1.7);7.404(3.6);7.400(3.4);7.385(2.4);7.381(2.1);7.375(3.1); 7.370(3.4);7.355(3.4);7.351(4.1);7.337(1.1);7.333(1.0);7.319(0.4);7.315(0.5);7.300(0.5);5.903(0.5);5.888(0.5);4.908(13.0);4.884(3.1);4.870(3.1);4.856(1.0);3.333(58.7);2.889(0.9);2.731(0.8);2.541(2.6);2.526(2.8);2.519(1.0);2.511(13.9);2.506(28.9);2.502(38.8); 2.497(28.9);2.493(14.5);2.267(16.0);2.254(15.8);0.000(7.3)

### Example No. A4-5

### ¹H-NMR (400 MHz, d₆-DMSO δ, ppm)

7.850(0.3);7.837(0.4);7.834(0.3);7.827(0.4);7.744(3.2);7.724(4.0);7.710(1.5);7.691(3.5); 7.674(9.2);7.662(1.1);7.652(0.7);7.592(2.2);7.573(3.1);7.553(1.3);7.542(0.9);7.537(1.5); 7.515(1.3);7.506(4.0);7.494(15.4);7.491(16.0);7.471(0.8);7.465(0.7);7.450(0.4);7.444(0.4);7.371(0.4);7.362(0.4);7.349(0.3);5.914(0.4);5.899(0.4);4.933(1.0);4.920(3.2);4.906(3.3);4.884(14.3);3.330(32.1);2.890(0.9);2.731(0.8);2.539(2.3);2.524(2.7);2.511(13.1);2.506(26.4);2.502(34.9);2.498(25.7);2.493(12.7);2.265(14.6);2.251(14.3);0.000(6.5)

### Example No. A4-612

### ¹H-NMR (400 MHz, d₆-DMSO δ, ppm)

7.779(1.7);7.760(2.2);7.710(3.3);7.690(4.2);7.669(1.5);7.650(3.7);7.631(3.2);7.611(1.8); 7.593(1.0);7.558(2.3);7.538(4.5);7.520(3.3);7.501(0.9);7.468(5.5);7.463(3.4);7.449(7.5); 7.360(1.4);7.357(1.8);7.342(4.0);7.338(4.7);7.329(6.5);7.324(3.5);7.320(2.9);7.311(6.4); 7.290(5.6);7.281(2.3);7.272(4.1);7.268(2.9);7.255(0.7);7.249(0.5);7.230(2.1);7.225(1.3); 7.221(1.0);7.214(1.1);7.207(1.0);6.537(1.8);6.518(1.7);5.780(0.5);5.766(1.6);5.751(1.6);5.737(0.5);5.069(1.0);5.055(3.2);5.041(3.2);5.028(1.0);3.803(2.6);3.784(3.7);3.764(2.9);3.330(62.6);3.142(0.3);3.123(0.7);3.105(0.7);3.086(4.2);3.066(5.0);3.047(3.5);3.019(0.5);3.005(0.6);2.987(0.9);2.968(0.4);2.903(0.6);2.890(1.8);2.875(0.6);2.862(0.4);2.731(1.0);2.672(0.4);2.588(8.6);2.573(8.5);2.524(0.9);2.507(47.9);2.502(64.5);2.498(48.7); 2.329(0.4);2.325(0.3);2.273(16.0);2.260(16.0);1.234(0.4);0.000(9.0)

### Example No. A4-615

### ¹H-NMR (400 MHz, d₆-DMSO δ, ppm)

7.795(1.4);7.776(1.7);7.708(3.3);7.688(4.2);7.671(1.6);7.653(4.2);7.634(3.8);7.622(5.6); 7.617(6.6);7.588(1.1);7.570(1.6);7.558(2.4);7.539(3.3);7.520(1.4);7.502(5.8);7.481(7.8); 7.443(4.6);7.437(4.4);7.422(2.3);7.417(2.4);7.371(1.2);7.366(1.2);7.351(1.7);7.345(1.7); 7.325(3.8);7.306(3.4);7.263(3.0);7.243(2.1);6.747(1.5);6.728(1.4);5.774(0.4);5.759(1.4); 5.745(1.4);5.730(0.4);5.086(1.0);5.073(3.3);5.059(3.3);5.045(1.0);3.797(2.7);3.778(4.0);3.759(3.0);3.336(98.2);3.122(0.6);3.104(0.5);3.086(0.5);3.070(3.8);3.051(4.9);3.032(3.3);2.998(0.4);2.985(0.5);2.965(0.7);2.946(0.3);2.898(0.4);2.891(1.3);2.869(0.5);2.732(0.7);2.583(7.0);2.569(7.0);2.526(0.6);2.512(16.6);2.508(34.8);2.504(47.0);2.499(35.1); 2.495(17.6);2.271(16.0);2.258(15.9);0.000(4.0)

### Example No. A7-3

### ¹H-NMR (400 MHz, CDCl₃ δ, ppm)

7.501(1.4);7.496(0.5);7.485(0.8);7.479(3.0);7.447(0.5);7.442(3.3);7.436(0.8);7.425(0.6); 7.420(1.6);7.414(1.9);7.408(1.8);7.280(1.5);7.262(22.1);7.259(2.8);7.189(1.7);7.183(1.6); 7.168(1.1);7.161(1.0);5.019(4.3);3.359(16.0);2.006(0.8);1.605(0.7);0.000(10.0)

### Example No. A7-4

### ¹H-NMR (400 MHz, d₆-DMSO δ, ppm)

7.841(0.6);7.837(0.7);7.821(0.7);7.817(0.8);7.769(0.7);7.766(0.7);7.750(0.8);7.746(0.7); 7.597(0.5);7.525(2.4);7.505(1.3);7.485(0.6);5.279(2.0);3.529(3.9);2.512(2.1);2.508(4.5); 2.503 (6.3);2.499(4.4);2.494(2.0);2.075(16.0);0.000(4.0)

### Example No. A7-5

### ¹H-NMR (400 MHz, d₆-DMSO δ, ppm)

7.805(1.8);7.798(1.4);7.785(1.5);7.581(0.8);7.576(0.8);7.560(0.7);7.555(0.6);7.481(0.6); 7.460(0.9);7.447(0.6);5.140(1.8);3.426(3.3);2.511(3.9);2.507(8.5);2.502(11.8);2.497(8.3); 2.493(3.8);2.075(16.0);0.000(6.1)

### Example No. A7-7

### ¹H-NMR (400 MHz, d₆-DMSO δ, ppm)

7.689(2.1);7.687(2.4);7.668(5.0);7.603(1.8);7.585(1.3);7.581(1.1);7.563(0.8);7.529(2.7); 5.403(3.9);4.686(0.6);3.593(5.2);2.575(0.8);2.511(6.7);2.506(14.3);2.502(19.7);2.497(14.1);2.493(6.5);2.074(16.0);0.000(11.4)

### Example No. A7-21

### ¹H-NMR (400 MHz, CDCl₃ δ, ppm)

7.792(0.3);7.788(0.3);7.774(0.4);7.770(0.4);7.768(0.4);7.765(0.5);7.763(0.5);7.761(0.4); 7.596(0.8);7.593(1.0);7.592(1.1);7.589(1.0);7.373(0.3);7.336(1.9);7.334(2.2);7.331(3.6); 7.328(1.1);7.310(0.4);7.302(0.4);7.297(1.1);7.290(0.8);7.260(45.5);7.247(0.6);7.235(1.0); 7.212(0.4);7.163(0.6);7.157(0.5);7.142(0.4);7.136(0.3);5.171(2.0);3.493(6.4);2.733(16.0);2.512(5.8);1.554(5.4);0.008(0.5)

### Example No. A7-30

### ¹H-NMR (400 MHz, d₆-DMSO δ, ppm)

7.520(1.2);7.514(0.7);6.413(1.9);5.145(1.4);3.926(16.0);3.741(2.8);2.511(2.1);2.507(4.8); 2.502(6.8);2.498(4.9);2.493(2.3);0.000(5.0)

### Example No. A7-33

### ¹H-NMR (400 MHz, d₆-DMSO δ, ppm)

8.686(2.8);8.673(2.9);7.776(3.2);7.772(3.7);7.719(1.5);7.714(1.4);7.705(1.5);7.700(1.4); 7.485(2.4);5.181(3.2);3.569(4.5);3.168(1.0);2.564(0.9);2.524(1.0);2.519(1.7);2.510(34.1); 2.506(75.4);2.501(106.0);2.496(75.2);2.492(34.3);2.412(0.7);2.328(0.7);2.323(0.5);2.074(16.0);0.008(1.6);0.000(55.3);-0.009(1.7)

### Example No. A7-40

### ¹H-NMR (400 MHz, d₆-DMSO δ, ppm)

8.616(2.7);8.603(2.8);7.917(1.7);7.904(1.6);7.496(1.6);5.377(1.5);3.665(3.4);2.524(0.7); 2.519(1.0);2.511(12.9);2.506(27.5);2.501(37.8);2.497(26.8);2.492(12.2);2.074(16.0);1.243(0.5);0.008(0.8);0.000(24.8);−0.009(0.8)

### Example No. A7-59

### ¹H-NMR (400 MHz, d₆-DMSO δ, ppm)

7.661(0.7);7.645(0.8);7.640(0.6);7.625(1.3);7.554(1.2);7.540(2.3);7.537(2.5);7.443(0.8); 7.440(0.5);5.274(2.0);3.569(4.4);2.511(2.7);2.507(5.9);2.502(8.4);2.498(6.0);2.493(2.7); 2.074(16.0);0.000(5.7)

### Example No. A7-86

### ¹H-NMR (400 MHz, d₆-DMSO δ, ppm)

8.077(0.7);8.072(0.8);7.908(0.6);7.903(0.6);7.881(1.0);7.479(1.1);7.471(0.6);7.465(0.7); 7.432(0.5);5.241(1.9);3.495(4.4);2.513(2.0);2.508(4.4);2.504(6.1);2.499(4.3);2.495(1.9); 2.076(16.0);0.000(4.1)

### Example No. A7-137

### ¹H-NMR (400 MHz, d₆-DMSO δ, ppm)

7.846(0.6);7.830(0.6);7.824(0.6);7.808(0.6);7.672(0.6);7.666(0.6);7.650(0.6);7.643(0.6); 7.525(1.7);7.404(0.5);5.193(1.9);3.508(3.9);2.512(2.4);2.507(5.4);2.503(7.5);2.498(5.3); 2.494 (2.4);2.075(16.0);0.000(4.2)

### Example No. A7-144

### ¹H-NMR (400 MHz, d₆-DMSO δ, ppm)

8.494(2.9);8.480(3.0);7.583(0.8);7.573(2.0);7.566(5.6);7.558(5.7);7.550(0.7);7.534(0.7); 7.289(2.8);7.274(2.7);5.227(5.1);4.999(1.1);4.977(3.5);4.956(3.7);4.934(1.3);3.690(15.2); 2.511(7.4);2.507(16.6);2.502(23.5);2.497(16.8);2.493(7.8);2.322(16.5);2.074(16.0);0.000(19.3);−0.009(0.7)

### Example No. A7-147

### ¹H-NMR (400 MHz, d₆-DMSO δ, ppm)

7.631(0.8);7.553(2.9);7.280(4.9);7.192(5.0);5.103(2.7);3.836(16.0);3.774(0.8);3.760(1.1); 3.755(1.3);3.746(14.3);3.527(7.0);2.571(0.9);2.511(7.9);2.507(17.5);2.502(24.6);2.497(17.5);2.493(8.0);2.413(0.6);2.075(0.7);0.000(17.5);-0.009(0.6)

### Example No. A7-148

### ¹H-NMR (400 MHz, d₆-DMSO δ, ppm)

8.455(3.2);8.442(3.4);7.820(2.4);7.807(2.3);7.560(3.4);5.254(2.6);3.920(16.0);3.664(6.7); 2.512(3.1);2.507(6.9);2.502(9.7);2.498(6.9);2.493(3.2);0.000(7.9)

### Example No. A7-634

### ¹H-NMR (400 MHz, CDCl₃ δ, ppm)

7.456(1.8);7.450(1.9);7.276(1.5);7.262(11.4);7.255(2.3);7.182(1.8);7.179(0.9);7.176(2.0); 7.164(0.6);7.161(1.9);7.158(2.8);7.154(1.8);7.140(2.3);7.120(0.7);4.046(1.0);4.032(0.5); 4.025(0.8);4.017(0.5);4.004(1.1);3.443(16.0);3.183(0.9);3.162(0.7);3.141(0.8);2.327(5.9);2.003(0.8);1.663(1.2);0.000(4.9)

### Example No. A7-639

### ¹H-NMR (400 MHz, CDCl₃ δ, ppm)

7.463(0.8);7.457(0.8);7.356(0.8);7.335(1.1);7.277(0.7);7.262(3.5);7.256(1.0);7.221(0.9); 7.200(0.7);7.182(0.7);7.176(0.7);3.447(6.5);2.001(1.5);1.305(16.0);0.000(1.5)

### Example No. A7-663

### ¹H-NMR (400 MHz, CDCl₃ δ, ppm)

7.457(1.9);7.451(2.0);7.276(1.6);7.261(10.8);7.254(2.3);7.181(1.8);7.175(1.7);7.160(1.2); 7.153(1.1);7.092(0.7);7.073(1.0);7.046(1.0);7.010(0.7);7.006(0.6);4.031(1.0);4.017(0.5); 4.010(0.8);4.003(0.5);3.989(1.1);3.443(16.0);3.151(0.8);3.130(0.7);3.109(0.8);2.241(5.7);2.234(5.5);2.003(1.7);1.677(0.9);0.000(4.7)

### Example No. A7-664

### ¹H-NMR (400 MHz, CDCl₃ δ, ppm)

7.474(1.9);7.468(1.9);7.273(1.5);7.261(8.9);7.252(2.4);7.181(1.7);7.174(1.6);7.159(1.1); 7.153(1.1);7.098(0.9);7.079(1.2);6.994(1.1);6.983(0.7);3.954(1.0);3.941(0.5);3.934(0.7); 3.932(0.7);3.924(0.5);3.911(1.1);3.467(16.0);3.182(0.9);3.160(0.7);3.139(0.8);2.351(6.2); 2.291(5.7);0.000(4.0)

### Example No. A7-665

### ¹H-NMR (400 MHz, CDCl₃ δ, ppm)

7.488(2.0);7.482(2.0);7.272(1.7);7.260(46.0);7.251(2.5);7.182(1.8);7.176(1.7);7.161(1.2); 7.155(1.1);6.861(2.8);3.859(0.9);3.846(0.6);3.838(0.6);3.836(0.6);3.826(0.6);3.815(1.0); 3.493(16.0);3.218(0.9);3.206(0.5);3.195(0.6);3.174(0.8);2.362(13.5);2.251(5.9);1.573(2.7);0.008(0.6);0.000(19.9);-0.009(0.5)

### Example No. A7-666

### ¹H-NMR (400 MHz, CDCl₃ δ, ppm)

7.4811(1.8);7.4806(1.9);7.474(1.8);7.276(1.5);7.261(12.5);7.255(2.4);7.254(2.4);7.184(1.8);7.177(1.7);7.162(1.47.156(1.47.111(0.9);7.091(1.3);7.020(1.0);6.988(0.6);6.985(0.5);3.959(0.9);3.945(0.5);3.939(0.7);3.937(0.7);3.929(0.5);3.916(1.1);3.469(16.0);3.198(0.9);3.176(0.7);3.155(0.8);2.710(0.5);2.691(1.7);2.672(1.7);2.653(0.6);2.309(5.8);2.003(1.2);1.652(1.0);1.258(2.9);1.240(6.5);1.221(2.8);0.000(5.8)

### Example No. A8-2

### ¹H-NMR (400 MHz, CDCl₃ δ, ppm)

7.648(1.0);7.644(1.8);7.639(1.1);7.494(0.5);7.491(0.8);7.487(0.6);7.475(0.7);7.472(1.1); 7.468(0.7);7.441(0.6);7.438(0.7);7.436(0.6);7.433(0.6);7.421 (1.1);7.418(1.0);7.416(1.2); 7.413(0.9);7.372(1.9);7.359(0.8);7.353(2.0);7.333(0.7);7.265(1.5);7.260(60.6);7.246(1.0); 7.242(0.9);7.240(0.8);7.226(0.8);7.222(0.7);6.902(3.2);6.882(3.5);6.866(0.9);6.863(0.7);5.121(4.3);3.894(16.0);3.383(15.2);2.017(0.6);0.008(0.8);0.000(25.4);−0.009(0.7)

### Example No. A8-4

### ¹H-NMR (400 MHz, CDCl₃ δ, ppm)

7.991(1.4);7.987(1.4);7.971(1.5);7.967(1.4);7.564(1.5);7.560(1.5);7.544(1.8);7.540(1.7); 7.381(0.7);7.377(0.7);7.363(0.7);7.358(0.7);7.310(1.7);7.291(2.7);7.271(1.9);7.268(0.8); 7.267(0.9);7.265(1.8);7.264(2.0);7.261(60.3);7.257(0.9);7.256(0.6);7.251(0.9);7.249(0.8); 7.247(0.8);7.244(0.7);7.230(0.8);7.226(0.7);6.906(2.2);6.887(3.9);6.869(0.9);6.867(0.7);5.450(4.2);4.130(1.1);4.113(1.1);3.899(16.0);3.424(11.9);2.170(0.7);2.044(5.3);1.277(1.6);1.259(3.2);1.241(1.5);0.008(0.8);0.000(27.4);−0.009(0.8)

### Example No. A8-6

### ¹H-NMR (400 MHz, CDCl₃ δ, ppm)

8.053(1.4);8.047(1.4);7.401(0.8);7.396(0.9);7.383(1.3);7.378(0.9);7.363(2.4);7.350(1.8); 7.344(1.6);7.322(0.5);7.270(0.6);7.264(7.1);7.252(0.7);7.249(0.7);7.247(0.7);7.245(0.6); 7.231(0.7);7.226(0.6);6.911(1.2);6.907(1.0);6.889(2.1);6.886(1.0);6.870(0.6);6.868(0.5); 5.352(4.2);5.295(8.6);3.912(11.2);3.411(16.0);1.951(0.6);0.000(3.0)

### Example No. A8-7

### ¹H-NMR (400 MHz, CDCl₃ δ, ppm)

7.486(1.9);7.484(1.9);7.465(3.4);7.446(0.8);7.442(0.9);7.428(0.8);7.423(1.0);7.367(1.6); 7.349(1.3);7.345(1.1);7.327(0.9);7.262(27.5);7.254(0.6);7.240(0.7);7.238(0.7);7.235(0.7); 7.234(0.7);7.219(0.7);7.215(0.7);6.919(0.7);6.917(0.8);6.901(1.0);6.899(1.9);6.896(1.3); 6.882(0.5);6.880(0.9);6.876(1.0);5.658(4.5);3.861(12.5);3.474(16.0);2.044(0.9);1.623(1.1);1.259(0.6);0.000(10.4)

### Example No. A8-10

### ¹H-NMR (400 MHz, CDCl₃ δ, ppm)

7.502(10.1);7.436(0.9);7.431(1.1);7.417(0.9);7.413(1.1);7.261(65.7);7.244(0.8);7.242(0.8); 7.238(0.9);7.223(0.8);7.219(0.7);6.917(0.8);6.915(1.1);6.896(3.1);6.880(0.7);6.878(1.3);6.875(1.3);5.632(4.6);3.856(15.3);3.459(16.0);1.581(1.4);0.008(0.8);0.000(27.8);−0.009(0.8)

### Example No. A8-11

### ¹H-NMR (400 MHz, CDCl₃ δ, ppm)

7.851(0.9);7.847(0.9);7.446(0.6);7.426(0.7);7.360(1.0);7.359(1.0);7.356(1.1);7.355(1.0); 7.342(1.0);7.341(1.1);7.338(1.1);7.337(1.1);7.268(0.5);7.267(0.7);7.266(0.8);7.265(1.0); 7.261(45.5);7.258(0.6);7.251(1.2);7.247(1.4);7.231(2.0);7.228(2.1);7.226(1.0);7.212(1.6); 7.209(0.8);7.208(0.9);7.156(0.6);7.153(0.6);7.135(0.6);7.131(0.8);7.128(0.6);7.110(0.5);6.894(1.5);6.891(1.5);6.888(0.6);6.873(3.4);6.870(1.3);6.854(0.9);6.852(0.7);5.234(3.3);5.232(3.3);3.884(16.0);3.389(11.8);3.386(11.7);0.008(0.6);0.000(17.6);−0.003(0.7);−0.009(0.5)

### Example No. A8-15

### ¹H-NMR (400 MHz, CDCl₃ δ, ppm)

7.987(0.6);7.966(1.2);7.949(1.2);7.944(0.8);7.928(0.6);7.408(1.0);7.391(1.1);7.296(0.8); 7.292(0.8);7.276(1.7);7.273(1.7);7.271(1.7);7.263(17.8);7.253(1.2);6.997(0.6);6.990(0.7); 6.975(1.1);6.969(1.2);6.953 (0.7);6.949(0.7);6.914 (3.3);6.900(2.4);6.898(2.4);6.892(3.0); 6.882(1.9);6.866(0.9);6.860(0.8);5.273(4.3);3.962(0.6);3.907(16.0);3.834(0.5);3.455(12.9);2.729(1.1);0.008(0.5);0.000(6.9)

### Example No. A8-17

### ¹H-NMR (400 MHz, CDCl₃ δ, ppm)

7.501(0.6);7.479(1.1);7.458(0.6);7.436(0.9);7.435(0.9);7.431(1.1);7.418(0.9);7.417(1.0); 7.413(1.2);7.269(0.6);7.268(0.6);7.267(0.7);7.266(0.8);7.2654(1.0);7.2646(1.3);7.264(1.7);7.263(2.5);7.260(67.6);7.257(1.1);7.2562(1.1);7.2556(1.1);7.251(0.8);7.237(1.0);7.235(0.9);7.232(0.9);7.231(1.0);7.217(1.0);7.212(0.9);7.210(0.6);7.072(1.5);7.053(2.1);7.032(1.3);6.915(0.9);6.913(1.1);6.897(1.3);6.895(3.0);6.878(0.7);6.876(1.3);6.873(1.3); 5.267(3.2);3.857(16.0);3.418(10.1);2.044(0.7);1.580(0.7);0.008(0.9);0.000(30.7);−0.009(0.8)

### Example No. A8-21

### ¹H-NMR (400 MHz, CDCl₃ δ, ppm)

8.634(0.6);8.631(0.6);8.629(0.7);8.627(0.6);8.622(0.6);8.619(0.7);8.617(0.6);8.615(0.6); 7.921(0.5);7.918(0.9);7.915(0.6);7.901(0.7);7.899(1.1);7.896(0.7);7.776(0.6);7.771(0.6); 7.756(1.1);7.752(1.0);7.737(0.5);7.733(0.5);7.355(0.6);7.352(0.6);7.343(0.7);7.340(0.6); 7.336(0.6);7.333(0.6);7.324(0.6);7.321(0.6);7.260(49.1);7.254(0.8);7.249(0.9);7.234(0.9);7.231(1.0);7.215(0.8);7.213(0.7);7.210(0.6);7.208(0.6);7.194(0.7);7.190(0.6);6.877(1.0);6.862(0.9);6.859(1.3);6.843(1.4);6.841(1.2);6.825(0.6);6.822(0.6);5.252(4.7);3.864(11.9);3.463(16.0);1.653 (0.5);1.256(0.7);0.008(0.5);0.000(18.7);-0.009(0.6)

### Example No. A8-30

### ¹H-NMR (400 MHz, CDCl₃ δ, ppm)

7.378(0.5);7.363(0.6);7.260(34.3);6.882(1.5);6.862(1.3);6.053(2.1);5.189(2.7);3.963(16.0); 3.945(1.0);3.850(6.8);3.825(2.8);3.614(9.8);1.676(0.7);0.000(12.5)

### Example No. A8-40

### ¹H-NMR (400 MHz, CDCl₃ δ, ppm)

8.488(2.9);8.475(3.0);7.506(3.0);7.493(2.8);7.426(0.8);7.422(1.0);7.408(0.9);7.403(1.0); 7.262(41.2);7.254(0.7);7.239(0.8);7.237(0.8);7.235(0.8);7.233(0.8);7.219(0.8);7.214(0.8); 6.914(0.8);6.912(1.1);6.896(1.3);6.893(2.1);6.890(1.6);6.877(0.7);6.875(1.0);6.869(1.3); 5.542(5.6);3.854(15.5);3.576(16.0);2.000(1.0);0.008(0.5);0.000(17.6)

### Example No. A8-62

### ¹H-NMR (400 MHz, CDCl₃ δ, ppm)

8.039(0.7);8.036(1.3);8.032(0.8);7.869(0.6);7.853(0.5);7.849(0.7);7.845(0.5);7.734(0.5); 7.731(0.8);7.727(0.5);7.715(0.7);7.711(0.9);7.708(0.6);7.542(0.8);7.541(0.8);7.523(1.4); 7.522(1.4);7.503(0.6);7.502(0.6);7.331(0.9);7.327(1.0);7.312(1.0);7.308(1.0);7.269(0.8); 7.262(21.1);7.250(0.8);7.248(0.8);7.246(0.7);7.243(0.7);7.229(0.8);7.225(0.6);6.919(1.1);6.898(1.0);6.895(0.9);6.893(0.7);6.877(1.5);6.874(1.3);6.858(0.7);6.856(0.6);5.184(3.4);3.937(11.8);3.379(16.0);0.000(9.3)

### Example No. A8-63

### ¹H-NMR (400 MHz, CDCl₃ δ, ppm)

7.800(1.6);7.795(0.6);7.784(0.8);7.779(2.8);7.723(0.6);7.719(3.2);7.714(0.8);7.703(0.7); 7.698(1.8);7.311(0.9);7.307(1.0);7.293(1.1);7.289(1.2);7.271(0.6);7.268(1.4);7.260(88.9); 7.250(0.9);7.248(0.8);7.245(0.7);7.243(0.7);7.229(0.7);7.225(0.6);6.907(1.1);6.898(1.0); 6.896(0.7);6.886(1.0);6.880(1.6);6.877(1.2);6.861(0.7);6.859(0.6);5.199(3.8);4.131(0.7);4.113(0.7);3.892(12.5);3.385(16.0);2.257(0.6);2.050(0.5);2.044(3.3);1.277(1.0);1.259(2.1);1.241(1.0);0.008(1.1);0.000(39.9);−0.009(1.1)

### Example No. A8-81

### ¹H-NMR (400 MHz, CDCl₃ δ, ppm)

8.461(0.9);8.441(0.9);7.745(0.8);7.727(1.0);7.630(0.9);7.611(0.6);7.551(0.6);7.532(0.9); 7.423(0.8);7.419(0.9);7.404(1.0);7.400(0.9);7.282(0.7);7.278(0.7);7.269(0.5);7.268(0.6); 7.267(0.7);7.266(0.9);7.260(56.8);7.257(1.7);7.256(0.7);7.243(0.8);7.239(0.7);6.939(0.8); 6.937(1.2);6.927(1.4);6.925(1.6);6.921(2.1);6.918(1.5);6.906(1.1);6.904(1.5);6.902(1.6);6.900(0.5);5.408(3.1);3.910(16.0);3.367(12.0);2.044(1.0);1.259(0.7);0.008(0.7);0.000(25.2);−0.009(0.7)

### Example No. A8-94

### ¹H-NMR (400 MHz, CDCl₃ δ, ppm)

8.490(1.6);8.485(1.6);8.194(1.1);8.188(1.1);8.174(1.2);8.167(1.1);7.400(1.8);7.399(1.7); 7.379(1.7);7.378(1.6);7.347(0.8);7.343(1.0);7.328(0.9);7.324(1.0);7.282(0.7);7.277(0.7); 7.263(31.4);7.257(0.9);7.242(0.8);7.238(0.8);6.909(2.4);6.906(1.2);6.893(1.6);6.890(1.9); 6.888(1.5);6.885(1.1);6.874(0.9);6.872(0.7);5.171(3.2);3.963(0.8);3.895(16.0);3.834(0.7);3.413(13.9);3.117(0.8);2.954(0.7);2.883(0.7);2.881(0.7);2.847(0.8);2.728(1.7);0.000(12.0)

### Example No. A8-462

### ¹H-NMR (400 MHz, CDCl₃ δ, ppm)

7.352(0.8);7.347(0.9);7.333(0.9);7.329(1.0);7.262(14.1);7.226(0.5);7.222(0.5);7.208(0.7); 7.206(0.7);7.203(0.7);7.201(0.7);7.187(0.7);7.182(0.8);7.178(0.8);6.892(1.0);6.887(0.8); 6.885(0.9);6.873(1.3);6.869(1.6);6.866(1.6);6.856(1.4);6.854(1.4);6.850(0.8);6.848(0.7);6.836(1.0);3.880(0.9);3.859(0.6);3.838(0.9);3.799(11.2);3.399(16.0);2.807(0.6);2.788(1.2);2.770(0.7);2.246(0.6);2.225(0.6);1.932(0.5);0.000(5.2)

### Example No. A8-603

### ¹H-NMR (400 MHz, CDCl₃ δ, ppm)

8.112(0.7);8.109(0.7);8.092(0.8);8.089(0.8);8.033(0.6);8.030(0.6);8.013(0.7);8.010(0.6); 7.598(0.7);7.594(0.7);7.492(0.7);7.489(0.7);7.267(0.6);7.2664(0.8);7.2656(0.9);7.265(1.2);7.261(48.2);7.258(1.3);7.257(0.7);6.905(1.7);6.885(1.6);5.742(1.1);3.911(16.0);3.902(10.3);3.405(1.7);2.955(0.7);2.884(0.6);2.883(0.6);2.044(0.7);0.008(0.7);0.003(0.6);0.002(1.0);0.000(20.9);−0.0027(0.9);−0.0034(0.6);−0.008(0.6)

### Example No. A8-679

### ¹H-NMR (400 MHz, CDCl₃ δ, ppm)

8.754(1.1);8.750(1.3);8.743(1.2);8.739(1.2);8.695(1.48.691(0.9);8.675(1.1);8.671(0.9); 7.597(1.1);7.586(1.1);7.577(1.0);7.565(1.0);7.334(0.9);7.330(1.0);7.315(1.0);7.311(1.1); 7.285(0.8);7.280(0.7);7.266(2.2);7.260(64.7);7.246(0.9);7.241(0.7);6.920(1.2);6.911(1.0); 6.908(0.7);6.899(1.0);6.892(1.6);6.890(1.2);6.874(0.7);6.871(0.6);5.481(3.0);3.912(12.3);3.480(16.0);2.051(0.7);2.044(0.7);1.564(0.7);1.259(0.6);0.008(1.0);0.000(28.5);−0.009(0.9)

### Example No. A9-3

### ¹H-NMR (400 MHz, CDCl₃ δ, ppm)

7.890(2.9);7.885(0.9);7.874(0.9);7.868(3.1);7.389(4.5);7.386(1.9);7.385(2.0);7.382(4.9); 7.360(0.5);7.309(3.1);7.304(1.0);7.292(0.9);7.287(2.9);7.264(10.6);4.972(4.5);3.369(16.0); 0.000(4.4)

### Example No. A9-4

### ¹H-NMR (400 MHz, d₆-DMSO δ, ppm)

7.841(1.7);7.834(12.9);7.829(4.7);7.818(5.8);7.813(16.0);7.807(3.1);7.804(2.8);7.610(3.8); 7.606(4.0);7.590(5.4);7.587(5.1);7.563(2.2);7.480(2.3);7.460(3.5);7.440(1.6);5.313(5.0);3.622(7.5);2.525(0.5);2.520(0.9);2.511(16.6);2.507(37.0);2.502(52.2);2.498(37.2); 2.493(17.2);2.075(1.3);0.008(1.2);0.000(46.9);−0.009(1.6)

### Example No. A9-5

### ¹H-NMR (400 MHz, d₆-DMSO δ, ppm)

7.835(2.7);7.830(0.9);7.819(1.1);7.814(4.1);7.807(1.7);7.633(1.1);7.612(2.1);7.566(1.5); 7.561(1.3);7.546(1.0);7.541 (0.8);5.243(1.4);3.619(2.6);2.513(1.9);2.508(4.2);2.504(5.9); 2.499(4.2);2.495(1.9);2.076(16.0);0.000(5.6)

### Example No. A9-7

### ¹H-NMR (400 MHz, d₆-DMSO δ, ppm)

7.845(1.1);7.823(1.4);7.673(0.6);7.669(0.7);7.651(1.4);7.602(0.5);5.392(0.6);3.702(0.8); 3.170(16.0);2.512(1.3);2.507(2.8);2.503(3.9);2.498(2.7);2.494(1.2);0.000(2.5)

### Example No. A9-30

### ¹H-NMR (400 MHz, CDCl₃ δ, ppm)

7.918(1.6);7.913(0.5);7.901(0.5);7.896(1.7);7.296(1.8);7.291(0.5);7.279(0.5);7.274(1.6); 7.263(6.6);6.047(2.2);5.298(0.5);5.102(2.8);3.927(16.0);3.610(10.1);0.000(2.5)

### Example No. A9-35

### ¹H-NMR (400 MHz, CD3CN 6, ppm)

8.587(1.3);8.582(1.3);8.032(1.3);8.026(1.2);7.839(2.1);7.834(0.7);7.822(0.7);7.817(2.3); 7.293(2.3);7.288(0.7);7.276(0.7);7.272(2.0);5.093(4.9);3.498(16.0);1.972(0.6);1.964 (1.3); 1.955(13.4);1.952(9.6);1.946(14.7);1.940(19.2);1.934(13.3);1.927(6.9);0.000(0.6)

### Example No. A9-36

### ¹H-NMR (400 MHz, CD3CN 6, ppm)

8.888(0.9);8.885(0.9);8.261(1.0);8.257(0.9);7.825(2.3);7.820(0.7);7.808(0.8);7.803(2.5); 7.288(2.2);7.284(0.7);7.272(0.7);7.267(2.0);5.175(4.5);4.068(0.8);4.050(0.8);3.553(16.0); 2.322(0.3);2.290(0.4);1.972(3.7);1.965(0.9);1.958(1.5);1.953(8.0);1.946(9.0);1.940(11.6); 1.934(8.0);1.928(4.1);1.222(1.0);1.204(1.9);1. 186(1.0);0.000(0.4)

### Example No. A9-40

### ¹H-NMR (400 MHz, d₆-DMSO δ, ppm)

8.538(6.6);8.525(6.9);8.413(0.8);8.400(0.8);8.019(0.8);8.006(0.7);7.904(5.4);7.891(5.1); 7.780(12.5);7.775(4.5);7.763(5.0);7.758(16.0);7.752(1.9);7.562(1.6);5.384(1.8);3.741(7.5);3.627(1.2);3.601(1.0);3.167(0.6);2.675(0.8);2.670(1.2);2.666(0.8);2.562(0.6);2.557(0.8);2.524(3.4);2.519(4.9);2.510(67.0);2.506(144.8);2.501(201.3);2.497(141.9);2.492(64.5);2.471(1.0);2.466(1.2);2.462(1.4);2.458(1.2);2.453(0.6);2.381(0.9);2.333(0.9);2.328(1.2);2.323(0.9);0.146(0.5);0.050(0.6);0.008(4.8);0.000(160.9);−0.009(4.9);−0.049(0.5);−0.150(0.5)

### Example No. A9-59

### ¹H-NMR (400 MHz, d₆-DMSO δ, ppm)

7.838(1.7);7.832(13.1);7.827(4.3);7.815(4.7);7.810(16.0);7.804(1.8);7.670(1.3);7.654(1.7); 7.649(3.1);7.634(3.3);7.629(2.4);7.613(2.3);7.583(2.9);7.565(2.5);7.533(5.9);7.513(4.1);7.422(2.5);7.420(2.5);7.401(2.5);7.398(3.8);7.395(2.8);7.376(2.1);7.374(2.0);5.284(5.1);3.691(10.4);2.525(0.5);2.520(0.8);2.512(11.1);2.507(24.3);2.502(34.0);2.498(24.0); 2.493(10.9);2.075(5.3);0.008(0.8);0.000(29.6);-0.009(0.9)

### Example No. A9-137

### ¹H-NMR (400 MHz, d₆-DMSO δ, ppm)

7.845(1.6);7.839(13.1);7.834(4.3);7.822(4.6);7.817(16.0);7.811(1.9);7.688(3.4);7.672(3.7); 7.666(3.9);7.654(3.7);7.650(5.1);7.632(3.3);7.626(3.0);7.572(3.6);7.552(3.1);7.387(1.5);7.380(1.4);7.366(2.6);7.359(2.4);7.345(1.4);7.338(1.2);5.244(7.1);3.621(13.3);2.513(8.6);2.509(19.6);2.504(27.9);2.499(20.2);2.495(9.5);2.076(7.7);0.008(0.5);0.000(22.9); −0.009(0.8)

### Example No. A9-144

### ¹H-NMR (400 MHz, d₆-DMSO δ, ppm)

8.404(2.6);8.390(2.7);7.787(0.6);7.781(5.2);7.776(1.7);7.765(1.9);7.760(7.0);7.754(0.9); 7.619(4.4);7.598(3.4);7.248(2.6);7.233(2.5);5.266(5.1);4.989(0.9);4.968(3.1);4.946(3.3); 4.924(1.2);3.726(16.0);2.512(6.5);2.507(14.6);2.503(20.6);2.498(14.6);2.493(6.7);2.307(14.2);2.075(11.3);0.000(16.3);−0.009(0.5)

### Example No. A9-147

### ¹H-NMR (400 MHz, d₆-DMSO δ, ppm)

7.868(0.6);7.862(4.8);7.857(1.6);7.845(1.7);7.840(5.7);7.834(0.7);7.583(0.8);7.158(3.4); 7.066(3.3);5.174(1.9);3.861(0.5);3.810(16.0);3.743(0.8);3.606(2.6);3.570(4.9);2.520(0.6); 2.511(8.6);2.507(18.6);2.502(25.9);2.497(18.4);2.493(8.4);2.075(0.8);0.008(0.6);0.000(19.3);−0.009(0.6)

### Example No. A9-148

### ¹H-NMR (400 MHz, d₆-DMSO δ, ppm)

8.369(3.2);8.356(3.5);7.783(3.8);7.781(3.7);7.776(1.2);7.770(3.3);7.764(1.3);7.759(4.5); 7.753(0.6);7.631(2.2);7.609(1.7);5.307(3.4);3.892(16.0);3.730(8.6);2.512(2.7);2.508(6.4); 2.503(9.1);2.498(6.6);2.494(3.2);2.076(2.7);0.000(7.6)

### Example No. A9-151

### ¹H-NMR (400 MHz, CDCl₃ δ, ppm)

7.950(0.9);7.944(7.5);7.939(2.4);7.927(2.5);7.922(8.4);7.916(1.0);7.751(0.7);7.730(0.7); 7.582(2.0);7.578(2.7);7.573(1.4);7.568(1.0);7.563(4.3);7.558(4.9);7.542(2.3);7.535(0.8); 7.531 (2.5);7.527(3.5);7.520(5.4);7.516(2.1);7.509(1.5);7.506(1.7);7.502(3.5);7.496(0.7); 7.491(0.5);7.487(1.0);7.480(0.7);7.427(1.2);7.420(8.7);7.415(2.6);7.404(2.6);7.399(7.9); 7.392(1.0);7.272(0.6);7.269(18.9);6.185(1.5);6.167(1.5);3.613(16.0);3.595(13.1);3.226(2.3);2.940(3.1);2.002(10.5);1.913(12.3);1.895(12.2);1.506(1.0);1.490(1.0);1.445(1.0); 1.428(1.0);0.000(6.2)

### Example No. A9-186

### ¹H-NMR (400 MHz, d₆-DMSO δ, ppm)

7.959(1.9);7.909(0.6);7.904(0.5);7.887(1.4);7.883(1.3);7.861(2.5);7.840(1.0);7.822(0.7); 7.816(4.9);7.811(1.5);7.799(1.7);7.794(5.8);7.788(0.6);7.518(1.4);7.497(1.2);5.301(2.9); 3.614(5.9);2.514(3.7);2.510(8.2);2.505(11.6);2.501(8.3);2.496(3.8);2.077(16.0);0.000(6.7)

### Example No. A9-615

### ¹H-NMR (400 MHz, CDCl₃ δ, ppm)

7.937(2.6);7.933(0.9);7.921(0.9);7.916(2.8);7.394(1.6);7.388(1.7);7.316(3.0);7.311(0.9); 7.299(0.8);7.295(2.7);7.267(1.1);7.263(11.2);7.247(2.1);7.222(1.6);7.217(1.5);7.201 (0.6); 7.196(0.6);3.932(1.0);3.918(0.5);3.912(0.8);3.904(0.5);3.891(1.1);3.502(16.0);3.249(1.1);3.236(0.5);3.228(0.8);3.221(0.5);3.207(1.0);0.000(4.1)

### Example No. A9-617

### ¹H-NMR (400 MHz, CDCl₃ δ, ppm)

7.943(2.7);7.938(0.9);7.926(0.9);7.921(3.0);7.318(3.47.313(0.9);7.301(0.9);7.296(2.7); 7.264(13.6);7.257(0.6);7.247(0.5);7.244(0.6);7.118(0.6);7.115(0.7);7.099(0.8);7.097(1.0); 7.048(0.5);3.961(1.0);3.946(0.5);3.941(0.9);3.934(0.5);3.920(1.1);3.512(16.0);3.478(5.1);3.211(0.7);3.190(0.7);3.170(0.7);0.000(4.0)

### Example No. A9-619

### ¹H-NMR (400 MHz, CDCl₃ δ, ppm)

7.936(2.7);7.931(0.8);7.920(0.9);7.914(2.8);7.319(2.9);7.314(0.9);7.302(0.9);7.297(2.6); 7.264(19.0);7.224(0.9);7.211(0.9);7.203(1.1);7.189(1.0);7.028(1.3);7.006(2.3);6.984(1.0); 3.975(1.0);3.960(0.5);3.954(0.8);3.946(0.5);3.933(1.1);3.484(16.0);3.129(0.9);3.108(0.7);3.087(0.8);1.720(2.1);0.000(5.8)

### Example No. A9-624

### ¹H-NMR (400 MHz, CDCl₃ δ, ppm)

7.940(2.8);7.935(0.9);7.923(1.0);7.918(3.1);7.402(2.0);7.398(1.3);7.394(1.1);7.391(0.7); 7.381(0.7);7.319(3.0);7.314(0.9);7.302(0.9);7.297(2.8);7.262(18.7);7.198(1.4);7.197(1.4); 7.194(1.1);7.189(0.8);7.186(2.0);7.182(2.3);3.977(1.1);3.962(0.6);3.956(0.9);3.949(0.6); 3.935(1.2);3.487(16.0);3.133(1.1);3.119(0.5);3.112(0.9);3.091(1.0);0.000(5.3)

### Example No. A9-625

### ¹H-NMR (400 MHz, CDCl₃ δ, ppm)

7.925(1.9);7.921(0.6);7.909(0.6);7.904(2.1);7.451(1.7);7.446(0.5);7.435(0.6);7.430(2.0); 7.316(2.1);7.311(0.6);7.299(0.6);7.295(2.0);7.266(7.4);7.133(1.6);7.112(1.4);3.972(0.8); 3.951 (0.6);3.930(0.8);3.474(16.0);3.105(0.7);3.084(0.6);3.064(0.6);1.776(0.6);0.000(2.2)

### Example No. A9-627

### ¹H-NMR (400 MHz, CDCl₃ δ, ppm)

7.946(2.6);7.941(0.8);7.929(0.9);7.924(2.8);7.311(2.8);7.306(0.9);7.294(0.8);7.289(2.6); 7.264(10.1);7.246(0.6);7.243(0.6);7.223(0.5);7.202(0.7);7.198(0.6);7.183(0.8);7.179(0.6); 6.928(0.5);6.925(0.6);6.909(0.9);6.907(1.1);6.877(0.9);6.857(0.8);3.887(1.0);3.873(0.6); 3.867(0.9);3.859(0.6);3.844(11.4);3.502(16.0);3.474(3.2);3.167(1.0);3.154(0.5);3.146(0.8);3.139(0.5);3.126(0.9);0.000(3.0)

### Example No. A9-633

### ¹H-NMR (400 MHz, CDCl₃ δ, ppm)

7.935(2.7);7.930(0.8);7.918(0.9);7.913(2.8);7.588(1.4);7.568(1.7);7.382(1.6);7.362(1.3); 7.320(2.9);7.315(0.9);7.303 (0.8);7.299(2.6);7.263(15.3);4.033(1.1);4.019(0.6);4.013 (1.0); 4.006(0.6);3.992(1.2);3.495(16.0);3.484(1.5);3.218(0.9);3.204(0.5);3.197(0.8);3.176(0.8);0.000(4.2)

### Example No. A9-639

### ¹H-NMR (400 MHz, CDCl₃ δ, ppm)

7.940(1.2);7.919(1.2);7.351(0.9);7.330(1.2);7.315(1.47.293(1.47.263(6.1);7.183(1.0); 7.163(0.8);3.493(6.6);3.479(2.6);1.302(16.0);0.000(1.9)

### Example No. A9-652

### ¹H-NMR (400 MHz, CDCl₃ δ, ppm)

7.942(2.6);7.937(0.8);7.925(0.8);7.920(2.8);7.312(2.8);7.308(0.8);7.296(0.8);7.291(2.6); 7.265(5.6);7.238(0.7);7.235(0.9);7.216(0.5);6.836(0.7);6.818(0.8);6.800(0.7);6.782(2.3); 6.780(1.9);3.962(1.1);3.948(0.6);3.942(0.9);3.935(0.6);3.921(1.2);3.797(11.7);3.480(16.0); 3.467(2.3);3.138(1.0);3.124(0.5);3.117(0.9);3.111(0.5);3.096(0.9);0.000(1.6)

### Example No. A9-661

### ¹H-NMR (400 MHz, CDCl₃ δ, ppm)

7.939(1.9);7.934(0.6);7.922(0.6);7.917(2.0);7.327(2.1);7.322(0.6);7.310(0.6);7.305(1.9); 7.269(6.4);6.449(4.2);4.012(0.7);3.991(0.6);3.970(0.7);3.857(1.0);3.851(16.0);3.817(9.4); 3.516(10.2);3.474(8.3);3.099(0.7);3.078(0.6);3.058(0.6);0.000(2.0)

### Example No. A9-662

### ¹H-NMR (400 MHz, CDCl₃ δ, ppm)

7.945(3.0);7.940(0.9);7.929(1.0);7.924(3.1);7.319(3.2);7.314(0.9);7.303(0.9);7.298(2.9); 7.267(9.2);6.822(0.7);6.802(2.0);6.785(1.1);6.780(1.4);6.759(2.2);3.974(1.0);3.959(0.6); 3.953(0.9);3.946(0.6);3.932(1.1);3.875(12.1);3.859(12.2);3.492(16.0);3.472(8.6);3.106(1.0);3.092(0.5);3.085(0.9);3.079(0.5);3.064(0.9);0.000(2.8)

### Example No. A9-663

### ¹H-NMR (400 MHz, CDCl₃ δ, ppm)

7.945(2.8);7.940(0.9);7.928(0.9);7.923(3.0);7.311(3.0);7.306(0.9);7.294(0.9);7.289(2.8); 7.261(15.9);7.087(0.7);7.068(1.0);7.008(1.0);6.978(0.7);6.973(0.5);3.917(1.0);3.903(0.5); 3.897(0.8);3.889(0.5);3.875(1.1);3.478(16.0);3.095(0.8);3.074(0.7);3.053(0.8);2.240(5.7);2.232(5.5);0.000(6.9)

### Example No. A9-665

### ¹H-NMR (400 MHz, CDCl₃ δ, ppm)

7.946(2.8);7.941(0.9);7.929(0.9);7.924(3.1);7.519(0.5);7.312(3.1);7.307(1.0);7.295(1.1); 7.290(2.8);7.260(96.8);6.996(0.5);6.861(2.8);3.688(0.9);3.676(0.6);3.666(0.6);3.656(0.6); 3.645(1.1);3.540(16.0);3.201(0.9);3.190(0.5);3.179(0.6);3.158(0.8);2.352(13.4);2.251(5.9);1.568(4.3);0.008(1.3);0.005(0.5);0.000(40.9);−0.009(1.1)

### Example No. A9-666

### ¹H-NMR (400 MHz, CDCl₃ δ, ppm)

7.955(2.9);7.949(0.9);7.938(1.0);7.933(3.3);7.314(3.1);7.308(1.0);7.297(0.9);7.292(3.0); 7.260(21.7);7.069(0.9);7.050(1.3);7.024(1.0);6.983(0.6);6.980(0.6);3.792(0.9);3.779(0.5); 3.770(0.7);3.762(0.5);3.750(1.0);3.514(16.0);3.173(0.9);3.152(0.6);3.131(0.8);2.705(1.6);2.687(1.6);2.668(0.5);2.310(5.6);1.625(1.5);1.248(2.8);1.229(6.2);1.210(2.7);0.000(9.1)

### Example No. A9-668

### ¹H-NMR (400 MHz, CDCl₃ δ, ppm)

8.129(0.7);8.126(0.8);8.122(0.8);8.117(1.3);8.112(1.7);7.934(2.8);7.929(0.9);7.917(0.9); 7.912(3.0);7.637(0.6);7.617(0.8);7.529(1.0);7.510(0.8);7.507(1.0);7.488(0.7);7.326(3.1); 7.321(0.9);7.309(0.9);7.304(2.8);7.264(32.0);4.088(1.0);4.073(0.6);4.067(0.9);4.060(0.5); 4.046(1.1);3.515(16.0);3.487(10.9);3.272(0.9);3.251(0.8);3.231(0.9);1.652(1.8);0.000(8.9)

### Example No. A9-670

### ¹H-NMR (400 MHz, CDCl₃ δ, ppm)

7.967(3.0);7.962(0.9);7.950(0.9);7.945(3.0);7.827(1.5);7.813(0.7);7.806(1.9);7.797(0.7); 7.791(0.6);7.774(0.7);7.698(1.2);7.490(1.0);7.485(0.9);7.482(1.1);7.474(2.2);7.466(1.0); 7.463(0.9);7.459(0.9);7.381(0.9);7.377(0.9);7.360(0.8);7.356(0.8);7.331(3.2);7.326(1.0); 7.314(0.9);7.309(2.9);7.262(22.8);4.084(1.1);4.070(0.6);4.064(1.0);4.056(0.6);4.043(1.2);3.532(16.0);3.483(6.5);3.331(1.0);3.317(0.5);3.310(0.9);3.304(0.6);3.289(0.9);1.736(1.3);0.000(6.7)

### Example No. A10-2

### ¹H-NMR (400 MHz, CDCl₃ δ, ppm)

7.587(1.5);7.583(0.9);7.467(0.8);7.463(1.3);7.459(0.8);7.452(1.0);7.445(2.3);7.442(2.4); 7.436(0.8);7.429(1.0);7.401 (1.4);7.384(0.9);7.379(0.7);7.362(0.6);7.351(0.8);7.344(0.6); 7.337(0.7);7.328(1.0);7.308(0.5);7.260(48.5);7.213(2.3);7.203(1.3);7.200(1.3);7.197(1.1); 7.190(1.7);5.066(4.7);3.389(16.0);1.559(3.0);0.008(0.8);0.000(20.6);−0.009(0.9)

### Example No. A10-4

### ¹H-NMR (400 MHz, d₆-DMSO δ, ppm)

7.863(4.4);7.860(4.8);7.843(5.1);7.840(5.2);7.776(0.6);7.756(5.1);7.753(4.9);7.737(5.8); 7.733(5.2);7.576(2.3);7.559(8.3);7.539(10.8);7.531 (4.7);7.520(6.1);7.495(2.4);7.442(2.4); 7.422(2.9);7.406(1.4);5.342(12.3);5.263(0.8);3.602(16.0);2.574(1.5);2.524(0.7);2.520(1.1);2.511(24.1);2.506(54.0);2.502(76.0);2.497(54.0);2.493(24.7);2.471(0.5);2.397(0.8); 2.075(8.1);0.008(1.6);0.000(61.7);-0.009(2.1)

### Example No. A10-5

### ¹H-NMR (400 MHz, d₆-DMSO δ, ppm)

7.841(7.9);7.836(8.2);7.786(7.7);7.765(10.0);7.695(0.9);7.693(1.0);7.674(1.8);7.638(5.7); 7.633(5.5);7.618(4.5);7.612(4.9);7.594(0.7);7.589(0.8);7.572(0.8);7.521(4.1);7.500(4.3); 7.479(2.2);7.462(2.5);7.445(1.7);7.415(2.6);7.398(2.9);7.382(1.3);5.435(1.5);5.242(10.8);3.636(1.4);3.547(15.2);2.568(0.9);2.524(1.1);2.519(1.6);2.511(21.3);2.506(46.1); 2.502(64.2);2.497(45.1);2.492(20.2);2.074(16.0);0.008(1.7);0.000(53.7);−0.009(1.5)

### Example No. A10-7

### ¹H-NMR (400 MHz, d₆-DMSO δ, ppm)

7.706(7.5);7.702(8.5);7.683(16.0);7.636(2.1);7.627(8.0);7.620(2.4);7.609(5.3);7.604(4.4); 7.586(3.2);7.569(1.3);7.549(4.5);7.529(2.0);7.508(0.9);7.477(1.7);7.473(1.7);7.466(1.4); 7.465(1.4);7.458(2.3);7.445(1.6);7.441(1.0);7.395(0.7);7.355(0.6);7.337(0.8);5.458(14.9);4.686(1.8);3.706(15.3);2.583(3.7);2.524(0.7);2.520(1.1);2.511(16.0);2.506(35.0); 2.502(48.8);2.497(34.2);2.493(15.3);2.397(1.8);2.074(5.8);0.008(1.1);0.000(41.7);−0.009(1.2)

### Example No. A10-10: ¹H-NMR (400 MHz, CDCl₃ δ, ppm)

7.512(7.7);7.509(1.2);7.502(0.7);7.499(0.6);7.495(0.7);7.490(0.8);7.485(1.0);7.355(0.8); 7.350(0.6);7.345(0.6);7.340(0.6);7.338(0.7);7.332(1.1);7.261(33.5);7.226(1.4);7.221(1.9); 7.212(2.8);7.203(1.7);7.198(1.0);5.601(5.1);3.474(16.0);1.583(2.7);0.000(14.4)

### Example No. A10-17

### ¹H-NMR (400 MHz, CDCl₃ δ, ppm)

7.519(0.7);7.513(2.3);7.507(1.9);7.498(1.5);7.494(1.8);7.491(2.4);7.475(0.9);7.470(1.0); 7.454(0.5);7.349(1.5);7.344(1.3);7.341(1.1);7.333(1.5);7.331 (1.5);7.326(2.1);7.310(0.9); 7.260(72.5);7.241(1.0);7.236(1.2);7.223(2.6);7.218(2.8);7.216(2.9);7.210(3.3);7.208(4.6); 7.200(2.2);7.198(2.0);7.192(1.7);7.179(0.6);7.082(2.2);7.063(3.6);7.042(2.1);5.247(5.6);3.435(16.0);3.385(0.7);2.955(1.7);2.884(1.5);2.735(0.5);1.576(1.7);0.008(1.9);0.000(31.5);−0.008(1.4);−0.050(1.0)

### Example No. A10-21

### ¹H-NMR (400 MHz, CDCl₃ δ, ppm)

8.539(0.5);8.527(0.5);7.707(0.6);7.702(0.6);7.689(0.7);7.684(0.7);7.677(0.6);7.674(0.8); 7.673(0.8);7.671(0.6);7.380(0.7);7.377(0.7);7.299(1.0);7.289(1.4);7.283(2.2);7.279(0.8); 7.275(3.5);7.273(1.8);7.270(0.7);7.266(1.0);7.264(7.2);4.942(2.6);2.900(0.5);2.425(16.0); 0.000(3.0)

### Example No. A10-30

### ¹H-NMR (400 MHz, d₆-DMSO δ, ppm)

7.547(0.8);7.543(1.0);7.538(0.9);7.449(0.5);7.440(0.9);7.436(1.0);7.431(0.9);6.419(2.8); 5.246(1.8);3.915(16.0);3.878(4.3);2.511(1.9);2.507(4.1);2.502(5.9);2.498(4.2);2.493(1.9); 0.000(5.4)

### Example No. A10-33

### ¹H-NMR (400 MHz, d₆-DMSO δ, ppm)

8.711(0.9);7.792(3.2);7.787(3.5);7.714(0.8);7.437(0.7);7.388(0.9);7.371(0.7);7.336(0.7); 5.216(0.8);3.586(1.1);3.391(8.0);3.166(16.0);2.674(1.4);2.670(1.9);2.665(1.3);2.558(2.1); 2.552(0.6);2.539(2.5);2.534(3.7);2.529(3.6);2.523(6.2);2.519(8.4);2.510(111.5);2.505(238.7);2.501(331.4);2.496(232.1);2.492(103.3);2.452(0.6);2.395(1.3);2.337(0.7);2.332(1.5);2.328(2.0);2.323(1.4);2.318(0.7);2.073(0.6);0.033(1.1);0.008(4.6);0.000(151.0);−0.009(4.5);−0.150(0.5)

### Example No. A10-35

### ¹H-NMR (400 MHz, CD₃CN 6, ppm)

8.632(1.4);8.627(1.4);8.028(1.4);8.022(1.4);7.358(0.6);7.349(0.8);7.345(0.5);7.342(0.5); 7.335(0.9);7.320(0.5);7.311(0.5);7.305(0.7);7.297(0.9);7.246(0.3);7.236(2.0);7.228(1.5); 7.222(1.3);7.213(1.4);5.239(4.9);3.437(16.0);2.143(1.0);1.964(0.7);1.957(1.0);1.952(4.5); 1.945(7.9);1.939(10.2);1.933(7.0);1.927(3.6);0.000(1.2)

### Example No. A10-36

### ¹H-NMR (400 MHz, CD₃CN δ, ppm)

8.930(0.9);8.928(0.9);8.251(0.9);8.247(0.9);7.343(0.6);7.334(0.6);7.329(0.7);7.320(0.9); 7.314(0.5);7.305(0.6);7.301(0.4);7.298(0.4);7.291(0.9);7.234(1.8);7.225(1.3);7.220(1.1); 7.211(1.2);5.332(4.0);4.068(0.5);4.050(0.5);3.498(16.0);2.617(0.7);2.455(0.4);2.153(16.5); 1.972(2.2);1.964(1.4);1.958(2.1);1.952(9.9);1.946(17.4);1.940(22.7);1.934(15.6);1.928(8.0);1.222(0.6);1.204(1.2);1.186(0.6);0.000(1.1)

### Example No. A10-40

### ¹H-NMR (400 MHz, CDCl₃ δ, ppm)

8.494(1.9);8.481(1.9);7.513(1.9);7.500(1.9);7.494(0.9);7.488(0.7);7.486(0.7);7.480(0.7); 7.476(0.8);7.471(1.0);7.344(0.8);7.340(0.7);7.335(0.6);7.328(0.8);7.327(0.8);7.321(1.1); 7.261(29.6);7.230(0.6);7.217(1.4);7.21 1(1.9);7.202(2.4);7.194(1.4);7.187(1.0);5.512(4.9); 3.584(16.0);2.043(0.7);1.578(1.0);1.259(0.5);0.008(0.8);0.000(13.2);−0.008(0.6)

### Example No. A10-59

### ¹H-NMR (400 MHz, d₆-DMSO δ, ppm)

7.706(1.3);7.691(1.5);7.686(3.0);7.670(3.1);7.665(2.1);7.649(2.0);7.598(2.1);7.578(3.7); 7.571(5.6);7.556(5.1);7.553(5.1);7.551(5.3);7.538(2.1);7.518(0.9);7.489(2.3);7.486(2.7); 7.479(1.8);7.465(5.2);7.461(4.2);7.443(2.5);5.341(8.7);3.684(16.0);2.582(0.5);2.520(0.6); 2.511(10.5);2.506(23.2);2.502(32.5);2.497(22.9);2.493(10.3);2.074(4.8);0.008(0.8); 0.000(25.3);−0.009(0.8)

### Example No. A10-62

### ¹H-NMR (400 MHz, CDCl₃ δ, ppm)

7.892(1.6);7.890(1.4);7.873(0.6);7.870(0.7);7.772(0.5);7.768(0.8);7.752(0.7);7.749(0.9); 7.745(0.6);7.592(0.7);7.590(0.7);7.573(1.1);7.571(1.1);7.420(0.8);7.412(0.8);7.405(0.8); 7.397(0.9);7.359(0.7);7.350(1.0);7.346(0.6);7.344(0.7);7.336(1.0);7.261(34.6);7.221(2.5); 7.213(2.0);7.207(1.8);7.198(2.1);5.1 19(4.0);3.416(16.0);2.044(1.4);1.590(0.6);1.259(0.9);0.000(14.7)

### Example No. A10-63

### ¹H-NMR (400 MHz, CDCl₃ δ, ppm)

7.735(11.0);7.394(0.8);7.388(0.6);7.383(0.7);7.381(0.7);7.378(0.6);7.371(1.0);7.357(0.8); 7.351(0.6);7.348(0.6);7.344(0.6);7.338(0.7);7.334(1.1);7.262(17.2);7.233(0.5);7.221(1.4);7.216(1.6);7.211(1.47.207(2.47.200(1.2);7.197(1.2);7.194(1.1);5.143(5.0);3.412(16.0);3.362(0.7);1.627(0.9);0.000(7.1)

### Example No. A10-81

### ¹H-NMR (400 MHz, CDCl₃ δ, ppm)

8.291(0.7);8.271(0.8);7.764(0.6);7.746(0.8);7.646(0.7);7.553(0.7);7.515(0.9);7.508(0.7); 7.505(0.8);7.504(0.8);7.498(0.8);7.492(1.1);7.379(0.9);7.373(0.7);7.367(0.7);7.366(0.7); 7.362(0.8);7.356(1.2);7.262(20.2);7.246(1.8);7.244(1.7);7.240(1.3);7.234(2.1);7.227(1.1); 7.223(1.3);7.220(1.2);5.328(3.4);3.404(16.0);2.955(0.8);2.884(0.7);2.882(0.7);2.731(1.1);0.000(8.6)

### Example No. A10-86

### ¹H-NMR (400 MHz, d₆-DMSO δ, ppm)

8.105(5.9);8.101(6.1);7.942(1.9);7.937(1.8);7.920(4.4);7.916(4.4);7.891(7.4);7.869(3.0); 7.488(1.8);7.470(3.2);7.439(2.9);7.422(3.9);7.360(1.9);7.341(2.1);7.325(0.9);5.287(8.6); 3.547(16.0);2.520(0.8);2.512(12.9);2.507(28.7);2.503(40.5);2.498(28.8);2.493(13.2);2.075(7.8);0.008(1.1);0.000(37.4);−0.007(0.5);−0.009(1.2)

### Example No. A10-137

### ¹H-NMR (400 MHz, d₆-DMSO δ, ppm)

7.829(3.0);7.813(3.3);7.807(3.4);7.791(3.2);7.702(3.1);7.695(3.1);7.680(3.1);7.673(3.0); 7.592(1.8);7.573(2.4);7.544(3.6);7.533(1.7);7.514(1.9);7.494(0.9);7.472(2.0);7.466(2.0); 7.452(4.5);7.445(3.4);7.437(2.6);7.430(2.6);7.423(2.0);5.276(9.5);3.606(16.0);2.512(9.0); 2.507(20.6);2.502(29.4);2.498(21.2);2.493(10.0);2.075(9.1);0.008(0.6);0.000(22.9);−0.009(0.8)

### Example No. A10-144

### ¹H-NMR (400 MHz, d₆-DMSO δ, ppm)

7.558(0.6);7.555(0.7);7.549(1.0);7.543(0.5);5.317(1.0);4.989(0.5);4.967(0.6);3.757(2.8); 2.511(0.9);2.506(2.0);2.502(2.8);2.497(2.0);2.492(0.9);2.321(2.5);2.074(16.0);0.000(1.9)

### Example No. A10-147

### ¹H-NMR (400 MHz, d₆-DMSO δ, ppm)

7.609(0.8);7.591(1.5);7.572(1.7);7.550(0.8);7.485(0.8);7.483(0.8);7.466(1.0);7.269(4.6); 7.214(5.3);5.209(3.1);4.479(0.7);3.843(16.0);3.776(0.9);3.760(15.5);3.629(7.6);2.580(1.6);2.511(3.6);2.507(8.1);2.502(11.4);2.498(8.1);2.493(3.8);2.398(0.8);2.075(4.9);0.000(10.1)

### Example No. A10-148

### ¹H-NMR (400 MHz, d₆-DMSO δ, ppm)

8.516(3.0);8.503(3.2);7.836(3.1);7.823(3.0);7.598(0.9);7.595(0.7);7.579(1.3);7.568(2.0); 7.566(2.0);7.561(1.7);7.553(1.3);7.550(0.8);7.505(0.8);7.498(0.6);7.490(0.5);7.487(0.6); 7.483(0.5);7.480(0.6);5.345(3.8);3.940(16.0);3.748(10.6);2.511(2.8);2.507(6.3);2.502(8.9);2.498(6.3);2.493(2.9);2.075(4.0);0.000(6.8)

### Example No. A10-189

### ¹H-NMR (400 MHz, CDCl₃ δ, ppm)

8.847(0.9);8.845(0.9);8.841(0.9);8.824(2.6);8.820(2.5);8.818(2.6);7.944(1.0);7.939(1.0); 7.923(1.8);7.918(1.8);7.906(0.7);7.901(0.6);7.885(0.8);7.874(2.2);7.853(1.2);7.590(0.7); 7.583(0.6);7.578(0.7);7.572(0.7);7.567(0.9);7.519(0.9);7.461(1.0);7.440(0.8);7.426(0.6); 7.421(0.5);7.415(0.6);7.414(0.6);7.410(0.6);7.404(1.0);7.372(1.8);7.365(1.5);7.361(2.6); 7.358(0.9);7.355(2.0);7.349(2.9);7.345(2.0);7.343(2.0);7.341(1.3);7.339(1.4);7.333(1.9);7.326(1.0);7.322(1.1);7.319(1.0);7.283(0.5);7.279(0.5);7.276(1.2);7.275(0.7);7.274(0.7);7.273(0.8);7.2723(0.9);7.2715(1.0);7.271(1.0);7.270(1.1);7.269(1.2);7.2683(1.4);7.2675(1.7);7.267(2.1);7.264(6.2);7.263(8.8);7.260(161.8);7.255(2.3);7.253(3.3);7.252(3.0); 7.249(1.0);7.247(3.2);7.241(4.2);7.228(0.8);7.175(2.2);7.169(1.3);7.163(1.8);7.159(1.0);7.152(1.3);6.996(0.9);4.884(1.5);4.867(1.5);4.849(0.5);4.772(0.7);4.755(0.7);3.040(1.9);2.577(10.1);2.556(16.0);2.472(1.7);2.045(0.6);2.034(13.8);2.017(13.6);1.721(4.4); 1.704(4.4);1.651(0.8);1.634(0.8);1.547(7.4);0.882(0.9);0.008(2.0);0.0063(0.7);0.0055(0.7); 0.005(0.9);0.004(1.2);0.000(65.8);−0. 005(1.1);−0.006(0.8);−0.007(0.6);−0.009(1.9)

### Example No. A10-462

### ¹H-NMR (400 MHz, CDCl₃ δ, ppm)

7.428(0.7);7.427(0.7);7.421(0.6);7.418(0.5);7.415(0.6);7.410(0.7);7.405(0.9);7.320(0.7); 7.319(0.7);7.314(0.6);7.309(0.6);7.305(0.6);7.302(0.7);7.297(1.0);7.262(14.2);7.204(0.7); 7.191(1.3);7.187(1.7);7.185(1.3);7.182(0.9);7.177(2.7);7.169(1.1);7.168(1.3);7.163(1.1); 7.161(0.6);6.896(0.9);6.876(1.2);6.856(0.7);3.845(0.8);3.825(0.6);3.804(0.8);3.415(16.0);2.810(0.5);2.791(1.1);2.773(0.6);2.245(0.5);2.226(0.5);2.224(0.6);0.000(5.2)

### Example No. A10-603

### ¹H-NMR (400 MHz, CDCl₃ δ, ppm)

8.032(0.8);8.028(0.8);8.012(0.9);8.009(0.9);7.954(0.8);7.952(0.9);7.935(1.0);7.932(1.0); 7.607(1.0);7.603(1.0);7.588(0.7);7.584(0.6);7.525(0.7);7.522(0.8);7.506(1.0);7.502(1.0); 7.487(0.6);7.483(0.5);7.461(0.9);7.452(1.0);7.448(0.7);7.445(0.7);7.438(1.0);7.348(0.8); 7.341(0.7);7.338(0.6);7.334(1.0);7.325(1.1);7.261(19.5);7.211(0.9);7.206(2.6);7.198(1.5);7.196(1.5);7.192(1.5);7.190(1.4);7.182(2.2);5.637(4.8);3.889(13.6);3.839(0.6);3.403(16.0);3.353(0.8);1.612(1.4);0.000(8.4)

### Example No. A10-639

### ¹H-NMR (400 MHz, CDCl₃ δ, ppm)

7.352(0.8);7.347(0.6);7.336(0.5);7.331(1.2);7.324(0.6);7.261(3.2);7.230(0.9);7.213(0.8); 7.209(1.2);7.199(1.0);3.454(6.3);1.999(1.1);1.311(3.1);1.304(16.0);0.000(1.5)

### Example No. A10-663

### ¹H-NMR (400 MHz, CDCl₃ δ, ppm)

7.485(0.8);7.479(0.6);7.476(0.6);7.472(0.6);7.467(0.7);7.462(0.9);7.346(0.7);7.341(0.6); 7.337(0.6);7.332(0.6);7.329(0.7);7.323(1.0);7.261(9.7);7.214(1.3);7.209(1.7);7.208(1.3); 7.200(2.6);7.191(1.4);7.186(1.0);7.088(0.7);7.068(1.1);7.054(1.0);7.018(0.7);7.013(0.6); 6.997(0.5);6.994(0.5);4.047(1.0);4.033(0.6);4.026(0.8);4.018(0.5);4.005(1.1);3.452(16.0); 3.174(0.9);3.153(0.7);3.132(0.8);2.246(1.3);2.239(6.0);2.232(6.4);2.000(0.8);0.000(4.3)

### Example No. A10-664

### ¹H-NMR (400 MHz, CDCl₃ δ, ppm)

7.496(0.8);7.490(0.6);7.487(0.6);7.483(0.6);7.478(0.8);7.473(1.0);7.344(0.8);7.339(0.6); 7.335(0.6);7.330(0.6);7.327(0.7);7.321(1.0);7.261(11.2);7.215(1.3);7.211(1.8);7.201(2.6); 7.192(1.6);7.187(1.0);7.108(0.9);7.089(1.2);6.991(1.3);6.980(0.7);6.961(0.5);3.979(1.0); 3.966(0.5);3.959(0.7);3.957(0.7);3.949(0.6);3.936(1.1);3.477(16.0);3.204(0.9);3.182(0.7);3.161(0.8);2.355(6.5);2.289(7.1);2.001(1.3);0.000(4.8)

### Example No. A10-665

### ¹H-NMR (400 MHz, CDCl₃ δ, ppm)

7.519(0.7);7.511(0.8);7.504(0.6);7.500(0.8);7.494(0.6);7.488(1.0);7.349(0.9);7.343(0.6); 7.337(0.9);7.334(0.6);7.326(1.2);7.260(121.6);7.253(0.7);7.224(1.9);7.218(1.1);7.213(1.3);7.212(1.4);7.207(0.9);7.201(1.3);6.996(0.7);6.861(3.5);6.849(1.8);3.904(0.9);3.891(0.6);3.881(0.7);3.871(0.6);3.859(1.0);3.764(1.0);3.745(1.4);3.726(1.1);3.531(9.7);3.241(1.1);3.229(0.6);3.218(0.7);3.209(0.6);3.197(0.9);2.947(0.8);2.928(1.2);2.909(0.7);2.366(16.0);2.318(9.1);2.251(7.8);1.701(1.3);0.008(1.3);0.000(41.8);−0.009(1.3)

### Example No. A10-666

### ¹H-NMR (400 MHz, CDCl₃ δ, ppm)

7.4981(0.8);7.4975(0.8);7.491(0.7);7.489(0.6);7.485(0.7);7.480(0.8);7.475(0.9);7.346(0.7); 7.341(0.7);7.336(0.6);7.331(0.6);7.328(0.7);7.323(1.0);7.322(1.0);7.261(12.6);7.215(1.4);7.210(1.8);7.201(2.7);7.192(1.6);7.187(1.1);7.125(1.0);7.105(1.3);7.016(1.1);6.986(0.6);6.982(0.5);6.967(0.5);3.986(1.0);3.972(0.5);3.965(0.7);3.964(0.7);3.956(0.5);3.943(1.1);3.477(16.0);3.219(0.9);3.197(0.7);3.176(0.8);2.714(0.5);2.695(1.7);2.676(1.7);2.657(0.6);2.308(6.2);1.253(2.9);1.234(6.6);1.215(2.9);1.209(0.6);0.000(5.5)

### Example No. A10-679

### ¹H-NMR (400 MHz, CDCl₃ δ, ppm)

8.783(1.0);8.778(1.1);8.771(1.0);8.767(1.1);8.519(0.8);8.514(0.8);8.498(0.9);8.494(0.9); 7.635(1.0);7.624(0.9);7.615(0.9);7.603(0.9);7.436(0.8);7.430(0.6);7.428(0.6);7.418(1.0); 7.412(0.9);7.370(0.7);7.365(0.9);7.353(0.7);7.351(0.7);7.347(1.0);7.260(43.6);7.250(0.6); 7.237(1.1);7.231(1.0);7.223(1.1);7.219(1.3);7.218(1.2);7.212(0.9);7.205(1.0);7.201(0.9);5.379(4.2);3.526(16.0);1.560(1.2);0.008(0.5);0.000(18.0);−0.008(0.7)

### Example No. Al2-1

### ¹H-NMR (400 MHz, CDCl₃ δ, ppm)

7.819(0.8);7.815(0.7);7.801(1.0);7.796(0.9);7.494(0.7);7.490(0.8);7.474(0.9);7.470(1.2); 7.434(1.9);7.425(0.5);7.420(0.6);7.413(2.2);7.406(1.1);7.402(0.9);7.387(0.8);7.382(0.8); 7.379(1.0);7.374(2.4);7.368(2.2);7.360(1.0);7.356(1.0);7.261(26.3);7.206(1.6);7.200(1.5); 7.185(1.3);7.180(1.2);5.284(4.8);3.422(16.0);2.955(2.4);2.884(2.0);2.883(2.0);0.000(11.2)

### Example No. A12-2

### ¹H-NMR (400 MHz, CDCl₃ δ, ppm)

7.574(0.7);7.570(1.3);7.565(0.8);7.466(0.5);7.451(0.6);7.447(0.9);7.442(0.8);7.436(0.5); 7.420(0.8);7.416(1.3);7.413(0.5);7.395(2.6);7.377(1.5);7.375(2.3);7.365(2.0);7.360(2.2); 7.267(0.5);7.266(0.6);7.260(51.1);7.256(0.6);7.203(1.6);7.198(1.5);7.183(1.3);7.178(1.2); 5.028(4.3);3.376(16.0);2.044(1.6);1.559(0.7);1.259(1.0);0.008(0.7);0.000(23.4);−0.009(0.7)

### Example No. A12-4

### ¹H-NMR (400 MHz, CDCl₃ δ, ppm)

7.784(0.9);7.780(1.0);7.764(1.0);7.760(1.0);7.593(1.1);7.589(1.1);7.573(1.3);7.569(1.2); 7.419(1.9);7.399(2.2);7.375(2.0);7.370(2.1);7.326(1.2);7.306(1.9);7.286(1.0);7.261(36.9); 7.205(1.6);7.199(1.5);7.184(1.3);7.179(1.2);5.341(4.8);3.431(16.0);1.574(0.8);0.000(15.8)

### Example No. A12-5

### ¹H-NMR (400 MHz, CDCl₃ δ, ppm)

7.810(1.8);7.789(2.0);7.504(1.9);7.498(2.0);7.409(1.9);7.388(2.4);7.375(2.2);7.370(2.3); 7.360(1.3);7.355(1.2);7.339(1.2);7.334(1.1);7.260(61.9);7.207(1.8);7.202(1.7);7.186(1.4); 7.181(1.3);5.235(4.7);3.417(16.0);1.555(3.2);0.008(0.8);0.000(26.6);−0.009(0.7)

### Example No. A12-6

### ¹H-NMR (400 MHz, CDCl₃ δ, ppm)

7.874(2.4);7.868(2.3);7.519(0.7);7.460(2.4);7.440(2.8);7.424(1.2);7.404(3.8);7.403(3.8); 7.390(3.0);7.384(2.9);7.380(3.3);7.375(3.4);7.369(0.8);7.363(0.9);7.269(0.7);7.267(0.9); 7.2663(1.0);7.2655(1.3);7.260(121.3);7.256(1.2);7.255(0.8);7.254(0.6);7.226(2.3);7.221 (2.0);7.210(0.8);7.206(1.9);7.201 (1.7);6.996(0.7);5.252(6.1);3.434(16.0);2.955(2.0);2.884(1.7);2.883(1.8);2.044(2.2);1.551(2.5);1.277(0.6);1.259(1.4);1.241(0.7);0.008(1.5); 0.005(0.5);0.004(0.6);0.002(1.9);0.000(51.8);−0.004(1.0);−0.005(0.7);−0.006(0.5);−0.009(1.6)

### Example No. A12-7

### ¹H-NMR (400 MHz, CDCl₃ δ, ppm)

7.497(1.9);7.495(2.0);7.476(3.6);7.474(2.8);7.453(2.47.386(1.8);7.371(2.1);7.367(3.3); 7.364(1.3);7.345(0.9);7.267(0.5);7.261(49.7);7.257(0.5);7.221(1.8);7.216(1.7);7.201(1.5); 7.196(1.4);5.593(5.1);3.477(16.0);1.573(0.7);0.008(0.6);0.000(20.9);−0.003(0.9);−0.009(0.6)

### Example No. A12-11

### ¹H-NMR (400 MHz, CDCl₃ δ, ppm)

7.709(0.6);7.705(0.7);7.690(1.3);7.686(1.3);7.672(0.7);7.667(0.7);7.478(0.8);7.476(0.6); 7.473(0.6);7.471(0.5);7.464(0.6);7.462(0.6);7.460(0.6);7.457(0.9);7.452(0.5);7.443(0.5); 7.400(3.4);7.379(4.0);7.364(3.5);7.359(3.8);7.262(39.3);7.245(1.7);7.242(1.8);7.226(0.8); 7.223(0.8);7.193(3.1);7.188(3.6);7.173(2.5);7.168(3.1);7.164(1.2);7.160(0.8);7.142(0.7);7.140(0.7);5.154(4.8);5.152(4.8);3.395(16.0);3.392(16.0);2.955(3.0);2.954(3.1);2.884(2.7);2.882(2.7);0.000(16.8)

### Example No. A12-15

### ¹H-NMR (400 MHz, CDCl₃ δ, ppm)

7.520(0.5);7.513(10.6);7.461(2.0);7.441(2.4);7.370(2.7);7.365(2.8);7.261(56.2);7.219(1.9); 7.214(1.8);7.199(1.6);7.194(1.5);5.556(5.9);3.463(16.0);2.956(2.3);2.955(2.3);2.884(2.0);2.883(2.0);1.568(1.0);0.008(0.7);0.000(24.0);−0.009(0.7)

### Example No. A12-17

### ¹H-NMR (400 MHz, CDCl₃ δ, ppm)

7.515(0.8);7.510(0.7);7.499(0.5);7.494(1.6);7.478(0.8);7.473(0.9);7.461(3.7);7.457(0.6); 7.440(4.3);7.362(3.8);7.357(4.0);7.261(68.1);7.216(3.1);7.211(3.3);7.195(2.7);7.190(2.5); 7.081(2.3);7.062(3.2);7.041(2.0);5.206(5.1);3.419(16.0);2.043(0.8);1.587(0.6);0.008(0.9);0.000(28.4);−0.009(0.8)

### Example No. A12-21

### ¹H-NMR (400 MHz, CDCl₃ δ, ppm)

8.659(0.8);8.656(0.9);8.655(0.9);8.652(0.8);8.647(0.8);8.643(1.2);8.640(0.7);7.774(1.2); 7.769(1.3);7.761(1.4);7.758(2.5);7.755(2.1);7.753(2.2);7.379(0.8);7.373(0.7);7.367(0.8); 7.363(0.7);7.361(0.8);7.357(0.7);7.351(0.6);7.344(2.6);7.339(2.6);7.278(2.0);7.260(80.4); 7.210(0.5);7.157(2.0);7.152(1.9);7.137(1.5);7.132(1.4);5.174(5.9);3.490(16.0);2.065(0.7);1.566(0.8);0.008(1.1);0.000(37.0);−0.009(1.0)

### Example No. A12-36

### ¹H-NMR (400 MHz, CDCl₃ δ, ppm)

8.865(0.6);8.066(0.6);7.355(0.8);7.262(46.0);7.260(48.9);7.209(0.6);5.486(1.3);3.602(3.4); 1.541(16.0);0.000(21.1)

### Example No. A12-40

### ¹H-NMR (400 MHz, CDCl₃ δ, ppm)

8.491(2.1);8.479(2.1);7.517(2.1);7.504(2.0);7.443(1.8);7.422(2.2);7.358(1.9);7.353(2.0); 7.261(27.8);7.211(1.7);7.206(1.5);7.190(1.4);7.185(1.2);5.464(4.6);3.572(16.0);3.553(0.5);0.000(12.2)

### Example No. A12-61

### ¹H-NMR (400 MHz, CDCl₃ δ, ppm)

8.038(1.1);8.020(1.3);7.775(1.1);7.772(1.1);7.756(1.47.753(1.4);7.723(0.7);7.719(0.6); 7.704(1.4);7.700(1.2);7.684(0.9);7.680(0.7);7.605(1.0);7.602(1.0);7.586(1.5);7.583(1.5); 7.567(0.6);7.564(0.6);7.406(1.8);7.386(2.2);7.377(3.4);7.372(3.5);7.260(80.8);7.200(1.8); 7.195(1.7);7.180(1.5);7.175(1.4);5.321(5.1);3.478(16.0);2.955(1.7);2.884(1.5);2.883(1.5);2.044(0.8);1.562(2.4);1.259(0.6);0.008(1.1);0.000(33.3);−0.009(0.9)

### Example No. A12-62

### ¹H-NMR (400 MHz, CDCl₃ δ, ppm)

7.874(1.3);7.871(0.9);7.858(0.5);7.855(0.7);7.838(0.6);7.835(0.8);7.776(0.5);7.772(0.8); 7.756(0.7);7.753(0.9);7.749(0.6);7.589(0.8);7.569(1.3);7.550(0.6);7.373(2.0);7.368(2.3); 7.366(2.1);7.345(2.3);7.261(27.7);7.208(1.5);7.203(1.4);7.188(1.2);7.183(1.1);5.080(4.1); 3.405(16.0);2.044(0.8);0.000(11.7)

### Example No. A12-63

### ¹H-NMR (400 MHz, CDCl₃ δ, ppm)

7.751(1.2);7.749(0.8);7.735(0.8);7.730(3.7);7.711(3.4);7.705(0.7);7.690(1.0);7.371(2.1); 7.366(2.2);7.346(1.7);7.326(2.4);7.267(0.6);7.266(0.7);7.2654(0.8);7.2646(1.0);7.261(46.4);7.257(0.6);7.200(1.8);7.195(1.6);7.180(1.3);7.174(1.3);5.105(4.3);3.402(16.0);1.566(0.8);0.008(0.5);0.000(20.4);−0.009(0.6)

### Example No. A12-81

### ¹H-NMR (400 MHz, CDCl₃ δ, ppm)

8.222(0.8);8.202(0.9);7.768(0.7);7.751(0.9);7.636(0.8);7.617(0.5);7.580(0.6);7.561(0.8); 7.462(2.2);7.441(2.6);7.393(2.6);7.388(2.7);7.260(86.8);7.237(2.0);7.232(1.9);7.216(1.7); 7.211(1.7);5.286(3.8);3.394(16.0);2.731(0.6);2.064(0.5);2.044(0.7);1.555(3.1);1.259(0.5);0.008(1.1);0.000(36.0);−0.009(1.0)

### Example No. A12-603

### ¹H-NMR (400 MHz, CDCl₃ δ, ppm)

8.037(0.9);8.033(0.9);8.017(1.3);8.014(1.1);7.893(0.9);7.890(0.9);7.873(1.0);7.871(1.0); 7.615(0.5);7.611(0.5);7.596(1.1);7.592(1.1);7.577(0.7);7.573(0.7);7.531(0.8);7.528(0.8); 7.512(1.0);7.509(1.0);7.493(0.5);7.403(2.0);7.383(2.4);7.363(2.2);7.358(2.3);7.260(68.0); 7.211(0.5);7.195(1.7);7.190(1.5);7.174(1.3);7.169(1.3);5.597(4.9);3.888(16.0);3.395(15.8);2.955(3.4);2.884(2.8);2.883(2.9);2.058(0.5);1.562(2.1);0.008(0.8);0.000(27.5);−0.009(0.9)

### Example No. A12-679

### ¹H-NMR (400 MHz, CDCl₃ δ, ppm)

8.790(1.1);8.786(1.1);8.778(1.2);8.774(1.2);8.440(0.9);8.436(0.9);8.420(0.9);8.416(0.9); 7.624(1.0);7.612(1.0);7.604(1.0);7.592(1.0);7.387(1.9);7.382(1.8);7.377(1.8);7.367(2.3); 7.266(0.6);7.261(49.6);7.205(1.6);7.200(1.4);7.184(1.2);7.179(1.2);5.330(3.7);3.519(16.0); 2.066(0.8);1.563(0.8);0.008(0.6);0.000(21.0);−0.003(0.9);−0.009(0.6)

### Example No. A13-1

### ¹H-NMR (400 MHz, CDCl₃ δ, ppm)

7.794(0.8);7.789(0.7);7.776(1.1);7.771(0.9);7.518(1.1);7.503(0.8);7.499(1.8);7.483(1.0); 7.480(1.2);7.431(0.5);7.427(0.6);7.413(1.1);7.408(1.0);7.394(1.0);7.388(2.0);7.383(2.2); 7.369(1.1);7.365(1.1);7.350(0.4);7.347(0.4);7.261(20.1);7.186(0.6);7.184(0.7);7.182(0.6); 7.166(0.6);7.164(0.6);7.162(0.5);5.297(1.0);5.277(5.0);3.407(16.0);2.374(5.1);2.373(5.1);0.000(6.4)

### Example No. A13-37

### ¹H-NMR (400 MHz, CDCl₃ δ, ppm)

8.298(1.5);7.497(0.8);7.477(0.9);7.365(0.8);7.364(0.9);7.362(0.8);7.264(18.1);5.301(3.9); 5.180(3.3);3.794(16.0);3.472(10.8);2.493(7.0);2.365(3.7);2.364(3.7);2.307(5.5);0.000(5.8)

### Example No. A14-1

### ¹H-NMR (400 MHz, CDCl₃ δ, ppm)

7.758(0.9);7.753(0.9);7.740(1.1);7.735(1.1);7.606(0.5);7.591(0.6);7.584(0.6);7.570(0.6); 7.507(0.8);7.504(1.0);7.488(1.2);7.484(1.4);7.436(0.6);7.431(0.6);7.417(1.2);7.412(1.1); 7.398(0.9);7.393(0.8);7.386(1.0);7.382(1.0);7.368(1.2);7.364(1.2);7.353(0.8);7.347(1.0); 7.330(0.8);7.324(0.8);7.269(0.3);7.268(0.4);7.267(0.5);7.266(0.5);7.261(38.2);7.208(0.4);7.201(0.4);7.187(0.7);7.180(0.7);7.166(0.4);7.160(0.3);5.298(1.4);5.253(5.2);3.405(16.0);0.008(0.4)

### Example No. A14-21

### ¹H-NMR (400 MHz, CDCl₃ δ, ppm)

8.677(0.7);8.675(0.7);8.672(0.8);8.670(0.7);8.665(0.7);8.662(0.8);8.660(0.8);8.658(0.7); 7.801(0.5);7.786(1.2);7.782(1.2);7.767(0.9);7.763(0.8);7.713(0.9);7.710(1.4);7.707(0.9); 7.693(0.6);7.691(0.8);7.688(0.5);7.395(0.7);7.392(0.9);7.383(1.2);7.380(1.1);7.376(0.7); 7.373(0.7);7.367(0.6);7.364(0.8);7.361(0.6);7.330(0.7);7.323(0.8);7.307(0.7);7.300(0.7); 7.268(0.6);7.267(0.8);7.264(31.3);7.133(0.6);7.126(0.6);5.147(5.1);3.474(16.0);0.000(9.5)

### Example No. A14-37

### ¹H-NMR (400 MHz, CDCl₃ δ, ppm)

8.298(1.6);7.332(0.6);7.325(0.6);7.308(0.6);7.302(0.6);7.265(19.5);7.182(0.5);5.301(1.1); 5.142(3.6);3.792(16.0);3.470(13.2);2.477(7.2);2.307(5.7);0.000(5.9)

### Example No. A14-38

### ¹H-NMR (400 MHz, CDCl₃ δ, ppm)

8.512(0.6);8.509(0.6);8.500(0.6);8.497(0.6);7.607(0.6);7.605(0.6);7.588(0.7);7.585(0.7); 7.555(0.5);7.333(0.7);7.326(0.7);7.309(0.7);7.303(0.7);7.281(0.8);7.269(1.2);7.268(1.1); 7.266(12.9);7.262(0.9);7.250(0.7);7.182(0.6);7.176(0.5);5.301(1.4);5.181(4.5);3.513(16.0); 2.573(7.0);0.000(3.7)

### Example No. A14-121

### ¹H-NMR (400 MHz, CDCl₃ δ, ppm)

8.259(1.2);8.238(1.4);8.114(0.9);8.095(1.0);8.092(1.0);7.898(0.8);7.880(0.9);7.878(1.0); 7.805(2.9);7.801(0.8);7.787(0.9);7.784(3.3);7.780(0.7);7.766(0.7);7.763(0.6);7.651(0.8); 7.648(0.8);7.634(0.7);7.631(1.3);7.627(0.7);7.613(0.6);7.610(0.6);7.445(0.5);7.431(0.6); 7.424(0.6);7.410(0.6);7.338(0.8);7.331(0.8);7.314(0.8);7.308(0.8);7.267(0.5);7.263(25.8); 7.134(0.7);7.128(0.7);5.325(5.4);5.300(3.3);3.553(16.0);2.708(1.1);2.046(0.5);1.432(1.5);1.260(0.7);1.256(0.6);0.000(8.0)

### Example No. A14-680

### ¹H-NMR (400 MHz, CDCl₃ δ, ppm)

7.632(0.7);7.617(0.9);7.596(0.8);7.355(1.0);7.331(1.0);7.260(25.6);7.233(0.6);7.209(1.1); 5.787(5.6);3.480(16.0);1.556(7.8);1.258(0.5);0.000(11.1)

### Example No. A14-681

### ¹H-NMR (400 MHz, CDCl₃ δ, ppm)

8.754(2.9);7.573(0.5);7.566(0.5);7.552(0.5);7.510(3.2);7.347(0.7);7.340(0.7);7.323(0.7); 7.317(0.7);7.262(15.0);7.207(0.6);7.201(0.6);5.200(4.7);3.439(16.0);1.595(0.9);0.000(6.3)

### Example No. A15-21

### ¹H-NMR (400 MHz, CDCl₃ δ, ppm)

8.658(0.7);8.655(0.8);8.653(0.9);8.651(0.8);8.645(0.8);8.643(0.9);8.641(0.8);8.639(0.8); 8.374(2.6);8.369(2.6);7.989(2.3);7.969(2.9);7.837(1.7);7.832(1.7);7.816(1.3);7.811(1.3); 7.741(0.6);7.736(0.6);7.722(1.3);7.717(1.3);7.703(0.8);7.698(0.8);7.566(0.8);7.564(1.4); 7.562(0.9);7.547(0.7);7.545(1.1);7.542(0.7);7.371(0.8);7.368(0.8);7.359(0.8);7.356(0.8); 7.352(0.8);7.349(0.7);7.340(0.7);7.337(0.7);7.273(0.6);7.264(74.0);5.092(5.2);3.498(16.0);1.609(2.3);0.008(0.6);0.000(22.1);−0.009(0.7)

### Example No. A16-21

### ¹H-NMR (400 MHz, CDCl₃ δ, ppm)

8.602(0.7);8.600(0.7);8.597(0.8);8.595(0.7);8.590(0.7);8.587(0.8);8.585(0.8);8.583(0.7); 7.742(0.5);7.737(0.5);7.722(1.2);7.718(1.2);7.703(0.8);7.699(0.8);7.591(0.8);7.588(1.3); 7.586(0.8);7.571(0.6);7.569(1.0);7.566(0.6);7.556(1.5);7.553(2.3);7.548(0.6);7.539(1.0); 7.538(0.9);7.535(2.1);7.532(1.8);7.424(0.6);7.421(0.8);7.418(0.7);7.407(0.8);7.405(0.9); 7.403(1.0);7.401(0.9);7.360(0.7);7.357(1.2);7.343(0.5);7.339(2.9);7.335(2.0);7.323(1.3);7.320(2.1);7.319(2.2);7.316(1.5);7.306(0.9);7.304(2.7);7.300(2.0);7.298(2.4);7.296(1.9);7.291(2.1);7.285(1.3);7.283(0.7);7.277(0.6);7.273(1.5);7.271(1.5);7.269(1.0);7.267(1.2);7.263(29.4);7.257(0.8);7.253(0.6);7.251(0.5);5.301(1.7);4.977(5.4);3.214(16.0);0.000(9.1)

### Example No. A17-1

### ¹H-NMR (400 MHz, CDCl₃ δ, ppm)

7.746(0.8);7.742(0.8);7.727(0.9);7.723(0.9);7.636(0.5);7.630(0.5);7.620(0.6);7.612(0.6); 7.505(0.7);7.501(0.8);7.485(1.1);7.481(1.2);7.426(0.5);7.412(1.0);7.408(0.9);7.393(0.7); 7.388(0.6);7.375(0.8);7.371(0.7);7.356(1.0);7.352(0.9);7.293(4.1);7.285(2.8);7.263(14.2); 5.300(0.5);5.293(4.9);4.071(0.6);4.043(1.8);4.014(1.9);3.986(0.6);3.419(16.0);1.684(0.7);0.000(4.3)

### Example No. A17-21

### ¹H-NMR (400 MHz, CDCl₃ δ, ppm)

8.674(0.6);8.671(0.7);8.669(0.7);8.667(0.7);8.661(0.7);8.659(0.7);8.657(0.7);8.655(0.6); 7.765(1.0);7.760(1.0);7.746(0.8);7.741(0.7);7.697(1.2);7.677(0.7);7.457(0.5);7.453(0.9); 7.437(0.9);7.433(0.7);7.389(0.6);7.386(0.6);7.377(0.6);7.374(0.6);7.370(0.6);7.367(0.6); 7.358(0.5);7.355(0.5);7.271(1.3);7.263(46.8);7.255(1.8);7.244(0.5);7.237(0.9);7.226(0.6);5.180(4.7);3.998(0.7);3.970(2.1);3.942(2.2);3.914(0.7);3.491(16.0);1.649(0.6);1.259(1.3);0.000(13.0)

### Example No. A17-30

### ¹H-NMR (400 MHz, CDCl₃ δ, ppm)

8.299(2.0);7.626(0.5);7.616(0.9);7.605(0.6);7.602(0.6);7.288(0.6);7.280(1.3);7.272(2.0); 7.265(23.4);7.257(0.8);5.149(4.6);4.031(0.6);4.002(1.8);3.974(1.9);3.946(0.6);3.791(16.0); 3.500(15.5);2.481(9.1);2.308(7.5);1.260(0.7);0.000(6.0)

### Example No. A18-21

### ¹H-NMR (400 MHz, CDCl₃ δ, ppm)

8.652(0.6);8.650(0.7);8.648(0.7);8.645(0.6);8.640(0.6);8.638(0.7);8.636(0.7);8.633(0.6); 7.951(1.3);7.947(1.6);7.946(1.6);7.943(1.3);7.839(1.3);7.836(1.4);7.834(1.5);7.831(1.3); 7.728(1.0);7.723(1.0);7.708(0.7);7.704(0.6);7.574(0.7);7.571(1.1);7.569(0.7);7.554(0.6); 7.552(0.9);7.549(0.5);7.483(1.4);7.478(2.4);7.473(1.3);7.366(0.6);7.363(0.6);7.354(0.6); 7.351(0.6);7.347(0.6);7.344(0.5);7.335(0.5);7.262(28.6);5.299(0.5);5.118(4.8);3.476(16.0);2.083(0.6);1.256(0.7);0.000(8.2)

### Example No. A19-1

### ¹H-NMR (400 MHz, CDCl₃ δ, ppm)

7.801(0.7);7.796(0.6);7.783(0.9);7.777(0.8);7.492(0.6);7.488(0.8);7.472(1.4);7.469(1.3); 7.456(0.6);7.453(0.7);7.401(1.0);7.396(0.9);7.382(0.8);7.378(1.0);7.373(0.9);7.359(0.9); 7.355(0.8);7.262(13.9);7.178(0.5);7.165(1.1);7.161(1.0);7.149(1.3);7.147(1.1);7.128(0.7); 7.123(0.5);5.324(4.4);5.297(1.2);3.430(16.0);2.519(6.8);2.044(0.5);0.000(4.4)

### Example No. A19-2

### ¹H-NMR (400 MHz, CDCl₃ δ, ppm)

7.564(0.7);7.559(1.2);7.555(0.7);7.445(0.8);7.442(1.3);7.436(1.0);7.431(0.8);7.427(0.9); 7.423(1.1);7.419(0.6);7.411(0.6);7.393(1.2);7.375(1.2);7.265(0.6);7.264(0.8);7.263(1.1); 7.260(39.8);7.257(0.6);7.171(0.7);7.158(1.1);7.154(0.9);7.142(1.2);7.140(1.6);7.125(0.6); 7.123(0.7);7.122(0.6);7.118(0.6);5.060(4.1);3.385(16.0);2.716(1.9);2.499(6.3);2.442(0.6);0.069(0.6);0.000(15.7)

### Example No. A19-5

### ¹H-NMR (400 MHz, CDCl₃ δ, ppm)

7.774(1.6);7.753(1.8);7.505(1.8);7.500(1.9);7.444(1.1);7.425(1.2);7.359(1.3);7.354(1.2); 7.339(1.2);7.333(1.2);7.311(0.5);7.260(27.3);7.209(0.7);7.187(1.1);7.172(1.3);7.168(1.2); 7.151(2.0);7.131(1.4);7.114(0.6);5.285(5.0);3.428(16.0);2.955(0.7);2.884(0.6);2.509(8.1);0.000(10.0)

### Example No. A19-6

### ¹H-NMR (400 MHz, CDCl₃ δ, ppm)

7.865(1.4);7.859(1.4);7.503(0.5);7.498(0.6);7.483(0.6);7.480(0.8);7.4204(0.7);7.4196(0.7); 7.399(2.4);7.385(1.9);7.379(1.7);7.363(0.5);7.357(0.6);7.260(33.2);7.173(1.2);7.168(1.1);7.160(1.1);7.157(1.0);7.156(0.9);7.153(0.9);7.152(0.8);7.146(0.8);7.139(0.5);5.286(4.5);3.439(16.0);2.528(7.1);0.000(11.7)

### Example No. A19-7

### ¹H-NMR (400 MHz, CDCl₃ δ, ppm)

7.519(1.3);7.514(0.8);7.496(3.5);7.495(3.4);7.475(4.7);7.379(2.2);7.361(1.7);7.358(1.5); 7.339(1.3);7.294(0.5);7.260(1 13.4);7.210(1.0);7.174(0.6);7.171(0.6);7.163(1.8);7.158(1.7);7.154(1.6);7.150(1.4);7.146(1.3);7.140(1.1);7.133(0.7);6.996(0.6);5.625(6.3);3.492(16.0);2.716(7.4);2.531(10.6);2.442(2.3);1.573(0.9);0.008(1.3);0.000(39.5);-0.009(1.1)

### Example No. A19-10

### ¹H-NMR (400 MHz, CDCl₃ δ, ppm)

7.519(1.1);7.513(11.8);7.507(0.9);7.502(0.9);7.483(1.0);7.318(0.7);7.311(0.8);7.303(0.7); 7.299(0.9);7.294(0.8);7.260(139.9);7.224(1.1);7.210(1.6);7.192(0.8);7.178(0.7);7.166(1.6);7.162(1.5);7.153(1.7);7.147(1.4);7.139(1.2);7.132(0.9);6.996(0.8);5.593(6.4);3.477(14.6);2.716(16.0);2.518(11.0);2.478(1.0);2.442(5.2);2.049(0.8);1.575(0.9);1.254(0.8); 0.008(1.4);0.000(50.3);−0.009(1.5)

### Example No. A19-11

### ¹H-NMR (400 MHz, CDCl₃ δ, ppm)

7.701(0.6);7.697(0.6);7.682(1.2);7.678(1.2);7.663(0.6);7.659(0.6);7.471(0.8);7.469(0.6); 7.466(0.6);7.464(0.5);7.457(0.6);7.455(0.6);7.453(0.6);7.450(0.9);7.445(0.5);7.436(1.4); 7.432(1.5);7.418(1.0);7.414(1.2);7.262(26.2);7.259(1.6);7.243(1.6);7.240(1.7);7.224(0.8); 7.221(0.8);7.186(1.0);7.183(0.8);7.174(0.6);7.171(0.9);7.168(1.0);7.165(0.9);7.161(1.3);7.157(1.0);7.155(2.0);7.151(1.8);7.143(1.1);7.140(2.5);7.138(2.6);7.137(2.1);7.133(1.2);7.131(1.47.126(0.9);7.124(0.8);7.123(0.6);7.115(1.1);7.113(1.47.111(0.8);7.110(0.9);5.297(1.4);5.192(4.5);5.190(4.6);3.404(15.6);3.401(16.0);2.503(11.8);0.000(7.9)

### Example No. A19-17

### ¹H-NMR (400 MHz, CDCl₃ δ, ppm)

7.519(0.9);7.507(1.0);7.502(1.5);7.495(1.3);7.486(2.0);7.481(1.7);7.477(1.6);7.470(0.8); 7.465(1.0);7.449(0.5);7.260(145.7);7.210(0.7);7.177(0.5);7.174(0.8);7.170(1.0);7.158(2.6);7.153(2.3);7.146(2.6);7.144(2.3);7.142(2.0);7.140(1.9);7.138(1.7);7.132(1.7);7.126(1.3);7.078(2.5);7.058(3.5);7.037(2.1);6.996(0.8);5.234(5.3);3.434(16.0);2.956(0.6);2.884(0.5);2.883(0.5);2.717(1.6);2.516(15.0);2.442(0.5);2.032(1.5);0.008(1.5);0.006(0.5);0.0054(0.6);0.0046(0.7);0.000(54.0);−0.003(2.5);−0.004(0.9);−0.005(0.6);−0.006(0.5);−0.007(0.5);−0.009(1.6)

### Example No. A19-21

### ¹H-NMR (400 MHz, CDCl₃ δ, ppm)

8.661(0.5);8.658(0.6);8.656(0.6);8.654(0.5);8.649(0.5);8.645(0.7);7.768(0.8);7.763(0.8); 7.751(1.7);7.746(1.6);7.743(1.0);7.375(0.5);7.370(0.5);7.363(0.5);7.358(0.9);7.300(0.6); 7.296(0.6);7.281(0.7);7.278(0.7);7.264(12.2);7.149(0.5);7.146(0.5);7.132(0.9);7.128(0.9); 7.120(0.7);7.117(0.9);7.116(1.0);7.095(0.6);7.076(0.6);5.298(1.4);5.211(4.6);3.496(16.0);2.454(6.5);0.000(3.5)

### Example No. A19-22

### ¹H-NMR (400 MHz, CDCl₃ δ, ppm)

8.840(10.1);8.827(10.1);8.748(1.5);8.736(1.5);7.518(4.6);7.413(1.1);7.395(1.2);7.370(1.9); 7.358(3.6);7.345(2.1);7.260(850.4);7.226(0.8);7.210(5.2);7.150(1.1);7.132(1.3);7.118(3.5);7.101(1.7);6.996(4.6);5.399(0.9);5.378(6.8);3.627(7.4);2.717(1.0);2.482(16.0);2.081(5.7);1.540(4.5);1.054(0.7);1.049(0.7);0.146(0.8);0.008(9.3);0.000(329.1);−0.009(8.8);−0.050(2.0);−0.150(0.9)

### Example No. A19-30

### ¹H-NMR (400 MHz, CDCl₃ δ, ppm)

8.297(1.6);7.431(0.8);7.415(0.5);7.411(0.6);7.263(21.9);7.157(0.8);7.144(1.1);7.139(1.0); 7.129(1.8);7.113(0.8);7.1074(0.6);7.1066(0.6);5.202(3.8);3.794(16.0);3.502(13.8);2.507(7.3);2.483(6.4);2.306(5.9);1.968(0.9);0.000(6.4)

### Example No. A19-40

### ¹H-NMR (400 MHz, CDCl₃ δ, ppm)

7.993(1.4);7.973(1.6);7.778(1.3);7.775(1.4);7.759(1.6);7.756(1.7);7.723(1.0);7.719(0.9); 7.704(2.0);7.700(1.8);7.684(1.2);7.681(1.1);7.603(1.4);7.600(1.4);7.584(2.1);7.581(2.1); 7.564(0.9);7.561(0.8);7.519(1.1);7.432(1.2);7.412(1.3);7.275(0.6);7.273(0.8);7.272(0.9); 7.271(1.0);7.2704(1.0);7.2696(1.1);7.269(1.3);7.268(1.4);7.267(1.7);7.2664(2.0);7.2656(2.3);7.265(2.9);7.260(184.0);7.257(3.0);7.256(2.0);7.255(1.4);7.254(1.1);7.2534(0.8); 7.2526(0.6);7.252(0.5);7.210(1.5);7.190(1.1);7.176(1.6);7.172(1.5);7.155(2.2);7.141(1.9);7.123(1.3);6.996(1.0);5.386(6.0);3.489(16.0);2.512(14.7);2.050(1.0);1.561(1.0);0.008(2.1);0.0064(0.7);0.0056(0.7);0.005(0.9);0.004(1.2);0.002(3.0);0.000(66.4);−0.004(1.2);−0.005(0.9);−0.006(0.7);−0.007(0.6);−0.008(1.9)

### Example No. A19-61

### ¹H-NMR (400 MHz, CDCl₃ δ, ppm)

7.987(0.7);7.985(0.7);7.968(0.8);7.966(0.8);7.776(0.6);7.773(0.7);7.757(0.8);7.754(0.9); 7.702(1.0);7.698(0.9);7.682(0.6);7.679(0.5);7.600(0.7);7.597(0.7);7.581(1.1);7.578(1.1); 7.429(0.6);7.426(0.7);7.411(0.7);7.407(0.7);7.263(10.7);7.190(0.5);7.187(0.6);7.173(1.0); 7.169(0.9);7.158(0.7);7.155(1.0);7.154(1.1);7.139(0.9);7.135(0.6);7.121(0.6);7.120(0.6);7.117(0.6);5.378(3.8);5.298(2.0);3.480(16.0);2.511(7.0);2.044(0.8);0.000(3.5)

### Example No. A19-62

### ¹H-NMR (400 MHz, CDCl₃ δ, ppm)

7.856(0.7);7.852(0.7);7.846(0.8);7.842(1.7);7.838(1.7);7.774(0.5);7.771(0.8);7.754(0.7); 7.751(1.1);7.747(0.6);7.588(1.0);7.567(1.2);7.548(0.7);7.391(0.7);7.372(0.8);7.260(61.9); 7.181(0.7);7.168(1.0);7.164(0.9);7.146(1.7);7.132(0.6);7.129(0.9);5.127(4.0);3.415(16.0); 2.495(7.0);2.037(0.5);0.008(0.7);0.000(21.1);−0.009(0.6)

### Example No. A19-63

### ¹H-NMR (400 MHz, CDCl₃ δ, ppm)

7.748(1.3);7.743(0.5);7.732(0.8);7.727(3.2);7.698(3.47.693(0.7);7.677(1.1);7.368(0.6); 7.353(0.6);7.349(0.7);7.260(57.9);7.183(0.6);7.169(0.9);7.166(0.8);7.148(1.0);7.146(1.1); 7.137(0.6);7.133(0.6);7.120(0.7);7.116(0.5);5.154(4.1);3.410(16.0);2.483(6.7);2.033(0.6);0.008(0.7);0.000(21.0);−0.009(0.5)

### Example No. A19-81

### ¹H-NMR (400 MHz, CDCl₃ δ, ppm)

8.182(0.8);8.163(0.9);7.768(0.7);7.751(0.9);7.636(0.8);7.617(0.5);7.574(0.6);7.554(0.8); 7.524(0.6);7.519(1.1);7.500(0.9);7.299(0.5);7.293(0.5);7.260(60.8);7.223(0.7);7.210(0.9); 7.205(0.9);7.203(1.2);7.190(1.9);7.186(1.5);7.176(2.0);7.175(2.0);7.173(1.6);7.172(1.3); 7.170(1.1);7.161(1.1);7.157(0.8);5.342(3.7);3.400(16.0);2.715(10.3);2.534(9.4);2.477(0.6);2.442(3.2);0.008(0.6);0.000(22.2);−0.009(0.6)

### Example No. A19-603

### ¹H-NMR (400 MHz, CDCl₃ δ, ppm)

8.034(0.8);8.018(0.8);7.872(1.1);7.853(1.1);7.619(0.6);7.596(1.0);7.581(0.7);7.527(0.7); 7.518(2.1);7.510(1.1);7.446(0.8);7.430(0.8);7.293(0.7);7.259(364.3);7.227(0.6);7.210(1.9);7.138(2.0);7.121(1.0);6.995(1.8);5.649(1.8);3.888(16.0);3.406(4.1);2.496(11.2);2.069(1.7);1.542(4.2);0.008(3.1);0.000(113.3);−0.009(3.3);−0.050(0.6)

### Example No. A19-679

### ¹H-NMR (400 MHz, CDCl₃ δ, ppm)

8.793(1.6);8.789(1.8);8.781(1.7);8.777(1.8);8.349(0.8);8.329(0.8);7.625(1.6);7.613(1.7); 7.605(1.5);7.593(1.5);7.519(3.7);7.392(1.1);7.373(1.3);7.311(0.8);7.293(0.6);7.291(1.0); 7.286(1.2);7.285(1.1);7.282(1.0);7.281(1.0);7.280(1.2);7.279(1.1);7.278(1.1);7.2774(1.3); 7.2766(1.4);7.276(1.3);7.275(1.5);7.274(1.6);7.2734(1.7);7.2726(2.0);7.272(2.4);7.271(2.6);7.270(3.0);7.2694(3.3);7.2686(3.6);7.268(4.3);7.267(4.6);7.266(5.0);7.2654(6.3); 7.2646(7.7);7.264(10.4);7.260(685.2);7.255(11.3);7.253(8.1);7.252(7.1);7.251(5.3);7.249(4.5);7.231(2.5);7.225(2.6);7.210(5.2);7.192(1.5);7.165(1.9);7.145(1.4);7.126(1.3);6.996(3.7);5.415(3.5);5.380(0.7);3.526(9.0);2.503(13.4);2.088(1.3);1.537(16.0);1.257(0.6); 0.146(0.6);0.008(6.8);0.000(266.2);−0.009(9.1);−0.050(1.7);−0.150(0.8)

### Example No. A20-1

### ¹H-NMR (400 MHz, CDCl₃ δ, ppm)

7.809(0.7);7.804(0.6);7.791(0.9);7.785(0.8);7.493(0.6);7.489(0.9);7.475(0.7);7.474(0.8); 7.470(1.1);7.446(0.6);7.443(0.7);7.426(0.7);7.424(0.8);7.423(0.8);7.403(1.0);7.398(0.9); 7.384(1.2);7.378(1.5);7.364(0.9);7.360(0.8);7.263(8.8);7.220(0.7);7.216(0.7);7.203(1.1); 7.199(1.5);7.193(1.2);7.146(0.6);7.141(0.5);7.127(0.6);7.122(0.5);5.321(4.5);5.295(2.1); 3.423(16.0);2.960(0.6);2.941(1.8);2.922(1.9);2.903(0.6);1.303(3.0);1.284(6.5);1.265(3.0);0.000(3.0)

### Example No. A21-1

### ¹H-NMR (400 MHz, CDCl₃ δ, ppm)

8.053(0.7);8.052(0.9);8.051(0.9);8.049(0.7);8.034(0.8);8.031(1.4);8.029(0.8);7.843(0.7); 7.838(0.5);7.825(0.9);7.819(0.8);7.575(1.0);7.573(1.2);7.570(1.2);7.569(1.3);7.562(2.9); 7.559(2.7);7.513(0.6);7.508(0.9);7.496(0.7);7.494(0.8);7.490(1.1);7.484(0.8);7.475(0.6); 7.470(0.6);7.464(0.8);7.462(0.6);7.455(0.5);7.451(0.6);7.442(0.6);7.440(0.5);7.427(1.1); 7.421(1.0);7.412(1.0);7.409(1.1);7.406(1.1);7.402(0.8);7.393(1.0);7.389(0.8);7.263(16.0);5.299(10.0);5.282(4.7);3.462(12.8);3.442(16.0);1.334(0.9);1.284(1.3);1.257(2.1); 0.880(0.5);0.000(7.5)

### Example No. A21-21

### ¹H-NMR (400 MHz, CDCl₃ δ, ppm)

8.686(0.5);8.684(0.6);8.681(0.7);8.679(0.6);8.674(0.6);8.671(0.6);8.669(0.6);8.667(0.5); 8.031(0.8);8.028(0.8);8.027(0.8);8.011(0.9);8.009(0.9);8.008(0.8);7.804(0.8);7.800(0.8); 7.786(0.9);7.781(0.8);7.766(0.8);7.763(1.1);7.760(0.9);7.522(1.1);7.519(1.1);7.504(0.9); 7.500(0.8);7.459(0.7);7.456(0.9);7.440(0.9);7.436(1.0);7.421(0.5);7.401(0.6);7.397(0.6); 7.389(1.5);7.385(1.5);7.383(0.7);7.379(0.6);7.371(1.2);7.367(1.2);7.265(14.0);5.180(4.5);3.501(16.0);3.450(12.2);1.255(0.6);0.070(1.6);0.000(6.4)

### Example No. A22-1

### ¹H-NMR (400 MHz, d₆-DMSO δ, ppm)

7.832(3.3);7.826(3.6);7.796(1.3);7.792(1.3);7.777(1.5);7.773(1.4);7.742(2.1);7.737(1.8); 7.722(2.4);7.716(2.3);7.605(1.3);7.602(1.4);7.585(2.0);7.582(2.0);7.527(0.9);7.522(1.0); 7.516(3.7);7.508(1.7);7.504(1.5);7.496(3.1);7.489(1.1);7.484(1.0);7.465(1.2);7.461(1.2); 7.446(1.6);7.442(1.6);7.427(0.7);7.424(0.6);5.119(6.0);3.876(0.5);3.507(16.0);3.326(44.9);3.322(21.2);3.303(1.6);2.520(0.6);2.511(6.7);2.507(14.1);2.502(19.3);2.498(13.6); 2.493(6.1)

### Example No. A22-21

### ¹H-NMR (400 MHz, CDCl₃ δ, ppm)

8.687(0.6);8.685(0.7);8.682(0.7);8.680(0.6);8.675(0.6);8.672(0.7);8.670(0.7);8.668(0.6); 8.017(2.0);8.011(2.0);7.795(1.0);7.791(1.0);7.776(0.8);7.772(0.8);7.724(0.7);7.721(1.2); 7.718(0.8);7.702(0.8);7.491(1.2);7.485(1.1);7.471(1.5);7.465(1.5);7.404(0.6);7.401(0.6); 7.392(0.6);7.389(0.6);7.385(0.6);7.382(0.6);7.373(0.6);7.370(0.5);7.332(2.1);7.311(1.7); 7.265(21.8);5.300(5.9);5.140(4.6);3.488(16.0);3.462(12.6);3.352(2.5);3.305(1.7);2.724(3.6);2.529(2.4);1.255(0.5);0.000(7.8)

### Example No. A23-1

### ¹H-NMR (400 MHz, CDCl₃ δ, ppm)

7.988(0.8);7.976(0.8);7.972(0.5);7.965(0.8);7.496(0.6);7.486(0.6);7.478(0.6);7.473(1.1); 7.412(1.5);7.409(1.4);7.405(1.2);7.399(1.6);7.392(1.0);7.388(1.2);7.386(1.2);7.267(6.4); 7.217(2.6);7.196(3.4);7.073(3.4);7.052(2.5);5.316(1.5);3.979(16.0);3.427(9.1);0.000(2.8)

### Example No. A24-1

### ¹H-NMR (400 MHz, CDCl₃ δ, ppm)

7.784(1.1);7.779(0.9);7.766(1.4);7.761(1.2);7.515(0.9);7.51 1(1.2);7.495(1.3);7.492(1.6); 7.447(0.6);7.442(0.7);7.428(1.4);7.424(1.2);7.409(1.0);7.403(1.3);7.398(1.2);7.384(1.3); 7.380(1.3);7.318(1.1);7.297(1.7);7.292(1.1);7.271(2.0);7.264(27.6);7.225(2.0);7.213(2.1); 7.204(1.3);7.192(1.1);5.300(10.6);5.285(1.9);4.047(9.4);4.043(9.4);3.451(9.0);3.332(16.0);0.000(9.6)

### Example No. A25-35

### ¹H-NMR (400 MHz, CD3CN 6, ppm)

8.619(1.4);8.613(1.4);8.021(1.4);8.016(1.3);7.535(0.7);7.530(0.7);7.516(0.8);7.511(0.8); 7.353(0.6);7.348(0.6);7.333(0.7);7.328(0.6);7.304(0.6);7.301(0.7);7.286(0.8);7.283(0.9); 7.267(0.3);7.234(0.5);7.231(0.7);7.227(0.5);7.214(0.4);7.210(0.5);7.207(0.3);5.188(4.9); 3.431(16.0);2.150(3.0);2.142(6.8);1.964(0.9);1.958(1.4);1.952(5.9);1.946(10.2);1.939(13.3);1.933(9.4);1.927(4.8);0.000(0.6)

### Example No. A25-36

### ¹H-NMR (400 MHz, CD3CN 6, ppm)

7.597(0.6);7.592(0.6);7.578(0.6);7.573(1.0);7.554(0.6);7.549(0.4);7.533(0.5);7.529(0.4); 7.449(0.5);7.447(0.5);7.430(0.8);7.428(0.8);7.419(0.3);7.412(0.4);7.383(0.6);7.380(0.5); 7.360(0.5);2.606(16.0);2.430(4.9);2.142(28.5);2.113(0.4);2.107(0.4);1.964(3.5);1.958(5.8);1.952(24.7);1.946(43.1);1.940(56.0);1.934(38.6);1.927(19.9);1.915(0.4);1.768(0.3); 0.008(1.2);0.000(32.8);−0.009(1.2)

### Example No. A26-35

### ¹H-NMR (400 MHz, CD3CN 6, ppm)

8.591(1.4);8.585(1.3);8.037(1.4);8.032(1.3);8.019(1.3);7.999(1.4);7.605(1.5);7.584(1.4); 5.110(4.9);3.516(16.0);2.141(20.4);1.964(2.0);1.958(3.3);1.952(13.0);1.946(22.3);1.940(28.6);1.934(19.5);1.927(9.9);0.000(0.9)

### Example No. A26-36

### ¹H-NMR (400 MHz, CD3CN 6, ppm)

8.891(0.9);8.267(0.9);8.263(0.9);8.003(1.2);7.983(1.3);7.601(1.4);7.581(1.2);5.191(4.3); 3.572(16.0);2.889(0.4);2.772(0.3);2.641(0.5);2.464(0.4);2.160(74.6);2.120(0.4);2.114(0.5);2.107(0.6);2.101(0.4);1.964(5.3);1.958(8.0);1.952(37.5);1.946(64.5);1.940(85.1);1.934(58.7);1.928(30.2);1.915(0.6);1.775(0.4);1.769(0.5);1.762(0.4);1.285(0.4);1.271(1.0); 0.000(3.0)

### Example No. A27-35

### ¹H-NMR (400 MHz, CD3CN 6, ppm)

8.588(1.3);8.583(1.3);8.148(0.8);8.111(0.5);8.092(0.6);8.038(1.3);8.032(1.3);7.639(0.4); 7.619(0.5);7.499(0.4);7.480(0.8);7.460(0.3);5.081(4.7);3.532(16.0);2.133(16.5);1.964(2.9);1.958(4.3);1.952(20.0);1.946(35.1);1.940(46.1);1.933(31.7);1.927(16.2);0.000(1.6)

### Example No. A27-36

### ¹H-NMR (400 MHz, CD3CN 6, ppm)

8.888(1.0);8.886(0.9);8.267(1.0);8.263(0.9);8.107(1.48.094(0.6);8.074(0.6);7.635(0.5); 7.616(0.6);7.495(0.5);7.476(0.9);7.456(0.4);5.156(4.4);3.592(16.0);2.171(28.4);1.965(1.7);1.959(2.6);1.953(11.3);1.947(19.9);1.940(26.2);1.934(18.1);1.928(9.2);0.000(0.8)

### Example No. A28-35

### ¹H-NMR (400 MHz, CD3CN 6, ppm)

8.590(1.4);8.584(1.4);8.039(1.4);8.033(1.3);7.847(0.7);7.843(1.1);7.839(0.8);7.785(0.5); 7.782(0.9);7.778(0.5);7.766(0.5);7.763(1.0);7.759(0.5);7.340(0.4);7.338(0.3);7.323(0.7); 7.320(0.7);7.318(0.8);7.315(0.6);7.292(1.0);7.273(1.2);7.253(0.5);5.448(0.7);5.088(5.0); 4.068(0.6);4.050(0.6);3.501(16.0);2.169(11.8);1.972(2.5);1.965(0.9);1.959(1.2);1.953(5.7);1.947(10.0);1.940(13.2);1.934(9.1);1.928(4.7);1.222(0.7);1.204(1.3);1.186(0.7);0.000(0.4)

### Example No. A28-36

### ¹H-NMR (400 MHz, CD3CN 6, ppm)

8.890(0.8);8.887(0.8);8.268(0.8);8.264(0.8);7.826(0.6);7.822(0.9);7.818(0.7);7.770(0.5); 7.767(0.8);7.763(0.4);7.751(0.5);7.748(0.9);7.744(0.5);7.337(0.3);7.320(0.6);7.317(0.6); 7.315(0.7);7.312(0.5);7.287(0.9);7.268(1.2);7.249(0.4);5.170(4.1);3.558(16.0);2.623(0.3); 2.141(20.5);2.120(0.3);2.113(0.4);2.107(0.4);1.964(3.3);1.958(4.9);1.952(22.2);1.946(39.1);1.940(51.3);1.934(35.3);1.927(18.0);1.921(0.5);0.000(1.6)

### Example No. A29-35

### ¹H-NMR (400 MHz, CD3CN 6, ppm)

8.918(0.9);8.915(1.0);8.255(1.0);8.252(1.0);7.301(1.5);7.299(1.7);7.280(2.9);7.208(1.3); 7.190(1.0);7.186(0.8);7.168(0.6);5.365(4.1);3.511(16.0);2.161(15.4);1.972(1.2);1.964(1.6);1.958(2.4);1.952(10.4);1.946(18.3);1.940(24.1);1.934(16.8);1.928(8.6);1.204(0.6); 0.000(0.5)

### Example No. A29-36

### ¹H-NMR (400 MHz, CD3CN 6, ppm)

8.624(1.4);8.618(1.4);8.037(1.4);8.032(1.4);7.308(1.6);7.306(1.6);7.287(3.0);7.211(1.3); 7.193(1.0);7.189(0.8);7.171(0.6);5.447(0.4);5.263(4.7);3.443(16.0);2.155(13.2);1.964(1.8);1.958(2.7);1.952(12.0);1.946(21.0);1.940(27.7);1.934(19.2);1.928(9.8);0.000(0.6)

### Example No. A30-35

### ¹H-NMR (400 MHz, CD3CN 6, ppm)

8.605(1.3);8.600(1.3);8.023(1.3);8.017(1.3);7.722(0.6);7.647(0.4);7.645(0.3);7.634(0.4); 7.625(0.5);7.616(0.5);7.607(0.5);7.603(0.5);7.593(0.5);7.582(1.2);7.441(0.8);7.432(1.5); 7.423 (1.1);7.417(1.1);7.409(1.2);5.447(1.1);5.193(4.8);3.476(16.0);2.137(18.7);2.107(0.4);1.972(1.3);1.964(3.2);1.958(5.0);1.952(22.5);1.946(39.4);1.939(51.8);1.933 (35.7); 1.927(18.3);1.204(0.6);0.008(1.1);0.000(30.1);−0.009(0.9)

### Example No. A30-462

### ¹H-NMR (400 MHz, CDCl₃ δ, ppm)

7.697(0.9);7.689(0.9);7.678(1.0);7.670(1.1);7.554(0.9);7.535(0.6);7.504(0.7);7.483(0.9); 7.283(0.8);7.261(23.4);7.215(1.0);7.194(0.6);7.143(1.5);7.003(0.8);6.912(1.3);6.893(1.8); 6.872(1.1);4.050(1.0);4.038(0.7);4.029(0.9);4.020(0.7);4.008(1.1);3.704(16.0);3.135(0.6);2.858(0.6);2.845(0.8);2.827(1.7);2.809(0.9);2.235(0.8);2.223(0.6);2.213(0.7);2.205(0.6);2.193(0.8);0.000(8.4)

### Example No. A31-1

### ¹H-NMR (400 MHz, CDCl₃ δ, ppm)

8.202(2.0);8.197(0.7);8.185(0.9);8.180(3.8);8.175(0.6);8.138(0.6);8.133(3.8);8.127(0.9); 8.116(0.7);8.110(2.0);7.633(0.7);7.628(0.8);7.613(0.9);7.609(0.9);7.503(0.7);7.500(0.8); 7.483(1.1);7.479(1.1);7.416(0.5);7.411(0.6);7.397(0.9);7.393(0.8);7.377(0.6);7.373(0.5); 7.316(0.7);7.313(0.7);7.297(0.9);7.294(0.9);7.264(11.6);5.299(4.3);5.240(4.6);3.463(16.0);1.259(0.8);1.257(0.7);1.219(1.4);0.000(3.9)

### Example No. A31-21

### ¹H-NMR (400 MHz, CDCl₃ δ, ppm)

8.730(0.6);8.728(0.7);8.725(0.7);8.723(0.7);8.718(0.6);8.716(0.7);8.713(0.7);8.711(0.6); 8.274(2.6);8.268(0.8);8.256(0.9);8.251(3.2);8.042(3.4);8.036(1.0);8.024(0.9);8.019(2.7); 7.859(1.1);7.855(1.1);7.840(0.7);7.835(0.6);7.665(1.2);7.662(0.8);7.645(1.0);7.509(0.6); 7.506(0.6);7.497(0.6);7.494(0.7);7.490(0.6);7.487(0.6);7.478(0.6);7.475(0.5);7.261(84.7); 5.368(4.6);3.844(16.0);3.317(2.1);0.008(0.9);0.000(29.2);−0.009(0.9)

### Example No. A31-61

### ¹H-NMR (400 MHz, CDCl₃ δ, ppm)

8.199(2.8);8.194(1.0);8.182(1.48.176(4.7);8.171(0.7);8.113(0.7);8.108(4.6);8.103(1.2); 8.091(1.0);8.086(2.8);7.789(0.9);7.786(0.8);7.785(0.8);7.771(1.3);7.767(1.1);7.748(0.7); 7.744(0.8);7.728(0.9);7.726(1.0);7.633(0.6);7.618(1.3);7.614(1.2);7.599(1.0);7.595(0.9); 7.592(1.2);7.588(1.2);7.573(1.3);7.570(1.2);7.265(16.8);5.285(4.6);3.519(16.0);1.432(0.6);1.259(0.9);1.257(0.8);1.220(2.2);0.000(6.0)

### Example No. A31-122

### ¹H-NMR (400 MHz, CDCl₃ δ, ppm)

8.335(1.4);8.314(1.4);8.275(2.9);8.270(0.9);8.258(1.0);8.253(3.8);8.135(0.9);8.116(1.1); 8.078(0.5);8.073(3.6);8.067(1.1);8.055(1.0);8.050(3.0);7.934(0.8);7.912(1.0);7.873(0.6); 7.870(0.6);7.856(0.9);7.852(1.2);7.849(0.6);7.835(0.7);7.831(0.6);7.732(2.0);7.720(0.9); 7.717(0.9);7.711(1.9);7.702(0.8);7.699(1.3);7.696(0.7);7.682(0.6);7.679(0.6);7.519(1.6); 7.295(1.3);7.260(293.4);7.227(1.7);7.210(1.0);6.996(1.6);5.559(4.8);3.899(16.0);3.158(0.6);0.008(3.2);0.000(101.4);−0.009(2.9);−0.033(0.6)

### Example No. A32-1

### ¹H-NMR (400 MHz, CDCl₃ δ, ppm)

7.809(0.8);7.806(0.8);7.789(0.9);7.786(0.9);7.758(0.7);7.753(0.6);7.740(0.9);7.735(0.8); 7.697(0.8);7.694(0.8);7.678(1.0);7.675(1.0);7.575(0.6);7.572(0.6);7.557(1.1);7.553(1.1); 7.538(0.6);7.535(0.6);7.492(0.6);7.488(0.8);7.472(0.8);7.468(1.1);7.434(0.7);7.431(0.7); 7.418(0.6);7.416(0.8);7.414(0.8);7.412(0.8);7.411 (0.8);7.405(1.0);7.400(0.9);7.396(0.6); 7.392(0.5);7.386(0.9);7.384(0.9);7.379(1.0);7.365(0.9);7.361(0.8);7.265(8.0);5.298(3.7);5.209(4.7);3.406(16.0);2.043(0.7);1.258(1.0);1.218(1.1);0.000(2.6)

### Example No. A32-4

### ¹H-NMR (400 MHz, CDCl₃ δ, ppm)

7.798(0.8);7.796(0.8);7.795(0.8);7.778(1.0);7.776(0.9);7.718(0.9);7.714(0.9);7.699(1.0); 7.695(1.0);7.686(0.7);7.685(0.7);7.682(0.8);7.667(1.0);7.666(1.0);7.663(1.0);7.591(1.0); 7.587(1.0);7.573(0.9);7.571(1.4);7.570(1.0);7.567(1.2);7.554(1.2);7.551(1.2);7.535(0.7); 7.532(0.6);7.440(0.8);7.436(0.8);7.421(0.7);7.420(0.8);7.418(0.7);7.416(0.8);7.401(0.6); 7.397(0.5);7.334(1.2);7.314(1.7);7.295(1.0);7.265(0.5);7.261(26.8);5.254(4.6);3.419(16.0);0.002(0.5);0.000(11.7)

### Example No. A32-5

### ¹H-NMR (400 MHz, CDCl₃ δ, ppm)

7.803(1.0);7.801(1.0);7.783(1.1);7.780(1.1);7.742(2.5);7.721(2.8);7.673(1.2);7.670(1.3); 7.655(1.7);7.654(1.8);7.651(1.7);7.579(0.9);7.576(0.9);7.560(1.7);7.557(1.6);7.541(0.9); 7.538(0.8);7.504(2.8);7.498(3.0);7.448(1.0);7.444(1.0);7.428(1.2);7.425(1.0);7.424(1.0); 7.409(0.7);7.405(0.6);7.370(1.8);7.365(1.7);7.349(1.6);7.344(1.5);7.262(30.4);5.158(5.1);3.417(16.0);2.9553(3.4);2.9547(3.4);2.884(3.0);2.882(3.0);0.000(13.0)

### Example No. A32-6

### ¹H-NMR (400 MHz, CDCl₃ δ, ppm)

8.019(0.7);7.835(1.3);7.815(4.7);7.809(3.7);7.722(1.8);7.719(1.8);7.703(2.4);7.700(2.4); 7.602(1.1);7.599(1.1);7.583(2.1);7.580(2.1);7.564(1.1);7.561 (1.1);7.448(1.1);7.444(1.1); 7.424(2.7);7.409(0.9);7.402(5.2);7.388(3.5);7.382(3.3);7.367(0.9);7.361(1.0);7.261(61.7); 5.212(6.9);4.130(0.5);4.113(0.5);3.416(16.0);2.955(7.0);2.884(5.7);2.883(5.8);2.410(1.6);2.044(2.5);1.581(0.5);1.277(0.7);1.259(1.5);1.241(0.7);0.008(0.7);0.000(26.1);−0.009(0.8)

### Example No. A32-7

### ¹H-NMR (400 MHz, CDCl₃ δ, ppm)

7.850(1.1);7.847(1.1);7.829(1.2);7.827(1.2);7.731(1.5);7.728(1.5);7.7121(1.8);7.7115(1.9); 7.709(1.9);7.593(1.0);7.590(1.0);7.574(1.9);7.571(1.8);7.555(1.0);7.552(1.0);7.493(3.2);7.491(3.4);7.472(6.47.439(1.47.435(1.0);7.418(1.2);7.416(1.2);7.415(1.1);7.400(0.7);7.396(0.8);7.382(2.8);7.363(2.1);7.360(1.9);7.342(1.5);7.267(0.6);7.266(0.7);7.265(0.9);7.261(55.0);7.2573(0.8);7.2565(0.6);5.564(6.7);3.456(16.0);3.386(0.6);2.955(2.0); 2.884(1.7);2.882(1.7);1.587(0.5);0.008(0.7);0.000(22.9);−0.009(0.7)

### Example No. A32-10

### ¹H-NMR (400 MHz, CDCl₃ δ, ppm)

7.843(1.0);7.840(1.0);7.822(1.2);7.820(1.1);7.712(1.2);7.709(1.3);7.694(1.5);7.693(1.6); 7.690(1.5);7.589(0.9);7.586(0.9);7.571(1.7);7.567(1.7);7.552(1.0);7.549(0.9);7.520(0.5); 7.507(11.4);7.439(1.0);7.436(1.47.421(0.9);7.419(1.1);7.417(1.47.415(1.0);7.400(0.7); 7.397(0.7);7.268(0.6);7.267(0.7);7.2664(0.8);7.2656(1.0);7.265(1.3);7.261(70.3);7.257(0.7);7.256(0.5);5.519(6.0);3.440(16.0);1.566(1.8);0.008(0.9);0.000(29.2);−0.004(0.5);−0.009(0.9)

### Example No. A32-11

### ¹H-NMR (400 MHz, CDCl₃ δ, ppm)

7.819(1.5);7.817(1.5);7.799(1.6);7.797(1.6);7.6691(1.4);7.6685(1.6);7.666(1.6);7.665(1.6); 7.658(0.8);7.653(0.9);7.650(2.2);7.649(2.3);7.647(2.3);7.646(2.2);7.639(1.4);7.634(1.4);7.620(0.8);7.616(0.8);7.571(1.4);7.568(1.4);7. 552(2.4);7.549(2.3);7.533(1.3);7.530(1.2);7.480(0.9);7.478(0.7);7.476(0.6);7.474(0.6);7.467(0.7);7.465(0.6);7.462(0.7);7.460(1.0);7.455(0.6);7.446(0.6);7.441(0.6);7.433(1.5);7.429(1.5);7.414(1.4);7.413(1.6);7.411(1.4);7.409(1.5);7.394(1.1);7.390(1.0);7.271(1.2);7.269(1.5);7.263(31.8);7.253(1.9);7.250(2.0);7.234(0.9);7.231(0.9);7.190(0.9);7.188(0.9);7.170(0.9);7.166(1.3);7.163(0.9); 7.144(0.8);7.142(0.8);5.091(5.1);3.386(15.9);3.384(16.0);2.955(1.4);2.954(1.4);2.884(1.2); 2.882(1.2);0.000(13.2)

### Example No. A32-17

### ¹H-NMR (400 MHz, CDCl₃ δ, ppm)

7.832(1.6);7.829(1.6);7.812(1.7);7.809(1.7);7.720(1.6);7.717(1.7);7.701(2.1);7.698(2.2); 7.587(1.4);7.584(1.4);7.568(2.5);7.565(2.4);7.549(1.4);7.546(1.3);7.509(0.9);7.504(0.8); 7.493(0.6);7.488(1.8);7.483(0.6);7.472(1.0);7.467(1.0);7.431(1.4);7.427(1.5);7.412(1.4); 7.410(1.6);7.408(1.5);7.407(1.5);7.392(1.1);7.388(1.0);7.262(53.4);7.074(2.5);7.054(3.5);7.033(2.2);5.174(5.4);3.453(0.7);3.393(16.0);2.955(5.1);2.884(4.4);2.883(4.4);1.595(1.0);0.008(0.6);0.000(21.8);−0.009(0.7)

### Example No. A32-21

### ¹H-NMR (400 MHz, CDCl₃ δ, ppm)

8.734(0.6);8.732(0.7);8.730(0.7);8.728(0.6);8.722(0.6);8.720(0.7);8.718(0.7);8.716(0.6); 8.164(1.5);8.161(1.5);8.144(1.8);8.141(1.7);7.921(0.9);7.916(0.9);7.901(0.6);7.897(0.5); 7.761(1.0);7.758(0.7);7.741(0.9);7.739(0.6);7.736(1.0);7.733(1.0);7.720(0.5);7.717(1.1); 7.714(0.8);7.706(0.6);7.701(1.2);7.698(1.1);7.683(1.6);7.679(1.5);7.661(1.2);7.659(1.0); 7.657(1.1);7.641(1.0);7.637(0.9);7.556(1.1);7.552(1.1);7.537(0.7);7.534(0.7);7.529(0.5);7.526(0.6);7.517(0.5);7.514(0.5);7.510(0.5);7.495(1.2);7.492(1.0);7.484(0.5);7.481(0.5);7.477(0.8);7.473(0.8);7.262(48.6);5.389(2.9);5.300(1.2);3.866(13.7);3.281(16.0);2.417(9.4);0.008(0.5);0.000(17.2)

### Example No. A32-40

### ¹H-NMR (400 MHz, CDCl₃ δ, ppm)

8.487(2.4);8.474(2.4);7.833(0.9);7.830(0.8);7.813(1.0);7.810(0.9);7.708(0.8);7.705(0.9); 7.690(1.1);7.689(1.1);7.686(1.1);7.583(0.7);7.580(0.7);7.564(1.3);7.561(1.3);7.545(0.7); 7.542(0.7);7.515(2.4);7.502(2.3);7.429(0.8);7.425(0.8);7.411(0.8);7.409(0.9);7.407(0.8); 7.405(0.8);7.390(0.6);7.387(0.6);7.262(29.9);5.424(5.0);3.550(16.0);0.000(12.1)

### Example No. A32-61

### ¹H-NMR (400 MHz, CDCl₃ δ, ppm)

7.975(1.8);7.955(2.1);7.803(1.6);7.779(2.9);7.776(2.5);7.760(2.4);7.756(2.7);7.741(1.2); 7.738(1.2);7.722(2.5);7.718(2.2);7.702(1.4);7.699(1.2);7.678(1.6);7.675(1.6);7.659(2.2); 7.656(2.2);7.612(1.7);7.609(1.7);7.593(2.6);7.590(2.6);7.581(1.3);7.578(1.4);7.574(1.4); 7.571(1.3);7.562(2.2);7.559(2.1);7.543(1.1);7.458(1.2);7.456(1.2);7.438(1.9);7.419(0.9); 7.263(49.4);5.300(9.1);5.228(5.3);4.130(0.8);4.113(0.9);3.497(16.0);2.411(0.9);2.045(4.0);1.277(1.1);1.259(2.2);1.241(1.1);0.008(0.6);0.000(17.3)

### Example No. A32-62

### ¹H-NMR (400 MHz, CDCl₃ δ, ppm)

7.839(1.7);7.806(0.9);7.801(1.3);7.798(1.1);7.787(1.1);7.781(1.4);7.778(1.2);7.775(0.9); 7.771(1.0);7.768(0.6);7.755(0.9);7.751(1.1);7.748(0.6);7.644(0.7);7.640(0.8);7.625(1.3); 7.621(1.3);7.594(1.0);7.585(0.9);7.582(0.8);7.574(1.7);7.567(1.3);7.564(1.2);7.555(0.7); 7.548(0.6);7.545(0.6);7.444(0.8);7.440(0.8);7.424(0.9);7.422(0.8);7.405(0.5);7.402(0.5); 7.261(36.5);4.997(4.3);3.386(16.0);2.170(0.7);1.259(0.5);1.256(0.6);0.000(15.0)

### Example No. A32-63

### ¹H-NMR (400 MHz, CDCl₃ δ, ppm)

7.799(1.0);7.797(1.0);7.779(1.1);7.777(1.1);7.754(2.47.749(0.9);7.738(1.2);7.733(3.4); 7.677(3.2);7.656(1.8);7.622(0.8);7.618(0.8);7.603(1.5);7.600(1.4);7.573(0.9);7.570(0.9); 7.555(1.3);7.552(1.2);7.536(0.6);7.533(0.6);7.447(0.8);7.443(0.8);7.427(1.0);7.425(0.9); 7.423(0.9);7.409(0.6);7.405(0.5);7.261(43.7);5.021(4.6);4.130(0.5);4.113(0.5);3.387(16.0);2.044(2.4);1.563(1.1);1.277(0.7);1.259(1.5);1.241(0.7);0.008(0.7);0.000(18.5);−0.009(0.8)

### Example No. A32-81

### ¹H-NMR (400 MHz, CDCl₃ δ, ppm)

8.138(1.3);8.118(1.3);7.826(1.1);7.808(1.2);7.766(1.1);7.747(1.3);7.730(1.4);7.726(1.5); 7.711(1.9);7.707(1.9);7.664(0.6);7.646(1.2);7.627(0.8);7.605(1.0);7.602(0.9);7.586(1.9); 7.583(2.1);7.567(1.1);7.564(1.4);7.560(1.4);7.541(0.6);7.462(1.0);7.458(0.9);7.442(1.2); 7.439(1.2);7.423(0.7);7.419(0.6);7.261(39.8);5.191(4.0);4.187(1.1);3.394(16.0);2.955(0.8);2.884(0.7);2.882(0.7);2.755(0.9);2.410(3.1);0.008(0.5);0.000(16.6);−0.009(0.5)

### Example No. A32-122

### ¹H-NMR (400 MHz, CDCl₃ δ, ppm)

8.400(1.2);8.379(1.3);8.172(1.1);8.169(1.1);8.152(1.2);8.149(1.48.133(1.0);8.112(1.1); 7.958(0.8);7.937(1.0);7.876(0.6);7.872(0.7);7.858(0.8);7.855(1.2);7.851(0.6);7.837(0.7); 7.833(0.7);7.817(1.8);7.796(1.7);7.728(0.9);7.725(1.3);7.722(1.1);7.719(0.8);7.711(0.8); 7.706(1.9);7.703(1.9);7.690(0.7);7.687(1.7);7.684(1.1);7.663(1.0);7.659(1.2);7.643(1.0); 7.639(1.1);7.624(0.6);7.544(1.2);7.540(1.2);7.525(1.0);7.519(1.8);7.260(280.6);7.227(2.2);6.996(1.6);5.599(4.5);3.941(16.0);3.253(3.3);3.182(6.3);3.075(0.7);2.847(6.3);2.418(2.0);0.008(3.1);0.000(96.6);−0.009(2.7);−0.034(0.7)

### Example No. A32-603

### ¹H-NMR (400 MHz, CDCl₃ δ, ppm)

8.026(0.9);8.023(0.9);8.006(0.9);8.003(0.9);7.828(0.8);7.825(0.9);7.806(1.5);7.784(0.7); 7.679(1.0);7.676(1.0);7.660(1.4);7.657(1.4);7.624(0.5);7.620(0.5);7.605(1.1);7.601(1.1); 7.586(0.7);7.582(0.6);7.567(0.5);7.564(0.6);7.548(1.0);7.545(1.0);7.532(0.9);7.529(1.2); 7.513(1.0);7.510(1.0);7.405(0.7);7.261(54.8);5.497(2.3);3.876(16.0);3.393(7.5);2.044(1.3);1.572(1.4);1.259(0.8);0.008(0.6);0.000(23.5);−0.009(0.7)

### Example No. A32-679

### ¹H-NMR (400 MHz, CDCl₃ δ, ppm)

8.787(1.0);8.783(1.0);8.775(1.1);8.771(1.0);8.352(0.8);8.348(0.8);8.332(0.9);8.328(0.9); 7.745(0.8);7.743(0.8);7.725(0.9);7.722(0.9);7.665(0.7);7.662(0.7);7.646(1.0);7.642(1.6); 7.630(0.9);7.621(0.9);7.609(0.8);7.565(0.6);7.562(0.6);7.546(1.1);7.543(1.1);7.527(0.6); 7.524(0.6);7.460(0.7);7.456(0.8);7.439(0.8);7.437(0.7);7.436(0.8);7.261(29.1);5.174(4.1);3.519(16.0);1.573(1.5);0.000(12.1)

### Example No. A33-1

### ¹H-NMR (400 MHz, CDCl₃ δ, ppm)

8.822(0.8);8.818(1.0);8.813(0.9);8.336(0.6);8.333(0.9);8.330(0.6);8.317(0.6);8.314(0.9); 8.310(0.6);8.231(0.6);8.228(0.6);8.225(0.6);8.222(0.6);8.210(0.7);8.207(0.6);8.204(0.7); 8.201(0.6);7.670(0.7);7.665(0.8);7.651(0.8);7.646(0.8);7.536(0.9);7.516(1.5);7.503(0.7); 7.500(0.8);7.496(0.9);7.483(1.0);7.480(1.1);7.416(0.5);7.412(0.5);7.398(0.8);7.394(0.8); 7.378(0.6);7.373(0.5);7.335(0.7);7.332(0.7);7.317(0.9);7.313(0.9);7.265(9.5);5.300(3.2);5.230(4.6);3.473(16.0);2.044(0.8);1.259(1.0);1.219(1.2);0.000(3.3)

### Example No. A33-21

### ¹H-NMR (400 MHz, CDCl₃ δ, ppm)

8.744(0.7);8.742(0.7);8.740(0.8);8.738(0.7);8.731(1.6);8.726(2.0);8.722(1.0);8.390(0.6); 8.388(0.7);8.384(0.6);8.382(0.7);8.370(0.7);8.367(0.7);8.364(0.7);8.361(0.6);8.228(0.7); 8.225(0.8);8.221(0.6);8.208(0.7);8.206(0.8);8.204(0.8);8.202(0.6);7.886(0.5);7.871(1.1); 7.867(1.1);7.852(0.7);7.848(0.7);7.696(1.2);7.694(0.8);7.677(1.0);7.646(0.9);7.626(1.6); 7.606(0.8);7.512(0.6);7.509(0.6);7.499(0.6);7.497(0.7);7.492(0.6);7.490(0.6);7.480(0.6);7.477(0.5);7.261(82.4);5.366(4.6);3.846(16.0);3.275(0.6);0.008(0.9);0.000(28.3);−0.009(0.8)

### Example No. A33-61

### ¹H-NMR (400 MHz, CDCl₃ δ, ppm)

8.776(0.9);8.772(1.1);8.771(1.1);8.767(0.9);8.317(0.6);8.314(0.9);8.310(0.7);8.298(0.7); 8.294(1.0);8.291(0.7);8.236(0.6);8.233(0.7);8.230(0.7);8.227(0.6);8.215(0.7);8.212(0.7); 8.209(0.7);8.206(0.6);7.792(1.4);7.789(0.9);7.787(0.8);7.772(1.6);7.769(1.4);7.640(1.0); 7.636(0.9);7.621(0.6);7.616(0.6);7.594(0.8);7.591(0.8);7.575(1.1);7.572(1.0);7.539(0.9); 7.519(1.6);7.499(0.8);7.265(14.2);5.300(2.0);5.266(4.3);3.530(16.0);2.045(2.1);1.277(0.7);1.259(1.7);1.241(0.7);1.220(1.4);0.000(5.0)

### Example No. A33-122

### ¹H-NMR (400 MHz, CDCl₃ δ, ppm)

8.764(1.0);8.759(1.6);8.755(1.0);8.391(0.7);8.388(0.8);8.385(0.7);8.383(0.8);8.370(0.8); 8.368(0.9);8.364(1.0);8.362(1.8);8.340(1.3);8.256(0.7);8.254(0.9);8.250(0.7);8.237(0.8); 8.233(0.9);8.230(0.7);8.153(0.9);8.132(1.0);7.938(0.8);7.918(1.0);7.874(0.6);7.870(0.6); 7.856(0.8);7.853(1.1);7.849(0.6);7.835(0.7);7.831(0.6);7.768(2.0);7.747(1.8);7.720(0.8); 7.717(0.8);7.703(0.7);7.700(1.2);7.697(0.7);7.682(0.6);7.679(0.6);7.648(1.0);7.628(1.8);7.608(0.9);7.519(1.2);7.260(210.2);7.227(1.6);6.996(1.2);5.566(4.8);3.911(16.0);0.008(2.4);0.000(73.7);−0.009(2.0);−0.034(0.6)

### Example No. A34-1

### ¹H-NMR (400 MHz, CDCl₃ δ, ppm)

8.285(0.7);8.284(0.8);8.281(1.4);8.280(1.5);8.277(0.9);8.276(0.8);8.230(0.7);8.226(0.9); 8.222(0.6);8.210(0.7);8.207(0.9);8.203(0.6);7.653(0.6);7.649(0.8);7.646(0.6);7.634(0.7); 7.630(1.0);7.626(0.8);7.624(0.8);7.619(0.8);7.604(0.9);7.600(0.9);7.499(0.7);7.496(0.8); 7.479(1.1);7.476(1.2);7.474(1.1);7.472(0.9);7.454(1.4);7.453(1.4);7.435(0.7);7.433(0.6); 7.414(0.5);7.409(0.6);7.395(0.9);7.391(0.8);7.375(0.6);7.371(0.5);7.322(0.7);7.318(0.7);7.303(0.9);7.300(0.9);7.264(10.8);5.299(3.7);5.237(4.6);3.449(16.0);1.258(0.7);1.219(1.2);0.000(3.6)

### Example No. A34-21

### ¹H-NMR (400 MHz, CDCl₃ δ, ppm)

8.724(0.6);8.722(0.7);8.720(0.7);8.717(0.6);8.712(0.6);8.710(0.7);8.707(0.7);8.705(0.6); 8.169(0.9);8.166(1.5);8.165(1.5);8.162(1.0);8.119(0.7);8.116(0.9);8.112(0.6);8.099(0.7); 8.096(0.9);8.095(0.8);8.092(0.6);7.860(0.5);7.845(1.1);7.840(1.2);7.825(0.7);7.821(0.7); 7.803(0.6);7.800(0.8);7.796(0.6);7.783(0.7);7.780(0.9);7.776(0.7);7.653(1.2);7.651(0.8); 7.634(1.0);7.631(0.7);7.563(0.8);7.562(0.8);7.544(1.5);7.524(0.7);7.523(0.7);7.490(0.6);7.488(0.6);7.478(0.6);7.475(0.6);7.471(0.6);7.468(0.6);7.459(0.6);7.456(0.5);7.261(47.8);5.347(4.8);5.300(0.7);3.822(16.0);3.231(0.8);0.000(16.3)

### Example No. A34-61

### ¹H-NMR (400 MHz, CDCl₃ δ, ppm)

8.245(0.7);8.244(0.8);8.241(1.5);8.240(1.5);8.237(1.0);8.236(0.8);8.209(0.7);8.205(1.0); 8.201(0.6);8.189(0.8);8.186(1.0);8.182(0.7);7.785(0.7);7.782(0.7);7.781(0.6);7.767(0.9); 7.763(0.9);7.738(0.6);7.735(0.7);7.718(0.8);7.716(0.9);7.661(0.6);7.6572(0.8);7.6567(0.8);7.653(0.6);7.643(0.6);7.641(0.9);7.638(1.2);7.634(0.8);7.624(1.1);7.620(1.0);7.605(0.7);7.601(0.6);7.591(0.9);7.587(0.9);7.572(1.1);7.568(1.0);7.476(0.9);7.475(0.9);7.457(1.5);7.455(1.4);7.437(0.7);7.436(0.7);7.265(12.3);5.300(2.4);5.285(4.2);4.130(0.6);4.113(0.6);3.502(16.0);2.045(3.0);1.277(0.9);1.259(1.7);1.241(0.8);0.000(4.5)

### Example No. A34-122

### ¹H-NMR (400 MHz, CDCl₃ δ, ppm)

8.372(1.2);8.351(1.3);8.209(0.9);8.205(1.7);8.202(1.1);8.146(0.7);8.144(0.9);8.139(0.7); 8.131(1.0);8.127(0.9);8.123(1.0);8.122(0.9);8.119(0.8);8.1 12(1.0);8.110(1.1);7.947(0.8); 7.927(1.0);7.886(0.6);7.882(0.6);7.869(0.8);7.865(1.2);7.861(0.6);7.847(0.9);7.844(1.1); 7.842(1.0);7.838(0.7);7.825(0.8);7.822(1.1);7.818(0.8);7.740(2.0);7.731(0.8);7.728(0.9); 7.719(1.9);7.714(0.8);7.711(1.3);7.708(0.8);7.693(0.5);7.690(0.5);7.599(0.9);7.598(0.9);7.579(1.6);7.560(0.7);7.559(0.7);7.519(0.5);7.260(98.7);6.996(0.5);5.584(5.0);3.936(16.0);0.008(1.1);0.000(34.8);−0.009(1.0)

### Example No. A35-1

### ¹H-NMR (400 MHz, CDCl₃ δ, ppm)

8.076(2.7);8.071(0.9);8.059(0.9);8.054(2.9);7.647(0.5);7.643(3.0);7.639(0.9);7.626(1.0); 7.623(2.5);7.622(3.1);7.619(1.1);7.604(0.9);7.600(0.9);7.497(0.7);7.494(0.8);7.477(1.1); 7.473(1.1);7.410(0.6);7.406(0.6);7.392(0.9);7.388(0.8);7.372(0.6);7.367(0.5);7.312(0.7); 7.309(0.7);7.293(1.0);7.290(0.9);7.275(0.5);7.271(0.5);7.265(11.6);5.299(4.2);5.234(4.6);3.449(16.0);1.432(0.8);1.258(0.8);1.218(1.1);0.071(1.5);0.000(3.9)

### Example No. A35-61

### ¹H-NMR (400 MHz, CDCl₃ δ, ppm)

8.048(2.9);8.047(1.8);8.044(1.0);8.032(1.0);8.028(2.1);8.027(3.1);8.023(0.5);7.782(0.7); 7.779(0.7);7.778(0.6);7.764(1.1);7.7601(0.9);7.7595(0.9);7.736(0.6);7.733(0.8);7.716(0.8);7.714(0.9);7.645(0.6);7.642(3.2);7.641(2.1);7.637(1.1);7.633(0.5);7.629(0.6);7.625(1.0);7.621(1.9);7.620(2.9);7.617(0.6);7.614(1.2);7.610(1.0);7.595(0.9);7.591(0.7);7.586(1.0);7.582(1.0);7.567(1.1);7.564(1.0);7.264(17.9);5.300(3.5);5.280(4.3);3.502(16.0);2.044(1.3);1.259(0.8);0.000(6.2)

### Example No. A35-122

### ¹H-NMR (400 MHz, CDCl₃ δ, ppm)

8.338(1.2);8.317(1.3);8.117(0.9);8.096(1.0);8.010(2.7);8.005(1.0);7.993(1.0);7.988(3.3); 7.933(0.9);7.913(1.0);7.872(0.6);7.868(0.7);7.854(0.8);7.851 (1.1);7.847(0.6);7.833(0.7); 7.829(0.6);7.743(0.6);7.739(3.3);7.734(1.0);7.720(3.6);7.718(4.0);7.703(0.9);7.700(2.9); 7.682(0.6);7.680(0.5);7.519(1.1);7.260(196.3);7.227(0.7);6.996(1.1);5.566(4.8);3.920(16.0);0.008(2.1);0.000(68.2);−0.009(2.0)

### Example No. A36-1

### ¹H-NMR (400 MHz, CDCl₃ δ, ppm)

7.846(0.7);7.841(0.6);7.839(0.5);7.829(1.0);7.825(1.47.823(1.7);7.805(1.0);7.803(1.0); 7.496(0.6);7.490(0.8);7.479(0.6);7.476(0.7);7.472(1.1);7.456(0.9);7.451(0.9);7.437(1.1); 7.433 (1.1);7.421(0.6);7.407(1.1);7.402(1.1);7.394(1.0);7.390(1.3);7.389(1.3);7.383(0.9); 7.376(1.0);7.372(0.9);7.314(0.8);7.311(0.9);7.295(1.2);7.292(1.2);7.276(0.7);7.273(0.8); 7.261(18.5);7.211(0.6);6.983(0.7);6.979(0.7);6.964(0.8);6.963(0.8);6.960(0.8);6.959(0.8);6.945(0.6);6.940(0.6);5.310(4.7);3.447(16.0);3.396(0.7);1.612(0.7);0.000(7.6)

### Example No. A36-2

### ¹H-NMR (400 MHz, CDCl₃ δ, ppm)

7.812(0.9);7.810(0.9);7.792(1.0);7.790(1.0);7.582(0.7);7.578(1.3);7.574(0.8);7.467(0.5); 7.465(0.5);7.462(0.7);7.458(0.7);7.454(0.5);7.451(0.5);7.448(1.2);7.442(1.7);7.438(1.2); 7.419(0.8);7.415(0.8);7.405(1.4);7.400(1.1);7.396(1.2);7.386(1.2);7.311(0.7);7.308(0.8); 7.292(1.2);7.289(1.2);7.274(0.8);7.271(0.8);7.260(29.6);6.978(0.7);6.974(0.7);6.959(0.7);6.958(0.8);6.955(0.7);6.954(0.8);6.940(0.6);6.935(0.6);5.057(4.3);3.400(16.0);2.044(1.0);2.029(0.5);1.259(0.7);0.000(11.2)

### Example No. A36-5

### ¹H-NMR (400 MHz, CDCl₃ δ, ppm)

7.842(1.7);7.830(1.1);7.828(1.1);7.821(1.9);7.810(1.47.808(1.47.506(1.8);7.501(1.9); 7.424(0.8);7.420(0.8);7.405(1.2);7.401(1.1);7.380(1.2);7.374 (1.1);7.359(1.1);7.353(1.0); 7.315(0.9);7.312(0.9);7.310(1.0);7.296(1.4);7.294(1.5);7.278(0.8);7.275(0.9);7.260(63.6); 7.211(1.9);6.991(0.7);6.987(0.7);6.973(0.8);6.971(0.9);6.968(0.8);6.967(0.8);6.953(0.6);6.948(0.6);5.258(4.7);3.444(16.0);3.395(0.6);2.044(0.9);1.560(9.4);1.259(0.5);0.008(0.9);0.000(26.5);−0.009(1.0);−0.050(0.8)

### Example No. A36-6

### ¹H-NMR (400 MHz, CDCl₃ δ, ppm)

7.894(1.4);7.888(1.4);7.825(0.9);7.823(0.9);7.805(0.9);7.803(0.9);7.489(0.7);7.485(0.8); 7.470(1.0);7.466(0.9);7.424(0.7);7.4032(2.2);7.4027(2.2);7.388(1.8);7.382(1.6);7.361(0.5);7.335(0.7);7.332(0.7);7.316(1.2);7.313(1.2);7. 297(0.6);7.295(0.6);7.261(24.8);6.992(0.7);6.988(0.7);6.973(0.7);6.972(0.8);6.969(0.7);6.968(0.8);6.954(0.6);6.949(0.6);5.269(4.5);3.460(16.0);2.030(0.9);0.000(9.2)

### Example No. A36-7

### ¹H-NMR (400 MHz, CDCl₃ δ, ppm)

7.812(1.0);7.810(1.0);7.809(1.0);7.792(1.1);7.790(1.0);7.789(1.0);7.498(2.1);7.496(2.8); 7.492(0.9);7.477(4.5);7.473(1.2);7.383(1.7);7.364(1.3);7.361(1.2);7.342(0.9);7.328(0.8); 7.325(0.8);7.310(1.4);7.307(1.3);7.291(0.8);7.288(0.7);7.265(0.6);7.261(31.5);6.979(0.8); 6.975(0.8);6.961(0.8);6.960(0.9);6.956(0.8);6.955(0.9);6.941(0.7);6.937(0.6);5.642(5.0);3.502(16.0);3.493(0.8);2.9553(0.8);2.9546(0.8);2.884(0.7);2.883(0.7);1.598(1.3);0.000(14.2)

### Example No. A36-10

### ¹H-NMR (400 MHz, CDCl₃ δ, ppm)

7.812(1.6);7.810(1.5);7.792(1.7);7.790(1.6);7.519(1.8);7.515(11.8);7.483(1.2);7.479(1.2); 7.465(1.8);7.460(1.5);7.328(1.1);7.325(1.1);7.310(3.5);7.307(2.1);7.291(1.2);7.288(1.3); 7.260(154.8);7.210(5.1);6.996(0.9);6.984(0.9);6.980(0.9);6.965(1.2);6.960(1.2);6.946(0.9);6.942(0.8);5.611(7.0);3.491(16.0);3.440(0.8);1.552(15.5);1.502(1.0);1.284(0.6);1.256(1.1);1.124(0.8);0.050(0.8);0.008(2.2);0.000(67.0);—0.009(3.3);—0.050(2.1)

### Example No. A36-11

### ¹H-NMR (400 MHz, CDCl₃ δ, ppm)

7.815(1.6);7.813(1.7);7.795(1.7);7.793(1.7);7.735(0.6);7.730(0.6);7.716(1.2);7.712(1.2); 7.697(0.7);7.693(0.7);7.478(0.8);7.476(0.6);7.473(0.6);7.471(0.5);7.464(0.6);7.462(0.6); 7.460(0.6);7.457(0.9);7.421(1.3);7.417(1.4);7.402(1.7);7.398(1.8);7.300(1.2);7.298(1.3); 7.282(2.2);7.279(3.0);7.276(1.3);7.261(44.4);7.241(0.8);7.238(0.8);7.190(0.8);7.188(0.8);7.170(0.8);7.166(1.1);7.163(0.8);7.144(0.7);7.142(0.7);6.974(1.1);6.969(1.2);6.955(1.4);6.954(1.46.951(1.4);6.950(1.46.936(1.0);6.931(1.0);5.182(4.9);3.419(15.7);3.416(16.0);2.955(0.8);2.884(0.7);2.883(0.7);2.170(3.2);2.017(1.4);0.000(15.9)

### Example No. A36-17

### ¹H-NMR (400 MHz, CDCl₃ δ, ppm)

7.805(1.8);7.803(1.9);7.785(1.9);7.783(1.9);7.512(0.9);7.507(0.8);7.496(0.5);7.491(1.7); 7.484(1.8);7.480(1.7);7.475(0.9);7.470(1.1);7.465(2.0);7.461(1.9);7.323(1.4);7.320(1.5); 7.304(2.5);7.301(2.5);7.286(1.4);7.283(1.4);7.261(62.1);7.083(2.4);7.063(3.3);7.042(2.0); 6.975(1.4);6.970(1.4);6.956(1.6);6.955(1.6);6.952(1.5);6.951(1.6);6.936(1.2);6.932(1.2);5.244(5.2);3.443(16.0);2.026(1.5);0.008(0.7);0.000(23.9);−0.009(0.7)

### Example No. A36-21

### ¹H-NMR (400 MHz, CDCl₃ δ, ppm)

8.664(0.7);8.660(1.1);8.657(0.8);8.652(0.8);8.649(1.2);8.645(0.7);7.801(1.2);7.800(1.4); 7.798(1.3);7.797(1.2);7.792(1.8);7.790(1.9);7.788(2.3);7.781(3.3);7.780(3.5);7.778(3.0); 7.776(1.9);7.382(0.8);7.370(1.2);7.359(1.2);7.348(0.7);7.281(0.7);7.280(0.7);7.271(0.5); 7.270(0.6);7.267(2.2);7.266(2.5);7.265(1.9);7.261(63.9);7.257(2.3);7.255(0.5);7.242(1.3);7.240(1.3);7.210(0.5);6.956(0.8);6.951(0.8);6.939(0.8);6.937(0.9);6.934(0.8);6.931(0.8);6.919(0.7);6.914(0.7);5.199(5.7);3.516(16.0);2.044(0.6);1.259(0.5);0.008(0.8);0.000(28.1);-0.009(0.8)

### Example No. A36-27

### ¹H-NMR (400 MHz, CDCl₃ δ, ppm)

8.851(4.1);8.838(4.2);7.785(0.9);7.782(1.0);7.765(1.47.763(1.0);7.420(0.8);7.416(0.8); 7.401(1.0);7.397(1.0);7.379(1.0);7.366(1.9);7.354(1.0);7.296(0.8);7.293(0.7);7.277(1.4); 7.274(1.3);7.261(40.6);7.256(0.8);6.957(0.7);6.952(0.7);6.938(0.8);6.937(0.8);6.934(0.7); 6.933(0.8);6.918(0.6);6.914(0.6);5.378(4.7);3.624(16.0);1.608(0.5);0.008(0.5);0.000(17.6)

### Example No. A36-40

### ¹H-NMR (400 MHz, CDCl₃ δ, ppm)

8.497(4.8);8.484(4.9);7.806(2.0);7.803(2.1);7.786(2.1);7.783(2.1);7.519(1.7);7.515(4.6); 7.503(4.4);7.472(1.2);7.468(1.2);7.453(1.5);7.449(1.5);7.322(1.4);7.319(1.3);7.303(2.4); 7.300(2.3);7.291(0.5);7.285(1.5);7.282(1.5);7.260(268.5);7.256(2.3);7.255(1.5);7.254(1.0);7.253(0.6);7.2524(0.6);7.2517(0.6);7.210(1.5);6.996(1.4);6.977(1.0);6.973(1.0);6.957(1.4);6.954(1.3);6.939(1.0);6.934(0.8);5.531(8.6);4.131(0.9);4.113(0.9);3.603(16.0);2.044(4.1);1.549(8.7);1.277(1.2);1.259(2.8);1.241(1.2);0.008(3.3);0.0054(1.1);0.0046(1.3); 0.000(113.5);−0.005(1.2);−0.006(1.0);−0.007(0.8);−0.009(3.3);-0.050(0.6)

### Example No. A36-61

### ¹H-NMR (400 MHz, CDCl₃ δ, ppm)

8.078(0.8);8.077(0.8);8.059(0.9);7.837(1.0);7.835(1.1);7.817(1.1);7.815(1.2);7.776(0.7); 7.774(0.8);7.757(0.9);7.754(1.0);7.745(0.6);7.741(0.5);7.725(1.2);7.722(1.0);7.706(0.7); 7.702(0.6);7.605(0.8);7.602(0.8);7.586(1.2);7.583(1.2);7.417(0.8);7.413(0.9);7.398(1.2); 7.394(1.1);7.307(0.8);7.304(0.8);7.288(1.4);7.285(1.3);7.269(0.8);7.266(1.1);7.261(44.5); 6.993(0.7);6.989(0.7);6.974(0.8);6.973(0.9);6.970(0.8);6.969(0.9);6.955(0.6);6.950(0.6);5.339(4.4);3.505(16.0);2.956(1.3);2.884(1.1);2.883(1.1);2.044(1.1);1.570(1.9);1.259(0.7);0.000(19.1);−0.009(0.6)

### Example No. A36-62

### ¹H-NMR (400 MHz, CDCl₃ δ, ppm)

7.885(2.1);7.881(2.7);7.878(0.7);7.867(0.6);7.863(0.8);7.823(1.0);7.821(1.0);7.820(1.0); 7.803(1.1);7.801(1.1);7.778(0.6);7.775(1.0);7.771(0.5);7.759(0.7);7.755(1.0);7.752(0.6); 7.602(0.7);7.583(0.9);7.581(0.8);7.561(0.6);7.382(0.7);7.378(0.7);7.363(1.2);7.359(1.2); 7.319(0.9);7.316(0.9);7.301(1.2);7.298(1.2);7.282(0.7);7.279(0.6);7.267(0.5);7.266(0.6); 7.265(0.8);7.264(1.1);7.261(44.7);7.258(0.8);6.991(0.8);6.987(0.8);6.973(0.8);6.971(0.8);6.968(0.8);6.967(0.8);6.953(0.7);6.949(0.6);5.101(4.1);3.434(16.0);2.044(2.4);1.593(0.6);1.277(0.7);1.259(1.5);1.241(0.7);0.008(0.5);0.000(19.7);−0.009(0.6)

### Example No. A36-63

### ¹H-NMR (400 MHz, CDCl₃ δ, ppm)

7.825(1.1);7.823(1.1);7.805(1.1);7.803(1.1);7.764(1.0);7.762(0.6);7.749(0.8);7.743(4.8); 7.732(4.4);7.726(0.6);7.711(0.8);7.355(0.6);7.351(0.7);7.336(1.3);7.332(1.3);7.309(1.0); 7.306(1.0);7.290(1.3);7.288(1.3);7.272(0.9);7.270(0.7);7.269(1.2);7.268(0.9);7.267(0.9); 7.266(1.1);7.2654(1.3);7.2645(1.6);7.264(2.3);7.260(84.9);7.2564(1.0);7.2556(0.7);7.210(0.6);6.996(0.6);6.995(0.8);6.990(0.8);6.977(0.8);6.975(0.9);6.972(0.8);6.970(0.8); 6.957(0.7);6.952(0.6);5.133(4.7);3.432(16.0);2.044(1.0);1.573(1.7);1.259(0.7);0.008(1.0); 0.000(36.5);−0.009(1.0)

### Example No. A36-81

### ¹H-NMR (400 MHz, CDCl₃ δ, ppm)

8.267(1.0);8.247(1.0);7.843(1.3);7.841(1.3);7.824(1.4);7.821(1.3);7.770(0.9);7.750(1.1); 7.655(1.0);7.636(0.6);7.578(0.7);7.560(1.0);7.486(1.0);7.482(1.0);7.468(1.3);7.463(1.3); 7.346(0.9);7.344(0.9);7.328(1.5);7.325(1.5);7.309(1.1);7.306(0.8);7.261(35.1);7.211(1.2); 7.010(0.8);7.005(0.8);6.990(1.1);6.986(1.1);6.971(0.7);6.967(0.7);5.304(4.0);3.428(16.0);3.378(0.8);2.753(1.4);2.044(0.8);1.581(1.2);1.259(0.5);0.008(0.6);0.000(14.8);−0.008(0.9)

### Example No. A36-603

### ¹H-NMR (400 MHz, CDCl₃ δ, ppm)

8.030(0.8);8.026(0.8);8.010(0.9);8.006(0.9);7.925(0.8);7.922(0.8);7.906(0.9);7.903(0.9); 7.814(1.0);7.811(1.0);7.794(1.1);7.791(1.0);7.627(0.5);7.612(1.0);7.608(1.0);7.593(0.7); 7.589(0.6);7.530(0.7);7.527(0.7);7.511(0.9);7.507(0.9);7.429(0.8);7.425(0.8);7.410(1.1); 7.406(1.0);7.302(0.8);7.299(0.8);7.283(1.3);7.281(1.3);7.265(1.2);7.261(36.8);6.972(0.7);6.967(0.7);6.953(0.8);6.952(0.8);6.949(0.8);6.947(0.8);6.933(0.6);6.929(0.6);5.603(4.1);3.878(16.0);3.422(13.1);2.018(0.5);0.000(13.7)

### Example No. A36-679

### ¹H-NMR (400 MHz, CDCl₃ δ, ppm)

8.791(1.0);8.787(1.1);8.780(1.2);8.775(1.2);8.487(0.8);8.483(0.8);8.467(0.9);8.463(0.9); 7.848(0.9);7.846(0.9);7.828(1.1);7.826(1.0);7.647(1.0);7.635(1.0);7.627(0.9);7.615(0.9); 7.382(0.6);7.378(0.7);7.363(1.1);7.359(1.0);7.308(0.8);7.305(0.8);7.290(1.2);7.287(1.1); 7.271(0.8);7.268(0.9);7.261 (39.4);7.211(0.7);7.008(0.7);7.004(0.7);6.990(0.8);6.989(0.8);6.986(0.7);6.984(0.8);6.970(0.6);6.966(0.6);5.380(0.7);5.333(3.8);3.551(16.0);1.575(1.4);0.000(16.9);−0.009(0.5)

### Example No. A38-1

### ¹H-NMR (600 MHz, d₆-DMSO δ, ppm)

7.916(0.6);7.903(0.6);7.615(0.6);7.604(0.7);7.601(0.7);7.532(0.5);7.519(0.6);7.451(0.5); 7.439(0.6);7.426(0.6);7.413(1.1);7.401(1.0);7.389(0.5);7.380(0.5);7.360(0.4);7.283(0.4); 7.271(0.4);7.203(0.5);7.200(0.5);7.190(0.9);7.188(0.9);7.177(0.5);7.175(0.4);3.376(5.1); 3.373(5.8);3.368(8.1);3.363(9.8);3.360(7.6);3.356(14.0);2.968(0.5);2.838(0.5);2.671(0.7);2.515(11.5);2.512(15.5);2.509(11.5);1.404(0.5);1.361(0.6);1.306(16.0);1.240(1.3)

### Example No. A38-5

### ¹H-NMR (600 MHz, d₆-DMSO δ, ppm)

7.918(0.5);7.905(0.5);7.643(1.0);7.639(0.5);7.636(0.4);7.629(1.2);7.599(0.4);7.596(0.4); 7.514(0.5);7.500(0.4);7.488(0.4);7.474(0.6);7.459(0.3);7.456(0.3);7.428(0.3);7.425(0.3); 7.206(0.5);7.204(0.5);7.194(0.8);7.191(0.7);7.181(0.4);7.178(0.4);3.978(0.4);3.970(0.4); 3.377(4.1);3.375(4.0);3.369(4.5);3.363(5.8);3.359(6.4);3.354(5.6);3.352(6.7);3.331(0.4); 3.002(0.5);2.841(1.2);2.739(0.7);2.596(0.3);2.518(4.9);2.515(11.0);2.512(15.6);2.509(11.3);2.506(5.2);1.508(1.3);1.387(0.4);1.353(1.2);1.294(16.0);1.240(1.3)

### Example No. A38-612

### ¹H-NMR (600 MHz, d₆-DMSO δ, ppm)

7.937(0.8);7.923(1.0);7.909(0.6);7.896(0.5);7.567(0.6);7.555(0.3);7.523(0.9);7.510(1.0); 7.489(0.7);7.476(0.9);7.455(0.4);7.443(0.6);7.431(0.5);7.423(0.4);7.410(0.5);7.401(0.5); 7.388(0.5);7.366(0.4);7.353(0.7);7.323(1.1);7.310(0.9);7.298(0.6);7.284(0.6);7.274(0.7); 7.260(1.1);7.245(1.8);7.235(3.1);7.232(3.4);7.211(0.9);7.193(0.9);7.179(0.5);3.452(0.4); 3.432(0.4);3.372(10.4);3.369(9.7);3.367(10.5);3.362(14.4);3.357(15.0);3.355(16.1); 3.352(24.1);3.302(0.4);3.204(2.2);3.161(0.6);3.137(6.4);3.054(0.4);3.036(0.4);2.932(1.2); 2.898(1.2);2.878(8.1);2.803(0.4);2.739(0.9);2.530(0.4);2.518(11.5);2.515(25.8);2.512(36.5);2.509(27.0);2.506(12.6);1.506(5.0);1.475(16.0);1.439(0.8);1.397(2.5);1.368(0.9); 1.337(1.2);0.006(0.3)

### Example No. A38-615

### ¹H-NMR (600 MHz, d₆-DMSO δ, ppm)

7.962(0.4);7.935(0.9);7.922(1.0);7.914(0.6);7.903(0.8);7.891(0.5);7.644(1.0);7.629(0.3); 7.572(2.0);7.558(2.2);7.492(1.5);7.444(1.1);7.431(1.1);7.380(0.5);7.376(0.5);7.366(0.4); 7.363(0.4);7.344(1.3);7.328(1.1);7.297(1.9);7.283(1.3);7.261(0.8);7.248(0.9);7.235(0.5); 7.220(0.7);7.207(1.0);7.191(0.9);7.178(0.5);3.974(0.4);3.686(0.4);3.676(0.5);3.671(0.4); 3.664(0.4);3.660(0.5);3.652(0.5);3.648(0.4);3.637(0.4);3.460(0.4);3.361(12.2);3.360(12.0);3.357(13.8);3.354(20.6);3.352(17.2);3.351(18.7);3.294(0.3);3.193(3.1);3.128(7.1); 3.035(0.4);2.964(0.5);2.954(0.5);2.942(0.4);2.919(2.3);2.898(3.2);2.888(12.8);2.797(0.5); 2.785(0.7);2.773(0.3);2.739(2.6);2.530(0.3);2.518(11.6);2.515(25.8);2.512(35.9);2.509(25.7);2.506(11.6);1.500(6.1);1.468(16.0);1.424(0.8);1.394(4.4);1.333(1.2);1.293(2.1); 1.281(0.4)

### Example No. A39-1

### ¹H-NMR (600 MHz, d₆-DMSO δ, ppm)

7.677(0.4);7.667(0.6);7.652(0.4);7.567(0.6);7.553(1.4);7.542(2.9);7.532(4.7);7.528(4.2); 7.519(2.7);7.516(4.3);7.468(0.7);7.465(0.8);7.453(1.1);7.435(0.8);7.426(2.3);7.422(2.4); 7.420(3.5);7.415(5.0);7.408(3.3);7.405(2.7);7.396(1.1);7.390(1.0);7.385(1.0);7.377(0.9); 7.375(1.1);7.364(1.4);7.351(0.4);7.330(0.6);7.315(1.2);7.301(0.6);7.232(1.1);7.218(2.4); 7.203(1.9);5.034(0.5);4.928(0.5);4.689(2.6);3.391(19.0);3.386(21.3);3.381(23.1);3.378(23.8);3.374(27.6);3.368(33.4);3.364(30.5);3.362(42.2);3.358(50.9);3.357(48.9);3.335(0.4);2.797(10.9);2.716(1.3);2.624(0.6);2.533(0.8);2.530(1.0);2.527(1.1);2.518(26.4);2.515(57.7);2.512(80.2);2.509(57.2);2.506(25.5);2.396(0.5);1.511(0.6);1.414(2.8);1.306(1.2);1.283(16.0);1.268(0.7);1.238(0.7);1.133(7.1);0.008(0.5)

### Example No. A39-5

### ¹H-NMR (400 MHz, d₆-DMSO δ, ppm)

7.715(3.2);7.681(0.3);7.670(0.5);7.657(1.6);7.653(1.6);7.570(1.4);7.557(2.7);7.545(2.1); 7.532(4.4);7.528(4.1);7.518(2.0);7.515(1.9);7.504(0.6);7.500(0.6);7.490(0.8);7.487(0.9); 7.395(1.3);7.381(1.0);7.328(0.6);7.314(1.0);7.303(0.6);7.235(1.1);7.221(2.3);7.207(1.8); 5.094(0.5);4.830(0.5);4.678(2.8);3.374(16.8);3.365(20.0);3.359(27.5);3.355(20.8);3.352(34.4);3.332(1.0);2.899(0.9);2.861(2.2);2.849(8.7);2.794(1.3);2.739(0.8);2.624(0.4); 2.530(0.6);2.515(43.2);2.512(60.9);2.509(46.1);2.396(0.3);1.508(2.8);1.397(2.5);1.269(16.0);1.237(0.5);1.121(8.2);0.008(0.5)

### Example No. A39-612

### ¹H-NMR (600 MHz, d₆-DMSO δ, ppm)

7.638(0.4);7.626(0.5);7.612(0.4);7.553(0.4);7.541(0.5);7.528(0.4);7.489(1.1);7.476(2.2); 7.463(1.0);7.356(0.6);7.344(1.2);7.340(0.8);7.332(1.3);7.324(1.5);7.314(1.2);7.311(1.3); 7.302(0.9);7.298(0.8);7.294(1.4);7.278(0.6);7.265(0.8);7.255(1.4);7.248(2.1);7.242(1.2); 7.240(1.0);7.226(0.7);7.210(0.9);7.197(0.5);3.921(0.4);3.906(0.3);3.780(0.3);3.769(0.3); 3.583(0.5);3.505(0.4);3.494(0.4);3.372(14.9);3.365(17.3);3.359(24.3);3.355(26.9);3.353(25.7);3.352(29.6);3.159(1.1);3.145(0.4);3.130(0.5);3.118(0.4);3.111(0.4);3.098(7.0); 3.016(0.6);3.002(0.8);2.988(0.5);2.862(7.2);2.533(0.4);2.530(0.5);2.527(0.5);2.518(18.3); 2.515(41.4);2.512(58.5);2.509(42.8);2.506(19.9);1.493(2.5);1.431(16.0);1.406(0.6); 1.372(0.6);1.332(2.7);1.265(0.4);0.008(0.4)

### Example No. A39-615

### ¹H-NMR (600 MHz, d₆-DMSO δ, ppm)

7.650(1.6);7.647(1.6);7.640(0.4);7.626(0.5);7.612(0.4);7.540(0.4);7.530(0.5);7.522(1.8); 7.508(2.3);7.498(1.3);7.495(1.3);7.441(1.0);7.438(1.0);7.428(0.7);7.424(0.7);7.371(0.4); 7.358(1.8);7.353(1.3);7.349(1.2);7.344(0.6);7.339(0.3);7.336(0.3);7.303(0.4);7.288(0.8); 7.274(0.4);7.254(0.4);7.240(0.8);7.226(0.9);7.212(0.9);3.939(0.4);3.933(0.4);3.924(0.6); 3.916(0.5);3.909(0.4);3.900(0.4);3.784(0.4);3.773(0.5);3.768(0.6);3.761(0.4);3.757(0.5);3.750(0.4);3.745(0.4);3.734(0.4);3.581(0.4);3.501(0.4);3.475(0.4);3.373(2.5);3.361(15.8);3.358(17.1);3.356(27.9);3.354(22.9);3.352(33.6);3.154(1.2);3.129(0.4);3.115(0.4); 3.095(7.2);3.083(0.4);3.077(0.4);3.068(0.4);3.006(0.7);3.001(0.7);2.988(1.1);2.974(0.6); 2.899(1.3);2.886(5.8);2.739(0.9);2.530(0.4);2.527(0.4);2.518(13.2);2.515(29.5);2.512(41.4);2.509(29.9);2.506(13.4);1.488(2.7);1.425(16.0);1.394(0.4);1.325(2.1);1.269(0.7); 0.007(0.4)

### Example No. A40-1

### ¹H-NMR (400 MHz, d₆-DMSO δ, ppm)

7.901(1.5);7.881(1.5);7.830(4.3);7.811(4.5);7.662(2.6);7.657(2.9);7.643(2.9);7.639(3.2); 7.509(3.0);7.505(3.0);7.490(4.1);7.486(4.2);7.461(1.0);7.439(2.9);7.420(4.4);7.413(1.4); 7.409(1.7);7.403(2.8);7.401(2.9);7.394(3.6);7.391(3.5);7.376(3.7);7.370(4.6);7.364(4.1); 7.359(1.9);7.357(2.2);7.350(4.4);7.346(4.5);7.335(1.5);7.332(1.5);7.327(1.0);7.312(4.1); 7.308(5.3);7.293(3.4);7.289(3.6);7.270(0.4);7.173(1.7);7.167(1.1);7.164(0.6);7.157(1.0);7.154(1.4);7.148(1.5);7.144(1.1);7.129(0.8);7.124(2.5);7.120(2.2);7.105(3.5);7.101(3.5);7.086(1.8);7.082(1.8);5.885(0.3);5.870(1.1);5.856(1.1);5.842(0.3);4.901(15.1);4.870(1.1);4.856(3.6);4.843(3.6);4.829(1.1);3.332(58.9);2.889(1.1);2.730(1.0);2.600(5.8);2.585(5.8);2.524(0.5);2.510(13.8);2.506(28.4);2.501(38.2);2.497(28.2);2.493(14.1);2.317(16.0);2.304(15.9);0.000(5.4)

### Example No. A40-5

### ¹H-NMR (400 MHz, d₆-DMSO δ, ppm)

7.953(0.6);7.901(1.3);7.882(1.3);7.824(4.4);7.804(4.6);7.686(4.9);7.667(8.0);7.663(11.0); 7.534(1.8);7.529(2.0);7.501(1.1);7.484(4.5);7.479(5.4);7.463(3.2);7.458(3.3);7.447(1.7); 7.442(1.6);7.437(2.0);7.435(2.1);7.427(1.0);7.418(4.3);7.416(4.5);7.399(2.7);7.397(3.0);7.374(1.4);7.358(0.8);7.355(0.8);7.298(4.0);7.294(4.5);7.279(3.2);7.275(3.3);7.175(2.4);7.156(2.8);7.137(0.7);7.133(0.6);7.123(2.1);7.119(2.2);7.103(3.5);7.100(3.5);7.084(1.9);7.080(1.9);5.886(1.0);5.872(1.0);4.913(1.1);4.900(3.7);4.886(4.2);4.874(16.0);3.330(38.7);2.889(3.9);2.731(3.4);2.598(5.1);2.584(5.1);2.510(15.0);2.506(30.1);2.502(39.9); 2.497(30.0);2.493(15.5);2.315(15.8);2.301(15.7);0.000(6.4)

### Example No. A40-612

### ¹H-NMR (400 MHz, d₆-DMSO δ, ppm)

7.829(3.6);7.811(3.7);7.809(3.8);7.791(3.4);7.773(3.4);7.771(3.5);7.484(2.1);7.480(2.4); 7.470(2.9);7.466(5.8);7.461(3.2);7.450(3.3);7.447(3.4);7.397(1.5);7.395(1.6);7.378(3.3); 7.376(3.5);7.360(3.1);7.357(3.3);7.342(3.0);7.338(3.0);7.324(2.5);7.320(3.2);7.313(1.6); 7.307(3.6);7.303(5.9);7.295(1.4);7.288(3.7);7.283(2.8);7.276(3.7);7.271(5.7);7.262(4.0); 7.253(5.9);7.249(4.0);7.235(4.0);7.232(4.2);7.216(2.2);7.213(2.3);7.203(0.5);7.196(3.3);7.191(1.9);7.186(1.5);7.181(1.7);7.173(1.8);7.141(2.9);7.137(3.5);7.131(2.2);7.126(2.4);7.122(2.9);7.118(3.0);7.111(3.1);7.107(3.2);7.092(1.6);7.087(2.4);7.082(1.8);7.067(2.9);7.063(2.7);7.048(1.6);7.044(1.4);6.369(2.6);6.365(3.0);6.350(2.6);6.346(2.8);5.768(0.8);5.754(2.7);5.739(2.7);5.724(0.8);5.032(0.7);5.018(2.5);5.004(2.5);4.991(0.7);3.804(2.2);3.785(2.8);3.765(2.4);3.421(0.7);3.407(0.8);3.329(46.1);3.202(0.4);3.182(0.9); 3.153(3.5);3.132(3.6);3.114(2.7);2.979(0.9);2.959(1.5);2.940(1.6);2.928(1.4);2.913(1.0); 2.890(1.6);2.731(1.2);2.676(0.3);2.671(0.5);2.666(0.5);2.655(16.0);2.640(15.9);2.525(0.8);2.511(21.1);2.507(43.9);2.502(58.6);2.498(42.6);2.493(20.6);2.322(13.1);2.308(12.9); 1.234(0.4);0.008(0.3);0.000(11.2);−0.009(0.4)

### Example No. A40-615

### ¹H-NMR (400 MHz, d₆-DMSO δ, ppm)

7.844(3.7);7.842(4.2);7.824(4.1);7.822(4.3);7.789(4.5);7.771(4.4);7.769(4.6);7.619(6.3); 7.614(6.9);7.518(4.5);7.497(7.7);7.477(5.8);7.472(6.5);7.446(5.0);7.441(4.8);7.426(2.9); 7.420(2.9);7.399(2.0);7.397(2.2);7.380(4.3);7.378(4.6);7.361(2.9);7.358(4.7);7.352(3.1); 7.337(4.2);7.332(4.1);7.290(1.8);7.287(2.0);7.271(4.1);7.269(4.4);7.253(2.5);7.250(2.6); 7.233(6.8);7.212(5.0);7.148(2.2);7.144(2.6);7.134(3.8);7.130(6.8);7.125(4.1);7.115(3.5);7.111(4.5);7.106(2.1);7.087(2.4);7.082(2.3);7.067(3.7);7.063(3.5);7.048(2.1);7.044(2.0);6.524(3.0);6.520(3.3);6.505(3.0);6.501(3.0);5.763(0.9);5.748(3.1);5.734(3.1);5.719(0.9);5.049(1.0);5.036(3.4);5.022(3.5);5.008(1.0);3.798(2.8);3.779(4.0);3.760(3.1);3.419(0.9);3.404(0.9);3.332(92.5);3.193(0.5);3.176(1.0);3.156(0.9);3.134(4.1);3.114(4.9);3.096(3.3);2.964(1.0);2.944(1.7);2.925(1.7);2.911(1.6);2.891(2.6);2.732(1.5);2.677(0.3); 2.672(0.5);2.668(0.4);2.646(16.0);2.631(15.9);2.526(0.9);2.512(21.9);2.508(45.9);2.503(61.8);2.499(46.0);2.494(23.1);2.330(0.7);2.318(15.7);2.304(15.7);1.233(0.5);0.000(8.8); −0.009(0.4)

### Example No. A41-1

### ¹H-NMR (400 MHz, d₆-DMSO δ, ppm)

7.521(3.3);7.502(4.0);7.490(1.6);7.486(1.5);7.469(3.1);7.452(1.5);7.448(1.9);7.431(0.9); 7.424(0.6);7.421(0.7);7.409(0.9);7.405(2.2);7.402(2.3);7.389(6.3);7.386(6.9);7.381(6.9); 7.375(4.5);7.368(2.8);7.357(2.9);7.350(1.8);7.345(0.9);7.341(1.2);7.334(0.9);7.325(0.5); 7.311(0.4);7.216(0.4);7.195(0.8);7.182(4.4);7.162(7.2);7.141(3.7);5.165(1.0);5.151(3.3); 5.138(3.5);5.124(1.0);4.928(15.8);4.549(1.2);3.335(51.8);3.274(1.7);2.889(1.1);2.731(0.9);2.519(2.1);2.511(9.5);2.506(19.9);2.502(25.8);2.497(18.9);2.493(9.3);2.345(16.0); 2.331(15.9);0.000(3.3)

### Example No. A41-5

### ¹H-NMR (400 MHz, d₆-DMSO δ, ppm)

7.687(6.0);7.681(6.6);7.549(0.5);7.544(0.6);7.530(3.1);7.525(3.1);7.509(4.2);7.504(4.8); 7.488(1.6);7.483(1.6);7.471(1.2);7.466(3.3);7.450(1.9);7.446(2.4);7.435(0.8);7.429(0.9); 7.393(6.3);7.372(4.7);7.226(0.4);7.206(0.6);7.177(4.6);7.156(7.4);7.136(3.8);5.183(1.1); 5.169(3.6);5.156(3.6);5.142(1.1);5.118(0.6);4.908(15.7);4.530(1.2);3.331(38.5);3.267(1.5);2.890(1.0);2.731(0.9);2.519(2.2);2.511(11.0);2.507(23.4);2.502(31.1);2.498(22.7); 2.493(11.2);2.340(16.0);2.326(15.9);0.000(5.4)

### Example No. A41-612

### ¹H-NMR (400 MHz, d₆-DMSO δ, ppm)

7.565(0.5);7.560(0.4);7.543(0.7);7.526(0.4);7.522(0.4);7.461(2.9);7.455(2.7);7.444(3.7); 7.438(6.1);7.430(3.4);7.417(5.6);7.412(5.0);7.400(1.7);7.396(1.9);7.379(0.8);7.338(0.4); 7.327(0.9);7.323(1.4);7.318(0.5);7.309(3.7);7.304(3.7);7.297(3.7);7.291(5.9);7.286(3.1); 7.279(3.8);7.274(2.9);7.260(1.0);7.255(1.0);7.237(0.9);7.227(1.2);7.222(1.6);7.214(2.9); 7.206(0.7);7.199(0.4);7.178(1.1);7.158(1.6);7.137(1.0);7.123(4.3);7.102(7.1);7.082(3.7);5.850(0.7);5.836(0.7);5.263(1.0);5.249(3.3);5.235(3.3);5.222(1.0);4.062(0.4);3.855(3.7);3.837(4.9);3.817(4.1);3.445(0.7);3.427(1.1);3.408(0.9);3.332(44.2);3.288(2.2);3.089(4.0);3.070(4.9);3.051(3.6);2.986(0.4);2.952(0.9);2.933(1.1);2.915(0.8);2.890(1.3);2.731(1.1);2.612(4.1);2.597(4.1);2.525(0.5);2.512(13.2);2.507(27.4);2.503(36.8);2.498(27.1); 2.494(13.2);2.345(16.0);2.332(15.9);0.000(5.5)

### Example No. A41-615

### ¹H-NMR (400 MHz, d₆-DMSO δ, ppm)

7.605(5.3);7.600(5.7);7.580(0.5);7.564(0.4);7.559(0.4);7.543(0.6);7.526(0.3);7.522(0.4); 7.458(4.2);7.452(1.1);7.445(1.4);7.437(8.6);7.414(3.0);7.395(5.9);7.390(4.8);7.374(2.8); 7.369(2.4);7.340(0.3);7.322(0.6);7.318(0.6);7.302(0.9);7.296(0.9);7.277(0.3);7.271(0.6); 7.251(1.6);7.230(0.9);7.165(1.0);7.145(1.6);7.119(4.5);7.098(7.3);7.078(3.7);5.844(0.7); 5.829(0.7);5.272(1.0);5.259(3.3);5.245(3.3);5.232(1.0);4.067(0.4);3.862(3.5);3.845(5.7);3.826(3.8);3.453(0.7);3.435(1.3);3.417(0.8);3.336(51.8);3.290(2.0);3.071(3.8);3.053(6.0);3.035(3.5);2.979(0.4);2.935(0.8);2.917(1.3);2.900(0.7);2.892(1.1);2.733(0.9);2.603(3.9);2.588(3.9);2.526(0.5);2.513(11.5);2.509(23.6);2.504(31.6);2.500(23.2);2.495(11.3); 2.347(16.0);2.333(15.9);0.000(3.3)

### Example No. A48-1

### ¹H-NMR (400 MHz, CDCl₃ δ, ppm)

7.932(2.3);7.794(1.0);7.775(1.5);7.731(1.2);7.704(1.9);7.683(1.1);7.521(1.0);7.501(1.6); 7.451(0.7);7.432(1.2);7.413(0.7);7.390(1.0);7.372(1.2);7.262(7.9);5.207(5.4);3.416(16.0); 1.591(3.1);0.000(3.5)

### Example No. A48-36

### ¹H-NMR (400 MHz, CDCl₃ δ, ppm)

8.875(0.9);8.873(1.0);8.870(1.0);8.868(0.9);8.076(1.1);8.072(1.1);7.916(1.2);7.914(1.2); 7.912(1.3);7.797(0.6);7.794(0.5);7.777(0.8);7.774(0.8);7.709(1.3);7.689(0.8);7.261(43.6); 5.420(4.0);3.592(16.0);1.555(4.5);0.008(0.5);0.000(18.9);−0.009(0.6)

### Example No. A48-61

### ¹H-NMR (400 MHz, CDCl₃ δ, ppm)

7.935(1.3);7.934(1.2);7.932(1.2);7.931(1.3);7.918(0.7);7.916(0.7);7.898(0.9);7.896(0.9); 7.806(0.7);7.803(0.7);7.788(1.3);7.787(1.3);7.784(1.5);7.769(0.8);7.765(0.8);7.730(0.5); 7.715(1.1);7.711(0.9);7.695(0.7);7.692(0.6);7.651(1.3);7.638(0.8);7.635(0.9);7.631(1.0); 7.619(1.2);7.616(1.1);7.262(27.0);5.205(4.5);3.483(16.0);1.570(5.4);0.000(11.6)

### Example No. A49-36

### ¹H-NMR (400 MHz, CDCl₃ δ, ppm)

8.876(0.9);8.874(1.0);8.871(1.0);8.869(0.8);8.076(1.48.075(1.0);8.071(1.0);7.878(1.0); 7.753(0.7);7.720(1.0);7.266(0.6);7.265(0.7);7.264(1.0);7.260(59.9);7.256(0.5);5.450(3.8); 3.596(16.0);1.550(7.3);0.008(0.8);0.002(1.0);0.000(27.4);−0.009(0.7)

### Example No. A50-1

### ¹H-NMR (400 MHz, CDCl₃ δ, ppm)

7.786(0.7);7.782(0.6);7.768(0.9);7.763(0.8);7.494(0.6);7.489(0.9);7.479(0.9);7.475(0.9); 7.474(0.9);7.470(1.1);7.460(1.0);7.434(1.1);7.403(1.1);7.398(0.9);7.384(0.9);7.379(1.0); 7.378(0.9);7.375(0.9);7.361(1.0);7.357(0.9);7.294(0.6);7.277(0.5);7.2754(0.6);7.2745(0.6);7.273(0.5);7.262(13.3);5.269(4.7);3.397(16.0);2.373(6.1);0.000(5.8)

### Example No. A50-36

### ¹H-NMR (400 MHz, CDCl₃ δ, ppm)

8.863(1.0);8.860(1.0);8.060(1.1);8.057(1.0);7.475(0.8);7.456(1.0);7.417(1.2);7.299(0.7); 7.280(0.6);7.261(25.1);5.487(3.9);3.573(16.0);2.370(6.1);0.000(10.8)

### Example No. A50-40

### ¹H-NMR (400 MHz, CDCl₃ δ, ppm)

8.489(2.7);8.476(2.8);7.519(0.6);7.508(2.7);7.500(1.1);7.495(2.7);7.480(1.3);7.420(1.5); 7.301(0.9);7.282(0.8);7.269(0.6);7.268(0.7);7.267(0.8);7.266(1.0);7.260(98.4);7.210(0.7); 6.996(0.6);5.463(4.9);3.549(16.0);2.370(8.2);1.571(1.4);1.256(0.5);0.008(1.1);0.000(44.8);−0.009(1.3)

### Example No. A50-61

### ¹H-NMR (400 MHz, CDCl₃ δ, ppm)

7.992(0.8);7.990(0.8);7.972(0.9);7.971(0.9);7.776(0.7);7.773(0.8);7.757(0.8);7.754(1.0); 7.721(0.5);7.705(1.1);7.702(0.9);7.686(0.6);7.682(0.5);7.602(0.7);7.599(0.8);7.583(1.1); 7.580(1.1);7.441(1.2);7.428(0.9);7.409(1.1);7.288(0.7);7.266(0.7);7.262(18.4);5.300(4.2); 3.455(16.0);2.375(6.5);1.613(1.8);0.000(7.8)

### Example No. A51-1

### ¹H-NMR (400 MHz, CDCl₃ δ, ppm)

7.750(0.7);7.746(0.7);7.731(0.9);7.727(0.8);7.624(1.2);7.619(1.3);7.541(0.7);7.521(1.4); 7.507(0.6);7.503(0.8);7.487(0.9);7.483(1.1);7.474(0.8);7.473(0.8);7.469(0.8);7.431(0.5); 7.417(1.0);7.412(0.9);7.397(0.7);7.392(0.6);7.383(0.7);7.379(0.8);7.364(0.9);7.360(0.9); 7.262(16.8);5.234(4.7);3.401(16.0);1.605(0.8);0.000(7.1)

### Example No. A51-36

### ¹H-NMR (400 MHz, CDCl₃ δ, ppm)

8.868(0.9);8.866(1.0);8.863(1.0);8.861(0.8);8.068(1.0);8.067(1.0);8.063(1.0);7.606(1.2); 7.601(1.2);7.543(0.7);7.522(1.3);7.477(0.8);7.476(0.8);7.472(0.8);7.261(28.7);5.446(3.8); 3.577(16.0);1.255(0.5);0.000(12.3)

### Example No. A51-61

### ¹H-NMR (400 MHz, CDCl₃ δ, ppm)

7.944(0.7);7.942(0.7);7.925(0.9);7.923(0.9);7.790(0.7);7.787(0.7);7.771(0.8);7.768(0.9); 7.707(1.0);7.703(0.9);7.687(0.6);7.683(0.5);7.629(1.2);7.624(1.2);7.619(0.8);7.616(0.8); 7.600(1.1);7.596(1.1);7.471(2.0);7.464(1.4);7.459(1.2);7.262(17.6);5.251(4.3);3.463(16.0); 1.594(0.5);0.000(7.5)

### Example No. A52-36

### ¹H-NMR (400 MHz, CDCl₃ δ, ppm)

8.852(0.8);8.850(0.9);8.847(0.9);8.846(0.8);8.062(0.9);8.061(0.9);8.057(0.9);8.056(0.9); 7.695(0.7);7.678(0.7);7.263(14.7);6.816(0.5);5.439(3.7);3.608(16.0);0.000(6.8)

### Example No. A53-1

### ¹H-NMR (400 MHz, CDCl₃ δ, ppm)

8.653(1.2);8.650(1.3);8.633(1.4);8.630(1.3);8.376(1.0);8.373(1.0);8.358(1.1);8.355(1.0); 7.708(1.4);7.690(1.3);7.687(1.3);7.669(1.2);7.542(0.6);7.538(0.7);7.523(0.7);7.519(1.0); 7.510(0.7);7.507(0.7);7.490(0.9);7.487(0.9);7.389(0.5);7.385(0.5);7.371(0.7);7.366(0.7); 7.351(0.5);7.267(0.6);7.266(0.7);7.2654(0.8);7.2646(1.0);7.264(1.5);7.261(50.2);7.258(1.3);7.257(0.8);7.256(0.5);7.213(0.6);7.210(0.7);7.194(0.8);7.190(0.8);5.349(3.9);3.520(14.3);1.556(16.0);0.008(0.6);0.000(23.0);−0.009(0.6)

### Example No. A53-3

### ¹H-NMR (400 MHz, CDCl₃ δ, ppm)

8.649(1.3);8.646(1.4);8.628(1.4);8.626(1.4);8.348(1.1);8.346(1.2);8.330(1.2);8.328(1.2); 7.698(1.3);7.680(1.3);7.678(1.4);7.659(1.2);7.406(1.0);7.390(0.7);7.384(3.8);7.380(0.8); 7.372(0.9);7.368(4.1);7.362(0.7);7.346(1.1);7.262(23.2);5.076(4.5);3.456(16.0);2.044(0.6);0.000(9.5)

### Example No. A53-4

### ¹H-NMR (400 MHz, CDCl₃ δ, ppm)

8.658(1.4);8.655(1.4);8.637(1.5);8.635(1.5);8.365(1.2);8.362(1.2);8.347(1.3);8.344(1.2); 7.707(1.5);7.689(1.5);7.687(1.5);7.669(1.4);7.550(1.1);7.546(1.2);7.530(1.2);7.526(1.3); 7.457(0.9);7.453(0.9);7.438(1.1);7.434(0.9);7.268(0.5);7.267(0.7);7.266(0.8);7.2653(1.0); 7.2645(1.2);7.260(55.7);7.257(0.9);7.256(0.6);7.138(1.2);7.119(1.9);7.099(1.0);5.418(4.7);3.529(16.0);2.044(0.5);1.554(11.9);0.008(0.7);0.000(25.2);−0.009(0.7)

### Example No. A53-21

### ¹H-NMR (400 MHz, CDCl₃ δ, ppm)

8.671(0.5);8.668(0.6);8.666(0.6);8.664(0.5);8.659(0.6);8.656(0.6);8.654(0.6);8.652(0.5); 8.627(1.2);8.624(1.3);8.607(1.4);8.604(1.3);8.333(1.2);8.330(1.2);8.315(1.3);8.312(1.2); 7.683(1.3);7.665(1.3);7.662(1.3);7.644(1.2);7.609(0.9);7.605(0.9);7.590(0.6);7.586(0.6); 7.521(0.7);7.519(1.1);7.516(0.7);7.499(0.7);7.330(0.6);7.327(0.6);7.318(0.6);7.315(0.6); 7.311(0.5);7.266(0.5);7.262(25.1);5.220(4.4);3.581(16.0);1.599(0.6);0.000(10.9)

### Example No. A53-22

### ¹H-NMR (400 MHz, CDCl₃ δ, ppm)

8.802(8.8);8.780(8.8);7.878(1.4);7.737(9.5);7.718(10.3);7.716(10.9);7.698(7.8);7.520(3.6); 7.358(0.6);7.297(1.0);7.289(1.8);7.280(2.2);7.2743(3.2);7.2735(3.3);7.272(4.0);7.271(4.3);7.2703(4.8);7.2695(5.4);7.269(5.9);7.268(6.7);7.267(7.5);7.266(8.8);7.261(623.4); 7.257(5.2);7.256(4.0);7.255(2.7);7.254(2.0);7.2532(1.7);7.2525(1.4);7.252(1.2);7.251(1.1);7.250(1.0);7.2493(1.1);7.2485(1.2);7.248(0.9);7.247(0.7);7.246(0.6);7.2453 (0.7);7.2445(0.7);7.244(0.7);7.243(0.6);7.242(0.6);7.227(1.0);7.223(0.7);7.211(3.6);6.997(3.5); 4.772(2.5);2.808(16.0);2.006(0.6);1.601(1.7);1.334(0.7);1.284(1.0);1.256(1.2);0.146(0.7); 0.008(7.6);0.0063(2.0);0.0055(2.2);0.000(266.3);−0.005(3.3);-0.006(2.7);−0.007(2.3);−0.009(7.8);−0.034(0.6);−0.050(1.5);−0.149(0.8)

### Example No. A53-61

### ¹H-NMR (400 MHz, CDCl₃ δ, ppm)

8.660(1.3);8.658(1.4);8.640(1.5);8.637(1.4);8.363(1.3);8.360(1.4);8.345(1.5);8.342(1.4); 7.790(0.7);7.787(0.7);7.772(0.7);7.771(0.7);7.768(0.8);7.708(1.4);7.690(1.4);7.688(1.4); 7.670(1.3);7.630(0.6);7.628(0.6);7.611(0.8);7.608(0.8);7.532(1.1);7.529(1.0);7.513(0.8); 7.510(0.6);7.490(0.8);7.486(0.8);7.470(0.9);7.466(0.9);7.262(23.2);5.404(3.9);3.567(16.0);2.044(0.8);1.583(2.2);1.259(0.5);0.000(10.4)

### Example No. B1-2

### ¹H-NMR (400 MHz, CDCl₃ δ, ppm)

8.835(1.7);8.703(0.8);8.690(0.8);7.548(0.7);7.544(1.3);7.540(0.8);7.502(1.1);7.488(1.4); 7.473(0.7);7.468(1.1);7.463(0.7);7.435(0.9);7.431(1.4);7.427(0.6);7.422(1.4);7.421(1.4); 7.403(1.1);7.261(40.8);4.992(4.5);3.384(16.0);2.064(0.5);2.044(1.8);1.277(0.5);1.259(1.1);0.008(0.5);0.000(18.0);−0.009(0.5)

### Example No. B1-4

### ¹H-NMR (400 MHz, CDCl₃ δ, ppm)

8.856(1.8);8.711(0.9);8.709(0.9);8.698(0.9);8.696(0.9);7.735(0.9);7.731(1.0);7.715(1.0); 7.711(1.0);7.615(1.1);7.611(1.1);7.595(1.3);7.591(1.2);7.514(1.1);7.501(1.0);7.352(1.2); 7.332(1.8);7.312(1.0);7.267(0.5);7.266(0.7);7.265(0.8);7.261(53.3);5.283(4.7);4.131(0.5); 4.113(0.5);3.448(16.0);2.071(0.7);2.044(2.6); 1.558(1.4);1.277(0.8); 1.259(1.7); 1.241(0.8);0.008(0.6);0.000(24.6);−0.009(0.7)

### Example No. B1-5

### ¹H-NMR (400 MHz, CDCl₃ δ, ppm)

8.841(1.2);8.712(0.6);8.710(0.6);8.699(0.6);8.697(0.6);7.752(1.0);7.731(1.1);7.527(1.1); 7.521(1.2);7.514(0.7);7.501 (0.7);7.385(0.7);7.380(0.7);7.364(0.6);7.359(0.6);7.265(0.7); 7.260(52.2);5.170(3.0);3.435(10.3);2.044(0.5);1.552(16.0);0.008(0.6);0.000(22.2);−0.009(0.7)

### Example No. B1-6

### ¹H-NMR (400 MHz, CDCl₃ δ, ppm)

8.908(1.9);8.722(0.9);8.721(0.9);8.709(0.9);8.708(0.9);7.827(1.4);7.822(1.4);7.513(1.1); 7.500(1.1);7.447(0.6);7.426(2.4);7.414(2.0);7.408(1.8);7.387(0.5);7.261(32.7);5.203(4.9); 3.442(16.0);2.044(1.4);1.259(0.9);0.000(14.2)

### Example No. B1-7

### ¹H-NMR (400 MHz, CDCl₃ δ, ppm)

8.924(1.9);8.705(0.9);8.703(0.9);8.692(0.9);8.690(0.9);7.507(1.9);7.505(2.3);7.486(3.5); 7.397(1.7);7.379(1.3);7.375(1.1);7.357(0.9);7.261(36.7);5.552(5.0);3.485(16.0);1.579(3.1);0.000(14.5)

### Example No. B1-10

### ¹H-NMR (400 MHz, CDCl₃ δ, ppm)

8.907(1.1);8.706(0.6);8.693(0.6);7.522(7.8);7.503(1.0);7.490(1.0);7.261(44.1);7.253(0.6); 5.507(5.2);3.473(16.0);1.568(9.5);0.008(0.6);0.000(19.1);−0.009(0.7)

### Example No. B1-11

### ¹H-NMR (400 MHz, CDCl₃ δ, ppm)

8.836(3.2);8.698(1.5);8.686(1.5);7.681(0.6);7.676(0.7);7.662(1.3);7.657(1.3);7.643(0.7); 7.639(0.7);7.519(0.7);7.503 (2.2);7.490(2.0);7.481(0.7);7.479(0.9);7.474(0.5);7.465(0.6); 7.290(1.0);7.287(1.2);7.271(2.2);7.268(2.5);7.266(1.4);7.261(92.2);7.253(1.0);7.249(1.0); 7.210(1.3);7.207(0.8);7.189(0.8);7.185(1.2);7.182(0.9);7.164(0.7);7.161(0.8);5.114(5.1);3.407(15.8);3.404(16.0);2.069(1.1);2.044(1.4);1.563 (0.7);1.259(0.9);0.008(1.2);0.000(41.9);−0.009(1.1)

### Example No. B1-15

### ¹H-NMR (400 MHz, CDCl₃ δ, ppm)

8.805(2.9);8.702(1.4);8.689(1.4);7.716(0.6);7.701(0.7);7.695(1.2);7.679(1.2);7.674(0.7); 7.658(0.6);7.504(1.8);7.491(1.8);7.262(26.9);7.028(0.5);7.026(0.5);7.013(0.9);7.006(1.0); 6.965(0.8);6.959(0.6);6.944 (0.9);6.939(1.0);6.933(0.8);6.918(0.8);6.912(0.7);5.426(0.5); 5.060(4.8);3.405(15.9);3.403(16.0);3.166(0.5);2.954(0.5);1.432(0.7);1.284(0.8);1.257(3.1);0.880(0.8);0.874(0.5);0.853(0.5);0.000(11.1)

### Example No. B1-17

### ¹H-NMR (400 MHz, CDCl₃ δ, ppm)

9.099(0.5);8.908(3.5);8.698(1.6);8.696(1.6);8.685(1.7);8.683(1.6);7.525(0.9);7.520(0.8); 7.509(0.6);7.504(1.7);7.499(0.7);7.493(2.2);7.488(1.0);7.481(2.2);7.263(32.8);7.090(2.3); 7.071(3.3);7.050(1.9);5.513 (0.9);5.177(5.1);3.424 (16.0);3.166(0.8);2.955(3.3);2.884(2.9);2.882(2.8);2.842(0.6);1.622(0.7); 1.285(0.8); 1. 257(3.8);0.880(1.0);0.874(0.7);0.870(0.6);0.863(0.7);0.853(0.8);0.837(0.5);0.008(0.5);0.000(14.1)

### Example No. B1-21

### ¹H-NMR (400 MHz, CDCl₃ δ, ppm)

8.682(0.6);8.680(0.7);8.678(0.7);8.675(0.8);8.670(1.3);8.668(1.0);8.666(0.8);8.663(0.7); 8.658(0.8);8.647(1.5);7.801(1.0);7.796(1.0);7.782(0.7);7.777(0.7);7.726(0.7);7.723(1.1); 7.721 (0.8);7.704(0.7);7.484(1.0);7.471(1.0);7.404(0.6);7.401(0.6);7.392(0.6);7.388(0.6); 7.385(0.6);7.382(0.5);7.373(0.5);7.370(0.5);7.265(26.9);5.301(1.8);5.135(4.6);3.503(16.0);2.736(1.2);2.483(1.8);1.651(0.6);1.432(1.9);1.256(0.7);0.070(0.9);0.000(8.5)

### Example No. B1-35

### ¹H-NMR (400 MHz, CD3CN 6, ppm)

8.724(1.3);8.668(0.6);8.654(0.6);8.632(1.4);8.627(1.4);8.029(1.3);8.023(1.3);7.531(0.9); 7.518(0.9);5.447(0.5);5.185(4.8);4.068(0.8);4.050(0.8);3.448(16.0);2.142(11.0);1.972(3.4);1.964(1.2);1.958(1.8);1.952(8.2);1.946(14.5);1.940(19.2);1.934(13.3);1.927(6.8);1.222(0.9);1.204(1.8);1.186(0.9);0.000(0.4)

### Example No. B1-36

### ¹H-NMR (400 MHz, CD3CN 6, ppm)

8.931(0.9);8.928(0.9);8.708(1.4);8.666(0.7);8.653(0.7);8.250(1.0);8.247(0.9);7.527(1.0); 7.514(0.9);5.270(4.3);3.511 (16.0);2.175(15.2);1.972(0.4);1.965(0.9);1.959(1.3);1.953(5.9); 1.947(10.3);1.941(13.4);1.934(9.2);1.928(4.7)

### Example No. B1-40

### ¹H-NMR (400 MHz, CDCl₃ δ, ppm)

8.885(1.8);8.695(0.9);8.694(0.9);8.682(0.9);8.681(0.9);8.506(2.0);8.494(2.1);7.526(2.0); 7.513(1.9);7.491(1.1);7.478(1.0);7.262(29.4);5.432(4.7);3.582(16.0);1.587(4.0);0.000(12.2)

### Example No. B1-62

### ¹H-NMR (400 MHz, CDCl₃ δ, ppm)

8.782(1.8);8.711(0.8);8.710(0.9);8.698(0.9);8.697(0.9);7.878(0.6);7.858(0.7);7.855(0.6); 7.801(1.9);7.798(1.6);7.782(0.8);7.779(0.9);7.623(0.7);7.621 (0.7);7.604(1.1);7.602(1.0); 7.508(1.1);7.495(1.0);7.261(69.6);7.256(0.5);7.211(0.6);5.014(4.1);3.421(16.0);2.044(1.5); 1.556(8.0); 1.259(1.0);0.008(0.8);0.000(30.1);−0.009(0.8)

### Example No. B1-63

### ¹H-NMR (400 MHz, CDCl₃ δ, ppm)

8.780(1.9);8.712(0.9);8.711(0.9);8.699(0.9);8.698(0.9);7.773(1.6);7.768(0.7);7.757(0.8); 7.751(3.0);7.697(2.7);7.692(0.8);7.681(0.6);7.676(1.5);7.510(1.1);7.497(1.1);7.260(65.6); 7.210(0.6);5.046(4.4);3.414(16.0);2.044(1.7);1.552(11.1);1.277(0.5);1.259(1.2);1.241(0.5);0.008(0.8);0.000(28.6);−0.009(1.1)

### Example No. B1-81

### ¹H-NMR (400 MHz, CDCl₃ δ, ppm)

8.918(2.0);8.729(0.9);8.727(1.0);8.716(0.9);8.714(1.48.160(0.7);8.141(0.7);7.787(0.6); 7.768(0.7);7.654(0.7);7.600(0.5);7.581(0.7);7.531(1.2);7.518(1.1);7.261(32.5);5.209(3.6); 3.416(16.0);1.579(4.1);0.000(13.8)

### Example No. B1-122

### ¹H-NMR (400 MHz, CDCl₃ δ, ppm)

8.698(2.0);8.676(0.9);8.675(0.9);8.663(0.9);8.662(0.9);8.278(1.0);8.258(1.0);8.116(0.7); 8.114(0.7);8.095(0.8);8.092(0.8);7.902(0.6);7.899(0.6);7.882(0.7);7.879(0.7);7.821(2.0); 7.805(0.6);7.800(1.9);7.788(0.7);7.784(1.0);7.767(0.6);7.763(0.6);7.652(0.7);7.649(0.7); 7.635(0.6);7.632(1.2);7.629(0.7);7.614(0.5);7.611(0.5);7.492(1.1);7.479(1.1);7.263(49.6); 5.310(4.9);5.301(4.4);3.581(16.0);2.046(0.6);1.603(1.7);1.432(1.0);1.260(0.7);0.002(0.6);0.000(15.2);−0.003(0.8)

### Example No. B1-461

### ¹H-NMR (400 MHz, CDCl₃ δ, ppm)

8.829(1.9);8.721(0.9);8.720(0.9);8.708(0.9);7.519(1.1);7.506(1.1);7.264(12.9);7.198(0.8); 6.907(1.0);6.888(1.4);6.867(0.9);3.810(0.9);3.798(0.6);3.790(0.7);3.780(0.6);3.769(1.0); 3.468(16.0);2.817(0.7);2.799(1.4);2.780(0.7);2.217(0.6);2.196(0.7);2.176(0.6);0.000(4.7)

### Example No. B1-603

### ¹H-NMR (400 MHz, CDCl₃ δ, ppm)

8.836(1.7);8.694(0.8);8.682(0.8);8.051(0.8);8.048(0.8);8.032(0.9);8.028(0.9);7.838(0.7); 7.835(0.7);7.819(0.8);7.816(0.8);7.621(1.0);7.617(0.9);7.601(0.6);7.598(0.6);7.552(0.7); 7.548(0.7);7.533(0.9);7.529(0.9);7.500(1.1);7.487(1.0);7.266(0.6);7.265(0.7);7.264(1.0); 7.261(42.9);7.257(0.5);5.537(4.1);3.878(16.0);3.415(13.4);2.044(1.5);1.573(0.5);1.259(1.0);0.008(0.5);0.000(20.1);−0.009(0.6)

### Example No. B1-679

### ¹H-NMR (400 MHz, CDCl₃ δ, ppm)

8.772(0.8);7.260(53.7);5.223(1.7);3.550(6.8);2.044(0.9);1.543(16.0);1.259(0.6);0.008(0.7); 0.000(21.9);−0.009(0.6)

### Example No. B3-21

### ¹H-NMR (400 MHz, CDCl₃ δ, ppm)

8.620(0.7);8.618(0.8);8.617(0.7);8.609(0.7);8.607(0.7);7.802(0.9);7.798(0.8);7.783(0.6); 7.779(0.5);7.709(0.7);7.686(1.0);7.682(0.8);7.666(0.8);7.662(0.8);7.393(1.1);7.381(0.9); 7.374(0.9);7.362(0.6);7.261(25.1);5.119(3.8);3.491(16.0);1.597(2.9);0.000(9.3)

### Example No. B4-1

### ¹H-NMR (400 MHz, CDCl₃ δ, ppm)

9.000(1.9);8.488(1.6);8.474(1.6);7.701(0.7);7.697(0.7);7.682(0.8);7.677(0.8);7.502(0.7); 7.498(0.8);7.493(0.6);7.491(0.6);7.485(0.8);7.484(0.9);7.481(1.3);7.478(1.7);7.475(1.0); 7.470(0.7);7.4614(0.6);7.4606(0.7);7.425(0.6);7.421(0.7);7.417(0.6);7.409(0.7);7.407(1.1);7.404(0.8);7.402(1.1);7.401(0.8);7.394(0.8);7.387(0.6);7.382(0.6);7.346(0.7);7.343(0.7);7.327(0.9);7.324(0.9);7.292(2.0);7.284(1.0);7.283(1.1);7.279(1.0);7.277(1.0);7.269(2.5);7.265(29.4);5.210(4.3);4.712(6.0);3.477(1.7);3.464(16.0);1.255(0.6);1.220(1.1);0.000(10.8)

### Example No. B4-21

### ¹H-NMR (400 MHz, CDCl₃ δ, ppm)

8.893(1.3);8.656(0.5);8.654(0.6);8.644(0.5);8.642(0.5);8.453(0.9);8.440(0.9);7.725(0.8); 7.721(0.8);7.706(0.6);7.702(0.6);7.653(0.6);7.650(1.1);7.648(0.7);7.631(0.7);7.448(0.5); 7.447(0.5);7.263(11.2);5.098(4.5);3.525(16.0);0.000(3.5)

### Example No. B4-37

### ¹H-NMR (400 MHz, CDCl₃ δ, ppm)

8.958(1.8);8.457(1.4);8.443(1.4);8.297(1.6);7.452(0.6);7.450(0.6);7.438(0.5);7.436(0.6); 7.263(16.7);5.055(4.5);3.790(14.2);3.550(16.0);2.462(7.9);2.303(6.3);0.000(4.7)

### Example No. B4-61

### ¹H-NMR (400 MHz, CDCl₃ δ, ppm)

7.859(0.6);7.856(0.7);7.839(0.8);7.837(0.8);7.785(0.6);7.782(0.7);7.766(0.8);7.763(0.8); 7.655(0.9);7.651(0.9);7.635(0.6);7.631(0.5);7.600(0.7);7.597(0.7);7.581(1.0);7.578(1.0); 7.263(15.7);5.298(2.0);5.246(4.1);3.516(16.0);2.078(0.9);0.000(5.2)

### Example No. B6-21

### ¹H-NMR (400 MHz, CDCl₃ δ, ppm)

9.108(0.9);9.105(0.9);9.103(0.8);8.652(0.5);8.650(0.6);8.648(0.5);8.643(0.5);8.640(0.6); 8.638(0.6);8.331(0.6);8.326(0.6);8.311(0.6);8.306(0.6);7.713(0.9);7.708(0.9);7.694(0.6); 7.689(0.6);7.611(0.6);7.608(1.1);7.606(0.7);7.595(1.0);7.589(0.8);7.575(0.9);7.360(0.5); 7.357(0.5);7.348(0.5);7.345(0.5);7.265(8.7);6.784(0.6);6.645(1.4);6.506(0.7);5.088(4.5); 3.518(16.0);1.703(0.5);0.000(3.2)

### Example No. B6-61

### ¹H-NMR (400 MHz, CDCl₃ δ, ppm)

8.344(1.2);8.339(1.2);8.332(1.2);8.327(1.2);8.068(0.7);8.066(0.7);8.048(0.8);8.046(0.8); 7.786(1.9);7.781(1.9);7.767(2.0);7.762(2.1);7.721(1.0);7.717(0.9);7.702(0.6);7.698(0.5); 7.616(0.7);7.613(0.7);7.597(1.1);7.594(1.1);7.266(0.5);7.262(29.8);7.210(1.4);7.198(1.4); 7.192(1.3);7.180(1.4);5.304(4.1);3.499(16.0);1.588(1.6);0.000(12.6)

### Example No. B7-1

### ¹H-NMR (400 MHz, CDCl₃ δ, ppm)

9.247(1.0);9.244(1.0);8.400(0.7);8.396(0.7);8.380(0.7);8.375(0.7);7.686(0.7);7.681(0.7); 7.667(0.8);7.662(0.8);7.649(1.3);7.647(1.2);7.628(1.2);7.626(1.1);7.501(0.7);7.497(0.7); 7.481(1.1);7.477(1.2);7.469(0.6);7.460(0.7);7.416(0.5);7.413(0.9);7.405(0.7);7.398(1.2); 7.393(0.8);7.390(0.9);7.378(0.6);7.336(0.6);7.332(0.6);7.317(0.9);7.313(0.8);7.298(0.7); 7.288(2.0);7.279(1.5);7.273(1.4);7.264(2.2);7.260(26.5);5.173(4.6);4.709(5.8);3.477(16.0);1.568(2.7);1.257(0.8);1.222(0.5);0.000(11.1)

### Example No. B7-21

### ¹H-NMR (400 MHz, CDCl₃ δ, ppm)

9.164(1.0);9.161(1.0);8.655(0.6);8.653(0.6);8.642(0.6);8.640(0.6);8.343(0.7);8.338(0.7); 8.323(0.7);8.318(0.7);7.725(0.9);7.720(0.8);7.706(0.6);7.701(0.5);7.620(1.8);7.601(1.5); 7.599(1.7);7.350(0.5);7.260(33.0);5.064(4.7);3.523(16.0);1.564(1.9);0.000(13.2)

### Example No. B8-5

### ¹H-NMR (400 MHz, CDCl₃ δ, ppm)

8.027(1.7);8.006(1.8);7.470(1.9);7.465(2.0);7.360(0.7);7.356(0.8);7.342(0.9);7.337(1.1); 7.335(1.3);7.329(1.0);7.314(1.0);7.308(1.0);7.265(0.9);7.262(14.1);7.247(0.7);7.244(0.7); 7.242(0.7);7.240(0.6);7.226(0.7);7.221(0.6);6.907(1.3);6.904(1.3);6.886(2.7);6.867(0.6); 6.865(0.5);5.336(4.1);5.296(3.1);3.898(11.6);3.395(16.0);2.041(0.5);0.000(5.4)

### Example No. B9-35

### ¹H-NMR (400 MHz, CD3CN 6, ppm)

8.639(1.3);8.634(1.3);8.590(0.6);8.580(0.7);8.027(1.3);8.022(1.3);7.877(0.6);7.860(0.7); 7.534(0.6);7.522(0.6);7.514(0.6);7.503(0.5);5.191(5.0);3.429(16.0);2.171(23.9);2.164(20.1);1.965(1.7);1.958(2.7);1.953(11.2);1.946(19.7);1.940(25.6);1.934(17.8);1.928(9.2)

### Example No. B9-36

### ¹H-NMR (400 MHz, CD3CN 6, ppm)

8.936(1.1);8.934(1.0);8.590(0.6);8.588(0.6);8.579(0.7);8.245(1.1);8.242(1.0);7.864(0.7); 7.861(0.6);7.844(0.7);7.842(0.7);7.529(0.6);7.517(0.7);7.509(0.6);7.497(0.6);5.275(4.5); 3.491 (16.0);2.607(0.9);2.423(0.7);2.142(11.6);1.972(0.9);1.964(1.1);1.958(1.7);1.952(6.8); 1.946(12.0);1.940(15.5);1.934(10.7); 1.927(5.4); 1.204(0.4)

### Example No. B9-462

### ¹H-NMR (400 MHz, CDCl₃ δ, ppm)

8.710(0.8);8.707(0.8);8.698(0.8);8.695(0.8);7.925(0.8);7.922(0.8);7.905(0.8);7.902(0.8); 7.498(0.8);7.486(0.8);7.479(0.7);7.467(0.7);7.263(18.7);7.211(0.9);7.190(0.6);6.914(1.2); 6.894(1.6);6.874(1.0);3.904(1.0);3.892(0.7);3.884(0.8);3.874(0.6);3.862(1.0);3.579(16.0); 2.836(0.7);2.817(1.6);2.799(0.8);2.233(0.7);2.222(0.6);2.212(0.7);2.204(0.6);2.192(0.7);0.000(6.4)

### Example No. B13-1

### ¹H-NMR (400 MHz, CDCl₃ δ, ppm)

9.345(1.5);8.876(1.4);8.697(1.7);8.677(1.9);7.892(1.47.519(1.3);7.480(1.4);7.468(2.5); 7.456(3.6);7.445(2.2);7.362(0.6);7.347(4.2);7.338(4.9);7.331(3.1);7.326(3.7);7.312(0.6); 7.295(0.6);7.260(238.0);7.227(1.3);7.210(0.6);6.996(1.3);5.114(6.9);2.595(16.0);1.255(1.2);0.070(6.9);0.008(2.4);0.000(81.8);−0.009(2.4)

### Example No. B13-3

### ¹H-NMR (400 MHz, CDCl₃ δ, ppm)

9.413(1.0);8.908(1.3);8.744(2.0);8.725(2.2);7.944(1.4);7.932(1.4);7.520(0.7);7.408(5.0); 7.387(8.1);7.323(5.8);7.311 (2.0);7.302(3.8);7.261(131.6);6.997(0.7);5.300(4.8);4.954(7.1);2.597(16.0);1.255(0.9);0.008(1.3);0.000(43.9);−0.009(1.3)

### Example No. B14-2

### ¹H-NMR (400 MHz, CDCl₃ δ, ppm)

8.912(1.2);8.910(1.2);8.906(1.48.905(1.1);8.188(1.2);8.183(1.48.168(1.3);8.162(1.2); 7.474(1.2);7.470(0.8);7.449(0.7);7.436(0.6);7.431(1.1);7.426(0.6);7.371(1.1);7.366(2.2); 7.364(1.6);7.347(1.0);7.297(1.4);7.295(1.4);7.277(1.4);7.275(1.4);7.262(25.7);4.919(4.3); 3.406(16.0);2.072(0.6);0.000(9.3)

### Example No. B14-6

### ¹H-NMR (400 MHz, CDCl₃ δ, ppm)

8.921(1.2);8.920(1.3);8.916(1.48.914(1.2);8.196(1.48.190(1.48.176(1.3);8.170(1.3); 7.688(1.5);7.682(1.6);7.426(1.0);7.405(2.3);7.377(1.6);7.371(1.5);7.355(0.7);7.349(0.7); 7.308(1.5);7.306(1.5);7.287(1.4);7.285(1.4);7.262(23.7);5.131(4.6);3.466(16.0);2.070(0.6);2.044(1.3);1.259(0.8);0.000(8.4)

### Example No. B14-40

### ¹H-NMR (400 MHz, CDCl₃ δ, ppm)

8.914(1.1);8.912(1.2);8.908(1.48.906(1.2);8.471(2.48.459(2.1);8.206(1.3);8.200(1.3); 8.185(1.3);8.180(1.3);7.515(2.1);7.503(2.0);7.284(1.5);7.282(1.5);7.263(22.4);5.303(4.6); 3.613(16.0);2.067(0.7);0.000(8.3)

### Example No. B14-61

### ¹H-NMR (400 MHz, CDCl₃ δ, ppm)

8.899(1.2);8.897(1.3);8.893(1.48.891(1.2);8.195(1.48.189(1.48.174(1.3);8.168(1.2); 7.780(0.7);7.777(0.6);7.761 (1.5);7.758(1.5);7.741(0.8);7.739(0.9);7.621(1.0);7.617(0.9); 7.602(0.7);7.598(0.6);7.587(0.8);7.583(0.8);7.568(1.0);7.564(0.9);7.303(1.5);7.302(1.4); 7.283(1.5);7.281(1.4);7.262(30.4);5.221(4.2);3.509(16.0);1.576(0.9);0.000(10.9)

### Example No. B14-679

### ¹H-NMR (400 MHz, CDCl₃ δ, ppm)

8.873(1.2);8.871(1.3);8.867(1.3);8.866(1.2);8.776(1.0);8.772(1.1);8.764(1.1);8.760(1.1); 8.175(1.3);8.169(1.2);8.155(1.4);8.149(1.3);8.086(0.8);8.082(0.9);8.066(0.9);8.062(0.9); 7.543(1.0);7.531(1.0);7.523(1.0);7.511(0.9);7.310(1.5);7.309(1.5);7.290(1.4);7.288(1.4); 7.263(20.0);5.281(3.9);3.540(16.0);2.072(1.0);2.044(0.7);0.000(7.2)

### Example No. B15-462

### ¹H-NMR (400 MHz, CDCl₃ δ, ppm)

8.736(0.9);8.732(0.9);8.724(0.9);8.720(0.9);8.029(0.7);8.027(0.8);8.009(0.8);8.007(0.8); 7.449(0.7);7.437(0.7);7.429(0.7);7.417(0.6);7.261(44.1);7.217(0.9);7.195(1.2);7.058(1.6); 6.921(0.9);6.916(1.2);6.896(1.6);6.876(1.0);3.963(0.9);3.951(0.6);3.942(0.8);3.933(0.6); 3.921(1.0);3.633(16.0);2.846(0.7);2.828(1.5);2.810(0.8);2.237(0.7);2.226(0.6);2.216(0.7);2.208(0.5);2.196(0.7);0.000(14.1)

### Example No. B16-1

### ¹H-NMR (400 MHz, CDCl₃ δ, ppm)

8.337(1.2);8.332(1.2);8.325(1.3);8.320(1.2);7.839(0.7);7.834(0.6);7.820(0.9);7.815(0.8); 7.805(1.3);7.800(1.2);7.787(1.4);7.782(1.3);7.504(0.6);7.500(0.8);7.484(0.8);7.480(1.0); 7.431(0.5);7.417(1.1);7.412(0.9);7.398(0.8);7.393(1.2);7.389(0.9);7.375(1.0);7.371(0.9); 7.261(42.2);7.212(1.5);7.200(1.5);7.193(1.4);7.181(1.4);5.280(4.5);3.442(16.0);2.044(0.9);1.571(1.7);1.259(0.6);0.008(0.5);0.000(18.5)

### Example No. B16-2

### ¹H-NMR (400 MHz, CDCl₃ δ, ppm)

8.330(1.3);8.325(1.3);8.318(1.3);8.313(1.3);7.766(1.3);7.761(1.4);7.747(1.5);7.742(1.4); 7.582(0.7);7.578(1.3);7.574(0.8);7.477(0.5);7.461(0.6);7.457(1.0);7.456(0.8);7.452(1.0); 7.435(0.8);7.431(1.2);7.410(1.3);7.409(1.3);7.391(1.2);7.390(1.2);7.264(16.3);7.210(1.6); 7.198(1.5);7.191(1.5);7.179(1.4);5.033(4.2);3.397(16.0);0.000(7.2)

### Example No. B16-4

### ¹H-NMR (400 MHz, CDCl₃ δ, ppm)

8.341(1.2);8.336(1.3);8.329(1.2);8.324(1.2);7.808(0.9);7.804(1.0);7.794(1.4);7.789(2.3); 7.785(1.0);7.775(1.5);7.770(1.3);7.603(1.0);7.599(1.0);7.583(1.2);7.579(1.2);7.341(1.2); 7.322(1.8);7.302(1.0);7.261(44.6);7.212(1.5);7.200(1.3);7.193(1.3);7.181(1.3);5.334(4.7); 3.452(16.0);2.044(0.7);1.563(1.2);0.008(0.5);0.000(19.7);−0.009(0.6)

### Example No. B16-5

### ¹H-NMR (400 MHz, CDCl₃ δ, ppm)

8.343(1.4);8.338(1.5);8.331(1.5);8.326(1.4);7.830(1.9);7.809(2.1);7.785(1.2);7.780(1.2); 7.766(1.3);7.761(1.3);7.515(2.0);7.509(2.1);7.377(1.3);7.371 (1.2);7.356(1.2);7.350(1.1); 7.266(0.5);7.2653(0.7);7.2645(1.0);7.261(43.4);7.216(1.7);7.204(1.6);7.197(1.6);7.185(1.5);5.230(4.9);3.442(16.0);2.044(1.2);1.259(0.9);0.000(19.1);−0.009(0.5)

### Example No. B16-6

### ¹H-NMR (400 MHz, CDCl₃ δ, ppm)

8.345(1.3);8.340(1.4);8.333(1.4);8.328(1.4);7.878(1.7);7.872(1.7);7.831(1.3);7.826(1.3); 7.813(1.4);7.808(1.3);7.438(0.7);7.437(0.8);7.417(2.7);7.416(2.7);7.403(2.2);7.397(2.1); 7.381 (0.6);7.375(0.7);7.263(19.9);7.230(1.7);7.218(1.6);7.212(1.6);7.200(1.6);5.255(5.1); 3.454(16.0);2.955(0.7);2.884(0.6);2.882(0.6);0.000(9.0)

### Example No. B16-7

### ¹H-NMR (400 MHz, CDCl₃ δ, ppm)

8.337(1.0);8.332(1.0);8.325(1.0);8.320(1.0);7.848(1.1);7.844(1.0);7.830(1.1);7.825(1.1); 7.506(1.9);7.505(1.9);7.485(3.6);7.396(1.4);7.378(1.1);7.375(1.0);7.356(0.7);7.264(14.2); 7.226(1.1);7.214(1.2);7.208(1.1);7.196(1.0);5.596(5.3);3.497(16.0);0.000(5.9)

### Example No. B16-10

### ¹H-NMR (400 MHz, CDCl₃ δ, ppm)

8.338(1.4);8.333(1.4);8.326(1.4);8.321(1.4);7.835(1.3);7.830(1.3);7.816(1.4);7.811(1.4); 7.521(9.2);7.262(34.8);7.224(1.7);7.212(1.9);7.206(1.6);7.193(1.6);5.556(5.6);4.131 (0.5); 4.113(0.5);3.485(16.0);2.044(2.6);1.277(0.8);1.259(1.7);1.241(0.8);0.000(15.2)

### Example No. B16-11

### ¹H-NMR (400 MHz, CDCl₃ δ, ppm)

8.328(2.1);8.323(2.3);8.316(2.3);8.311(2.2);7.771(2.5);7.766(2.5);7.752(2.7);7.747(2.6); 7.724(0.6);7.719(0.6);7.705(1.2);7.700(1.2);7.686(0.6);7.681(0.6);7.489(0.8);7.486(0.6); 7.484(0.6);7.482(0.5);7.475(0.6);7.473(0.6);7.470(0.6);7.468(0.9);7.454(0.5);7.278(1.1); 7.275(1.2);7.268(0.5);7.2674(0.6);7.2666(0.8);7.266(1.0);7.263(40.7);7.260(1.9);7.259(2.2);7.256(1.9);7.240(0.8);7.237(0.8);7.200(3.2);7.197(0.8);7.188(2.8);7.181(2.6);7.178(0.8);7.174(1.1);7.169(2.9);7.153(0.7);7.150(0.6);5.153(4.6);5.151(4.6);3.414(15.8); 3.411(16.0);1.607(1.3);0.000(18.4)

### Example No. B16-17

### ¹H-NMR (400 MHz, CDCl₃ δ, ppm)

8.330(2.3);8.325(2.4);8.318(2.5);8.313(2.4);7.835(2.5);7.830(2.5);7.816(2.8);7.811(2.6); 7.525(0.8);7.520(0.7);7.504(1.6);7.488(0.7);7.483(0.9);7.263(45.2);7.221(2.9);7.209(2.8); 7.202(2.8);7.190(2.6);7.090(2.3);7.071(3.2);7.050(1.9);5.213(5.1);3.440(16.0);2.956(0.6);2.884(0.6);2.883(0.5);1.621(1.9);0.008(0.6);0.000(19.4);−0.009(0.5)

### Example No. B16-21

### ¹H-NMR (400 MHz, CDCl₃ δ, ppm)

8.668(2.0);8.656(2.4);8.653(1.7);8.321(1.9);8.317(2.1);8.309(2.1);8.304(2.0);8.020(1.7); 7.812(0.6);7.797(2.2);7.792(2.2);7.780(5.5);7.776(5.4);7.761(0.8);7.671(1.3);7.654(1.3); 7.519(1.7);7.398(1.4);7.393(1.3);7.386(1.3);7.380(1.9);7.376(1.4);7.370(1.3);7.364(1.2); 7.261(303.9);7.211(3.4);7.178(2.0);7.166(2.1);7.160(2.1);7.148(1.9);6.997(1.8);5.188(8.5);4.145(1.5);3.537(15.6);2.957(16.0);2.885(13.5);2.884(13.1);2.578(0.6);2.084(1.4); 1.658(1.2);1.255(0.9);0.008(3.8);0.000(129.4);−0.008(5.0);−0.050(1.6)

### Example No. B16-22

### ¹H-NMR (400 MHz, CDCl₃ δ, ppm)

8.846(5.0);8.833(5.1);7.815(1.0);7.794(1.1);7.672(1.1);7.668(1.1);7.653(1.5);7.649(1.5); 7.561(0.7);7.558(0.7);7.542(1.4);7.539(1.4);7.524(0.7);7.520(0.9);7.410(0.7);7.406(0.7); 7.389(1.1);7.380(1.3);7.368(2.8);7.356(1.2);7.261(48.6);7.211(0.7);5.276(6.0);3.588(16.0); 1.582(1.1);0.008(0.6);0.000(19.6);−0.009(0.7)

### Example No. B16-35

### ¹H-NMR (400 MHz, CDCl₃ δ, ppm)

8.586(2.0);8.580(2.0);8.326(1.2);8.321(1.2);8.314(1.2);8.309(1.2);7.868(2.1);7.862(2.0); 7.810(1.2);7.805(1.2);7.791(1.3);7.786(1.3);7.270(0.5);7.269(0.6);7.2684(0.7);7.2676(0.7);7.266(1.1);7.261(56.0);7.212(1.6);7.200(1.4);7.193(1.3);7.181(1.3);5.373(4.9);3.555(16.0);2.060(0.8);0.008(0.7);0.000(19.7);−0.009(0.6)

### Example No. B16-40

### ¹H-NMR (400 MHz, CDCl₃ δ, ppm)

8.501(3.3);8.488(3.3);8.330(1.5);8.325(1.6);8.318(1.6);8.313(1.6);7.822(1.0);7.817(1.0); 7.803(1.1);7.798(1.0);7.527(3.2);7.519(0.6);7.514(3.1);7.2694(0.6);7.2687(0.6);7.268(0.7);7.267(0.9);7.266(1.0);7.265(1.3);7.261(100.2);7.256(0.8);7.255(0.6);7.216(1.9);7.211(0.7);7.204(1.9);7.198(1.8);7.185(1.7);6.997(0.6);5.471(6.5);3.599(16.0);2.044(1.8);1.565(0.7);1.277(0.6);1.259(1.2);1.241(0.6);0.008(1.2);0.000(43.1);−0.009(1.2)

### Example No. B16-62

### ¹H-NMR (400 MHz, CDCl₃ δ, ppm)

8.340(1.2);8.335(1.3);8.328(1.3);8.323(1.3);7.887(0.6);7.884(0.6);7.868(0.9);7.864(1.3); 7.859(1.3);7.789(0.6);7.786(0.8);7.782(0.5);7.770(0.7);7.766(0.9);7.763(0.6);7.738(1.3); 7.733(1.3);7.720(1.4);7.715(1.4);7.608(0.7);7.607(0.7);7.589(1.2);7.588(1.2);7.570(0.5); 7.568(0.5);7.266(0.7);7.262(29.1);7.219(1.5);7.206(1.4);7.200(1.4);7.188(1.3);5.081(4.1);3.427(16.0);2.956(0.8);2.884(0.7);2.883(0.6);2.044(0.6);1.600(0.9);0.000(12.8)

### Example No. B16-63

### ¹H-NMR (400 MHz, CDCl₃ δ, ppm)

8.341(1.2);8.336(1.3);8.329(1.3);8.324(1.2);7.764(1.47.762(0.7);7.748(0.8);7.743(3.8); 7.727(3.5);7.721(0.7);7.718(1.4);7.713(1.5);7.707(0.7);7.705(1.0);7.699(1.5);7.694(1.4); 7.264(18.3);7.210(1.5);7.198(1.5);7.191(1.4);7.179(1.4);5.101(4.3);3.423(16.0);1.620(0.6);0.000(8.3)

### Example No. B16-81

### ¹H-NMR (400 MHz, CDCl₃ δ, ppm)

8.364(2.1);8.359(2.1);8.352(2.2);8.347(2.1);8.251(1.5);8.231(1.6);7.845(1.0);7.841(1.0); 7.826(1.1);7.822(1.0);7.780(1.4);7.763(1.7);7.673(0.7);7.656(1.5);7.637(1.0);7.595(1.1); 7.575(1.5);7.557(0.6);7.519(0.9);7.2734(0.5);7.2726(0.6);7.272(0.7);7.271(0.8);7.270(0.9);7.2694(1.0);7.2686(1.2);7.268(1.3);7.267(1.5);7.266(1.8);7.2654(2.2);7.2646(2.7); 7.264(3.8);7.260(155.9);7.257(2.4);7.2564(1.7);7.2556(1.2);7.255(1.0);7.254(0.7);7.253(0.6);7.252(0.5);7.251(0.5);7.250(0.6);7.247(2.7);7.235(2.5);7.228(2.4);7.216(2.3);7.211(1.3);6.996(0.9);5.281(4.7);3.427(16.0);2.044(1.5);1.557(0.7);1.259(1.1);1.256(0.8);0.008(2.2);0.000(71.6);−0.009(1.8);−0.050(0.6)

### Example No. B16-462

### ¹H-NMR (400 MHz, CDCl₃ δ, ppm)

8.440(1.3);8.435(1.3);8.428(1.3);8.423(1.3);7.802(1.3);7.797(1.3);7.783(1.4);7.778(1.4); 7.273(1.7);7.263(23.5);7.257(0.5);7.255(1.5);7.242(1.5);7.220(0.9);7.199(0.6);6.919(1.2); 6.899(1.6);6.879(1.0);4.023 (0.9);4.011(0.6);4.002(0.8);3.993(0.6);3.981(1.0);3.669(16.0); 2.855(0.7);2.837(1.5);2.819(0.8);2.278(0.7);2. 266(0.6);2.255(0.7);2.248(0.6);2.236(0.7);0.000(8.4)

### Example No. B16-603

### ¹H-NMR (400 MHz, CDCl₃ δ, ppm)

8.327(1.2);8.322(1.3);8.315(1.3);8.310(1.3);8.042(0.8);8.038(0.8);8.022(0.9);8.019(0.8); 7.904(0.7);7.901(0.8);7.885(0.8);7.882(0.8);7.772(1.3);7.767(1.3);7.754(1.4);7.749(1.3); 7.610(1.0);7.606(1.0);7.591(0.7);7.587(0.6);7.541(0.7);7.538(0.7);7.522(1.0);7.519(0.9); 7.262(31.3);7.203(1.5);7.191(1.4);7.184(1.4);7.172(1.4);5.578(4.5);3.878(16.0);3.421(15.5);1.599(0.8);0.000(13.3)

### Example No. B16-679

### ¹H-NMR (400 MHz, CDCl₃ δ, ppm)

8.801(1.2);8.797(1.2);8.789(1.2);8.785(1.2);8.485(0.9);8.481(0.9);8.464(1.0);8.460(0.9); 8.358(1.3);8.353(1.3);8.346(1.3);8.341(1.3);7.780(1.3);7.775(1.3);7.761(1.4);7.756(1.3); 7.645(1.1);7.633(1.1);7.624(1.1);7.613(1.0);7.268(0.5);7.267(0.6);7.266(0.7);7.265(0.9); 7.261(61.3);7.258(0.9);7.257(0.6);7.220(1.6);7.208(1.6);7.201(1.5);7.189(1.5);5.305(4.1);3.547(16.0);1.570(0.9);0.008(0.8);0.003(0.5);0.002(0.9);0.000(24.4);−0.008(0.7)

### Example No. C1-2

### ¹H-NMR (400 MHz, d₆-DMSO δ, ppm)

8.534(9.3);8.530(5.5);8.523(5.7);8.519(9.6);7.671(12.8);7.667(7.0);7.660(6.8);7.656(12.2); 7.593(3.4);7.589(5.7);7.585(3.9);7.505(1.7);7.501(2.4);7.495(1.7);7.486(3.5);7.482(6.6);7.477(4.3);7.467(4.0);7.463(5.4);7.459(2.2);7.444(6.2);7.425(5.2);7.406(1.7);5.756(3.7);4.939(16.0);3.332(65.5);2.526(0.7);2.521(1.1);2.512(14.3);2.508(30.4);2.503(41.9); 2.498(29.4);2.494(13.2);0.000(0.9)

### Example No. C1-3

### ¹H-NMR (400 MHz, d₆-DMSO δ, ppm)

8.534(13.1);8.530(6.9);8.523(7.2);8.519(13.7);7.680(15.5);7.676(8.0);7.670(7.8);7.665(14.9);7.537(0.8);7.532(6.1);7.526(2.3);7.515(3.3);7.510(14.1);7.505(2.2);7.481(2.4);7.476(16.0);7.471(3.4);7.460(2.5);7.454(7.0);7.449(0.7);5.758(0.7);4.929(15.6);3.348(4.5); 3.323(54.4);2.522(0.7);2.513(9.5);2.509(20.3);2.504(28.1);2.500(19.4);2.495(8.4);0.000(0.5)

### Example No. C1-12

### ¹H-NMR (400 MHz, d₆-DMSO δ, ppm)

8.540(7.5);8.536(4.9);8.529(4.9);8.525(7.9);7.686(13.5);7.682(7.3);7.675(7.1);7.671(13.4); 7.476(1.4);7.460(1.6);7.456(3.4);7.441(2.7);7.435(2.7);7.421(2.3);7.364(6.4);7.357(3.0);7.353(2.1);7.348(2.0);7.344(3.1);7.341(2.2);7.338(2.2);7.331(2.3);7.328(1.7);7.294(1.4);7.291(1.4);7.288(1.2);7.285(1.47.271(2.4);7.269(2.1);7.264(2.47.251(1.2);7.248(1.2);7.244(1.0);7.242(0.9);5.760(0.9);4.960(16.0);3.339(58.6);3.290(0.6);2.515(5.7);2.511(12.5);2.506(17.6);2.501(12.3);2.497(5.5)

### Example No. C1-13

### ¹H-NMR (400 MHz, d₆-DMSO δ, ppm)

8.531(13.6);8.527(7.3);8.520(7.3);8.516(14.1);7.682(16.0);7.678(8.3);7.671(8.1);7.667(15.6);7.568(4.5);7.563(1.8);7.554(5.1);7.546(5.4);7.538(2.0);7.532(4.9);7.525(0.5);7.261(0.6);7.253(5.8);7.248(1.7);7.237(1.9);7.231(10.4);7.226(1.9);7.214(1.6);7.209(5.1);5.759(13.1);4.924(15.9);3.338(18.0);3.316(58.1);2.514(7.4);2.510(16.2);2.505(22.7);2.500(15.8);2.496(7.0);0.000(0.6)

### Example No. C1-21

### ¹H-NMR (400 MHz, d₆-DMSO δ, ppm)

8.648(2.3);8.646(2.6);8.644(2.7);8.642(2.4);8.636(2.4);8.634(2.7);8.632(2.6);8.630(2.3); 8.506(9.7);8.502(5.3);8.495(5.4);8.491(10.0);7.827(1.9);7.823(1.9);7.808(3.9);7.803(3.9); 7.789(2.4);7.784(2.3);7.633(11.7);7.629(6.2);7.622(6.1);7.618(11.3);7.574(2.5);7.571(4.3);7.569(2.7);7.555(2.3);7.552(3.7);7.550(2.2);7.438(2.3);7.435(2.3);7.426(2.3);7.423(2.4);7.419(2.3);7.416(2.1);7.407(2.2);7.404(2.0);5.025(16.0);3.414(51.6);3.362(8.7); 3.303(0.9);3.284(0.6);3.170(0.6);2.592(1.8);2.527(0.6);2.523(0.8);2.514(11.1);2.510(23.5); 2.505(31.8);2.500(22.1);2.496(9.9);0.000(0.9)

### Example No. C1-22

### ¹H-NMR (400 MHz, d₆-DMSO δ, ppm)

8.666(4.8);8.662(4.8);8.661(4.4);8.596(4.4);8.592(4.8);8.584(4.8);8.580(4.6);8.534(12.8); 8.530(6.9);8.523(6.9);8.519(13.4);7.916(1.9);7.911(2.9);7.906(2.0);7.896(2.2);7.891(3.1);7.886(2.0);7.676(16.0);7.672(8.4);7.665(8.0);7.661(15.3);7.449(2.7);7.447(2.8);7.436(2.7);7.434(2.7);7.429(2.6);7.427(2.6);7.417(2.5);7.415(2.5);5.758(2.7);4.969(16.0);3.354(59.1);3.333(20.3);3.314(1.8);2.594(0.9);2.526(0.5);2.521(0.8);2.513(11.7);2.508(25.4); 2.503(35.4);2.499(24.5);2.494(10.7);0.000(0.8)

### Example No. C1-30

### ¹H-NMR (400 MHz, d₆-DMSO δ, ppm)

8.503(1.8);8.499(1.1);8.492(1.1);8.488(1.9);7.638(2.2);7.634(1.2);7.627(1.3);7.623(2.1); 6.277(3.1);4.969(3.3);3.858(16.0);3.545(10.5);3.332(4.7);3.178(1.5);3.166(1.6);2.513(1.7);2.509(3.6);2.504(4.8);2.499(3.4);2.495(1.6)

### Example No. C1-37

### ¹H-NMR (400 MHz, d₆-DMSO δ, ppm)

8.502(3.2);8.498(1.8);8.491(1.9);8.487(3.4);8.319(2.4);7.608(3.6);7.604(2.0);7.597(1.9); 7.593(3.5);4.861(4.8);3.761(0.7);3.747(15.7);3.483(16.0);3.342(7.0);3.170(0.8);2.512(4.7);2.508(10.0);2.503(13.6);2.498(9.6);2.494(4.3);2.439(9.3);2.245(7.8)

### Example No. C1-62

### ¹H-NMR (400 MHz, d₆-DMSO δ, ppm)

8.537(9.6);8.533(5.9);8.526(5.5);8.522(10.4);7.956(2.8);7.952(5.2);7.949(3.4);7.911(2.1); 7.908(3.1);7.904(2.0);7.892(2.4);7.889(3.4);7.885(2.3);7.852(1.8);7.848(2.5);7.845(1.7); 7.832(2.3);7.828(3.0);7.825(1.9);7.685(1.2);7.681(0.7);7.671(13.7);7.667(6.7);7.660(6.6);7.656(12.6);7.636(3.1);7.616(5.3);7.597(2.3);5.758(16.0);4.997(1.4);4.982(13.2);3.813(4.0);3.349(50.2);2.515(7.6);2.510(16.5);2.506(23.0);2.501(16.1);2.496(7.1)

### Example No. C1-63

### ¹H-NMR (400 MHz, d₆-DMSO δ, ppm)

8.532(10.0);8.529(6.4);8.521(6.2);8.518(10.4);7.971(0.7);7.950(0.8);7.891(9.5);7.887(3.4); 7.874(3.8);7.870(12.0);7.707(10.8);7.686(8.7);7.670(14 .8);7.666(8.3);7.659(7.9);7.655(14.0);5.757(9.5);5.018(16.0);3.848(2.9);3.349(58.2);3.331(85.5);3.307(3.9);2.526(0.9); 2.521(1.4);2.513(18.7);2.508(39.5);2.503(54.2);2.499(38.1);2.494(17.3);0.000(0.8)

### Example No. C1-108

### ¹H-NMR (400 MHz, d₆-DMSO δ, ppm)

8.522(13.2);8.518(7.1);8.511(7.2);8.507(14.0);7.671(15.1);7.667(7.9);7.660(7.6);7.656(14.5);7.547(6.9);7.543(4.2);7.533(10.3);7.524(1.0);7.507(3.7);7.505(3.9);7.437(1.4);7.435(1.5);7.431(1.3);7.428(1.6);7.426(1.9);7.424(1.9);7.418(1.5);7.416(1.3);7.408(0.9);7.405(0.9);5.759(11.1);4.998(16.0);3.348(54.5);3.336(34.5);3.313(1.9);2.523(0.6);2.514(10.0);2.510(21.7);2.505(30.5);2.500(21.2);2.496(9.3);0.000(0.6)

### Example No. C1-111

### ¹H-NMR (400 MHz, d₆-DMSO δ, ppm)

8.531(8.2);8.527(4.9);8.520(4.9);8.516(8.5);8.458(3.2);8.453(4.7);8.448(3.4);8.291(2.3); 8.289(2.6);8.285(2.3);8.283(2.3);8.271(2.6);8.268(2.6);8.265(2.6);8.262(2.4);7.961(1.9); 7.958(2.7);7.955(2.0);7.942(2.3);7.939(3.2);7.936(2.2);7.724(3.8);7.720(0.6);7.704(5.5); 7.692(0.8);7.684(16.0);7.680(7.1);7.673(6.8);7.669(12.8);5.760(3.7);5.068(13.8);3.377(48.6);3.339(8.8);2.524(0.5);2.516(7.6);2.511(16.7);2.506(23.3);2.502(16.2);2.497(7.2)

### Example No. C1-112

### ¹H-NMR (400 MHz, d₆-DMSO δ, ppm)

8.535(12.7);8.531(6.9);8.524(7.1);8.520(13.4);8.315(0.7);8.293(0.9);8.271(1.3);8.265(11.5); 8.260(3.3);8.248(3.5);8.243(12.7);8.237(1.3);8.166(0.8);8.143(0.6);7.812(1.5);7.806(10.9);7.801(3.3);7.789(3.2);7.784(9.9);7.778(1.1);7.680(16.0);7.676(8.4);7.670(8.2);7.666(15.4);5.757(4.0);5.068(15.8);3.375(54.8);3.332(9.7);2.527(0.8);2.522(1.1);2.513(15.6); 2.509(33.7);2.504(46.9);2.500(32.5);2.495(14.3);0.000(1.0)

### Example No. C2-35

### ¹H-NMR (400 MHz, CD3CN 6, ppm)

8.808(1.5);8.706(0.9);8.694(0.9);8.638(1.5);8.633(1.5);8.028(1.5);8.022(1.4);7.425(0.9); 7.413(0.9);5.447(0.9);5.179(4.9);4.068(0.6);4.050(0.6);3.431(16.0);2.149(3.2);1.972(2.9); 1.964(0.7);1.958(1.0);1.952(4.5);1.946(7.9);1.940(10.3);1.934(7.1);1.928(3.7);1.221(0.8);1.204(1.6);1.186(0.8);0.008(0.4);0.000(9.9);−0.009(0.4)

### Example No. C2-36

### ¹H-NMR (400 MHz, CD3CN 6, ppm)

8.936(1.0);8.934(1.0);8.804(1.2);8.703(0.7);8.691(0.7);8.248(1.0);8.245(1.0);7.409(0.9); 7.397(0.9);5.265(4.3);4.068(0.4);4.050(0.4);3.493(16.0);2.607(0.5);2.433(0.4);2.134(16.8); 1.972(2.1);1.964(2.5);1.958(4.2);1.952(17.8);1.946(31.3);1.940(41.2);1.933(28.7); 1.927(15.0);1.222(0.5);1.204(1.0);1.186(0.5);0.008(1.6);0.000(36.1);-0.009(1.7)

### Example No. D1-1

### ¹H-NMR (400 MHz, d₆-DMSO δ, ppm)

8.497(3.1);8.486(3.4);8.319(0.8);7.732(4.4);7.728(7.4);7.719(8.5);7.716(9.0);7.710(4.3); 7.695(4.0);7.691(3.9);7.583(4.0);7.580(4.3);7.563(5.8);7.560(5.7);7.479(2.6);7.475(2.7); 7.460(4.0);7.456(3.9);7.440(2.7);7.436(2.5);7.368(2.9);7.365(2.9);7.349(4.2);7.346(4.2); 7.330(3.5);7.328(2.9);7.318(3.2);7.308(3.7);7.296(1.9);5.154(16.0);4.889(1.5);3.733(5.8);3.450(5.6);3.353(59.5);3.300(1.4);3.252(0.5);3.238(0.6);3.169(2.5);2.905(0.7);2.572(0.6);2.526(0.8);2.522(1.1);2.513(17.5);2.508(37.3);2.504(50.5);2.499(35.0);2.495(15.7); 2.442(2.8);2.241(2.7);0.000(1.8)

### Example No. D1-2

### ¹H-NMR (400 MHz, d₆-DMSO δ, ppm)

8.490(3.0);8.479(3.1);7.748(1.4);7.744(1.4);7.729(3.4);7.725(3.5);7.710(3.1);7.706(3.2); 7.684(4.7);7.665(2.3);7.624 (6.5);7.620(4.3);7.501(3.2);7.497(5.5);7.493(3.6);7.480(9.1); 7.476(8.5);7.438(6.1);7.422(3.6);7.416(2.7);7.400(2.2);7.327(2.1);7.324(2.2);7.315(2.3); 7.311(2.7);7.310(2.7);7.306(2.1);7.297(2.0);7.294 (1.9);5.005(16.0);4.098(0.7);3.339(7.7);3.309(55.9);3.169(6.5);2.525(0.6);2.512(13.8);2.507(28.9);2.503(39.4);2.498(27.9); 2.494(12.9);0.000(0.7)

### Example No. D1-4

### ¹H-NMR (400 MHz, d₆-DMSO δ, ppm)

8.747(1.1);8.745(1.2);8.734(1.2);8.732(1.1);8.324(0.6);8.320(0.6);8.305(1.3);8.301(1.2); 8.285(0.9);8.281(0.9);8.216(1.7);8.197(1.2);7.897(0.8);7.894 (0.8);7.884(0.8);7.881(0.9); 7.878(0.9);7.875(0.8);7.865(0.8);7.862(0.7);7.837(1.3);7.833(1.5);7.817(1.6);7.813(1.5); 7.654(1.2);7.650(1.3);7.635(1.5);7.631(1.4);7.481 (1.5);7.461(2.3);7.442(1.1);5.317(4.9); 3.707(13.6);2.512(7.1);2.508(16.2);2.503(23.1);2.498(16.7);2.494(7.9);2.075(16.0); 0.000(12.2)

### Example No. D1-5

### ¹H-NMR (400 MHz, d₆-DMSO δ, ppm)

8.496(3.2);8.484(3.6);7.757(9.1);7.751(9.8);7.733(3.8);7.726(7.8);7.717(8.2);7.713(8.4); 7.705(8.3);7.481(4.9);7.476(4.7);7.460(4.3);7.455(4.1);7.333(2.0);7.327(2.0);7.321(2.2); 7.316(2.7);7.311(2.1);7.305(1.8);7.299(1.7);5.123(16.0);3.355(70.7);3.346(249.4);3.323 (7.7);3.292(0.6);3.174(2.6);3.162(2.4);2.677(0.6);2.672(0.8);2.668(0.5);2.526(2.0);2.521(3.2);2.512(44.5);2.508(92.0);2.503(122.5);2.499(85.6);2.494(38.7);2.335(0.5);2.330(0.7);2.325(0.5);0.000(5.4)

### Example No. D1-7

### ¹H-NMR (400 MHz, d₆-DMSO δ, ppm)

8.759(1.4);8.748(1.5);8.320(0.6);8.316(0.6);8.300(1.4);8.297(1.4);8.281(1.0);8.277(0.9); 8.197(2.1);8.177(1.5);7.894(0.9);7.892(0.9);7.882(0.9);7.879(1.1);7.876(1.1);7.873(0.9); 7.863(0.8);7.860(0.8);7.672(2.0);7.668(2.4);7.650(5.7);7.611(2.5);7.594(1.6);7.588(1.2); 7.571(0.8);5.389(6.4);3.786(16.0);3.718(0.5);2.675(1.2);2.512(8.7);2.507(20.0);2.503(28.7);2.498(21.1);2.494(10.3);2.075(15.9);0.000(14.3);−0.009(0.6)

### Example No. D1-12

### ¹H-NMR (400 MHz, d₆-DMSO δ, ppm)

8.489(2.1);8.479(2.1);8.146(1.1);7.743(1.2);7.739(1.2);7.723(3.2);7.719(3.2);7.706(3.3); 7.701(3.5);7.690(3.7);7.671(1.4);7.468(1.4);7.448(2.9);7.433(2.9);7.428(3.1);7.412(2.5); 7.405(1.7);7.401(2.2);7.395(2.2);7.377(6.4);7.373(6.7);7.356(2.7);7.320(1.7);7.317(1.7); 7.309(1.8);7.304(2.4);7.299(1.8);7.287(2.9);7.281(1.6);7.278(1.4);7.265(2.7);7.260(3.1); 7.245(1.3);7.242(1.3);7.238(1.2);7.235(1.0);5.758(9.6);5.019(16.0);3.332(8.0);3.309(58.2);2.576(5.0);2.526(0.5);2.521(0.8);2.512(12.2);2.508(26.7);2.503(37.5);2.498(26.0); 2.494(11.5);0.000(0.9)

### Example No. D1-13

### ¹H-NMR (400 MHz, d₆-DMSO δ, ppm)

8.489(3.1);8.478(3.3);8.150(0.8);7.744(1.1);7.740(1.1);7.724(3.3);7.720(3.3);7.707(4.1); 7.702(5.9);7.697(5.2);7.679(1.6);7.588(4.2);7.573(5.2);7.566(5.4);7.552(4.6);7.325(2.1); 7.320(2.1);7.313(2.2);7.308(3.4);7.303(2.2);7.296(1.9);7.291(1.8);7.246(5.4);7.224(9.8); 7.201(4.8);4.991(15.6);3.366(2.4);3.294(54.4);3.172(16.0);2.513(7.6);2.509(15.6);2.504(21.0);2.500(15.1);2.495(7.1)

### Example No. D1-21

### ¹H-NMR (400 MHz, d₆-DMSO δ, ppm)

8.646(3.2);8.636(3.2);8.483(2.9);8.473(3.0);7.832(1.7);7.828(1.7);7.813(3.7);7.808(3.6); 7.794(2.2);7.789(2.0);7.743(1.2);7.727(2.7);7.709(2.1);7.666(3.9);7.647(2.2);7.611(4.4); 7.592(3.7);7.447(2.5);7.444(2.5);7.432(2.6);7.428(2.4);7.416(2.2);7.413(2.1);7.339(1.8); 7.323(2.1);7.309(1.5);5.096(16.0);3.740(1.6);3.508(1.2);3.413(46.0);3.323(109.5);3.006(0.8);2.924(0.8);2.670(1.9);2.540(1.5);2.524(5.1);2.510(110.9);2.506(233.0);2.501(317.3);2.497(223.5);2.492(101.5);2.429(1.2);2.332(1.4);2.328(1.9);2.246(1.0);0.008(1.3); 0.000(39.9);-0.009(1.3)

### Example No. D1-22

### ¹H-NMR (400 MHz, d₆-DMSO δ, ppm)

8.674(5.3);8.670(5.3);8.591(4.8);8.587(5.2);8.579(5.2);8.575(5.1);8.487(2.9);8.476(2.9); 7.940(2.0);7.935(3.1);7.930(2.0);7.920(2.2);7.915(3.3);7.911(2.0);7.741(1.3);7.736(1.3); 7.721(3.4);7.717(3.3);7.703(3.5);7.699(3.6);7.686(4.7);7.667(1.8);7.438(2.9);7.437(2.9); 7.426(3.0);7.425(3.0);7.419(2.9);7.417(2.8);7.407(2.7);7.405(2.7);7.322(2.1);7.318(2.1); 7.310(2.2);7.305(2.8);7.300(2.1);7.292(2.0);7.288(1.9);5.038(16.0);3.400(0.7);3.322(65.0);3.168(4.1);2.573(0.7);2.525(1.1);2.520(1.6);2.511(24.4);2.507(52.7);2.502(73.2); 2.498(51.3);2.493(22.9);0.000(1.2)

### Example No. D1-30

### ¹H-NMR (400 MHz, d₆-DMSO δ, ppm)

8.467(0.7);8.459(0.6);8.456(0.8);8.319(0.7);7.700(0.7);7.696(0.8);7.683(0.8);7.678(1.6); 7.674(1.2);7.671(0.9);7.662(0.6);7.659(0.7);7.294(0.5);7.289(0.8);7.284(0.6);7.277(0.5); 6.289(3.6);5.008(3.3);4.889(1.3);4.062(0.9);3.955(0.7);3.951(0.5);3.947(1.9);3.889(1.4); 3.884(0.9);3.881(0.8);3.872(16.0);3.850(1.0);3.835(0.8);3.734(4.5);3.518(11.0);3.450(4.7);3.359(7.9);2.977(0.6);2.813(0.6);2.522(0.6);2.513(8.1);2.509(17.0);2.504(23.2);2.500(16.5);2.495(7.6);2.442(2.5);2.241(2.4);2.236(1.7);0.000(0.9)

### Example No. D1-37

### ¹H-NMR (400 MHz, d₆-DMSO δ, ppm)

8.458(0.9);8.450(0.8);8.448(1.0);8.319(2.6);7.693(1.0);7.689(1.0);7.675(1.1);7.671(1.1); 7.662(1.1);7.659(1.5);7.657(1.2);7.643(0.5);7.641(0.6);7.295(0.7);7.291(0.7);7.283(0.7); 7.279(0.9);7.273(0.7);7.266(0.7);7.261(0.6);4.889(5.2);3.733(16.0);3.449(17.0);3.372(5.2);3.168(0.6);2.512(6.5);2.508(13.8);2.503(18.7);2.499(13.2);2.494(6.1);2.443(9.9);2.241(8.8);0.000(0.8)

### Example No. D1-59

### ¹H-NMR (400 MHz, d₆-DMSO δ, ppm)

8.741(1.5);8.739(1.6);8.737(1.3);8.730(1.4);8.728(1.6);8.726(1.5);8.724(1.2);8.290(0.8); 8.286(0.8);8.271(1.7);8.267(1.7);8.252(1.3);8.248(1.2);8.191(2.3);8.174(1.1);8.172(1.5); 8.008(0.7);8.005(0.6);7.869(1.1);7.866(1.2);7.857(1.1);7.854(1.2);7.851(1.2);7.847(1.0); 7.838(1.1);7.835(1.0);7.673(0.7);7.657(0.8);7.652(1.5);7.637(1.6);7.631(1.1);7.616(1.0); 7.528(2.3);7.507(1.7);7.407(1.0);7.404(1.0);7.382(1.5);7.360(0.8);7.358(0.8);5.289(4.9);3.766(16.0);3.007(1.0);2.925(1.0);2.627(5.6);2.525(0.7);2.511(15.1);2.507(32.2);2.502(44.4);2.498(31.3);2.493(14.2);2.075(15.1);0.008(0.7);0.000(22.5);-0.009(0.8)

### Example No. D1-61

### ¹H-NMR (400 MHz, d₆-DMSO δ, ppm)

8.488(3.3);8.485(2.5);8.476(3.2);7.965(3.6);7.962(3.7);7.946(4.2);7.943(4.1);7.829(3.0); 7.811(4.4);7.810(4.3);7.749(3.6);7.746(2.8);7.734(4.5);7.730(8.9);7.726(8.9);7.721(3.9); 7.708(5.1);7.704(3.8);7.688(1.4);7.684(1.4);7.661(2.9);7.658(3.0);7.642(4.3);7.639(4.3); 7.623(1.8);7.620(1.7);7.318(2.2);7.314(2.3);7.306(2.3);7.302(3.8);7.297(2.2);7.289(2.1); 7.285(2.0);5.091(16.0);3.424(50.7);3.354(8.4);3.169(8.1);2.521(0.8);2.513(13.4);2.508(28.2);2.504(37.9);2.499(26.7);2.495(12.2);0.000(1.8)

### Example No. D1-62

### ¹H-NMR (400 MHz, d₆-DMSO δ, ppm)

8.493(2.4);8.483(2.4);8.008(5.4);7.900(3.2);7.897(2.2);7.881(3.6);7.862(2.7);7.842(3.0); 7.746(1.2);7.742(1.2);7.727(2.9);7.722(2.8);7.708(2.3);7.704(2.3);7.668(3.6);7.649(2.0); 7.624(3.1);7.604(5.2);7.585(2.4);7.331(1.7);7.328(1.7);7.319(1.9);7.316(2.1);7.313(2.1); 7.301(1.6);7.298(1.4);5.758(16.0);5.046(12.3);3.338(8.3);3.320(43.9);2.576(1.3);2.512(10.1);2.508(20.3);2.503(27.1);2.499(19.4);2.494 (9.1)

### Example No. D1-63

### ¹H-NMR (400 MHz, d₆-DMSO δ, ppm)

8.486(3.5);8.476(3.6);7.910(0.9);7.883(9.4);7.862(12.4);7.809(0.9);7.788(1.0);7.733(12.0); 7.713(9.9);7.700(3.7);7.696(3.5);7.678(5.4);7.662(2.6);7.659(2.7);7.643(0.8);7.388(0.8);7.367(0.7);7.321(2.3);7.318(2.3);7.310(2.4);7.305(3.0);7.300(2.3);7.292(2.1);7.288(2.0);6.174(1.1);5.757(13.6);5.087(16.0);4.835(1.1);3.372(8.1);3.320(54.2);3.225(3.8);2.575(3.0);2.525(1.1);2.512(20.1);2.507(41.5);2.503(56.2);2.498(40.1);2.494(18.7);0.000(0.9)

### Example No. D1-86

### ¹H-NMR (400 MHz, d₆-DMSO δ, ppm)

8.745(1.1);8.744(1.1);8.732(1.1);8.350(0.5);8.346(0.6);8.330(1.2);8.326(1.2);8.311(0.8); 8.307(0.8);8.195(1.6);8.176(1.2);7.953(1.8);7.926(0.7);7.917(0.9);7.914(0.9);7.904(2.1); 7.900(2.0);7.884(0.8);7.881(0.8);7.871(2.1);7.850(0.9);5.311(4.6);3.714(13.0);2.513(6.7); 2.508(15.3);2.503(21.8);2.499(15.8);2.494(7.5);2.075(16.0);0.000(11.6)

### Example No. D1-108

### ¹H-NMR (400 MHz, d₆-DMSO δ, ppm)

8.477(3.2);8.465(3.4);8.145(0.8);7.728(1.1);7.724(1.1);7.709(3.6);7.704(3.6);7.692(7.6); 7.688(8.3);7.672(1.3);7.670(1.3);7.552(8.0);7.547(11.3);7.528(7.2);7.508(2.5);7.425(2.8); 7.408(2.1);7.316(2.1);7.311 (2.2);7.304(2.3);7.299(3.7);7.295(2.2);7.288(2.0);7.283(1.9); 5.052(16.0);3.337(18.9);3.323(58.2);3.175(3.0);3.169(3.2);2.526(0.5);2.522(0.9);2.513(11.5);2.508(24.3);2.504(33.5);2.499(23.6);2.495(10.8);0.000(0.6)

### Example No. D1-111

### ¹H-NMR (400 MHz, d₆-DMSO δ, ppm)

8.481(2.5);8.477(3.2);8.475(2.7);8.469(2.9);8.466(4.48.462(5.2);8.457(5.4);8.452(3.8); 8.287(2.3);8.284(2.7);8.281(2.4);8.278(2.3);8.266(2.6);8.263(2.7);8.260(2.7);8.258(2.3); 7.979(3.0);7.960(3.4);7.739(0.6);7.734(1.1);7.729(1.1);7.713(5.8);7.710(4.0);7.693(12.8); 7.673(3.9);7.317(2.1);7.312(2.1);7.306(2.2);7.300(3.7);7.296(2.0);7.289(1.9);7.284(1.8);6.216(1.0);5.757(16.0);5.135(14.1);4.947(1.1);3.378(6.7);3.344(49.2);3.327(5.1);2.526(0.7);2.512(15.3);2.508(31.8);2.503(43.0);2.499(30.5);2.494(14.0);0.000(0.5)

### Example No. D1-112

### ¹H-NMR (400 MHz, d₆-DMSO δ, ppm)

8.495(3.1);8.484(3.1);8.333(1.3);8.314(6.3);8.297(2.8);8.292(7.5);8.286(2.1);8.251(10.7); 8.229(11.6);8.179(1.4);8.157(1.1);8.142(0.7);8.108(6.9);8.103(2.3);8.091(2.2);8.086(5.5);7.828(9.9);7.806(9.0);7.748(1.5);7.744(1.6);7.728(4.6);7.725(4.6);7.711(4.3);7.707(4.4);7.697(4.9);7.678(1.9);7.327(2.1);7.314(3.0);7.301(1.8);5.139(16.0);4.097(0.6);3.887(1.0);3.857(0.8);3.544(0.7);3.346(66.2);3.170(9.1);2.513(28.8);2.508(57.6);2.504(76.8); 2.499(56.0);2.495(27.2);0.000(0.8)

### Example No. D1-137

### ¹H-NMR (400 MHz, d₆-DMSO δ, ppm)

8.745(0.8);8.733(0.8);8.293(0.8);8.289(0.8);8.274(0.6);8.270(0.5);8.213(1.3);8.193(0.9); 7.889(0.5);7.887(0.5);7.874(0.6);7.871(0.6);7.728(0.7);7.713(0.8);7.707(0.8);7.691(0.8); 7.655(0.7);7.649(0.7);7.633(0.8);7.627(0.7);7.357(0.7);7.350(0.6);5.239(3.1);3.689(9.0); 2.512(5.4);2.508(12.2);2.503(17.3);2.498(12.5);2.494(5.8);2.075(16.0);0.000(10.9)

### Example No. D1-144

### ¹H-NMR (400 MHz, d₆-DMSO δ, ppm)

8.795(0.7);8.793(0.8);8.791(0.9);8.789(0.7);8.783(0.7);8.780(0.9);8.779(0.9);8.776(0.7); 8.498(1.3);8.484(1.3);8.158(0.9);8.154(0.9);8.139(1.0);8.135(1.0);8.125(1.0);8.123(1.2); 8.122(1.3);8.119(1.0);8.104(0.5);7.809(0.7);7.805(0.7);7.797(0.6);7.793(0.7);7.791(0.7); 7.787(0.6);7.778(0.6);7.774(0.6);7.300(1.3);7.285(1.2);5.285(3.2);4.994(1.6);4.973(1.7); 4.951(0.6);3.808(10.2);2.512(5.1);2.507(12.3);2.503(18.1);2.498(13.4);2.494(6.6);2.332(7.2);2.074(16.0);0.000(9.8)

### Example No. D1-147

### ¹H-NMR (400 MHz, d₆-DMSO δ, ppm)

8.732(0.5);8.216(0.5);8.212(0.6);8.210(0.8);8.208(0.8);8.206(0.9);8.204(0.7);7.835(0.5); 7.168(1.6);7.075(1.4);5.201(1.8);3.816(5.6);3.755(0.5);3.731(0.6);3.718(5.0);3.540(5.6); 2.676(0.9);2.512(3.6);2.507(8.1);2.503(11.4);2.498(8.2);2.493(3.8);2.075(16.0);0.000(7.4)

### Example No. D1-148

### ¹H-NMR (400 MHz, d₆-DMSO δ, ppm)

8.714(0.9);8.711(1.2);8.708(0.9);8.702(0.9);8.699(1.5);8.696(0.9);8.341(3.2);8.328(3.3); 8.174(1.2);8.170(1.3);8.161(1.6);8.158(2.5);8.155(2.6);8.013(0.6);8.010(0.5);7.796(0.9); 7.790(0.9);7.784(4.2);7.780(1.2);7.778(1.0);7.771(3.5);7.768(1.0);7.762(0.7);5.310(5.1); 3.934(0.8);3.900(1.2);3.873 (16.0);3.794(14.3);3.725(0.5);3.143(1.0);2.649(5.1);2.524(0.7);2.520(1.0);2.511(13.5);2.506(29.3);2.502(40.8);2.497(28.7);2.493(12.9);2.074(9.0); 0.008(0.8);0.000(26.3);-0.009(0.8)

### Example No. D2-1

### ¹H-NMR (400 MHz, CDCl₃ δ, ppm)

8.173(3.1);7.943(0.9);7.936(0.6);7.932(0.9);7.926(0.6);7.920(0.9);7.482(0.8);7.476(0.5); 7.470(1.0);7.466(0.7);7.459(1.5);7.418(1.7);7.415(1.8);7.411 (1.1);7.405(1.9);7.404(1.8); 7.399(1.0);7.394(1.2);7.392(1.2);7.284(1.3);7.264(26.8);5.300(0.7);5.279(5.2);3.443(16.0); 1.545(39.9);1.256(1.4);0.000(9.8)

### Example No. D3-1

### ¹H-NMR (400 MHz, CDCl₃ δ, ppm)

7.957(0.7);7.951(0.5);7.947(0.6);7.934(0.8);7.624(2.2);7.603(2.4);7.483(0.5);7.470(0.6); 7.467(0.6);7.460(1.1);7.420(1.3);7.417(1.4);7.414(0.9);7.407(1.9);7.400(0.8);7.396(0.9); 7.394(0.9);7.264(11.0);7.209(2.4);7.188(2.2);5.299(4.8);3.448(16.0);0.000(4.3)

### Example No. D4-35

### ¹H-NMR (400 MHz, CD3CN 6, ppm)

8.701(0.6);8.699(0.6);8.689(0.6);8.624(1.6);8.619(1.6);8.027(1.6);8.021(1.6);8.013(0.6); 8.011(0.6);7.993(0.7);7.990(0.6);7.437(0.4);7.435(0.4);7.425(0.5);7.423(0.4);7.417(0.4); 7.415(0.4);7.405(0.4);7.403(0.4);5.447(1.3);5.176(4.7);3.444(16.0);2.155(19.7);1.972(0.5);1.964(1.4);1.958(2.1);1.952(9.4);1.946(16.3);1.940(21.3);1.934(14.7);1.928(7.5);1.372(0.5);1.340(0.3);1.285(0.5);1.277(0.8);1.270(0.6)

### Example No. D4-36

### ¹H-NMR (400 MHz, CD3CN 6, ppm)

8.921(1.1);8.918(1.1);8.698(0.7);8.688(0.7);8.246(1.48.242(1.0);8.011(0.6);8.008(0.6); 7.991(0.7);7.988(0.7);7.442(0.4);7.428(0.5);7.422(0.5);7.409(0.4);5.260(4.1);3.514(16.0); 2.213(84.8);2.121(0.5);2.115(0.5);2.109(0.6);2.103(0.5);2.096(0.3);1.973(1.5);1.966(3.3);1.960(5.1);1.954(23.4);1.947(40.7);1.941(53.3);1.935(36.9);1.929(19.1);1.916(0.4); 1.770(0.4);1.372(0.5);1.340(0.6);1.285(0.9);1.276(1.1);1.270(1.4);1.222(0.4);1.204(0.7); 1.186(0.4);0.000(0.8)

### Example No. E1-21

### ¹H-NMR (400 MHz, CDCl₃ δ, ppm)

9.243(1.7);8.815(2.3);8.691(0.5);8.688(0.6);8.686(0.6);8.684(0.5);8.679(0.5);8.676(0.6); 8.674(0.6);8.672(0.5);7.796(1.0);7.792(1.0);7.777(0.6);7.772(0.6);7.678(0.6);7.676(1.1); 7.673(0.7);7.656(0.8);7.413(0.6);7.410(0.6);7.400(0.5);7.397(0.6);7.394(0.5);7.391(0.5); 7.381(0.5);7.266(12.7);5.300(1.1);5.081(4.7);3.512(16.0);1.256(0.8);0.000(4.7)

### Example No. E2-1

### ¹H-NMR (400 MHz, CDCl₃ δ, ppm)

9.228(7.3);9.200(2.9);7.666(0.7);7.662(0.7);7.647(0.8);7.642(0.8);7.507(0.6);7.503(0.7); 7.487(1.0);7.483(1.0);7.423(0.5);7.419(0.5);7.405(0.8);7.400(0.8);7.385(0.5);7.333(0.6); 7.330(0.6);7.314(0.9);7.311(0.8);7.272(7.8);5.301(4.3);5.178(4.4);3.475(16.0);0.000(2.8)

### Example No. F1-1

### ¹H-NMR (400 MHz, CDCl₃ δ, ppm)

8.574(3.6);7.926(0.7);7.909(0.7);7.902(0.8);7.497(0.6);7.4913(0.6);7.4906(0.6);7.478(0.7); 7.473(1.1);7.418(1.2);7.413(1.1);7.409(1.1);7.402(2.1);7.394(0.9);7.391(1.0);7.386(0.8);7.265(19.3);5.300(3.0);5.283(4.6);3.462(16.0);2.735(12.3);0.000(6.7)

### Example No. F1-21

### ¹H-NMR (400 MHz, CDCl₃ δ, ppm)

8.666(2.2);8.661(0.6);8.657(1.1);8.654(0.6);8.649(0.6);8.646(0.7);8.645(0.7);8.642(0.6); 8.551(3.5);7.812(2.4);7.809(2.5);7.804(1.1);7.802(1.1);7.798(1.1);7.794(1.0);7.391(0.6); 7.379(0.6);7.377(0.6);7.369(0.6);7.364(0.5);7.357(0.5);7.268(19.3);5.301(3.5);5.179(4.7); 3.516(16.0);3.156(9.9);2.894(10.2);2.739(8.3);2.714(12.0);0.000(6.7)

### Example No. F2-1

### ¹H-NMR (400 MHz, CDCl₃ δ, ppm)

7.853(0.6);7.849(0.6);7.834(0.7);7.830(0.6);7.477(0.5);7.473(0.6);7.457(0.8);7.453(0.9); 7.383(0.7);7.379(0.7);7.363(0.5);7.337(0.6);7.333(0.6);7.318(0.7);7.314(0.7);7.266(13.1); 6.842(2.2);5.299(3.8);5.249(3.2);3.448(11.9);3.252(16.0);2.345(7.9);0.000(4.7)

### Example No. F2-21

### ¹H-NMR (400 MHz, CDCl₃ δ, ppm)

9.159(0.5);8.642(0.5);8.639(0.6);8.635(0.5);8.630(0.5);8.627(0.6);8.625(0.5);8.623(0.5); 7.756(0.6);7.750(0.7);7.745(0.6);7.732(0.6);7.727(0.6);7.267(14.6);6.755(1.7);5.300(2.8); 5.154(1.4);3.520(1.3);3.488(5.0);3.237(16.0);3.220(1.1);3.208(0.9);3.204(2.3);2.706(2.0);2.404(1.0);2.324(8.0);1.256(1.2);0.000(5.5)

### Example No. H1-462

### ¹H-NMR (400 MHz, CDCl₃ δ, ppm)

8.463(1.3);8.457(1.5);8.392(1.9);8.386(1.6);7.261(55.5);7.206(0.9);7.186(0.5);6.908(1.1); 6.889(1.5);6.869(1.0);3.948(0.9);3.936(0.6);3.927(0.8);3.918(0.6);3.906(0.9);3.606(16.0); 3.260(0.9);2.844(0.7);2.825(1.4);2.807(0.7);2.281(0.7);2.269(0.5);2.259(0.6);2.239(0.6);1.255(0.6);0.008(0.6);0.000(17.9);−0.009(0.5)

The present invention further provides for the use of at least one inventive compound selected from the group consisting of substituted aryl- and heteroarylcarbonyl hydrazides of the general formula (I), and of any desired mixtures of these inventively substituted aryl- and heteroarylcarbonyl hydrazides of the general formula (I), with further active agrochemical ingredients, for example fungicides, insecticides, herbicides, plant growth regulators or safeners, for enhancement of the resistance of plants to abiotic stress factors, preferably drought stress, and also for invigoration of plant growth and/or for increasing plant yield.

The present invention further provides a spray solution for treatment of plants, comprising an amount, effective for enhancement of the resistance of plants to abiotic stress factors, of at least one compound selected from the group consisting of the aryl- and heteroarylcarbonyl hydrazides of the general formula (I) having substitution in accordance with the invention. The abiotic stress conditions which can be relativized may include, for example, heat, drought, cold and aridity stress (stress caused by aridity and/or lack of water), osmotic stress, waterlogging, elevated soil salinity, elevated exposure to minerals, ozone conditions, strong light conditions, limited availability of nitrogen nutrients, limited availability of phosphorus nutrients.

In one embodiment, it is possible, for example, that the compounds envisaged in accordance with the invention, i.e. the appropriate aryl- and heteroarylcarbonyl hydrazides of the general formula (I) having substitution in accordance with the invention, are applied by spray application to appropriate plants or plant parts to be treated. The compounds of the general formula (I) or salts thereof are used as envisaged in accordance with the invention preferably with a dosage between 0.00005 and 3 kg/ha, more preferably between 0.0001 and 2 kg/ha, especially preferably between 0.0005 and 1 kg/ha, specifically preferably between 0.001 and 0.25 kg/ha.

The term “resistance to abiotic stress” is understood in the context of the present invention to mean various kinds of benefits for plants. Such advantageous properties are manifested, for example, in the following improved plant characteristics: improved root growth with regard to surface area and depth, increased stolon or tiller formation, stronger and more productive stolons and tillers, improvement in shoot growth, increased lodging resistance, increased shoot base diameter, increased leaf area, higher yields of nutrients and constituents, for example carbohydrates, fats, oils, proteins, vitamins, minerals, essential oils, dyes, fibers, better fiber quality, earlier flowering, increased number of flowers, reduced content of toxic products such as mycotoxins, reduced content of residues or disadvantageous constituents of any kind, or better digestibility, improved storage stability of the harvested material, improved tolerance to disadvantageous temperatures, improved tolerance to drought and aridity, and also oxygen deficiency as a result of waterlogging, improved tolerance to elevated salt contents in soils and water, enhanced tolerance to ozone stress, improved compatibility with respect to herbicides and other plant treatment compositions, improved water absorption and photosynthesis performance, advantageous plant properties, for example acceleration of ripening, more homogeneous ripening, greater attractiveness to beneficial animals, improved pollination, or other advantages well known to a person skilled in the art.

More particularly, the use of one or more inventive compounds of the general formula (I) exhibits the advantages described in spray application to plants and plant parts. In addition, the combined use of aryl- and heteroarylcarbonyl hydrazides of the general formula (I) having substitution in accordance with the invention with genetically modified cultivars with a view to increased tolerance to abiotic stress is likewise possible.

The further various benefits for plants mentioned above can be combined in a known manner in component form, and generally applicable terms can be used to describe them. Such terms are, for example, the following names: phytotonic effect, resistance to stress factors, less plant stress, plant health, healthy plants, plant fitness, plant wellness, plant concept, vigor effect, stress shield, protective shield, crop health, crop health properties, crop health products, crop health management, crop health therapy, plant health, plant health properties, plant health products, plant health management, plant health therapy, greening effect or regreening effect, freshness, or other terms with which a person skilled in the art is entirely familiar.

In the context of the present invention, a good effect on resistance to abiotic stress is understood to mean, without limitation,
at least an emergence improved by generally 3%, especially more than 5%, more preferably more than 10%,
at least a yield enhanced by generally 3%, especially more than 5%, more preferably more than 10%,
at least a root development improved by generally 3%, especially more than 5%, more preferably more than 10%,
at least a shoot size rising by generally 3%, especially more than 5%, more preferably more than 10%,
at least a leaf area increased by generally 3%, especially more than 5%, more preferably more than 10%,
at least a photosynthesis performance improved by generally 3%, especially more than 5%, more preferably more than 10%, and/or
at least a flower development improved by generally 3%, especially more than 5%, more preferably more than 10%,

and the effects may occur individually or else in any combination of two or more effects.

The present invention further provides a spray solution for treatment of plants, comprising an amount, effective for enhancement of the resistance of plants to abiotic stress factors, of at least one compound from the group of the aryl- and heteroarylcarbonyl hydrazides of the general formula (I) having substitution in accordance with the invention. The spray solution may comprise other customary constituents, such as solvents, formulation auxiliaries, especially water. Further constituents may include active agrochemical ingredients which are described in more detail below.

The present invention further provides for the use of corresponding spray solutions for increasing the resistance of plants to abiotic stress factors. The remarks which follow apply both to the use according to the invention of one or more compounds of the general formula (I) per se and to the corresponding spray solutions.

In accordance with the invention, it has additionally been found that the inventive application of one or more compounds of the general formula (I) in combination with at least one fertilizer as defined further below to plants or in their environment is possible.

Fertilizers which can be used in accordance with the invention together with the compounds of the general formula (I) elucidated in detail above are generally organic and inorganic nitrogen-containing compounds, for example ureas, urea/formaldehyde condensation products, amino acids, ammonium salts and ammonium nitrates, potassium salts (preferably chlorides, sulfates, nitrates), salts of phosphoric acid and/or salts of phosphorous acid (preferably potassium salts and ammonium salts). In this context, particular mention should be made of the NPK fertilizers, i.e. fertilizers which contain nitrogen, phosphorus and potassium, calcium ammonium nitrate, i.e. fertilizers which additionally contain calcium, or ammonium sulfate nitrate (general formula (NH₄)₂SO₄ NH₄NO₃), ammonium phosphate and ammonium sulfate. These fertilizers are generally known to the person skilled in the art; see also, for example, Ullmann's Encyclopedia of Industrial Chemistry, 5th edition, Vol. A 10, pages 323 to 431, Verlagsgesellschaft, Weinheim, 1987.

The fertilizers may additionally comprise salts of micronutrients (preferably calcium, sulfur, boron, manganese, magnesium, iron, boron, copper, zinc, molybdenum and cobalt) and of phytohormones (for example vitamin B1 and indole-(III)-acetic acid) or mixtures of these. Fertilizers used in accordance with the invention may also contain other salts such as monoammonium phosphate (MAP), diammonium phosphate (DAP), potassium sulfate, potassium chloride, magnesium sulfate. Suitable amounts for the secondary nutrients or trace elements are amounts of 0.5% to 5% by weight, based on the overall fertilizer. Further possible ingredients are crop protection agents, for example fungicides, insecticides, herbicides, plant growth regulators or safeners, or mixtures thereof. Further details of these are given further down.

The fertilizers can be used, for example, in the form of powders, granules, prills or compactates. However, the fertilizers can also be used in liquid form, dissolved in an aqueous medium. In this case, dilute aqueous ammonia can also be used as a nitrogen fertilizer. Further possible ingredients for fertilizers are described, for example, in Ullmann's Encyclopedia of Industrial Chemistry, 5th edition, 1987, volume A 10, pages 363 to 401, DE-A 41 28 828, DE-A 19 05 834 and DE-A 196 31 764. The general composition of the fertilizers, which, in the context of the present invention, may take the form of straight and/or compound fertilizers, for example composed of nitrogen, potassium or phosphorus, may vary within a wide range. In general, a content of 1% to 30% by weight of nitrogen (preferably 5% to 20% by weight), of 1% to 20% by weight of potassium (preferably 3% to 15% by weight) and a content of 1% to 20% by weight of phosphorus (preferably 3% to 10% by weight) is advantageous. The microelement content is usually in the ppm range, preferably in the range from 1 to 1000 ppm.

In the context of the present invention, the fertilizer and one or more inventive compounds of the general formula (I) may be administered simultaneously. However, it is also possible first to apply the fertilizer and then one or more inventive compounds of the general formula (I), or first to apply one or more compounds of the general formula (I) and then the fertilizer. In the case of nonsynchronous application of one or more compounds of the general formula (I) and the fertilizer, the application in the context of the present invention is, however, effected in a functional relationship, especially within a period of generally 24 hours, preferably 18 hours, more preferably 12 hours, specifically 6 hours, more specifically 4 hours, even more specifically within 2 hours. In very particular embodiments of the present invention, one or more compounds of the formula (I) according to the invention and the fertilizer are applied within a time frame of less than 1 hour, preferably less than 30 minutes, more preferably less than 15 minutes.

Preference is given to the use of inventive compounds of the general formula (I) on plants from the group of the useful plants, ornamentals, turfgrass types, commonly used trees which are used as ornamentals in the public and domestic sectors, and forestry trees. Forestry trees include trees for the production of timber, cellulose, paper and products made from parts of the trees. The term useful plants as used here refers to crop plants which are used as plants for obtaining foods, animal feeds, fuels or for industrial purposes.

The useful plants include, for example, the following types of plants: triticale, durum (hard wheat), turf, vines, cereals, for example wheat, barley, rye, oats, rice, corn and millet; beet, for example sugar beet and fodder beet; fruits, for example pome fruit, stone fruit and soft fruit, for example apples, pears, plums, peaches, almonds, cherries and berries, for example strawberries, raspberries, blackberries; legumes, for example beans, lentils, peas and soybeans; oil crops, for example oilseed rape, mustard, poppies, olives, sunflowers, coconuts, castor oil plants, cocoa beans and peanuts; cucurbits, for example pumpkin/squash, cucumbers and melons; fiber plants, for example cotton, flax, hemp and jute; citrus fruits, for example oranges, lemons, grapefruit and tangerines; vegetables, for example spinach, lettuce, asparagus, cabbage species, carrots, onions, tomatoes, potatoes and bell peppers; Lauraceae, for example avocado, Cinnamomum, camphor, or also plants such as tobacco, nuts, coffee, eggplant, sugar cane, tea, pepper, grapevines, hops, bananas, latex plants and ornamentals, for example flowers, shrubs, deciduous trees and coniferous trees. This enumeration does not constitute a limitation.

The following plants are considered to be particularly suitable target crops for the application of the method of the invention: oats, rye, triticale, durum, cotton, eggplant, turf, pome fruit, stone fruit, soft fruit, corn, wheat, barley, cucumber, tobacco, vines, rice, cereals, pears, pepper, beans, soybeans, oilseed rape, tomato, bell pepper, melons, cabbage, potatoes and apples.

Examples of trees which can be improved by the method of the invention include: Abies sp., Eucalyptus sp., Picea sp., Pinus sp., Aesculus sp., Platanus sp., Tilia sp., Acer sp., Tsuga sp., Fraxinus sp., Sorbus sp., Betula sp., Crataegus sp., Ulmus sp., Quercus sp., Fagus sp., Salix sp., Populus sp.

Preferred trees which can be improved by the method of the invention include: from the tree species Aesculus: A. hippocastanum, A. pariflora, A. carnea; from the tree species Platanus: P. aceriflora, P. occidentalis, P. racemosa; from the tree species Picea: P. abies; from the tree species Pinus: P. radiate, P. ponderosa, P. contorta, P. sylvestre, P. elliottii, P. montecola, P. albicaulis, P. resinosa, P. palustris, P. taeda, P. flexilis, P. jeffregi, P. baksiana, P. strobes; from the tree species Eucalyptus: E. grandis, E. globulus, E. camadentis, E. nitens, E. obliqua, E. regnans, E. pilularus.

Particularly preferred trees which can be improved by the method of the invention are: from the tree species Pinus: P. radiate, P. ponderosa, P. contorta, P. sylvestre, P. strobes; from the tree species Eucalyptus: E. grandis, E. globulus and E. camadentis.

Particularly preferred trees which can be improved by the method of the invention are: horse chestnut, Platanaceae, linden tree and maple tree.

The present invention can also be applied to any desired turfgrasses, including cool-season turfgrasses and warm-season turfgrasses. Examples of cool-season turfgrasses are bluegrasses (Poa spp.), such as Kentucky bluegrass (Poa pratensis L.), rough bluegrass (Poa trivialis L.), Canada bluegrass (Poa compressa L.), annual bluegrass (Poa annua L.), upland bluegrass (Poa glaucantha Gaudin), wood bluegrass (Poa nemoralis L.) and bulbous bluegrass (Poa bulbosa L.); bentgrasses (Agrostis spp.) such as creeping bentgrass (Agrostis palustris Huds.), colonial bentgrass (Agrostis tenuis Sibth.), velvet bentgrass (Agrostis canina L.), South German Mixed Bentgrass (Agrostis spp. including Agrostis tenius Sibth., Agrostis canina L., and Agrostis palustris Huds.), and redtop (Agrostis alba L.); fescues (Festuca spp.), such as red fescue (Festuca rubra L. spp. rubra), creeping fescue (Festuca rubra L.), chewings fescue (Festuca rubra commutata Gaud.), sheep fescue (Festuca ovina L.), hard fescue (Festuca longifolia Thuill.), hair fescue (Festucu capillata Lam.), tall fescue (Festuca arundinacea Schreb.) and meadow fescue (Festuca elanor L.);

ryegrasses (Lolium spp.), such as annual ryegrass (Lolium multiflorum Lam.), perennial ryegrass (Lolium perenne L.) and Italian ryegrass (Lolium multiflorum Lam.);

and wheatgrasses (Agropyron spp.), such as fairway wheatgrass (Agropyron cristatum (L.) Gaertn.), crested wheatgrass (Agropyron desertorum (Fisch.) Schult.) and western wheatgrass (Agropyron smithii Rydb.).

Examples of further cool-season turfgrasses are beachgrass (Ammophila breviligulata Fern.), smooth bromegrass (Bromus inermis Leyss.), cattails such as Timothy (Phleum pratense L.), sand cattail (Phleum subulatum L.), orchardgrass (Dactylis glomerata L.), weeping alkaligrass (Puccinellia distans (L.) Parl.) and crested dog's-tail (Cynosurus cristatus L.).

Examples of warm-season turfgrasses are Bermudagrass (Cynodon spp. L. C. Rich), zoysiagrass (Zoysia spp. Willd.), St. Augustine grass (Stenotaphrum secundatum Walt Kuntze), centipedegrass (Eremochloa ophiuroides Munro Hack.), carpetgrass (Axonopus affinis Chase), Bahia grass (Paspalum notatum Flugge), Kikuyugrass (Pennisetum clandestinum Hochst. ex Chiov.), buffalo grass (Buchloe dactyloids (Nutt.) Engelm.), Blue gramma (Bouteloua gracilis (H.B.K.) Lag. ex Griffiths), seashore paspalum (Paspalum vaginatum Swartz) and sideoats grama (Bouteloua curtipendula (Michx. Torr.)). Cool-season turfgrasses are generally preferred for the inventive use. Particular preference is given to bluegrass, bentgrass and redtop, fescues and ryegrasses. Bentgrass is especially preferred.

Particular preference is given to using the inventive compounds of the general formula (I) to treat plants of the respective commercially available or commonly used plant cultivars. Plant cultivars are understood to mean plants which have new properties (“traits”) and which have been obtained by conventional breeding, by mutagenesis or with the aid of recombinant DNA techniques. Crop plants may thus be plants which can be obtained by conventional breeding and optimization methods or by biotechnological and genetic engineering methods or combinations of these methods, including the transgenic plants and including the plant cultivars which are protectable or non-protectable by plant breeders' rights.

The treatment method according to the invention can thus also be used for the treatment of genetically modified organisms (GMOs), e.g. plants or seeds. Genetically modified plants (or transgenic plants) are plants in which a heterologous gene has been stably integrated into the genome. The expression “heterologous gene” essentially means a gene which is provided or assembled outside the plant and when introduced into the nuclear, chloroplastic or hypochondrial genome gives the transformed plant new or improved agronomic or other properties by expressing a protein or polypeptide of interest or by downregulating or silencing (an)other gene(s) which is/are present in the plant (using for example antisense technology, cosuppression technology or RNAi technology [RNA interference]). A heterologous gene that is located in the genome is also called a transgene. A transgene that is defined by its specific presence in the plant genome is called a transformation or transgenic event.

Plants and plant varieties which are preferably treated with the inventive compounds of the general formula (I) include all plants which have genetic material which imparts particularly advantageous, useful traits to these plants (whether obtained by breeding and/or biotechnological means or not).

Plants and plant varieties which can likewise be treated with the inventive compounds of the general formula (I) are those plants which are resistant to one or more abiotic stress factors. Abiotic stress conditions may include, for example, heat, drought, cold and aridity stress, osmotic stress, waterlogging, increased soil salinity, increased exposure to minerals, ozone conditions, strong light conditions, limited availability of nitrogen nutrients, limited availability of phosphorus nutrients or shade avoidance.

Plants and plant cultivars which can likewise be treated with the inventive compounds of the general formula (I) are those plants which are characterized by enhanced yield characteristics. Enhanced yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and nonstress conditions), including, but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and resistance to lodging. Further yield traits include seed composition, such as carbohydrate content, protein content, oil content and oil composition, nutritional value, reduction in antinutritional compounds, improved processibility and better storage stability.

Plants that may also be treated with the inventive compounds of the general formula (I) are hybrid plants that already express the characteristics of heterosis, or hybrid effect, which results in generally higher yield, higher vigor, better health and better resistance towards biotic and abiotic stress factors. Such plants are typically produced by crossing an inbred male-sterile parent line (the female crossbreeding parent) with another inbred male-fertile parent line (the male crossbreeding parent). Hybrid seed is typically harvested from the male-sterile plants and sold to growers. Male-sterile plants can sometimes (for example in corn) be produced by detasseling (i.e. mechanical removal of the male reproductive organs or male flowers); however, it is more typical for male sterility to be the result of genetic determinants in the plant genome. In that case, and especially when seed is the desired product to be harvested from the hybrid plants, it is typically beneficial to ensure that male fertility in hybrid plants, which contain the genetic determinants responsible for male sterility, is fully restored. This can be accomplished by ensuring that the male crossbreeding parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described for Brassica species (WO 92/005251, WO 95/009910, WO 98/27806, WO 05/002324, WO 06/021972 and U.S. Pat. No. 6,229,072). However, genetic determinants for male sterility can also be located in the nuclear genome. Male-sterile plants can also be obtained by plant biotechnology methods such as genetic engineering. A particularly useful means of obtaining male-sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar (e.g. WO 91/002069).

Plants or plant cultivars (obtained by plant biotechnology methods such as genetic engineering) which may also be treated with the inventive compounds of the general formula (I) are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. Thus, for example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., Science (1983), 221, 370-371), the CP4 gene of the bacterium Agrobacterium sp. (Barry et al., Curr. Topics Plant Physiol. (1992), 7, 139-145), the genes encoding a petunia EPSPS (Shah et al., Science (1986), 233, 478-481), a tomato EPSPS (Gasser et al., J. Biol. Chem. (1988), 263, 4280-4289) or an Eleusine EPSPS (WO 01/66704). It can also be a mutated EPSPS, as described, for example, in EP-A 0837944, WO 00/066746, WO 00/066747 or WO 02/026995. Glyphosate-tolerant plants can also be obtained by expressing a gene that encodes a glyphosate oxidoreductase enzyme as described in U.S. Pat. No. 5,776,760 and U.S. Pat. No. 5,463,175. Glyphosate-tolerant plants can also be obtained by expressing a gene that encodes a glyphosate acetyl transferase enzyme as described, for example, in WO 02/036782, WO 03/092360, WO 05/012515 and WO 07/024782. Glyphosate-tolerant plants can also be obtained by selecting plants containing naturally occurring mutations of the abovementioned genes, as described, for example, in WO 01/024615 or WO 03/013226.

Other herbicide-resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One example of such an effective detoxifying enzyme is an enzyme encoding a phosphinothricin acetyltransferase (such as the bar or pat protein from Streptomyces species). Plants expressing an exogenous phosphinothricin acetyltransferase are described, for example, in U.S. Pat. No. 5,561,236; U.S. Pat. No. 5,648,477; U.S. Pat. No. 5,646,024; U.S. Pat. No. 5,273,894; U.S. Pat. No. 5,637,489; U.S. Pat. No. 5,276,268; U.S. Pat. No. 5,739,082; U.S. Pat. No. 5,908,810 and U.S. Pat. No. 7,112,665.

Further herbicide-tolerant plants are also plants that have been made tolerant to the herbicides inhibiting the enzyme hydroxyphenylpyruvate dioxygenase (HPPD). Hydroxyphenylpyruvate dioxygenases are enzymes that catalyze the reaction in which para-hydroxyphenylpyruvate (HPP) is converted to homogentizate. Plants tolerant to HPPD inhibitors can be transformed with a gene encoding a naturally occurring resistant HPPD enzyme, or a gene encoding a mutated HPPD enzyme according to WO 96/038567, WO 99/024585 and WO 99/024586. Tolerance to HPPD inhibitors can also be obtained by transforming plants with genes encoding certain enzymes enabling the formation of homogentisate despite inhibition of the native HPPD enzyme by the HPPD inhibitor. Such plants and genes are described in WO 99/034008 and WO 2002/36787. Tolerance of plants to HPPD inhibitors can also be improved by transforming plants with a gene encoding a prephenate dehydrogenase enzyme in addition to a gene encoding an HPPD-tolerant enzyme, as described in WO 2004/024928.

Other herbicide-resistant plants are plants which have been rendered tolerant to acetolactate synthase (ALS) inhibitors. Known ALS inhibitors include, for example, sulfonylurea, imidazolinone, triazolopyrimidines, pyrimidinyloxy(thio)benzoates, and/or sulfonylaminocarbonyltriazolinone herbicides. Different mutations in the ALS enzyme (also known as acetohydroxy acid synthase, AHAS) are known to confer tolerance to different herbicides and groups of herbicides, as described, for example, in Tranel nd Wright, Weed Science (2002), 50, 700-712, and also in U.S. Pat. No. 5,605,011, U.S. Pat. No. 5,378,824, U.S. Pat. No. 5,141,870 and U.S. Pat. No. 5,013,659. The production of sulfonylurea-tolerant plants and imidazolinone-tolerant plants has been described in U.S. Pat. No. 5,605,011; U.S. Pat. No. 5,013,659; U.S. Pat. No. 5,141,870; U.S. Pat. No. 5,767,361; U.S. Pat. No. 5,731,180; U.S. Pat. No. 5,304,732; U.S. Pat. No. 4,761,373; U.S. Pat. No. 5,331,107; U.S. Pat. No. 5,928,937; and U.S. Pat. No. 5,378,824; and also in the international publication WO 96/033270. Further imidazolinone-tolerant plants have also been described, for example, in WO 2004/040012, WO 2004/106529, WO 2005/020673, WO 2005/093093, WO 2006/007373, WO 2006/015376, WO 2006/024351 and WO 2006/060634. Further sulfonylurea- and imidazolinone-tolerant plants have also been described, for example, in WO 2007/024782.

Further plants tolerant to ALS-inhibitors, in particular to imidazolinones, sulfonylureas and/or sulfamoylcarbonyltriazolinones can be obtained by induced mutagenesis, by selection in cell cultures in the presence of the herbicide or by mutation breeding, as described, for example, for soybeans in U.S. Pat. No. 5,084,082, for rice in WO 97/41218, for sugarbeet in U.S. Pat. No. 5,773,702 and WO 99/057965, for lettuce in U.S. Pat. No. 5,198,599 or for sunflower in WO 2001/065922.

Plants or plant cultivars (obtained by plant biotechnology methods such as genetic engineering) which may also be treated with the inventive compounds of the general formula (I) are insect-resistant transgenic plants, i.e. plants made resistant to attack by certain target insects. Such plants can be obtained by genetic transformation, or by selection of plants containing a mutation imparting such insect resistance.

The term “insect-resistant transgenic plant”, as used herein, includes any plant containing at least one transgene comprising a coding sequence encoding:

1) an insecticidal crystal protein from Bacillus thuringiensis or an insecticidal portion thereof, such as the insecticidal crystal proteins compiled by Crickmore et al., Microbiology and Molecular Biology Reviews (1998), 62, 807-813, updated by Crickmore et al. (2005) in the Bacillus thuringiensis toxin nomenclature (online at: http://www.lifesci.sussex.ac.uk/Home/Neil_Crickmore/Bt/), or insecticidal portions thereof, for example proteins of the Cry protein classes Cry1Ab, Cry1Ac, Cry1F, Cry2Ab, Cry3Ae or Cry3Bb or insecticidal portions thereof; or 2) a crystal protein from Bacillus thuringiensis or a portion thereof which is insecticidal in the presence of a second, other crystal protein than Bacillus thuringiensis or a portion thereof, such as the binary toxin made up of the Cy34 and Cy35 crystal proteins (Moellenbeck et al., Nat. Biotechnol. (2001), 19, 668-72; Schnepf et al., Applied Environm. Microb. (2006), 71, 1765-1774); or

3) a hybrid insecticidal protein comprising parts of two different insecticidal crystal proteins from Bacillus thuringiensis, such as a hybrid of the proteins of 1) above or a hybrid of the proteins of 2) above, for example the Cry1A.105 protein produced by corn event MON98034 (WO 2007/027777); or

4) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation, such as the Cry3Bb1 protein in maize events MON863 or MON88017, or the Cry3A protein in maize event MIR 604; or

5) an insecticidal secreted protein from Bacillus thuringiensis or Bacillus cereus, or an insecticidal portion thereof, such as the vegetative insecticidal proteins (VIPs) listed under the following link, for example proteins from the VIP3Aa protein class: http://www.lifesci.sussex.ac.uk/Home/Neil_Crickmore/Bt/vip.html; or

6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 94/21795); or

7) a hybrid insecticidal protein comprising parts from different secreted proteins from Bacillus thuringiensis or Bacillus cereus, such as a hybrid of the proteins in 1) above or a hybrid of the proteins in 2) above; or

8) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation (while still encoding an insecticidal protein), such as the VIP3Aa protein in cotton event COT 102.

Of course, insect-resistant transgenic plants, as used herein, also include any plant comprising a combination of genes encoding the proteins of any one of the abovementioned classes 1 to 8. In one embodiment, an insect-resistant plant contains more than one transgene encoding a protein of any one of the above classes 1 to 8, to expand the range of the target insect species affected or to delay insect resistance development to the plants, by using different proteins insecticidal to the same target insect species but having a different mode of action, such as binding to different receptor binding sites in the insect.

Plants or plant cultivars (obtained by plant biotechnology methods such as genetic engineering) which may also be treated with the compounds according to the invention of the general formula (I) are tolerant to abiotic stress factors. Such plants can be obtained by genetic transformation, or by selection of plants containing a mutation imparting such stress resistance. Particularly useful stress-tolerant plants include the following:

a. plants which contain a transgene capable of reducing the expression and/or the activity of the poly(ADP-ribose)polymerase (PARP) gene in the plant cells or plants, as described in WO 2000/004173 or EP 04077984.5 or EP 06109836.5;

b. plants which contain a stress tolerance-enhancing transgene capable of reducing the expression and/or the activity of the PARG-encoding genes of the plants or plant cells, as described, for example, in WO 2004/090140;

c. plants which comprise a stress-tolerance-enhancing transgene coding for a plant-functional enzyme of the nicotinamide adenine dinucleotide salvage biosynthetic pathway, including nicotinamidase, nicotinate phosphoribosyltransferase, nicotinic acid mononucleotide adenyltransferase, nicotinamide adenine dinucleotide synthetase or nicotinamide phosphoribosyltransferase as described e.g. in EP 04077624.7 or WO 2006/133827 or PCT/EP07/002433.

Plants or plant varieties (obtained by plant biotechnology methods such as genetic engineering) which may also be treated with the inventive compounds of the general formula (I) show altered quantity, quality and/or storage stability of the harvested product and/or altered properties of specific ingredients of the harvested product such as, for example:

1) Transgenic plants which synthesize a modified starch which, in its physicochemical characteristics, in particular the amylose content or the amylose/amylopectin ratio, the degree of branching, the average chain length, the side chain distribution, the viscosity behavior, the gelling strength, the starch granule size and/or the starch granule morphology, is changed in comparison with the synthesized starch in wild-type plant cells or plants, so that this modified starch is better suited to specific applications. These transgenic plants that synthesize a modified starch are described, for example, in EP 0571427, WO 95/004826, EP 0719338, WO 96/15248, WO 96/19581, WO 96/27674, WO 97/11188, WO 97/26362, WO 97/32985, WO 97/42328, WO 97/44472, WO 97/45545, WO 98/27212, WO 98/40503, WO 99/58688, WO 99/58690, WO 99/58654, WO 2000/008184, WO 2000/008185, WO 2000/28052, WO 2000/77229, WO 2001/12782, WO 2001/12826, WO 2002/101059, WO 2003/071860,

WO 2004/056999, WO 2005/030942, WO 2005/030941, WO 2005/095632, WO 2005/095617, WO 2005/095619, WO 2005/095618, WO 2005/123927, WO 2006/018319, WO 2006/103107, WO 2006/108702, WO 2007/009823, WO 2000/22140, WO 2006/063862, WO 2006/072603, WO 2002/034923, EP 06090134.5, EP 06090228.5, EP 06090227.7, EP 07090007.1, EP 07090009.7, WO 2001/14569, WO 2002/79410, WO 2003/33540, WO 2004/078983, WO 2001/19975, WO 95/26407, WO 96/34968, WO 98/20145, WO 99/12950, WO 99/66050, WO 99/53072, US 6,734,341, WO 2000/11192, WO 98/22604, WO 98/32326, WO 2001/98509, WO 2001/98509, WO 2005/002359, U.S. Pat. No. 5,824,790, U.S. Pat. No. 6,013,861, WO 94/004693, WO 94/009144, WO 94/11520, WO 95/35026 bzw. WO 97/20936 beschrieben.

2) Transgenic plants which synthesize non-starch carbohydrate polymers or which synthesize non-starch carbohydrate polymers with altered properties in comparison to wild-type plants without genetic modification. Examples are plants which produce polyfructose, especially of the inulin and levan type, as described in EP 0663956, WO 96/001904, WO 96/021023, WO 98/039460 and

WO 99/024593, plants which produce alpha-1,4-glucans, as described in WO 95/031553, US 2002/031826, U.S. Pat. No. 6,284,479, U.S. Pat. No. 5,712,107,

WO 97/047806, WO 97/047807, WO 97/047808 and WO 2000/14249, plants which produce alpha-1,6-branched alpha-1,4-glucans, as described in WO 2000/73422, and plants which produce alternan, as described in WO 2000/047727, EP 06077301.7, U.S. Pat. No. 5,908,975 and EP 0728213.

3) Transgenic plants which produce hyaluronan, as for example described in WO 06/032538, WO 2007/039314, WO 2007/039315, WO 2007/039316, JP 2006/304779 and WO 2005/012529.

Plants or plant cultivars (obtained by plant biotechnology methods such as genetic engineering) which may also be treated with the inventive compounds of the general formula (I) are plants, such as cotton plants, with altered fiber characteristics. Such plants can be obtained by genetic transformation, or by selection of plants containing a mutation imparting such altered fiber characteristics and include:

a) plants, such as cotton plants, which contain an altered form of cellulose synthase genes, as described in WO 98/000549;

b) plants, such as cotton plants, which contain an altered form of rsw2 or rsw3 homologous nucleic acids, as described in WO 2004/053219;

c) plants, such as cotton plants, with an increased expression of sucrose phosphate synthase, as described in WO 2001/017333;

d) plants, such as cotton plants, with an increased expression of sucrose synthase as described in WO 02/45485;

e) plants, such as cotton plants, wherein the timing of the plasmodesmatal gating at the basis of the fiber cell is altered, for example through downregulation of fiber-selective β-1,3-glucanase, as described in

WO 2005/017157;

f) plants, such as cotton plants, which have fibers with altered reactivity, for example through expression of the N-acetylglucosamine transferase gene including nodC and chitin synthase genes, as described in WO 2006/136351.

Plants or plant cultivars (obtained by plant biotechnology methods such as genetic engineering) which may also be treated with the inventive compounds of the general formula (I) are plants, such as oilseed rape or related Brassica plants, with altered oil profile characteristics. Such plants can be obtained by genetic transformation, or by selection of plants containing a mutation imparting such altered oil characteristics and include:

a) plants, such as oilseed rape plants, which produce oil having a high oleic acid content, as described, for example, in U.S. Pat. No. 5,969,169, U.S. Pat. No. 5,840,946 or U.S. Pat. No. 6,323,392 or U.S. Pat. No. 6,063,947;

b) plants, such as oilseed rape plants, which produce oil having a low linolenic acid content, as described in U.S. Pat. No. 6,270,828, U.S. Pat. No. 6,169,190 or U.S. Pat. No. 5,965,755;

c) plants, such as oilseed rape plants, which produce oil having a low level of saturated fatty acids, as described, for example, in U.S. Pat. No. 5,434,283.

Particularly useful transgenic plants which may be treated with the inventive compounds of the general formula (I) are plants containing transformation events, or a combination of transformation events, and that are listed for example in the databases of various national or regional regulatory agencies.

Particularly useful transgenic plants which may be treated with the inventive compounds of the general formula (I) are, for example, plants which comprise one or more genes which encode one or more toxins and are the transgenic plants available under the following trade names: YIELD GARD® (for example corn, cotton, soybeans), KnockOut® (for example corn), BiteGard® (for example corn), BT-Xtra® (for example corn), StarLink® (for example corn), Bollgard® (cotton), Nucotn® (cotton), Nucotn 33B® (cotton), NatureGard® (for example corn), Protecta® and NewLeaf® (potato). Examples of herbicide-tolerant plants include are corn varieties, cotton varieties and soya bean varieties which are available under the following trade names: Roundup Ready® (tolerance to glyphosates, for example corn, cotton, soybeans), Liberty Link® (tolerance to phosphinothricin, for example oilseed rape), IMI® (tolerance to imidazolinone) and SOS® (tolerance to sulfonylurea), for example corn. Herbicide-resistant plants (plants bred in a conventional manner for herbicide tolerance) which may be mentioned include the varieties sold under the name Clearfield® (for example corn).

The compounds of the formula (I) to be used in accordance with the invention can be converted to customary formulations, such as solutions, emulsions, wettable powders, water- and oil-based suspensions, powders, dusts, pastes, soluble powders, soluble granules, granules for broadcasting, suspoemulsion concentrates, natural compounds impregnated with active ingredient, synthetic substances impregnated with active ingredient, fertilizers, and also microencapsulations in polymeric substances. In the context of the present invention, it is especially preferred when the compounds of the general formula (I) are used in the form of a spray formulation.

The present invention therefore additionally also relates to a spray formulation for enhancing the resistance of plants to abiotic stress. A spray formulation is described in detail hereinafter:

The formulations for spray application are produced in a known manner, for example by mixing the compounds of the general formula (I) for use in accordance with the invention with extenders, i.e. liquid solvents and/or solid carriers, optionally with use of surfactants, i.e. emulsifiers and/or dispersants and/or foam formers. Further customary additives, for example customary extenders and solvents or diluents, dyes, wetting agents, dispersants, emulsifiers, antifoams, preservatives, secondary thickeners, stickers, gibberellins and also water, can optionally also be used. The formulations are produced either in suitable facilities or else before or during application.

The auxiliaries used may be those substances which are suitable for imparting, to the composition itself and/or to preparations derived therefrom (for example spray liquors), particular properties such as particular technical properties and/or else special biological properties. Typical auxiliaries include: extenders, solvents and carriers.

Suitable extenders are, for example, water, polar and nonpolar organic chemical liquids, for example from the classes of the aromatic and nonaromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulfones and sulfoxides (such as dimethyl sulfoxide).

If the extender utilized is water, it is also possible to use, for example, organic solvents as auxiliary solvents. Useful liquid solvents essentially include: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethyl sulfoxide, and also water.

It is possible to use colorants such as inorganic pigments, for example iron oxide, titanium oxide and Prussian blue, and organic colorants such as alizarin colorants, azo colorants and metal phthalocyanine colorants, and trace nutrients such as salts of iron, manganese, boron, copper, cobalt, molybdenum and zinc.

Suitable wetting agents which may be present in the formulations which can be used in accordance with the invention are all substances which promote wetting and which are conventionally used for the formulation of agrochemical active substances. Preference is given to using alkyl naphthalenesulfonates, such as diisopropyl or diisobutyl naphthalenesulfonates.

Suitable dispersants and/or emulsifiers which may be present in the formulations which can be used in accordance with the invention are all nonionic, anionic and cationic dispersants conventionally used for the formulation of active agrochemical ingredients. Preference is given to using nonionic or anionic dispersants or mixtures of nonionic or anionic dispersants. Suitable nonionic dispersants include in particular ethylene oxide/propylene oxide block polymers, alkylphenol polyglycol ethers and tristyrylphenol polyglycol ethers, and the phosphated or sulfated derivatives thereof. Suitable anionic dispersants are especially lignosulfonates, polyacrylic acid salts and arylsulfonate-formaldehyde condensates.

Antifoams which may be present in the formulations usable in accordance with the invention are all foam-inhibiting substances customary for the formulation of active agrochemical ingredients. Silicone antifoams and magnesium stearate can be used with preference.

Preservatives which may be present in the formulations usable in accordance with the invention are all substances usable for such purposes in agrochemical compositions. Examples include dichlorophene and benzyl alcohol hemiformal.

Secondary thickeners which may be present in the formulations usable in accordance with the invention are all substances usable for such purposes in agrochemical compositions. Preferred examples include cellulose derivatives, acrylic acid derivatives, xanthan, modified clays and finely divided silica.

Stickers which may be present in the formulations usable in accordance with the invention include all customary binders usable in seed-dressing products. Preferred examples include polyvinylpyrrolidone, polyvinyl acetate, polyvinyl alcohol and tylose. Suitable gibberellins which may be present in the formulations which can be used in accordance with the invention are preferably the gibberellins A1, A3 (=gibberellic acid), A4 and A7; gibberellic acid is especially preferably used. The gibberellins are known (cf. R. Wegler “Chemie der Pflanzenschutz- and Schadlingsbekampfungsmittel”, vol. 2, Springer Verlag, 1970, pp. 401-412).

Further additives may be fragrances, mineral or vegetable, optionally modified oils, waxes and nutrients (including trace nutrients), such as salts of iron, manganese, boron, copper, cobalt, molybdenum and zinc. Additionally present may be stabilizers, such as cold stabilizers, antioxidants, light stabilizers or other agents which improve chemical and/or physical stability.

The formulations contain generally between 0.01% and 98% by weight, preferably between 0.5% and 90%, of the compound of the general formula (I).

The inventive compounds of the general formula (I) may be present in commercially available formulations, and also in the use forms, prepared from these formulations, in a mixture with other active compounds, such as insecticides, attractants, sterilizing agents, bactericides, acaricides, nematicides, fungicides, growth-regulating substances, herbicides, safeners, fertilizers or semiochemicals.

In addition, the described positive effect of the compounds of the formula (I) on the plants' own defenses can be supported by an additional treatment with active insecticidal, fungicidal or bactericidal compounds.

Preferred times for the application of compounds of the general formula (I) to be used according to the invention or salts thereof for enhancing resistance to abiotic stress are treatments of the soil, stems and/or leaves with the approved application rates.

The inventive active ingredients of the general formula (I) or salts thereof may generally additionally be present in their commercial formulations, and in the use forms prepared from these formulations, in mixtures with other active ingredients, such as insecticides, attractants, sterilants, acaricides, nematicides, fungicides, bactericides, growth regulators, substances which influence plant maturity, safeners or herbicides.

The invention is to be illustrated by the biological examples which follow, but without restricting it thereto.

### Biological Examples

In Vivo Analyses—Part A:

Seeds of monocotyledonous and dicotyledonous crop plants were sown in sandy loam in wood-fiber or plastic pots, covered with soil or sand and cultivated in a greenhouse under good growth conditions. The trial plants were treated at the early leaf stage (BBCH10-BBCH13). To assure uniform water supply before commencement of stress, the potted plants were supplied with water by dam irrigation prior to substance application.

The inventive compounds formulated in the form of wettable powders (WP) were sprayed onto the green parts of the plants as an aqueous suspension at an equivalent water application rate of 600 l/ha with addition of 0.2% wetting agent (e.g. agrotin). Substance application was followed immediately by stress treatment of the plants. For this purpose, the wood-fiber pots were transferred in plastic inserts in order to prevent them from subsequently drying out too quickly.

Drought stress was induced by gradual drying out under the following conditions:

“Day”: 14 hours with illumination at ˜26-30° C.

“Night”: 10 hours without illumination at ˜18-20° C.

The duration of the respective stress phases was guided mainly by the condition of the stressed control plants. It was ended (by re-irrigating and transfer to a greenhouse with good growth conditions) as soon as irreversible damage was observed on the stressed control plants.

The end of the stress phase was followed by an about 4-7-day recovery phase, during which the plants were once again kept under good growth conditions in a greenhouse. The duration of the recovery phase was guided mainly by when the trial plants had attained a state which enabled visual scoring of potential effects, and is therefore variable.

Once this juncture was reached, the intensities of damage were scored visually in comparison to untreated, unstressed controls of the same age. The damage intensity was at first assessed in per cent. These values were then used to calculate the efficacy of the test compounds by the following formula:

$$EF = \frac{( {SI}_{s} - {SI}_{t} ) \times 100}{{DI}_{s}}$$

EF: Efficacy

DIs: Damage intensity of the stressed control plants

DIt: Damage intensity of the stressed plants treated with test compound

The values reported in tables A-1 to A-3 below are mean values from at least one trial with at least two repeats, showing the effects of selected compounds of the general formula (I) under drought stress on various crop plants.

TABLE A-1

| | | | | EF [%] |
|---|---|---|---|---|
| No. | Substance | Dosage | Unit | (BRSNS) |
| 1 | A1-21 | 25 | g/ha | >5% |
| 2 | A1-37 | 2.5 | g/ha | >5% |
| 3 | A1-40 | 2.5 | g/ha | >5% |
| 4 | A1-147 | 25 | g/ha | >5% |
| 5 | A7-1 | 25 | g/ha | >5% |
| 6 | A7-11 | 25 | g/ha | >5% |
| 7 | A7-122 | 25 | g/ha | >5% |
| 8 | A7-639 | 25 | g/ha | >5% |
| 9 | A8-1 | 2.5 | g/ha | >5% |
| 10 | A8-21 | 25 | g/ha | >5% |
| 11 | A9-3 | 250 | g/ha | >5% |
| 12 | A9-614 | 250 | g/ha | >5% |
| 13 | A10-7 | 250 | g/ha | >5% |
| 14 | A10-61 | 25 | g/ha | >5% |
| 15 | A12-5 | 25 | g/ha | >5% |
| 16 | A12-37 | 25 | g/ha | >5% |
| 17 | A12-44 | 25 | g/ha | >5% |
| 18 | A12-86 | 25 | g/ha | >5% |
| 19 | A13-21 | 25 | g/ha | >5% |
| 20 | A28-35 | 250 | g/ha | >5% |
| 21 | A33-122 | 250 | g/ha | >5% |
| 22 | B1-122 | 25 | g/ha | >5% |
| 23 | B1-144 | 25 | g/ha | >5% |
| 24 | B3-21 | 250 | g/ha | >5% |
| 25 | C1-3 | 250 | g/ha | >5% |
| 26 | C1-61 | 250 | g/ha | >5% |
| 27 | C1-62 | 2.5 | g/ha | >5% |
| 28 | C1-111 | 2.5 | g/ha | >5% |
| 29 | C2-36 | 25 | g/ha | >5% |
| 30 | D1-86 | 25 | g/ha | >5% |

TABLE A-2

| | | | | EF [%] |
|---|---|---|---|---|
| No. | Substance | Dosage | Unit | (ZEAMX) |
| 1 | A1-7 | 250 | g/ha | >5% |
| 2 | A1-40 | 250 | g/ha | >5% |
| 3 | A1-44 | 250 | g/ha | >5% |
| 4 | A1-147 | 250 | g/ha | >5% |
| 5 | A1-612 | 250 | g/ha | >5% |
| 6 | A1-613 | 250 | g/ha | >5% |
| 7 | A7-639 | 250 | g/ha | >5% |
| 8 | A8-21 | 250 | g/ha | >5% |
| 9 | A9-3 | 250 | g/ha | >5% |
| 10 | A9-144 | 25 | g/ha | >5% |
| 11 | A9-615 | 250 | g/ha | >5% |
| 12 | A9-639 | 250 | g/ha | >5% |
| 13 | A9-663 | 250 | g/ha | >5% |
| 14 | A10-37 | 250 | g/ha | >5% |
| 15 | A12-44 | 250 | g/ha | >5% |
| 16 | A13-21 | 250 | g/ha | >5% |
| 17 | A14-36 | 250 | g/ha | >5% |
| 18 | A28-35 | 250 | g/ha | >5% |
| 19 | A32-1 | 250 | g/ha | >5% |
| 20 | A33-122 | 25 | g/ha | >5% |
| 21 | A34-122 | 25 | g/ha | >5% |
| 22 | B1-1 | 25 | g/ha | >5% |
| 23 | B1-122 | 250 | g/ha | >5% |
| 24 | B3-21 | 25 | g/ha | >5% |
| 25 | C1-3 | 25 | g/ha | >5% |
| 26 | C1-111 | 25 | g/ha | >5% |
| 27 | D1-86 | 250 | g/ha | >5% |

TABLE A-3

| | | | | EF [%] |
|---|---|---|---|---|
| No. | Substance | Dosage | Unit | (TRZAS) |
| 1 | A1-37 | 250 | g/ha | >5% |
| 1 | A1-40 | 250 | g/ha | >5% |
| 2 | A8-21 | 250 | g/ha | >5% |
| 3 | A10-30 | 250 | g/ha | >5% |
| 4 | A10-144 | 250 | g/ha | >5% |
| 5 | A12-37 | 25 | g/ha | >5% |
| 6 | A12-44 | 250 | g/ha | >5% |
| 7 | A13-21 | 250 | g/ha | >5% |
| 8 | A13-144 | 25 | g/ha | >5% |
| 9 | B1-144 | 25 | g/ha | >5% |
| 10 | C1-111 | 25 | g/ha | >5% |

In Vivo-Analysen—Teil B

Seeds of monocotyledonous and dicotyledonous crop plants were sown in sandy loam in plastic pots, covered with soil or sand and cultivated in a greenhouse under good growth conditions. The test plants are treated at the early leaf stage (BBCH10-BBCH13). To assure uniform water supply before commencement of stress, the potted plants were supplied with water by dam irrigation prior to substance application.

The inventive compounds were first formulated as wettable powders (WP) or dissolved in a solvent mixture. The further dilution was effected with water supplemented with 0.2% wetting agent (e.g. agrotin). The finished spray liquor was sprayed onto the green parts of the plant at an equivalent water application rate of 600 l/ha. Substance application was followed immediately by stress treatment of the plants.

Drought stress was induced by gradual drying out under the following conditions:

“Day”: 14 hours with illumination at ˜26-30° C.

“Night”: 10 hours without illumination at ˜18-20° C.

The duration of the respective stress phases was guided mainly by the condition of the stressed control plants. It was ended (by re-irrigating and transfer to a greenhouse with good growth conditions) as soon as irreversible damage was observed on the stressed control plants.

The end of the stress phase was followed by an about 4-7-day recovery phase, during which the plants were once again kept under good growth conditions in a greenhouse. The duration of the recovery phase was guided mainly by when the trial plants had attained a state which enabled visual scoring of potential effects, and was therefore variable.

Once this juncture had been reached, the appearance of the plants treated with test substances was recorded in comparison to the stressed control plants by the following categories:
0 no positive effect
+ slight positive effect
++ clear positive effect
+++ strong positive effect

In order to rule out any influence on the effects observed by any fungicidal or insecticidal action of the test compounds, it was additionally ensured that the tests proceeded without fungal infection or insect infestation.

The values reported in tables B-1 and B-2 below are mean values of the results from at least three repeats.

Effects of selected compounds of the general formula (I) under drought stress according to the following tables B-1 and B-2:

TABLE B-1

| | | | | Effect |
|---|---|---|---|---|
| No. | Substance | Dosage | Unit | (BRSNS) |
| 1 | A1-17 | 25 | g/ha | ++ |
| 2 | A1-27 | 250 | g/ha | ++ |
| 3 | A1-62 | 25 | g/ha | ++ |
| 4 | A1-63 | 250 | g/ha | + |
| 5 | A1-81 | 25 | g/ha | + |
| 6 | A1-603 | 250 | g/ha | ++ |
| 7 | A1-612 | 25 | g/ha | + |
| 8 | A1-679 | 25 | g/ha | + |
| 9 | A5-40 | 250 | g/ha | + |
| 10 | A8-15 | 250 | g/ha | + |
| 11 | A10-2 | 25 | g/ha | ++ |
| 12 | A10-17 | 250 | g/ha | + |
| 13 | A10-40 | 250 | g/ha | ++ |
| 14 | A10-94 | 250 | g/ha | + |
| 15 | A10-679 | 250 | g/ha | ++ |
| 16 | A12-36 | 100 | g/ha | + |
| 17 | A12-94 | 25 | g/ha | + |
| 18 | A14-91 | 250 | g/ha | + |
| 19 | A14-99 | 250 | g/ha | + |
| 20 | A14-138 | 25 | g/ha | + |
| 21 | A14-608 | 25 | g/ha | + |
| 22 | A14-680 | 250 | g/ha | ++ |
| 23 | A36-1 | 250 | g/ha | + |
| 24 | A36-5 | 250 | g/ha | ++ |
| 25 | A36-27 | 250 | g/ha | + |
| 26 | A36-61 | 250 | g/ha | + |
| 27 | A36-679 | 250 | g/ha | + |
| 28 | A48-1 | 25 | g/ha | +++ |
| 29 | A48-61 | 25 | g/ha | + |
| 30 | A48-36 | 25 | g/ha | ++ |
| 31 | A50-36 | 25 | g/ha | + |
| 32 | A50-40 | 25 | g/ha | ++ |
| 33 | A50-61 | 25 | g/ha | + |
| 34 | A51-36 | 250 | g/ha | + |
| 35 | A51-61 | 25 | g/ha | ++ |
| 36 | A52-36 | 25 | g/ha | +++ |
| 37 | B1-5 | 250 | g/ha | +++ |
| 38 | B1-7 | 250 | g/ha | +++ |
| 39 | B1-10 | 250 | g/ha | +++ |
| 40 | B1-15 | 250 | g/ha | +++ |
| 41 | B1-17 | 250 | g/ha | ++ |
| 42 | B1-40 | 250 | g/ha | + |
| 43 | B1-61 | 250 | g/ha | ++ |
| 44 | B1-62 | 250 | g/ha | ++ |
| 45 | B1-63 | 250 | g/ha | +++ |
| 46 | B1-679 | 250 | g/ha | ++ |
| 47 | B5-21 | 250 | g/ha | + |
| 48 | B5-61 | 25 | g/ha | + |
| 49 | B6-94 | 250 | g/ha | + |
| 50 | B17-61 | 250 | g/ha | + |
| 51 | E3-1 | 25 | g/ha | + |
| 52 | G1-61 | 100 | g/ha | + |

TABLE B-2

| | | | | Effect |
|---|---|---|---|---|
| No. | Substance | Dosage | Unit | (TRZAS) |
| 1 | A1-40 | 250 | g/ha | ++ |
| 2 | A1-81 | 250 | g/ha | + |
| 3 | A1-94 | 250 | g/ha | + |
| 4 | A1-159 | 250 | g/ha | + |
| 5 | A1-612 | 25 | g/ha | + |
| 6 | A1-679 | 250 | g/ha | + |
| 7 | A2-4 | 250 | g/ha | + |
| 8 | A5-40 | 250 | g/ha | + |
| 9 | A5-61 | 250 | g/ha | + |
| 10 | A8-15 | 25 | g/ha | ++ |
| 11 | A8-36 | 25 | g/ha | ++ |
| 12 | A8-94 | 25 | g/ha | + |
| 13 | A10-2 | 250 | g/ha | + |
| 14 | A10-81 | 250 | g/ha | + |
| 15 | A12-94 | 250 | g/ha | + |
| 16 | A14-5 | 25 | g/ha | + |
| 17 | A14-91 | 25 | g/ha | + |
| 18 | A14-99 | 25 | g/ha | + |
| 19 | A14-138 | 25 | g/ha | + |
| 20 | A14-608 | 25 | g/ha | + |
| 21 | A36-40 | 250 | g/ha | + |
| 22 | A36-5 | 250 | g/ha | + |
| 23 | A36-7 | 250 | g/ha | + |
| 24 | A36-10 | 250 | g/ha | + |
| 25 | A36-61 | 250 | g/ha | + |
| 26 | A36-63 | 250 | g/ha | + |
| 27 | A36-81 | 250 | g/ha | + |
| 28 | A36-679 | 250 | g/ha | + |
| 29 | A47-21 | 250 | g/ha | + |
| 30 | A48-1 | 250 | g/ha | + |
| 31 | A50-40 | 250 | g/ha | + |
| 32 | A51-1 | 250 | g/ha | + |
| 33 | A51-36 | 25 | g/ha | + |
| 34 | A51-61 | 25 | g/ha | + |
| 35 | A52-36 | 250 | g/ha | + |
| 36 | B1-7 | 250 | g/ha | + |
| 37 | B1-15 | 250 | g/ha | + |
| 38 | B1-17 | 250 | g/ha | + |
| 39 | B1-62 | 250 | g/ha | + |
| 40 | B1-94 | 250 | g/ha | + |
| 41 | B2-21 | 250 | g/ha | + |
| 42 | B5-1 | 250 | g/ha | + |
| 43 | B5-61 | 25 | g/ha | + |
| 44 | B5-94 | 25 | g/ha | + |
| 45 | B6-1 | 25 | g/ha | ++ |
| 46 | B6-21 | 250 | g/ha | + |
| 47 | B6-61 | 250 | g/ha | + |
| 48 | B6-94 | 250 | g/ha | + |
| 49 | B17-40 | 250 | g/ha | + |
| 50 | B18-1 | 25 | g/ha | ++ |
| 51 | B18-21 | 25 | g/ha | + |
| 52 | E1-21 | 25 | g/ha | + |
| 53 | E1-94 | 250 | g/ha | + |
| 54 | E3-1 | 25 | g/ha | + |
| 55 | E3-61 | 250 | g/ha | + |
| 56 | G1-61 | 100 | g/ha | + |

In the above tables:

BRSNS=Brassica napus

ZEAMX=Zea mays

TRZAS=Triticum aestivum

1. A substituted aryl- or heteroarylcarbonyl hydrazide of the formula (I) or salt thereof 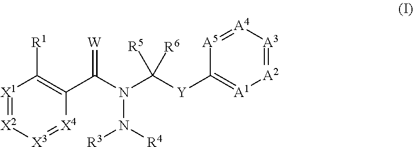
where
R¹, R² and R⁷ are independently hydrogen, halogen, cyano, nitro, NR²¹R²², OR²³, S(O)ₙR²⁴, thiocyanato, isothiocyanato, (C₁-C₈)-alkyl, (C₂-C₈)-alkenyl, (C₂-C₈)-alkynyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, pentafluorothio, (C₁-C₈)-alkoxy-(C₁-C₈)-haloalkyl, (C₁-C₈)-haloalkoxy-(C₁-C₈)-haloalkyl, (C₁-C₈)-haloalkoxy-(C₁-C₈)-alkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkylcarbonyl-(C₁-C₈)-alkyl, COOR²³, CONR²¹R²², COR²³, —C═NOR²³, R²³OOC-(C₁-C₈)-alkyl, aryl-(C₁-C₈)-alkynyl, heteroaryl-(C₁-C₈)-alkynyl, heterocyclyl-(C₁-C₈)-alkynyl, tris[(C₁-C₈)-alkyl]silyl-(C₂-C₈)-alkynyl, bis[(C₁-C₈)-alkyl](aryl)silyl-(C₂-C₈)-alkynyl, bisaryl[(C₁-C₈)-alkyl]silyl-(C₂-C₈)-alkynyl, (C₃-C₈)-cycloalkyl-(C₂-C₈)-alkynyl, aryl-(C₂-C₈)-alkenyl, heteroaryl-(C₂-C₈)-alkenyl, heterocyclyl-(C₂-C₈)-alkenyl, (C₃-C₈)-cycloalkyl-(C₂-C₈)-alkenyl, (C₁-C₈)-alkoxy-(C₁-C₈)-alkoxy-(C₁-C₈)-alkyl, (C₁-C₈)- alkylaminosulfonylamino, (C₃-C₈)-cycloalkylaminosulfonylamino, diazo, aryldiazo, tris[(C₁-C₈)-alkyl]silyl, bis[(C₁-C₈)-alkyl](aryl)silyl,
X¹, X², X³ and X⁴ are the same or different and are independently N (nitrogen) or the C—R² moiety, but there are never more than two adjacent nitrogen atoms, and where R² in each C—R² moiety is the same or different as defined above, and where R¹ and R² are not both hydrogen when X¹, X², X³ and X⁴ are C—R²,
W is O (oxygen) or S (sulfur),
A¹, A², A³, A⁴ and A⁵ are the same or different and are each independently N (nitrogen) or the C—R⁷ moiety, but there are never more than two adjacent nitrogen atoms, and where R⁷ in each C—R⁷ moiety is the same or different as defined above,
R³ is (C₁-C₈)-alkyl, cyano-(C₁-C₈)-alkyl, (C₂-C₈)-alkenyl, (C₂-C₈)-alkynyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₃-C₈)-cycloalkyl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio-(C₁-C₈)-alkyl, (C₁-C₈)-haloalkoxy- (C₁-C₈)-alkyl, (C₁-C₈)-haloalkylthio-(C₁-C₈)-alkyl, R²¹ R²²N—(C₁-C₈)-alkyl, (C₁-C₈)-alkoxy-(C₁-C₈)-alkoxy-(C₁-C₈)-alkyl,
R⁴ is hydrogen, (C₁-C₈)-alkyl, (C₂-C₈)-alkenyl, (C₂-C₈)-alkynyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, aryl, aryl-(C₁-C₈)-alkyl, heteroaryl, heteroaryl-(C₁-C₈)-alkyl, (C₃-C₈)-cycloalkyl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio-(C₁-C₈)-alkyl, (C₁-C₈)-haloalkoxy-(C₁-C₈)-alkyl, (C₁-C₈)-haloalkylthio-(C₁-C₅)-alkyl, (C₁-C₈)-alkylamino-(C₁-C₈)-alkyl, bis[(C₁-C₈)-alkyl]amino-(C₁-C₈)-alkyl, (C₃-C₈)-cycloalkylamino-(C₁-C₈)-alkyl, (C₁-C₈)-alkoxy-(C₁-C₈)-alkoxy-(C₁-C₈)-alkyl, (C₁-C₈)-alkoxycarbonyl, (C₂-C₈)-alkenyloxycarbonyl, (C₂-C₈)-alkynyloxycarbonyl, aryl-(C₁-C₈)-alkoxycarbonyl, heteroaryl-(C₁-C₈)-alkoxycarbonyl, (C₃-C₈)-cycloalkyl-(C₁-C₈)-alkoxycarbonyl, CONR²¹R²², SO₂R²⁴, hydroxycarbonyl-(C₁-C₈)-alkyl, (C₁-C8)-alkoxycarbonyl-(C₁-C₈)-alkyl, (C₂-C₈)-alkenyloxycarbonyl-(C₁-C₈)-alkyl, (C₂-C₈)-alkynyloxycarbonyl-(C₁-C₈)-alkyl, aryl-(C₁-C₈)-alkoxycarbonyl-(C₁-C₈)-alkyl, heteroaryl-(C₁-C₈)-alkoxycarbonyl-(C₁-C₈)-alkyl, heterocyclyl-(C₁-C₈)-alkoxycarbonyl-(C₁-C₈)-alkyl, (C₁-C₈)-alkylcarbonyl-(C₁-C₈)-alkyl, (C₁-C₈)-alkylsulfonyl-(C₁-C₈)-alkyl, (C₁-C₈)-alkylsulfynyl-(C₁-C₈)-alkyl,
R⁵ and R⁶ are independently hydrogen, (C₁-C₈)-alkyl, (C₂-C₈)-alkenyl, (C₂-C₈)-alkynyl, (C₁-C₁₀)-haloalkyl, (C₂-C₈)-haloalkenyl, (C₂-C₈)-haloalkynyl, (C₃-C₈)-cycloalkyl, (C₃-C₁₀)-halocycloalkyl, (C₄-C₁₀)-cycloalkenyl, (C₄-C₁₀)-halocycloalkenyl, aryl, aryl-(C₁-C₈)-alkyl, heteroaryl, heteroaryl-(C₁-C₈)-alkyl, (C₃-C₈)-cycloalkyl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio-(C₁-C₈)-alkyl, (C₁-C₈)-haloalkoxy-(C₁-C₈)-alkyl, (C₁-C₈)-haloalkylthio-(C₁-C₈)-alkyl, COOR²³, CONR²¹R²², hydroxycarbonyl-(C₁-C₈)-alkyl, (C₁-C₈)-alkoxycarbonyl-(C₁-C₈)-alkyl, (C₂-C₈)-alkenyloxycarbonyl-(C₁-C₈)-alkyl, (C₂-C₈)-alkynyloxycarbonyl-(C₁-C₈)-alkyl, aryl-(C₁-C₈)-alkoxycarbonyl-(C₁-C₈)-alkyl, heteroaryl-(C₁-C₈)-alkoxycarbonyl-(C₁-C₈)-alkyl, heterocyclyl-(C₁-C₈)-alkoxycarbonyl-(C₁-C₈)-alkyl,
R⁵ and R⁶ together with the atom to which they are bonded form a fully saturated or partly saturated 5-7-membered ring optionally interrupted by heteroatoms and optionally having further substitution,
R¹ and X¹, when X¹ is a C—R² group, together with the atoms to which they are bonded form a fully saturated, partly saturated or fully unsaturated 5-7-membered ring optionally interrupted by heteroatoms and optionally having further substitution,
X¹ and X², when each is a C—R² group, together with the atoms to which they are bonded form a fully saturated, partly saturated or fully unsaturated 5-7-membered ring optionally interrupted by heteroatoms and optionally having further substitution,
A¹ and A², when each is a C—R⁷ group, together with the atoms to which they are bonded form a fully saturated, partly saturated or fully unsaturated 5-7-membered ring optionally interrupted by heteroatoms and optionally having further substitution,
A² and A³, when each is a C—R⁷ group, together with the atoms to which they are bonded form a fully saturated, partly saturated or fully unsaturated 5-7-membered ring optionally interrupted by heteroatoms and optionally having further substitution,
A³ and A⁴, when each is a C—R⁷ group, together with the atoms to which they are bonded form a fully saturated, partly saturated or fully unsaturated 5-7-membered ring optionally interrupted by heteroatoms and optionally having further substitution,
Y is a bond or the Y-1 to Y-7 moieties 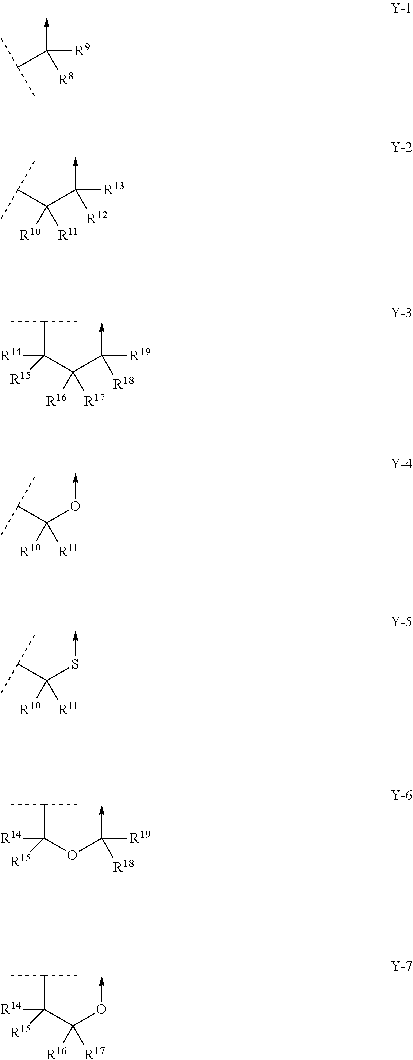
where R⁸, R⁹, R¹⁰, R″, R¹², R¹³, R¹⁴, R¹⁵, R¹⁶, R¹⁷, R¹⁸ and R¹⁹ are each as per the definition below and where the arrow represents a bond to the 6-membered ring with the A¹, A², A³, A⁴ and A⁵ moieties,
R⁸, R⁹, R¹⁰, R¹¹, R¹², R¹³, R¹⁴, R¹⁵, R¹⁶, R¹⁷, R¹⁸ and R¹⁹ are independently hydrogen, (C₁-C₈)-alkyl, (C₂-C₈)-alkenyl, (C₂-C₈)-alkynyl, (C₁-C₁₀)-haloalkyl, (C₂-C₈)-haloalkenyl, (C₂-C₈)-haloalkynyl, (C₃-C₁₀)-cycloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio-(C₁-C₈)-alkyl, (C₁-C₈)-haloalkoxy-(C₁-C₈)-alkyl, (C₁-C₈)-haloalkylthio-(C₁-C₈)-alkyl, COOR²³,
n is 0, 1 or 2,
R²¹ and R²² are the same or different and are independently hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, COR²³, SO₂R²⁴, (C₁-C₈)-alkyl-HNO₂S—, (C₃-C₈)-cycloalkyl-HNO₂S—, heterocyclyl, (C₁-C₈)-alkoxycarbonyl-(C₁-C₈)-alkyl, (C₁-C₈)-alkoxycarbonyl, aryl-(C₁-C₈)-alkoxycarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, heterocyclyl-(C₁-C₈)-alkyl,
R²³ is hydrogen, (C₁-C₈)-alkyl, (C₂-C₈)-alkenyl, (C₂-C₈)-alkynyl, (C₁-C₈-cyanoalkyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈-cycloalkyl-(C₁-C₈)-alkyl, (C₄-C₁₀)-cycloalkenyl-(C₁-C₈)-alkyl, (C₁-C₈)-alkoxycarbonyl-(C₁-C₈)-alkyl, (C₂-C₈)-alkenyloxycarbonyl-(C₁-C₈)-alkyl, aryl-(C₁-C₈)-alkoxycarbonyl-(C₁-C₈)-alkyl, hydroxycarbonyl-(C₁-C₈)-alkyl, heterocyclyl, heterocyclyl-(C₁-C₈)-alkyl and
R²⁴ is hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₈)-cycloalkyl-(C₁-C₈)-alkyl, (C₄-C₁₀)-cycloalkenyl-(C₁-C₈)-alkyl, NR²¹R²².

2. A substituted aryl- or heteroarylcarbonyl hydrazide or salt as claimed in claim 1, where
R¹, R² and R⁷ are independently hydrogen, halogen, cyano, nitro, NR²¹R²², OR²³, S(O)ₙR²⁴, thiocyanato, isothiocyanato, (C₁-C₇)-alkyl, (C₂-C₇)-alkenyl, (C₂-C₇)-alkynyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, pentafluorothio, (C₁-C₇)-alkoxy-(C₁-C₇)-haloalkyl, (C₁-C₇)-haloalkoxy-(C₁-C₇)-haloalkyl, (C₁-C₇)-haloalkoxy-(C₁-C₇)-alkyl, aryl, aryl-(C₁-C₇)-alkyl, heteroaryl, heteroaryl-(C₁-C₇)-alkyl, (C₃-C₇)-cycloalkyl-(C₁-C₇)-alkyl, (C₄-C₁₀)-cycloalkenyl-(C₁-C₇)-alkyl, heterocyclyl, heterocyclyl-(C₁-C₇)-alkyl, (C₁-C₇)-alkoxy-(C₁-C₇)-alkyl, (C₁-C₇)-alkylthio-(C₁-C₇)-alkyl, (C₁-C₇)-haloalkylthio-(C₁-C₇)-alkyl, (C₁-C₇)-alkylcarbonyl-(C₁-C₇)-alkyl, COOR²³, CONR²¹R²², COR²³, —C═NOR²³, R²³OOC-(C₁-C₇)-alkyl, aryl-(C₁-C₇)-alkynyl, heteroaryl-(C₁-C₇)-alkynyl, heterocyclyl-(C₁-C₇)-alkynyl, tris[(C₁-C₇)-alkyl]silyl-(C₂-C₇)-alkynyl, bis[(C₁-C₇)-alkyl](aryl)silyl-(C₂-C₇)-alkynyl, bisaryl[(C₁-C₇)-alkyl]silyl-(C₂-C₇)-alkynyl, (C₃-C₇)-cycloalkyl-(C₂-C₇)-alkynyl, aryl-(C₂-C₇)-alkenyl, heteroaryl-(C₂-C₇)-alkenyl, heterocyclyl-(C₂-C₇)-alkenyl, (C₃-C₇)-cycloalkyl-(C₂-C₇)-alkenyl, (C₁-C₇)-alkoxy-(C₁-C₇)-alkoxy-(C₁-C₇)-alkyl, (C₁-C₇)- alkylaminosulfonylamino, (C₃-C₇)-cycloalkylaminosulfonylamino, diazo, aryldiazo, tris[(C₁-C₇)-alkyl]silyl, bis[(C₁-C₇)-alkyl](aryl)silyl,
X¹, X², X³ and X⁴ are the same or different and are independently N (nitrogen) or the C—R² moiety, but there are never more than two adjacent nitrogen atoms, and where R² in each C—R² moiety is the same or different as defined above, and where R¹ and R² are not both hydrogen when X¹, X², X³ and X⁴ are C—R²,
W is O (oxygen) or S (sulfur),
A¹, A², A³, A⁴ and A⁵ are the same or different and are each independently N (nitrogen) or the C—R⁷ moiety, but there are never more than two adjacent nitrogen atoms, and where R⁷ in each C—R⁷ moiety is the same or different as defined above,
R³ is (C₁-C₇)-alkyl, cyano-(C₁-C₇)-alkyl, (C₂-C₇)-alkenyl, (C₂-C₇)-alkynyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₃-C₇)-cycloalkyl-(C₁-C₇)-alkyl, heterocyclyl, heterocyclyl-(C₁-C₇)-alkyl, (C₁-C₇)-al koxy-(C₁-C₇)-alkyl, (C₁-C₇)-alkylthio-(C₁-C₇)-alkyl, (C₁-C₇)-haloalkoxy- (C₁-C₇)-alkyl, (C₁-C₇)-haloalkylthio-(C₁-C₇)-alkyl, R²¹R²²N—(C₁-C₇)-alkyl, (C₁-C₇)-alkoxy-(C₁-C₇)-al koxy-(C₁-C₇)-alkyl,
R⁴ is hydrogen, (C₁-C₇)-alkyl, (C₂-C₇)-alkenyl, (C₂-C₇)-alkynyl, (C₁-C₁₀)-haloalkyl, (C₂-C₇)-haloalkenyl, (C₂-C₇)-haloalkynyl, (C₃-C₁₀)-cycloalkyl, (C₃-Cio)-halocycloalkyl, (C₄-C₁₀)-cycloalkenyl, (C₄-C₁₀)-halocycloalkenyl, aryl, aryl-(C₁-C₇)-alkyl, heteroaryl, heteroaryl-(C₁-C₇)-alkyl, (C₃-C₇)-cycloalkyl-(C₁-C₇)-alkyl, heterocyclyl, heterocyclyl-(C₁-C₇)-alkyl, (C₁-C₇)-alkoxy-(C₁-C₇)-alkyl, (C₁-C₇)-alkylthio-(C₁-C₇)-alkyl, (C₁-C₇)-haloalkoxy-(C₁-C₇)-alkyl, (C₁-C₇)-haloalkylthio-(C₁-C₇)-alkyl, (C₁-C₇)-alkylamino-(C₁-C₇)-alkyl, bis[(C₁-C₇)-alkyl]amino-(C₁-C₇)-alkyl, (C₃-C₇)-cycloalkylamino-(C₁-C₇)-alkyl, (C₁-C₇)-alkoxy-(C₁-C₇)-alkoxy-(C₁-C₇)-alkyl, (C₁-C₇)-alkoxycarbonyl, (C₂-C₇)-alkenyloxycarbonyl, (C₂-C₇)-alkynyloxycarbonyl, aryl-(C₁-C₇)-alkoxycarbonyl, heteroaryl-(C₁-C₇)-alkoxycarbonyl, (C₃-C₇)-cycloalkyl-(C₁-C₇)-alkoxycarbonyl, CONR²¹R²², SO₂R²⁴, hydroxycarbonyl-(C₁-C₇)-alkyl, (C₁-C₇)-alkoxycarbonyl-(C₁-C₇)-alkyl, (C₂-C₇)-alkenyloxycarbonyl-(C₁-C₇)-alkyl, (C₂-C₇)-alkynyloxycarbonyl-(C₁-C₇)-alkyl, aryl-(C₁-C₇)-alkoxycarbonyl-(C₁-C₇)-alkyl, heteroaryl-(C₁-C₇)-alkoxycarbonyl-(C₁-C₇)-alkyl, heterocyclyl-(C₁-C₇)-alkoxycarbonyl-(C₁-C₇)-alkyl, (C₁-C₇)-alkylcarbonyl-(C₁-C₇)-alkyl, (C₁-C₇)-alkylsulfonyl-(C₁-C₇)-alkyl, (C₁-C₇)-alkylsulfynyl-(C₁-C₇)-alkyl,
R⁵ and R⁶ are independently hydrogen, (C₁-C₇)-alkyl, (C₂-C₇)-alkenyl, (C₂-C₇)-alkynyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, aryl, aryl-(C₁-C₇)-alkyl, heteroaryl, heteroaryl-(C₁-C₇)-alkyl, (C₃-C₇)-cycloalkyl-(C₁-C₇)-alkyl, heterocyclyl, heterocyclyl-(C₁-C₇)-alkyl, (C₁-C₇)-alkoxy-(C₁-C₇)-alkyl, (C₁-C₇)-alkylthio-(C₁-C₇)-alkyl, (C₁-C₇)-haloalkoxy-(C₁-C₇)-alkyl, (C₁-C₇)-haloalkylthio-(C₁-C₇)-alkyl, COOR²³, CONR²¹R²², hydroxycarbonyl-(C₁-C₇)-alkyl, (C₁-C₇)-alkoxycarbonyl-(C₁-C₇)-alkyl, (C₂-C₇)-alkenyloxycarbonyl-(C₁-C₇)-alkyl, (C₂-C₇)-alkynyloxycarbonyl-(C₁-C₇)-alkyl, aryl-(C₁-C₇)-alkoxycarbonyl-(C₁-C₇)-alkyl, heteroaryl-(C₁-C₇)-alkoxycarbonyl-(C₁-C₇)-alkyl, heterocyclyl-(C₁-C₇)-alkoxycarbonyl-(C₁-C₇)-alkyl,
R⁵ and R⁶ together with the atom to which they are bonded form a fully saturated or partly saturated 5-7-membered ring optionally interrupted by heteroatoms and optionally having further substitution,
R¹ and X¹, when X¹ is a C—R² group, together with the atoms to which they are bonded form a fully saturated, partly saturated or fully unsaturated 5-7-membered ring optionally interrupted by heteroatoms and optionally having further substitution,
X¹ and X², when each is a C—R² group, together with the atoms to which they are bonded form a fully saturated, partly saturated or fully unsaturated 5-7-membered ring optionally interrupted by heteroatoms and optionally having further substitution,
A¹ and A², when each is a C—R⁷ group, together with the atoms to which they are bonded form a fully saturated, partly saturated or fully unsaturated 5-7-membered ring optionally interrupted by heteroatoms and optionally having further substitution,
A² and A³, when each is a C—R⁷ group, together with the atoms to which they are bonded form a fully saturated, partly saturated or fully unsaturated 5-7-membered ring optionally interrupted by heteroatoms and optionally having further substitution,
A³ and A⁴, when each is a C—R⁷ group, together with the atoms to which they are bonded form a fully saturated, partly saturated or fully unsaturated 5-7-membered ring optionally interrupted by heteroatoms and optionally having further substitution,
Y is a bond or the Y-1 to Y-7 moieties 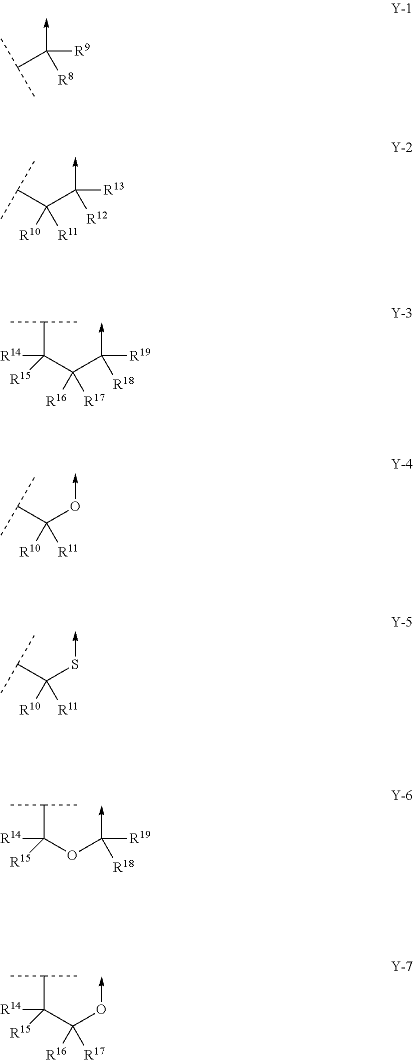
where R⁸, R⁹, R¹⁰, R¹¹, R¹², R¹³, R¹⁴, R¹⁵, R¹⁶, R¹⁷, R¹⁸ and R¹⁹ are each as per the definition below and where the arrow represents a bond to the 6-membered ring with the A¹, A², A³, A⁴ and A⁵ moieties,
R⁸, R⁹, R¹⁰, R¹¹, R¹², R¹³, R¹⁴, R¹⁵, R¹⁶, R¹⁷, R¹⁸ and R¹⁹ are independently hydrogen, (C₁-C₇)-alkyl, (C₂-C₇)-alkenyl, (C₂-C₇)-alkynyl, (C₁-C₁₀)-haloalkyl, (C₂-C₇)-haloalkenyl, (C₂-C₇)-haloalkynyl, (C₃-C₁₀)-cycloalkyl, aryl, aryl-(C₁-C₇)-alkyl, heteroaryl, heteroaryl-(C₁-C₇)-alkyl, (C₃-C₇)-cycloal kyl-(C₁-C₇)-alkyl, heterocyclyl, heterocyclyl-(C₁-C₇)-alkyl, (C₁-C₇)-alkoxy-(C₁-C₇)-alkyl, (C₁-C₇)-alkylthio-(C₁-C₇)-alkyl, (C₁-C₇)-haloalkoxy-(C₁-C₇)-alkyl, (C₁-C₇)-haloalkylthio-(C₁-C₇)-alkyl, COOR²³,
n is 0, 1 or 2,
R²¹ and R²² are the same or different and are independently hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-haloalkoxy-(C₁-C₇)-alkyl, (C₁-C₇)-alkylthio-(C₁-C₇)-alkyl, (C₁-C₇)-haloalkylthio-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, (C₃-C₇)-cycloalkyl-(C₁-C₇)-alkyl, (C₄-C₁₀)-cycloalkenyl-(C₁-C₇)-alkyl, COR²³, SO₂R²⁴, (C₁-C₇)-alkyl-HNO₂S—, (C₃-C₇)-cycloalkyl-HNO₂S—, heterocyclyl, (C₁-C₇)-alkoxycarbonyl-(C₁-C₇)-alkyl, (C₁-C₇)-alkoxycarbonyl, aryl-(C₁-C₇)-alkoxycarbonyl-(C₁-C₇)-alkyl, aryl-(C₁-C₇)-alkoxycarbonyl, heteroaryl-(C₁-C₇)-alkoxycarbonyl, (C₂-C₇)-alkenyloxycarbonyl, (C₂-C₇)-alkynyloxycarbonyl, heterocyclyl-(C₁-C₇)-alkyl,
R²³ is hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, (C₃-C₇)-cycloalkyl-(C₁-C₇)-alkyl, (C₄-C₁₀)-cycloalkenyl-(C₁-C₇)-alkyl, (C₁-C₇)-alkoxycarbonyl-(C₁-C₇)-alkyl, (C₂-C₇)-alkenyloxycarbonyl-(C₁-C₇)-alkyl, aryl-(C₁-C₇)-alkoxycarbonyl-(C₁-C₇)-alkyl, hydroxycarbonyl-(C₁-C₇)-alkyl, heterocyclyl, heterocyclyl-(C₁-C₇)-alkyl and
R²⁴ is hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, heterocyclyl-(C₁-C₇)-alkyl, (C₃-C₇)-cycloalkyl-(C₁-C₇)-alkyl, (C₄-C₁₀)-cycloalkenyl-(C₁-C₇)-alkyl, NR²¹R²².

3. A substituted aryl- or heteroarylcarbonyl hydrazide or salt as claimed in claim 1, where
R¹, R² and R⁷ are independently hydrogen, halogen, cyano, nitro, NR²¹R²², OR²³, S(O),R²⁴, thiocyanato, isothiocyanato, (C₁-C₆)-alkyl, (C₂-C₆)-alkenyl, (C₂-C₆)-alkynyl, (C₁-C₁₀)-haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, pentafluorothio, (C₁-C₆)-alkoxy-(C₁-C₆)-haloalkyl, (C₁-C₆)-haloalkoxy-(C₁-C₆)-haloalkyl, (C₁-C₆)-haloalkoxy-(C₁-C₆)-alkyl, aryl, aryl-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, heterocyclyl, heterocyclyl-(C₁-C₆)-alkyl, (C₁-C₆)-alkoxy-(C₁-C₆)-alkyl, (C₁-C₆)-alkylthio-(C₁-C₆)-alkyl, (C₁-C₆)-haloalkylthio-(C₁-C₆)-alkyl, (C₁-C₆)-alkylcarbonyl-(C₁-C₆)-alkyl, COOR²³, CONR²¹R²², COR²³, —C═NOR²³, R²³OOC-(C₁-C₆)-alkyl, aryl-(C₁-C₆)-alkynyl, heteroaryl-(C₁-C₆)-alkynyl, heterocyclyl-(C₁-C₆)-alkynyl, tris[(C₁-C₆)-alkyl]silyl-(C₂-C₆)-alkynyl, bis[(C₁-C₆)-alkyl](aryl)silyl-(C₂-C₆)-alkynyl, bisaryl[(C₁-C₆)-alkyl]silyl-(C₂-C₆)-alkynyl, (C₃-C₆)-cycloalkyl-(C₂-C₆)-alkynyl, aryl-(C₂-C₆)-alkenyl, heteroaryl-(C₂-C₆)-alkenyl, heterocyclyl-(C₂-C₆)-alkenyl, (C₃-C₆)-cycloalkyl-(C₂-C₆)-alkenyl, (C₁-C₆)-alkoxy-(C₁-C₆)-alkoxy-(C₁-C₆)-alkyl, (C₁-C₆)- alkylaminosulfonylamino, (C₃-C₆)-cycloalkylaminosulfonylamino, diazo, aryldiazo, tris[(C₁-C₆)-alkyl]silyl, bis[(C₁-C₆)-alkyl](aryl)silyl,
X¹, X², X³ and X⁴ are the same or different and are independently N (nitrogen) or the C—R² moiety, but there are never more than two adjacent nitrogen atoms, and where R² in each C—R² moiety is the same or different as defined above, and where R¹ and R² are not both hydrogen when X¹, X², X³ and X⁴ are C—R²,
W is O (oxygen) or S (sulfur),
A¹, A², A³, A⁴ and A⁵ are the same or different and are each independently N (nitrogen) or the C—R⁷ moiety, but there are never more than two adjacent nitrogen atoms, and where R⁷ in each C—R⁷ moiety is the same or different as defined above,
R³ is (C₁-C₆)-alkyl, cyano-(C₁-C₆)-alkyl, (C₂-C₆)-alkenyl, (C₂-C₆)-alkynyl, (C₁-C₆)-haloalkyl, (C₂-C₆)-haloalkenyl, (C₂-C₆)-haloalkynyl, (C₃-C₆)-cycloalkyl, (C₃-Cio)-halocycloalkyl, (C₄-C₁₀)-cycloalkenyl, (C₄-C₁₀)-halocycloalkenyl, (C₃-C₆)-cycloalkyl-(C₁-C₆)-alkyl, heterocyclyl, heterocyclyl-(C₁-C₆)-alkyl, (C₁-C₆)-alkoxy-(C₁-C₆)-alkyl, (C₁-C₆)-alkylthio-(C₁-C₆)-alkyl, (C₁-C₆)-haloalkoxy- (C₁-C₆)-alkyl, (C₁-C₆)-haloalkylthio-(C₁-C₆)-alkyl, R²¹R²²N—(C₁-C₆)-alkyl, (C₁-C₆)-alkoxy-(C₁-C₆)-alkoxy-(C₁-C₆)-alkyl,
R⁴ is hydrogen, (C₁-C₆)-alkyl, (C₂-C₆)-alkenyl, (C₂-C₆)-alkynyl, (C₁-C₁₀)-haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, aryl, aryl-(C₁-C₆)-alkyl, heteroaryl, heteroaryl-(C₁-C₆)-alkyl, (C₃-C₆)-cycloalkyl-(C₁-C₆)-alkyl, heterocyclyl, heterocyclyl-(C₁-C₆)-alkyl, (C₁-C₆)-alkoxy-(C₁-C₆)-alkyl, (C₁-C₆)-alkylthio-(C₁-C₆)-alkyl, (C₁-C₆)-haloalkoxy-(C₁-C₆)-alkyl, (C₁-C₆)-haloalkylthio-(C₁-C₆)-alkyl, (C₁-C₆)-alkylamino-(C₁-C₆)-alkyl, bis[(C₁-C₆)-alkyl]amino-(C₁-C₆)-alkyl, (C₃-C₆)-cycloalkylamino-(C₁-C₆)-alkyl, (C₁-C₆)-alkoxy-(C₁-C₆)-alkoxy-(C₁-C₆)-alkyl, (C₁-C₆)-alkoxycarbonyl, (C₂-C₆)-alkenyloxycarbonyl, (C₂-C₆)-alkynyloxycarbonyl, aryl-(C₁-C₆)-alkoxycarbonyl, heteroaryl-(C₁-C₆)-alkoxycarbonyl, (C₃-C₆)-cycloalkyl-(C₁-C₆)-alkoxycarbonyl, CONR²¹R²², SO₂R²⁴, hydroxycarbonyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, (C₂-C₆)-alkynyloxycarbonyl-(C₁-C₆)-alkyl, aryl-(C₁-C₆)-alkoxycarbonyl-(C₁-C₆)-alkyl, heteroaryl-(C₁-C₆)-alkoxycarbonyl-(C₁-C₆)-alkyl, heterocyclyl-(C₁-C₆)-alkoxycarbonyl-(C₁-C₆)-alkyl, (C₁-C₆)-alkylcarbonyl-(C₁-C₆)-alkyl, (C₁-C₆)-alkylsulfonyl-(C₁-C₆)-alkyl, (C₁-C₆)-alkylsulfynyl-(C₁-C₆)-alkyl,
R⁵ and R⁶ are independently hydrogen, (C₁-C₆)-alkyl, (C₂-C₆)-alkenyl, (C₂-C₆)-alkynyl, (C₁-C₁₀)-haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, aryl, aryl-(C₁-C₆)-alkyl, heteroaryl, heteroaryl-(C₁-C₆)-alkyl, (C₃-C₆)-cycloalkyl-(C₁-C₆)-alkyl, heterocyclyl, heterocyclyl-(C₁-C₆)-alkyl, (C₁-C₆)-alkoxy-(C₁-C₆)-alkyl, (C₁-C₆)-alkylthio-(C₁-C₆)-alkyl, (C₁-C₆)-haloalkoxy-(C₁-C₆)-alkyl, (C₁-C₆)-haloalkylthio-(C₁-C₆)-alkyl, COOR²³, CONR²¹R²², hydroxycarbonyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, (C₂-C₆)-alkynyloxycarbonyl-(C₁-C₆)-alkyl, aryl-(C₁-C₆)-alkoxycarbonyl-(C₁-C₆)-alkyl, heteroaryl-(C₁-C₆)-alkoxycarbonyl-(C₁-C₆)-alkyl, heterocyclyl-(C₁-C₆)-alkoxycarbonyl-(C₁-C₆)-alkyl,
R⁵ and R⁶ together with the atom to which they are bonded form a fully saturated or partly saturated 5-7-membered ring optionally interrupted by heteroatoms and optionally having further substitution,
R¹ and X¹, when X¹ is a C—R² group, together with the atoms to which they are bonded form a fully saturated, partly saturated or fully unsaturated 5-7-membered ring optionally interrupted by heteroatoms and optionally having further substitution,
X¹ and X², when each is a C—R² group, together with the atoms to which they are bonded form a fully saturated, partly saturated or fully unsaturated 5-7-membered ring optionally interrupted by heteroatoms and optionally having further substitution,
A¹ and A², when each is a C—R⁷ group, together with the atoms to which they are bonded form a fully saturated, partly saturated or fully unsaturated 5-7-membered ring optionally interrupted by heteroatoms and optionally having further substitution,
A² and A³, when each is a C—R⁷ group, together with the atoms to which they are bonded form a fully saturated, partly saturated or fully unsaturated 5-7-membered ring optionally interrupted by heteroatoms and optionally having further substitution,
A³ and A⁴, when each is a C—R⁷ group, together with the atoms to which they are bonded form a fully saturated, partly saturated or fully unsaturated 5-7-membered ring optionally interrupted by heteroatoms and optionally having further substitution,
Y is a bond or the Y-1 to Y-7 moieties 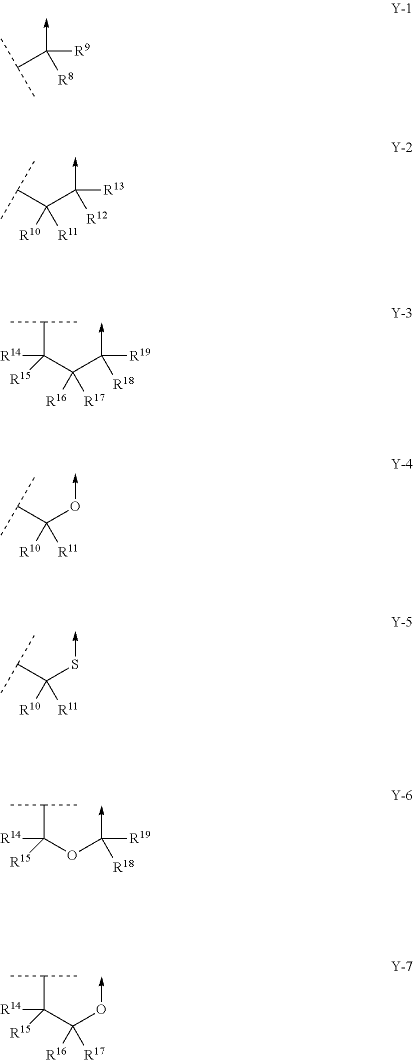
where R⁸, R⁹, R¹⁰, R¹¹, R¹², R¹³, R¹⁴, R¹⁵, R¹⁶, R¹⁷, R¹⁸ and R¹⁹ are each as per the definition below and where the arrow represents a bond to the 6-membered ring with the A¹, A², A³, A⁴ and A⁵ moieties,
R⁸, R⁹, R¹⁰, R¹¹, R¹², R¹³, R¹⁴, R¹⁵, R¹⁶, R¹⁷, R¹⁸ and R¹⁹ are independently hydrogen, (C₁-C₆)-alkyl, (C₂-C₆)-alkenyl, (C₂-C₆)-alkynyl, (C₁-C₁₀)-haloalkyl, (C₂-C₆)-haloalkenyl, (C₂-C₆)-haloalkynyl, (C₃-C₁₀)-cycloalkyl, aryl, aryl-(C₁-C₆)-alkyl, heteroaryl, heteroaryl-(C₁-C₆)-alkyl, (C₃-C₆)-cycloal kyl-(C₁-C₆)-alkyl, heterocyclyl, heterocyclyl-(C₁-C₆)-alkyl, (C₁-C₆)-alkoxy-(C₁-C₆)-alkyl, (C₁-C₆)-alkylthio-(C₁-C₆)-alkyl, (C₁-C₆)-haloalkoxy-(C₁-C₆)-alkyl, (C₁-C₆)-haloalkylthio-(C₁-C₆)-alkyl, COOR²³,
n is 0, 1 or 2,
R²¹ and R²² are the same or different and are independently hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-haloalkoxy-(C₁-C₆)-alkyl, (C₁-C₆)-alkylthio-(C₁-C₆)-alkyl, (C₁-C₆)-haloalkylthio-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, COR²³, SO₂R²⁴, (C₁-C₆)-alkyl-HNO₂S—, (C₃-C₆)-cycloalkyl-HNO₂S—, heterocyclyl, (C₁-C₆)-alkoxycarbonyl-(C₁-C₆)-alkyl, (C₁-C₆)-alkoxycarbonyl, aryl-(C₁-C₆)-alkoxycarbonyl-(C₁-C₆)-alkyl, aryl-(C₁-C₆)-alkoxycarbonyl, heteroaryl-(C₁-C₆)-alkoxycarbonyl, (C₂-C₆)-alkenyloxycarbonyl, (C₂-C₆)-alkynyloxycarbonyl, heterocyclyl-(C₁-C₆)-alkyl,
R²³ is hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, hydroxycarbonyl-(C₁-C₆)-alkyl, heterocyclyl, heterocyclyl-(C₁-C₆)-alkyl and
R²⁴ is hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, heterocyclyl-(C₁-C₆)-alkyl, (C₃-C₆)-cycloalkyl-(C₁-C₆)-alkyl, (C₄-C₁₀)-cycloalkenyl-(C₁-C₆)-alkyl, NR²¹R²².

4. A product comprising one or more compounds of the formula (I) or salts thereof as claimed in claim 1 for increasing tolerance to abiotic stress in plants.

5. A treatment of plants comprising applying a nontoxic amount, effective for enhancing the resistance of plants to abiotic stress factors, one or more of the compounds of formula (I) or salts thereof as claimed in claim 1.

6. The treatment as claimed in claim 5, wherein the abiotic stress conditions correspond to one or more conditions selected from the group of heat, drought, cold and drought stress, osmotic stress, waterlogging, elevated soil salinity, elevated exposure to minerals, ozone conditions, strong light conditions, limited availability of nitrogen nutrients, limited availability of phosphorus nutrients.

7. A product comprising one or more compounds of formula (I) or salts thereof as claimed in claim 1 adapted for use in a spray application to one or more plants and/or plant parts in combinations with one or more active ingredients selected from the group of insecticides, attractants, acaricides, fungicides, nematicides, herbicides, growth regulators, safeners, substances which influence plant maturity and bactericides.

8. A product comprising one or more compounds of formula (I) or salts thereof as claimed in claim 1 adapted for us in a spray application to plants and/or plant parts in combination with one or more fertilizers.

9. A product comprising one or more compounds of formula (I) or salts thereof as claimed in claim 1 for application to genetically modified cultivars, seed thereof, or to cultivated areas in which cultivars grow.

10. A spray solution for treatment of plants, comprising an amount, effective for enhancing the resistance of plants to abiotic stress factors, of one or more compounds of formula (I) as claimed in claim 1 or salts thereof.

11. A spray solution comprising one or more compounds of formula (I) as claimed in claim 1 or salts thereof for enhancing the resistance of one or more plants to one or more abiotic stress factors.

12. A method of increasing stress tolerance in one or more plants selected from the group of useful plants, ornamental plants, turfgrass types and trees, which comprises applying a sufficient, nontoxic amount of one or more compounds of formula (I) as claimed in claim 1 or salts thereof to an area where a corresponding effect is desired, to the plants, seed thereof or to an area in which plants grow.

13. The method as claimed in claim 12, wherein the resistance of the plants thus treated to abiotic stress is increased by at least 3% compared to untreated plants under otherwise identical physiological conditions.
